(19) United States
(12) Patent Application Publication
(10) Pub. No.: US 2025/0179526 A1
(43) Pub. Date: Jun. 5, 2025

(54) ADENOVIRUS VECTORS AND METHODS FOR USING ADENOVIRUS VECTORS

(71) Applicant: Mayo Foundation for Medical Education and Research, Rochester, MN (US)

(72) Inventor: Michael A. Barry, Rochester, MN (US)

(21) Appl. No.: 19/054,141

(22) Filed: Feb. 14, 2025

Related U.S. Application Data
(63) Continuation of application No. 17/469,569, filed on Sep. 8, 2021, now Pat. No. 12,258,572.
Application No. 17/469,569 is a continuation of application No. 16/996,740, filed on Aug. 18, 2020, now Pat. No. 11,149,286.
Application No. 17/469,569 is a continuation-in-part of application No. PCT/US2021/046333, filed on Aug. 17, 2021.
(60) Provisional application No. 63/066,740, filed on Aug. 17, 2020.
(60) Provisional application No. 63/066,740, filed on Aug. 17, 2020.

(51) Int. Cl. C12N 15/86; A61K 9/00; A61K 39/00; A61K 39/08; A61K 39/12; A61P 31/04; A61P 31/14; A61P 37/04; C07K 14/005; C12N 7/00

(52) U.S. Cl. CPC C12N 15/86; A61K 9/006; A61K 39/08; A61K 39/12; A61P 31/04; A61P 31/14; A61P 37/04; C07K 14/005; C12N 7/00; A61K 2039/5256; A61K 2039/541; C07K 2319/33; C12N 2750/14122; C12N 2750/14143; C12N 2770/20022

(57) ABSTRACT

This document provides adenovirus vectors and methods and materials related to using adenovirus vectors. For example, adenoviruses for delivering nucleic acid encoding one or more immunogens (e.g., one or more immunogens associated with a pathogen causing an infection) to cells within a mammal such that the mammal produces an effective immune response against the immunogen(s) are provided.

### CROSS-REFERENCE TO RELATED APPLICATIONS

This application is a continuation of U.S. application Ser. No. 17/469,569, filed on Sep. 8, 2021, which is a continuation of U.S. application Ser. No. 16/996,740, filed Aug. 18, 2020 (now U.S. Pat. No. 11,149,286), which claims the benefit of U.S. Provisional Application Ser. No. 63/066,740, filed on Aug. 17, 2020, and which is a continuation-in-part of International PCT Patent Application Serial No. PCT/US2021/046333, filed Aug. 17, 2021, which claims the benefit of U.S. Provisional Application Ser. No. 63/066,740, filed on Aug. 17, 2020. The disclosures of the prior applications are considered part of (and are incorporated by reference in) the disclosure of this application.

### SEQUENCE LISTING

This application contains a Sequence Listing that has been submitted electronically as an XML file named 07039-1964003_SL_ST26.xml. The XML file, created on Feb. 10, 2025, is 565,050 bytes in size. The material in the XML file is hereby incorporated by reference in its entirety.

### TECHNICAL FIELD

This document relates to adenovirus vectors and methods and materials related to using adenovirus vectors. For example, adenovirus vectors can be used to deliver one or more immunogens (e.g., one or more immunogens associated with a pathogen causing an infection) to cells within a mammal such that the mammal produces an effective immune response against the immunogen(s).

### BACKGROUND INFORMATION

An infectious disease caused by a coronavirus known as COVID-19 was first reported to the World Health Organization (WHO) Country Office in China on Dec. 31, 2019. As of Jun. 3, 2020, approximately 6,287,771 confirmed cases of COVID-19, including 379,941 deaths, have been reported to the WHO (covid19.who.int/).

### SUMMARY

This document relates to adenovirus vectors and methods and materials related to using adenovirus vectors. For example, this document provides adenovirus vectors, nucleic acid molecules encoding adenovirus vectors, cell lines containing adenovirus vectors, and methods for using adenovirus vectors to deliver nucleic acid to cells in vitro or in vivo. This document also provides methods and materials for using adenovirus vectors to induce immune responses within a mammal (e.g., a human). In some cases, adenovirus vectors (e.g., single-cycle adenovirus (SC-Ad) vectors) can be used to deliver one or more (e.g., one, two, three, four, five, six, seven, eight, nine, ten, or more) immunogens that trigger an immune response within a mammal (e.g., a human).

As demonstrated herein, SC-Ads can be engineered to express one or more immunogens as fused and secreted polypeptides that can induce an effective immune response against those immunogens. For example, administration of SC-Ads expressing C. difficile TcdA/B fusion polypeptides protected mice and Syrian Hamsters from lethal toxin challenge for extended periods (e.g., over 36 weeks) after a single immunization.

Having the ability to produce immune responses effectively against viral and/or bacterial pathogens (e.g., a coronavirus) in mammals (e.g., humans) can improve survival and minimize the impact of the infection. Adenovirus vectors encoding one or more immunogens can be used to provide a mammal with sustained, long-term immunity against an infectious pathogen (e.g., a coronavirus). For example, adenovirus vectors encoding one or more immunogens associated with COVID-19 (e.g., one or more immunogens derived from SARS-CoV-2) can be used as a robust vaccine in the COVID-19 pandemic to generate humoral immunity against both primary infections and recurrences.

In general, one aspect of this document features a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding a coronavirus immunogen. The adenovirus polypeptide can be a fiber polypeptide, a V polypeptide, a hexon polypeptide, a penton base polypeptide, or a pIIIa polypeptide. The coronavirus immunogen can include a coronavirus Spike polypeptide or an immunogenic fragment thereof. The coronavirus immunogen can consist of or can consist essentially of an amino acid sequence set forth in any one of SEQ ID NOs:1-4. The SC-Ad also can include a nucleic acid sequence encoding an adjuvant polypeptide. The adjuvant polypeptide can be a granulocyte-macrophage colony-stimulating factor (GM-CSF) polypeptide, an interleukin 4 (IL-4) polypeptide, an interleukin 21 (IL-21) polypeptide, a CD40 ligand (CD40L) polypeptide, a 4-1BB ligand (4-1BBL) polypeptide, a transforming growth factor beta (TGF-β) polypeptide, a Clostridium difficile TcdA polypeptide, a C. difficile TcdB polypeptide, or a biologically active fragment thereof. The coronavirus Spike polypeptide can be fused to the adjuvant polypeptide. The coronavirus Spike polypeptide fused to the adjuvant polypeptide can consist essentially of or can consist of an amino acid sequence set forth in SEQ ID NO:5. The SC-Ad also can include a nucleic acid sequence encoding a chaff polypeptide. The chaff polypeptide can be a fragment of an ACE2 polypeptide. The fragment of an ACE2 polypeptide can include the extracellular region of an ACE2 polypeptide and can lack a transmembrane domain. The chaff polypeptide can consist essentially of or can consist of an amino acid sequence set forth in SEQ ID NO:8 or SEQ ID NO:9. The coronavirus Spike polypeptide can be fused to the chaff polypeptide.

In another aspect, this document features a composition including a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding a coronavirus immunogen.

In another aspect, this document features methods for inducing an immune response against a coronavirus in a mammal. The methods can include, or consist essentially of, administering to a mammal i) a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding a coronavirus immunogen; or ii) a composition including a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding a coronavirus immunogen, under conditions where the SC-Ad infects a cell of the mammal, and where expression of the immunogen in the cell leads to induction of the immune response. The mammal can be a human. The coronavirus can be a betacoronavirus. The betacoronavirus can be SARS-CoV-2. The administering can include mucosal delivery of the SC-Ad.

In another aspect, this document features a SC-Ad, where the SC-Ad has a genome lacking at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding an adjuvant polypeptide. The adenovirus polypeptide can be a fiber polypeptide, a V polypeptide, a hexon polypeptide, a penton base polypeptide, or a pIIIa polypeptide. The immunogen can be a coronavirus immunogen. The coronavirus immunogen can include a coronavirus Spike polypeptide or an immunogenic fragment thereof. The coronavirus immunogen can consist of or can consist essentially of an amino acid sequence set forth in any one of SEQ ID NOs:1-4. The adjuvant polypeptide can be a GM-CSF polypeptide, an IL-4 polypeptide, an IL-21 polypeptide, a CD40L polypeptide, a 4-1BBL polypeptide, a TGF-β polypeptide, a Clostridium difficile TcdA polypeptide, a C. difficile TcdB polypeptide, or a biologically active fragment thereof. The coronavirus Spike polypeptide can be fused to the adjuvant polypeptide. The coronavirus Spike polypeptide fused to the adjuvant polypeptide can consist essentially of or can consist of an amino acid sequence set forth in SEQ ID NO:5. The SC-Ad also can include a nucleic acid sequence encoding a chaff polypeptide. The chaff polypeptide can be a fragment of an ACE2 polypeptide. The fragment of an ACE2 polypeptide can include the extracellular region of an ACE2 polypeptide and can lack a transmembrane domain. The chaff polypeptide can consist essentially of or can consist of an amino acid sequence set forth in SEQ ID NO:8 or SEQ ID NO:9.

In another aspect, this document features a composition including a SC-Ad, where the SC-Ad has a genome lacking at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding an adjuvant polypeptide.

In another aspect, this document features methods for inducing an immune response against a virus in a mammal. The methods can include, or consist essentially of, administering to a mammal i) a SC-Ad, where the SC-Ad has a genome lacking at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding an adjuvant polypeptide; or ii) a composition that includes a SC-Ad, where the SC-Ad has a genome lacking at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding an adjuvant polypeptide, under conditions where the SC-Ad infects a cell of the mammal, and where expression of the immunogen in the cell leads to induction of the immune response. The mammal can be a human. The virus can be a coronavirus, and the immunogen can be associated with the coronavirus. The coronavirus can be a betacoronavirus. The betacoronavirus can be SARS-CoV-2. The administering can include mucosal delivery of the SC-Ad.

In another aspect, this document features a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding a chaff polypeptide. The adenovirus polypeptide can be a fiber polypeptide, a V polypeptide, a hexon polypeptide, a penton base polypeptide, or a pIIIa polypeptide. The immunogen can be a coronavirus immunogen. The coronavirus immunogen can include a coronavirus Spike polypeptide or an immunogenic fragment thereof. The coronavirus immunogen can consist of or can consist essentially of an amino acid sequence set forth in any one of SEQ ID NOs:1-4. The chaff polypeptide can be a fragment of an ACE2 polypeptide. The fragment of an ACE2 polypeptide can include the extracellular region of an ACE2 polypeptide and can lack a transmembrane domain. The chaff polypeptide can consist essentially of or can consist of an amino acid sequence set forth in SEQ ID NO:8 or SEQ ID NO:9. The coronavirus Spike polypeptide can be fused to the chaff polypeptide. The SC-Ad also can include a nucleic acid sequence encoding an adjuvant polypeptide. The adjuvant polypeptide can be a GM-CSF polypeptide, an IL-4 polypeptide, an IL-21 polypeptide, a CD40L polypeptide, a 4-1BBL polypeptide, a TGF-β polypeptide, a Clostridium difficile TcdA polypeptide, a C. difficile TcdB polypeptide, or a biologically active fragment thereof.

In another aspect, this document features for a composition including a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding a chaff polypeptide.

In another aspect, this document features methods for inducing an immune response against a virus in a mammal. The methods can include, or consist essentially of, administering to a mammal i) a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding a chaff polypeptide, or ii) a composition including a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen and (b) a nucleic acid sequence encoding a chaff polypeptide, under conditions where the SC-Ad infects a cell of the mammal, and where expression of the immunogen in the cell leads to induction of the immune response. The mammal can be a human. The virus can be a coronavirus, and the immunogen can be associated with the coronavirus. The coronavirus can be a betacoronavirus. The betacoronavirus can be SARS-CoV-2. The administering can include mucosal delivery of the SC-Ad.

In another aspect, this document features for a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen, (b) a nucleic acid sequence encoding an adjuvant polypeptide, and (c) a nucleic acid sequence encoding a chaff polypeptide. The adenovirus polypeptide can be a fiber polypeptide, a V polypeptide, a hexon polypeptide, a penton base polypeptide, or a pIIIa polypeptide. The immunogen can be a coronavirus immunogen. The coronavirus immunogen can include a coronavirus Spike polypeptide or an immunogenic fragment thereof. The coronavirus immunogen can consist of or can consist essentially of an amino acid sequence set forth in any one of SEQ ID NOs:1-4. The adjuvant polypeptide can be a GM-CSF polypeptide, an IL-4 polypeptide, an IL-21 polypeptide, a CD40L polypeptide, a 4-1BBL polypeptide, a TGF-β polypeptide, a Clostridium difficile TcdA polypeptide, a C. difficile TcdB polypeptide, or a biologically active fragment thereof. The chaff polypeptide can be a fragment of an ACE2 polypeptide. The fragment of an ACE2 polypeptide can include the extracellular region of an ACE2 polypeptide and can lack a transmembrane domain. The chaff polypeptide can consist essentially of or can consist of an amino acid sequence set forth in SEQ ID NO:8 or SEQ ID NO:9.

In another aspect, this document features for a composition including a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen, (b) a nucleic acid sequence encoding an adjuvant polypeptide, and (c) a nucleic acid sequence encoding a chaff polypeptide.

In another aspect, this document features methods for inducing an immune response against a virus in a mammal. The methods can include, or consist essentially of, administering to a mammal i) a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen, (b) a nucleic acid sequence encoding an adjuvant polypeptide, and (c) a nucleic acid sequence encoding a chaff polypeptide, or ii) a composition including a SC-Ad, where the SC-Ad has a genome which lacks at least a portion of a nucleic acid sequence that encodes an adenovirus polypeptide, where the SC-Ad includes the adenovirus polypeptide, and where the SC-Ad includes (a) a nucleic acid sequence encoding an immunogen, (b) a nucleic acid sequence encoding an adjuvant polypeptide, and (c) a nucleic acid sequence encoding a chaff polypeptide, under conditions where the SC-Ad infects a cell of the mammal, and where expression of the immunogen in the cell leads to induction of the immune response. The mammal can be a human. The virus can be a coronavirus, and the immunogen can be associated with the coronavirus. The coronavirus can be a betacoronavirus. The betacoronavirus can be SARS-CoV-2. The administering can include mucosal delivery of the SC-Ad.

In another aspect, this document features for a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by or shed by an allergen. The adenovirus polypeptide can be a fiber polypeptide, a V polypeptide, a hexon polypeptide, a penton base polypeptide, or a pIIIa polypeptide. The SC-Ad also can include a nucleic acid sequence encoding an adjuvant polypeptide. The adjuvant polypeptide can be a GM-CSF polypeptide, an IL-4 polypeptide, an IL-21 polypeptide, a CD40L polypeptide, a 4-1BBL polypeptide, a TGF-β polypeptide, a Clostridium difficile TcdA polypeptide, a C. difficile TcdB polypeptide, or a biologically active fragment thereof. The SC-Ad also can include a nucleic acid sequence encoding a chaff polypeptide. The chaff polypeptide can be a fragment of an ACE2 polypeptide. The fragment of an ACE2 polypeptide can include the extracellular region of an ACE2 polypeptide and can lack a transmembrane domain. The chaff polypeptide can consists essentially of or consists of an amino acid sequence set forth in SEQ ID NO:8 or SEQ ID NO:9.

In another aspect, this document features for a composition including a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by or shed by an allergen.

In another aspect, this document features methods for inducing an immune response against an allergen in a mammal. The methods can include, or consist essentially of, administering to a mammal i) a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by or shed by an allergen, or ii) a composition including a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by or shed by an allergen, under conditions where the SC-Ad infects a cell of the mammal, and where expression of the immunogen in the cell leads to induction of the immune response. The mammal can be a human.

In another aspect, this document features for a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by a cancer cell. The adenovirus polypeptide can be a fiber polypeptide, a V polypeptide, a hexon polypeptide, a penton base polypeptide, or a pIIIa polypeptide. The SC-Ad also can include a nucleic acid sequence encoding an adjuvant polypeptide. The adjuvant polypeptide can be a GM-CSF polypeptide, an IL-4 polypeptide, an IL-21 polypeptide, a CD40L polypeptide, a 4-1BBL polypeptide, a TGF-polypeptide, a Clostridium difficile TcdA polypeptide, a C. difficile TcdB polypeptide, or a biologically active fragment thereof. The SC-Ad also can include a nucleic acid sequence encoding a chaff polypeptide. The chaff polypeptide can be a fragment of an ACE2 polypeptide. The fragment of an ACE2 polypeptide can include the extracellular region of an ACE2 polypeptide and can lack a transmembrane domain. The chaff polypeptide can consist essentially of or can consist of an amino acid sequence set forth in SEQ ID NO:8 or SEQ ID NO:9.

In another aspect, this document features for a composition including a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by a cancer cell.

In another aspect, this document features methods for inducing an immune response against a cancer cell in a mammal. The methods can include, or consist essentially of, administering to a mammal i) a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by a cancer cell, or ii) a composition including a SC-Ad, where the SC-Ad has a genome lacking at least a portion of a nucleic acid sequence that encodes an adenovirus polypeptide, where the SC-Ad includes the adenovirus polypeptide, and where the SC-Ad includes a nucleic acid sequence encoding an immunogen expressed by a cancer cell, under conditions where the SC-Ad infects a cell of the mammal, and where expression of the immunogen in the cell leads to induction of the immune response. The mammal can be a human.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1B. Schematic of an exemplary TcdA/B fusion protein and an exemplary SC-Ad6 plasmid expressing a fusion protein according to some embodiments. (FIG. 1A) A TcdA/B fusion protein includes a human alpha 1-antitrypsin (AAT) secretory leader sequence and the RBDs of TedA and TedB that are separated by two furin cleavage sites. (FIG. 1B) A SC-Ad6 plasmid expressing the TedA/B fusion using the CMV promoter.

FIGS. 2A-2B. Single-cycle adenovirus expression of C. difficile toxins A and B fusion protein. Western blot detecting expression of (FIG. 2A) TcdA and (FIG. 2B) TedB fragments in the cell lysates and media taken from uninfected cells, cells infected with an Ad control (SC-Ad6-GFP-luciferase (GL)), and cells infected with SC-Ad6-TcdA/B.

FIGS. 3A-3C. Serum antibody responses in immunized CD1 mice and toxin A challenge. Male and female CD1 mice (n=10) were vaccinated intramuscularly (i.m.) with 1×10¹⁰ virus particles of SC-Ad6-PEB1, SC-Ad6-TcdA/B, or PBS. Serum collected at week 6 was assayed by ELISA and a neutralization assay. (FIG. 3A) Serum endpoint titers at week 6 were significantly (p=0.0066) higher in female mice immunized with SC-Ad-TcdA/B than in males (Geometric mean with 95% CI). (FIG. 3B) Neutralizing titers for Toxin A were significantly higher in the female and male groups compared to sex matched controls (Adjusted p =0.0001 and 0.0238 for females and males by Dunn's). (FIG. 3C) Combined male and female toxin A survival curve shows significant survival compared to PBS or PEB1 control animals (p<0.0001).

FIGS. 4A-4D. SC-Ad6-TcdA/B provides protection against lethal challenge long after a single immunization. Female CD1 mice (n=5) were vaccinated i.m. with 1×10¹⁰ virus particles of SC-Ad6-PEB1, SC-Ad6-TcdA/B, or PBS. Serum collected at weeks 3, 6, 14, 26, and 36 were titrated to determine binding endpoint titers for (FIG. 4A) toxin A and (FIG. 4B) toxin B represented as geometric mean with standard deviation. (FIG. 4C) Mean neutralizing titers for toxin B were significantly higher in the SC-Ad-TcdA/B than SC-Ad-PEB1 immunized animals (p>0.0079 by Mann Whitney). (FIG. 4D) Survival curve for SC-Ad-TcdA/B vaccinated mice challenged with toxin A shows significant protection compared to PBS or PEB1 control animals (p=0.0019 and 0.0021, respectively).

FIGS. 5A-5C. Serum neutralizing antibody responses in immunized Syrian Hamsters and protection from lethal spore challenge. Female Syrian Hamsters (n=10) were vaccinated intranasally (i.n.) or i.m. with 1×10¹¹ virus particles of SC-Ad6-TcdA/B, or i.n. with PBS. Serum collected at 6, 12, and 18 weeks after immunization were assayed to determine mean neutralizing titers for (FIG. 5A) toxin A and (FIG. 5B) toxin B (*Adjusted p<0.05 compared to control, **Adjusted p<0.05 compared to control and i.n. route). (FIG. 5C) Survival curve for SC-Ad-TedA/B vaccinated animals challenged with UK1 spores shows significant protection compared to PBS control animals (p<0.0001).

FIGS. 6A-6D. SC-Ad6-TcdA/B provides protection against lethal spore challenge 45 weeks after single immunization. Female Syrian Hamsters (n=10) were vaccinated i.n. or i.m. with 1×10¹¹ virus particles of SC-Ad6-TcdA/B, or i.n. with PBS. Serum collected at weeks 6, 12, and 18, 25, and 36 weeks after immunization were assayed to determine mean neutralizing titers for (FIG. 6A) toxin A and (FIG. 6B) toxin B (*Adjusted p<0.05 compared to control, **Adjusted p<0.05 compared to control and i.n. route). X-axis break represents the termination of the low dose challenge study; serum collected at week 36 from remaining animals in each group (n=5). (FIG. 6C) Survival curve for i.n. (n=5) and i.m. (n=4) SC-Ad-TcdA/B vaccinated animals challenged with UK1 spores 45 weeks after single immunization shows significant protection compared to PBS control animals (n=4) (p=0.0027 vs p=0.0067, respectively). (FIG. 6D) Week 36 neutralizing toxin A and toxin B neutralizing titers of i.n. immunized animals that survived the challenge compared to non-survivors.

FIG. 7. Blood chemistry of SC-Ad6-TcdA/B vaccinated animals. Groups of 10 Syrian hamsters were immunized a single time with 10¹¹ virus particles of SC-Ad6-TcdA/B by the i.n. or i.m. route. Control animals received i.n. PBS. Blood was collected 3 days after immunization for clinical chemistry.

FIGS. 8A-8C. Blood chemistry and CBC of SC-Ad6-TcdA/B vaccinated animals. Groups of 10 Syrian hamsters were immunized a single time with 10¹¹ virus particles of SC-Ad6-TcdA/B by the i.n. or i.m. route. Control animals received i.n. PBS. Blood was collected ½ of the cohort (n=5 per group) (FIG. 8A) 3 and the other ½ of the cohort (n=5 per group) (FIG. 8B) 4 days after immunization for clinical chemistry. (FIG. 8C) Blood was collected on day 4 from ½ of the hamsters (n=5 per group) for CBC.

FIG. 9. Titration of toxin on Vero cells.

FIG. 10. Titer of Toxin B neutralizing antibodies (nAbs) 26 weeks post challenge.

FIG. 11. Survival curve 8 weeks after Toxin A challenge.

FIGS. 12A-12B. Schematic representations of exemplary SC-Ad vectors that can be used to deliver nucleic acid encoding one or more immunogens according to some embodiments. (FIG. 12A) A schematic of an exemplary SC-Ad vector encoding a SARS-CoV-2 Spike variant immunogen and, optionally, one or more adjuvants and/or one more chaff polypeptides. (FIG. 12B) A schematic of an exemplary SC-Ad vector encoding a SARS-CoV-2 Spike variant immunogen and, optionally, one or more adjuvants and/or one more chaff polypeptides. The polypeptide encoding sequence of pIIIA, which is normally located between 52K and penton, is deleted.

FIG. 13. SARS-CoV-2 Spike gene and RBD-Sb subdomain genes with restriction sites.

FIG. 14. Western blot of Spike polypeptide expressed by SC-Ad vector. 1°=anti-spike polyclonal (1:1000); 2°=protein A/G-HRP (1:10,000); Substrate=Pico.

FIG. 15. SC-Ad-Spike infects ACE2+cells and forms cell fusion events.

FIG. 16. SC-Ad-Spike infects ACE2+cells, forms cell fusion events, and expresses adenovirus DNA and adenovirus protein adjuvants.

FIG. 17. Western blot of ACE2 expression in 293-IIIA-ACE2 cells. 1°=anti-ACE2 (1:1000); 2°=protein A/G-HRP (1:10,000).

FIG. 18. Western blot of Spike polypeptide expressed by various plasmid expression vectors.

FIG. 19. Antibody responses in mice 2 weeks after administration of plasmid vectors expressing Spike polypeptide or negative control GFP-Luciferase (GL) vector.

FIGS. 20A-20F. IgA and IgG antibody responses in mice 2 (FIGS. 20A, 20C, and 20D) or 6 weeks (FIGS. 20B) after intranasal (IN) or intramuscular (IM) administration of SC-Ad expressing Spike polypeptide or negative control SC-Ad expressing Zika protein or buffer (PBS). IFNγ (FIG. 20E) and CD8 T cell counts (FIG. 20F) were also measured.

FIG. 21. Western blot of Spike polypeptide expression by replication-defective adenovirus (RD-Ad) and SC-Ad expressing SARS-CoV-2 spike.

FIG. 22. An exemplary SC-Ad carrying SARS-CoV-2 Spike or RBD with centralized influenza HA gene H1-CON.

FIG. 23. An exemplary SC-Ad carrying centralized influenza HA gene H1-CON covering H1 hemagglutinins and H1-5 CON covering H1-H5 hemagglutinin sequences.

FIG. 24. Serum antibodies generated by plasmid vaccines are increased by co-immunization with granulocyte-macrophage colony-stimulating factor (GM-CSF) adjuvant plasmid.

FIG. 25. Serum antibodies 6 weeks after a single i.n. administration of SC-Ad HIV Env in combination with SC-Ads expressing genetic adjuvants.

FIG. 26. Vaginal antibodies 6 weeks after a single i.n. administration of SC-Ad HIV Env in combination with SC-Ads expressing genetic adjuvants.

FIG. 27. Vaginal antibodies after an i.m. administration of SC-Ad HIV Env in combination with SC-Ads expressing genetic adjuvants and followed by i.m. or i.n. administration of HIV-1 Envelope SOSIP polypeptide.

FIG. 28. A sequence listing for an amino acid sequence (SEQ ID NO:1) of a SARS-CoV-2 Spike polypeptide.

FIG. 29. A sequence listing for an amino acid sequence (SEQ ID NO:2) of a SARS-CoV-2 Spike polypeptide lacking an ER retention sequence.

FIG. 30. A sequence listing for an amino acid sequence (SEQ ID NO:3) of an ectodomain of a SARS-CoV-2 Spike polypeptide.

FIG. 31. A sequence listing for an amino acid sequence (SEQ ID NO:4) of a receptor binding domain of a SARS-CoV-2 Spike polypeptide.

FIG. 32. A sequence listing for an amino acid sequence (SEQ ID NO:5) of a receptor binding domain of a SARS-CoV-2 Spike polypeptide fused to an Ig polypeptide.

FIG. 33. A sequence listing for an amino acid sequence (SEQ ID NO:6) of a receptor binding domain of a SARS-CoV-2 Spike polypeptide fused to a streptavidin polypeptide.

FIG. 34. A sequence listing for an amino acid sequence (SEQ ID NO:7) of a receptor binding domain of a SARS-CoV-2 Spike polypeptide fused to a sigma coil polypeptide.

FIG. 35. A sequence listing for an amino acid sequence (SEQ ID NO:8) of an ectodomain of an ACE2 chaff polypeptide.

FIG. 36. A sequence listing for an amino acid sequence (SEQ ID NO:9) of an inactivated ectodomain of an ACE2 chaff polypeptide.

FIGS. 37A-37C. Sequence listings for amino acid sequences of adjuvant polypeptides derived from a C. difficile toxin. (FIG. 37A) A sequence listing for an amino acid sequence (SEQ ID NO:22) of a TcdA polypeptide. (FIG. 37B) A sequence listing for an amino acid sequence (SEQ ID NO:23) of a TcdB polypeptide. (FIG. 37C) A sequence listing for an amino acid sequence (SEQ ID NO:10) of a TcdA/B fusion polypeptide.

FIG. 38. A sequence listing for an amino acid sequence (SEQ ID NO:11) of a SARS-CoV-2 ORF1ab polypeptide.

FIG. 39. A sequence listing for an amino acid sequence (SEQ ID NO:12) of a SARS-CoV-2 S polypeptide.

FIG. 40. A sequence listing for an amino acid sequence (SEQ ID NO:13) of a SARS-CoV-2 ORF3 polypeptide.

FIG. 41. A sequence listing for an amino acid sequence (SEQ ID NO:14) of a SARS-CoV-2 E polypeptide.

FIG. 42. A sequence listing for an amino acid sequence (SEQ ID NO:15) of a SARS-CoV-2 M polypeptide.

FIG. 43. A sequence listing for an amino acid sequence (SEQ ID NO:16) of a SARS-CoV-2 ORF3 polypeptide.

FIG. 44. A sequence listing for an amino acid sequence (SEQ ID NO:17) of a SARS-CoV-2 ORF7 polypeptide.

FIG. 45. A sequence listing for an amino acid sequence (SEQ ID NO:18) of a SARS-CoV-2 ORF8 polypeptide.

FIG. 46. A sequence listing for an amino acid sequence (SEQ ID NO:19) of a SARS-CoV-2 N polypeptide.

FIG. 47. A sequence listing for an amino acid sequence (SEQ ID NO:20) of a centralized H1 influenza hemagglutinin polypeptide.

FIG. 48. A sequence listing for an amino acid sequence (SEQ ID NO:21) of a centralized H1-5 influenza hemagglutinin polypeptide.

FIG. 49. A sequence listing for a nucleic acid sequence (SEQ ID NO:24) that can encode a TcdA polypeptide derived from a C. difficile toxin.

FIG. 50. A sequence listing for a nucleic acid sequence (SEQ ID NO:25) that can encode a TcdB polypeptide derived from a C. difficile toxin.

FIG. 51. A sequence listing for nucleic acid sequence (SEQ ID NO:26) that can encode a SC-Ad-Spike virus.

FIG. 52. A sequence listing for nucleic acid sequence (SEQ ID NO:27) that can encode a SC-Ad-TcdA/B virus.

FIG. 53. A sequence listing for a SC-Ad6-ΔIII-ΔE3-CMV-Spike-3X-LZL nucleic acid (SEQ ID NO:28).

FIG. 54. A sequence listing for a SC-Ad6-□III-□E3-CMV-Spike-PP-3X-LZL nucleic acid (SEQ ID NO:29).

FIG. 55. A sequence listing for a SC-Ad6-□III-□E3ADP-I-CMV-Spike-3X-L nucleic acid (SEQ ID NO:30).

FIG. 56. A sequence listing for a SC-Ad6-□III-□E3ADP-I-CMV-Spike-PP-3X-L nucleic acid (SEQ ID NO:31).

FIG. 57. A sequence listing for a SC-Ad-FZF-657-ΔIIIF-ΔE3-Spike-3X-L nucleic acid (SEQ ID NO:32).

FIG. 58. A sequence listing for a SC-Ad-FZF-657-ΔIIIF-ΔE3-SpikePP-3X-L nucleic acid (SEQ ID NO:33).

FIG. 59. A sequence listing for a SC-Ad-FZF-C68-ΔIIIF-ΔE3-Spike-3X-L nucleic acid (SEQ ID NO:34).

FIG. 60. A sequence listing for a SC-Ad-FZF-C68-ΔIIIF-ΔE3-SpikePP-3X-L nucleic acid (SEQ ID NO:35).

FIG. 61. A sequence listing for a SC-Ad-F-657-ΔIIIF-ΔE3-Spike-3X-L nucleic acid (SEQ ID NO:36).

FIG. 62. A sequence listing for a SC-Ad-F-657-ΔIIIF-ΔE3-SpikePP-3X-L nucleic acid (SEQ ID NO:37).

FIG. 63. A sequence listing for a SC-Ad-F-C68-ΔIIIF-ΔE3-Spike-3X-L nucleic acid (SEQ ID NO:38).

FIG. 64. A sequence listing for a SC-Ad-F-C68-ΔIIIF-ΔE3-SpikePP-3X-L nucleic acid (SEQ ID NO:39).

### DETAILED DESCRIPTION

This document provides adenovirus vectors and methods and materials for using adenovirus vectors. In some cases, adenovirus vectors encoding one or more (e.g., one, two, three, four, five, six, seven, eight, nine, ten, or more) immunogens can be used to deliver immunogens to a mammal (e.g., a human) such that the mammal produces an effective immune response (e.g., an immune response against those immunogens). For example, adenovirus vectors encoding one or more immunogens can be used to deliver immunogens to a mammal (e.g., a human) such that the mammal produces antibodies against a pathogen, allergen, and/or cancer cell associated with those immunogens. In some cases, nucleic acid molecules that can encode an adenovirus vector encoding one or more immunogens can be used to deliver immunogens to a mammal (e.g., a human) such that the mammal produces an effective immune response (e.g., an immune response against those immunogens). For example, nucleic acid molecules that can encode an adenovirus vector encoding one or more immunogens can be used to deliver immunogens to a mammal (e.g., a human) such that the mammal produces antibodies against a pathogen, allergen, and/or cancer cell associated with those immunogens.

This document also provides adenovirus vectors encoding one or more (e.g., one, two, three, four, five, six, seven, eight, nine, ten, or more) immunogens (e.g., SC-Ads encoding one or more immunogens), nucleic acid molecules encoding adenovirus vectors encoding one or more immunogens, cell lines containing adenovirus vectors encoding one or more immunogens, methods for using adenovirus vectors encoding one or more immunogens to deliver the immunogen(s) to cells in vitro or in vivo, and methods for using adenovirus vectors encoding one or more immunogens to induce immune responses within a mammal (e.g., a human).

An adenovirus vector provided herein (e.g., an adenovirus vector encoding one or more immunogens) can be derived from any adenovirus. An adenovirus used to create an adenovirus vector provided herein can be any appropriate serotype (e.g., Ad1-Ad57). In some cases, an adenovirus can be a replication competent adenovirus. In some cases, an adenovirus can be a replication defective adenovirus. In some cases, an adenovirus can be capable of infecting a human cell (e.g., can be a human adenovirus). In some cases, an adenovirus can be capable of infection a non-human cell (e.g., can be a non-human adenovirus) such as chimpanzee cells. Examples of adenoviruses that can be used to make an adenovirus vector provided herein include, without limitation, Ad5 adenoviruses, Ad6 adenoviruses, ChAdOx1, and ChAdOx2.

In some cases, an adenovirus vector provided herein (e.g., an adenovirus vector encoding one or more immunogens) can be a SC-Ad. A SC-Ad can have a genome which lacks all or a portion of at least of one of the following adenovirus nucleic acid sequences: fiber protein-encoding sequence, V protein-encoding sequence, hexon-encoding sequence, penton base-encoding sequence (also referred to as a pIII-encoding sequence), VA RNA-encoding sequence, pIIIa protein-encoding sequence (also referred to as a minor capsid protein-encoding sequence), or other early or late gene product-encoding sequences. Examples of nucleic acid sequences that encode adenoviral polypeptides include, without limitation, those set forth in GenBank gi numbers 209842, 58478, or 2935210, and/or annotated in GenBank accession numbers M73260, X17016, or AF030154. In some cases, a deletion of all or a portion of the nucleic acid encoding one or more of the following polypeptides can be engineered into a nucleic acid encoding an adenovirus such that the adenovirus vector does not encode that full-length adenovirus polypeptide or a fully functional version of that adenovirus polypeptide: fiber protein-encoding sequence, V protein-encoding sequence, hexon-encoding sequence, penton base-encoding sequence, VA RNA-encoding sequence, pIIIa protein-encoding sequence, or other early or late gene product-encoding sequences. Such deletions can be any length that results in the deletion of one or more encoded amino acids and in a reduction or elimination of the normal function of that polypeptide. For example, portions of a nucleic acid sequence of an adenovirus can be removed such that the otherwise encoded polypeptide lacks 5, 6, 7, 8, 9, 10, 15, 20, 25, 30, or more amino acid residues and lacks its normal activity. The portion or portions to be deleted can be removed from any location along the length of the sequence. For example, a portion of an adenovirus nucleic acid sequence can be removed at the 5′ end, the 3′ end, or an internal region of an adenovirus nucleic acid such as a fiber protein-encoding sequence, V protein-encoding sequence, hexon-encoding sequence, penton base-encoding sequence, VA RNA-encoding sequence, pIIIa protein-encoding sequence, or other early or late gene product-encoding sequences. In some cases, a SC-Ad can be as described elsewhere (see, e.g., Matchett et al., J. of Virol., 2019 93(10):e02016-18 (2019); and International PCT Patent Application Publication No. WO 2009/111738).

An adenovirus vector provided herein (e.g., an adenovirus vector encoding one or more immunogens) can include a nucleic acid sequence encoding any appropriate immunogen (e.g., a nucleic acid that drives expression of any appropriate immunogen). In some cases, an immunogen can be an antigen. An immunogen can be a full-length immunogenic polypeptide or a portion thereof (e.g., can be derived from an immunogenic polypeptide).

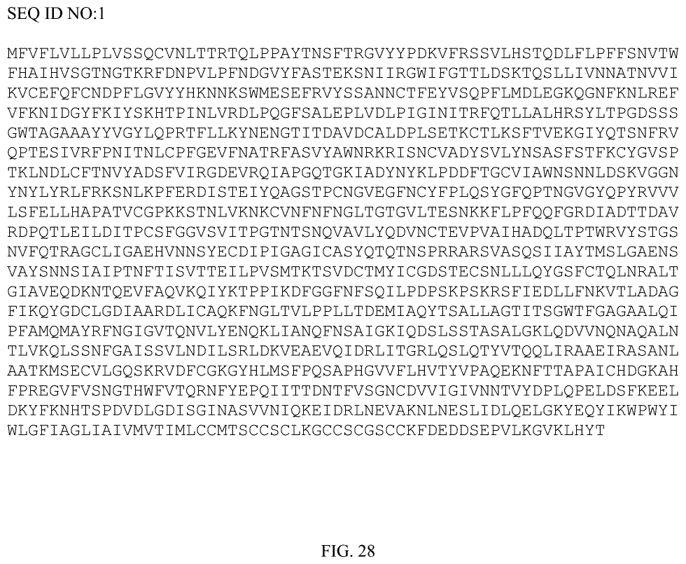

When an immunogenic polypeptide is from a pathogen, the immunogenic polypeptide can be from any type of pathogen (e.g., a virus, a bacterium, a protozoan, a prion, a viroid, or a fungus). In some cases, an immunogenic polypeptide can be a polypeptide expressed by a virus (e.g., a viral polypeptide). For example, an immunogenic polypeptide can be a polypeptide expressed by a coronavirus (e.g., a beta-coronavirus). Examples of viruses that can express an immunogenic polypeptide include, without limitation, SARS-CoV, HCoV NL63, HKU1, MERS-CoV, SARS-CoV-2, HIV-1, hepatitis B virus, hepatitis C virus, hepatitis D virus, hepatitis E virus, influenza, Ebola virus, Chiningunya virus, Zika virus, cytomegalovirus, West Nile virus, and those described in Table 3-1 of “Learning from SARS: Preparing for the Next Disease Outbreak: Workshop Summary.” Institute of Medicine (US) Forum on Microbial Threats; Knobler S, Mahmoud A, Lemon S, et al., editors. Washington (DC): National Academies Press (US); 2004. In some cases, an immunogen can be derived from a polypeptide expressed by a bacterium (e.g., a bacterial polypeptide). Examples of bacteria that express polypeptides from which an immunogen can be derived include, without limitation, Clostridium (e.g., C. difficile), Staphylococcus aureus (e.g. methicillin-resistant S. aureus), Campylobacter (e.g. Campylobacter jejuni), Mycobacteria (e.g. M. tuberculosis), and Borrelia (B. burgdorferi). Examples of immunogenic polypeptides that can be expressed by a pathogen include, without limitation, C. difficile Toxin A (TcdA) polypeptides, C. difficile Toxin B (TcdB) polypeptides, coronavirus Spike polypeptides, the amino acid sequence set forth in SEQ ID NO:1 (see, e.g., FIG. 28), coronavirus nucleoproteins, coronavirus membrane proteins, coronavirus envelope proteins, and coronavirus non-structural proteins (e.g., coronavirus non-structural proteins 1-16). For example, an immunogenic polypeptide associated with a pathogen can have, or can be encoded by, a sequence set forth in, for example, National Center for Biotechnology Information (NCBI) Accession Nos: MN938384 and AY772062.

When an immunogenic polypeptide is from an allergen, the immunogenic polypeptide can be from any type of allergen (e.g., a substance capable of triggering an immune response that results in an allergic reaction). Examples of allergens that can express and/or shed an immunogenic polypeptide include, without limitation, Fel d 7, Can f1, beta-lactoglobulin, prolamin, parvalbumin, gliadin, Fel d1, chitinase, glutenin, cupin, prolamin, profilins, polcalcins, bet v-1-related proteins, 2S albumins, vicilins, legumins, nsLTPs, and Aed a 2. For example, adenovirus vectors encoding one or more immunogens described herein can be used to deliver immunogens to a mammal (e.g., a human) such that the mammal produces antibodies against an allergen associated with those immunogens. For example, nucleic acid molecules that can encode an adenovirus vector encoding one or more immunogens can be used to deliver immunogens to a mammal (e.g., a human) such that the mammal produces antibodies against an allergen associated with those immunogens. For example, an immunogenic polypeptide associated with an allergen can have, or can be encoded by, a sequence set forth in, for example, NCBI Accession Nos: NP_001363134.1,AAD56719, NP_001363136.1, NP_001363139.1, P27762.1, P10414.2, P15494.2, P43176.2, NP_001191706.1, NP_001003190.1, XP_030099003.1, or XP_001657779.1.

When an immunogenic polypeptide is from a cancer cell (e.g., a cancer cell within a mammal having cancer), the immunogenic polypeptide can be expressed by any cancer cell. For example, an immunogenic polypeptide expressed by a cancer cell can be a tumor antigen. In some cases, an immunogenic polypeptide expressed by a cancer cell can be a cell surface tumor antigen. In some cases, an immunogenic polypeptide expressed by a cancer cell can be a tumor-associated antigen (TAA; e.g., an antigen, such as an abnormal protein, present on tumor cells). In some cases, an immunogenic polypeptide can be a tumor-specific antigen (TSA; e.g., an antigen present only on tumor cells). Examples of immunogenic polypeptides that can be expressed by a cancer cell and used as described herein include, without limitation, folate receptor alpha, mucin 1 (MUC-1), human epidermal growth factor receptor 2 (HER-2), estrogen receptor (ER), epidermal growth factor receptor (EGFR), folate receptor alpha, mesothelin, alphafetoprotein (AFP), carcinoembryonic antigen (CEA), CA-125, epithelial tumor antigen (ETA), melanoma-associated antigen (MAGE), antigens produced by Epstein-Barr Viruses, and antigens produced by human papilloma viruses. For example, adenovirus vectors encoding one or more immunogens as described herein can be used to deliver immunogens to a mammal (e.g., a human) such that the mammal produces antibodies against cancer cells associated with those immunogens. For example, nucleic acid molecules that can encode an adenovirus vector encoding one or more immunogens as described herein can be used to deliver immunogens to a mammal (e.g., a human) such that the mammal produces antibodies against cancer cells associated with those immunogens. For example, an immunogenic polypeptide associated with a cancer cell can have, or can be encoded by, a sequence set forth in, for example, NCBI Accession Nos: XP_002754883.1, AAA03229.1, Q02496.2, AAD33253.1, CEQ32409.1, YP_401631.1, YP_401632.1, or QAR15051.1.

When an immunogenic polypeptide is from a cancer cell (e.g., a cancer cell within a mammal having cancer), the immunogenic polypeptide can be expressed by any type of cancer cell. Examples of such cancers include, without limitation, lung cancers, breast cancers, prostate cancers, liver cancer, kidney cancers, brain cancers, B cell cancers, T cell cancers, ovarian cancers, and skin cancers.

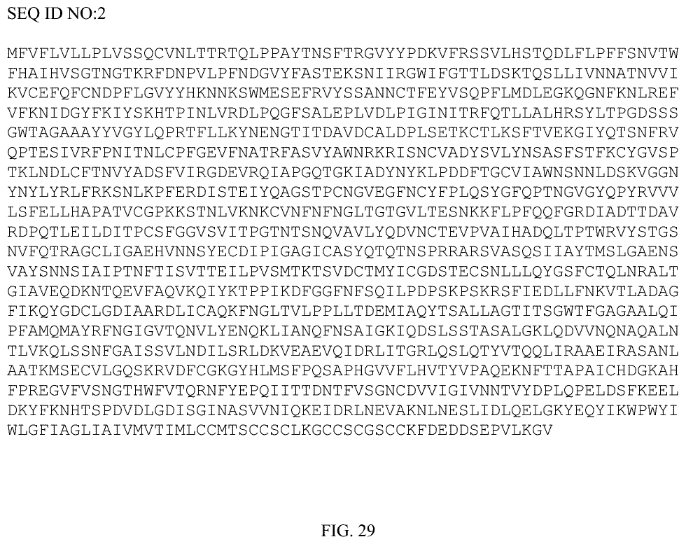

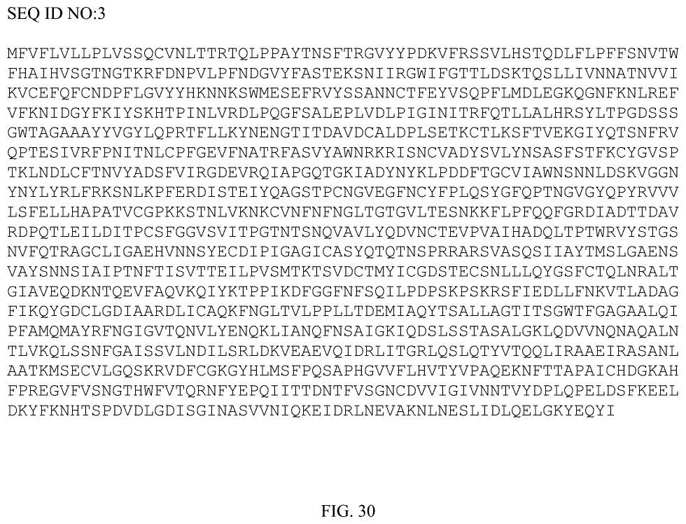

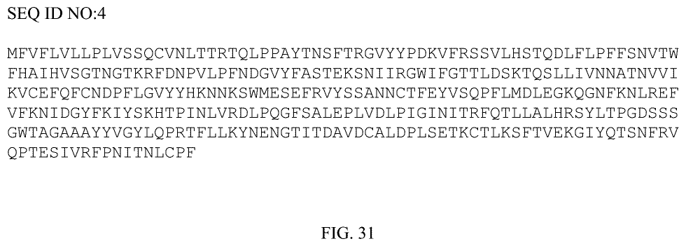

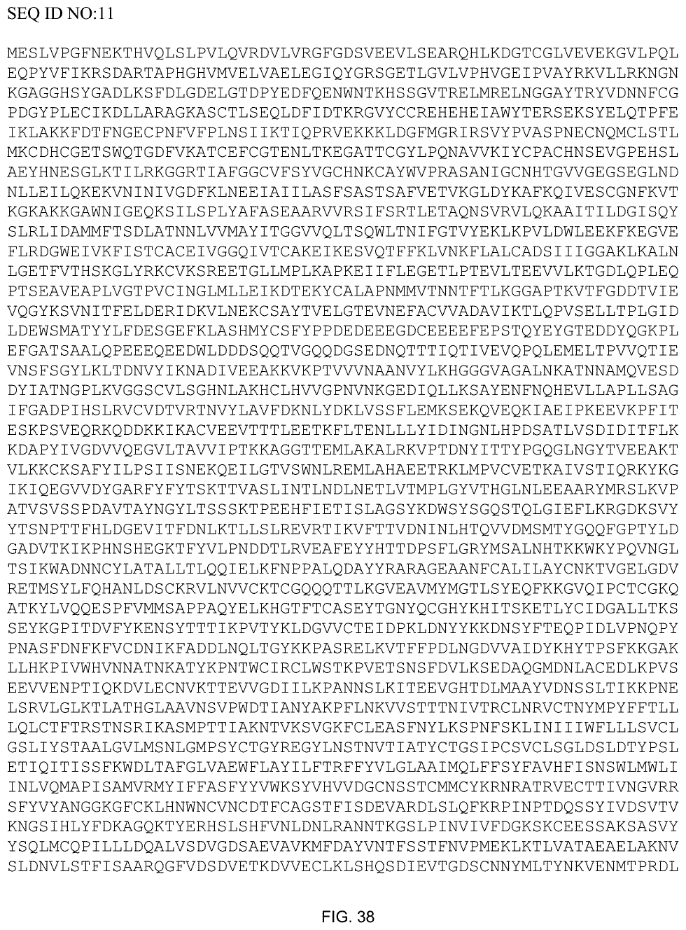

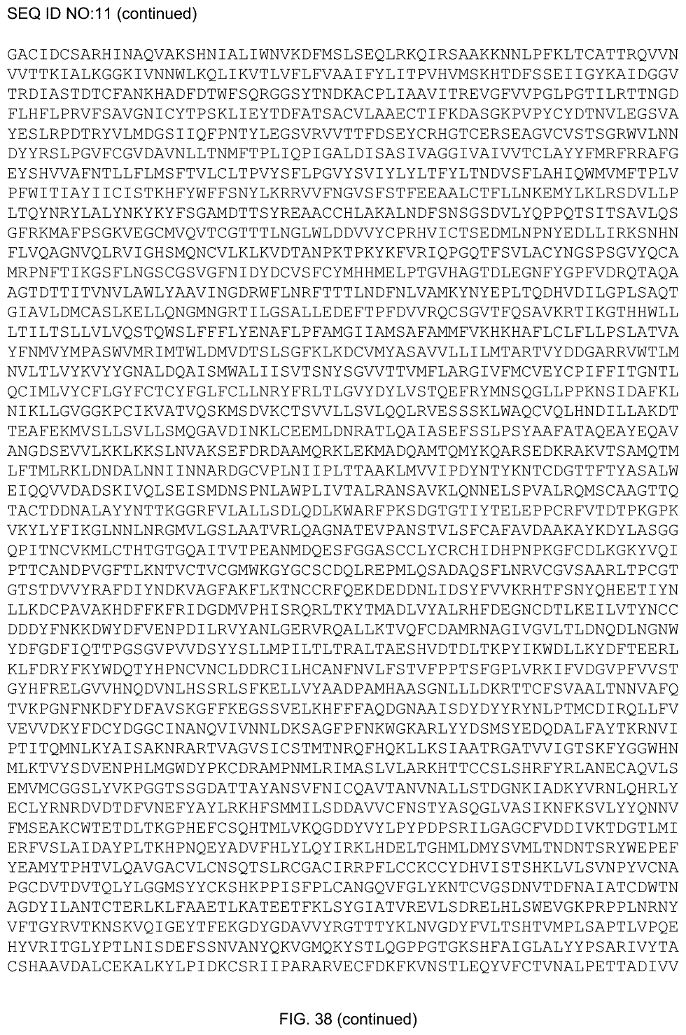

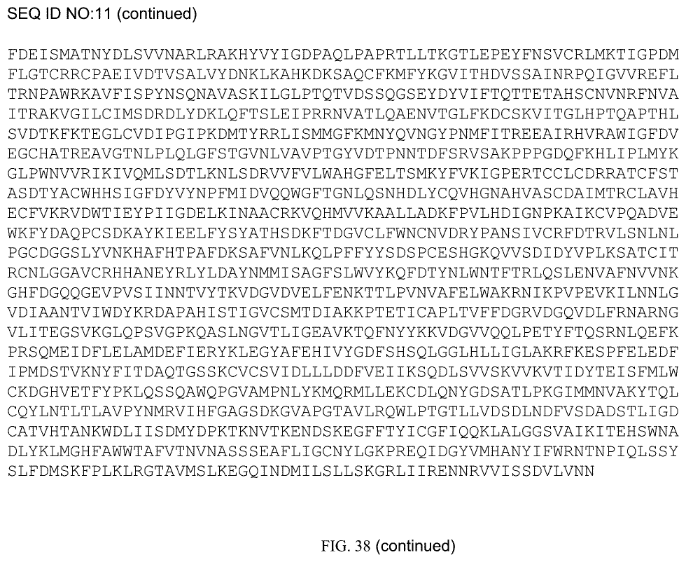

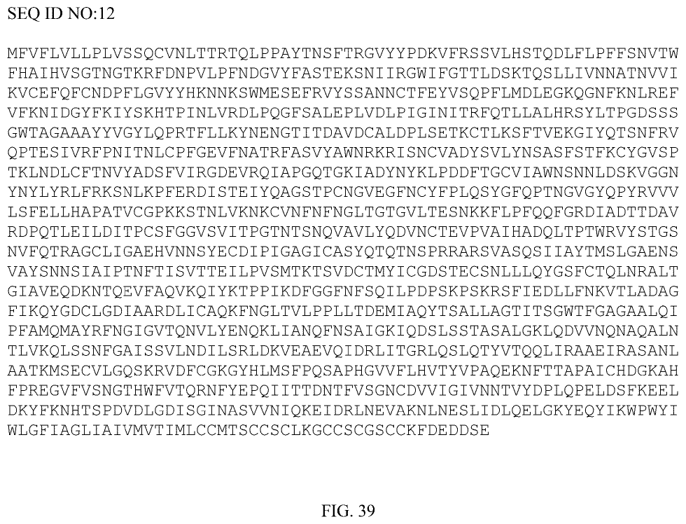

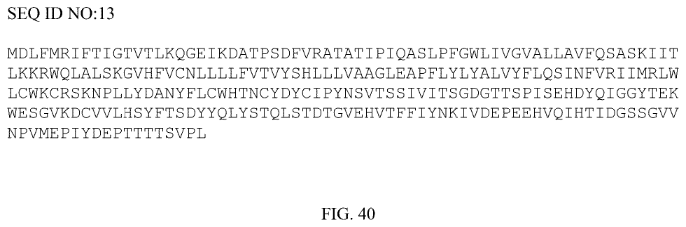

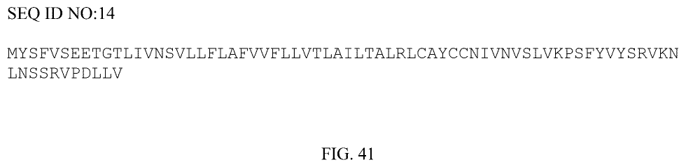

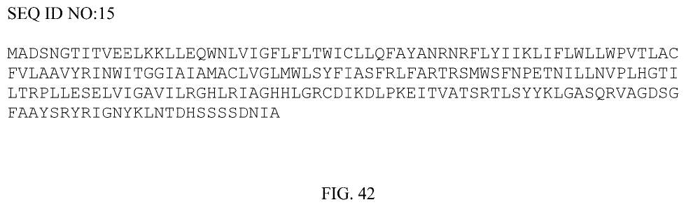

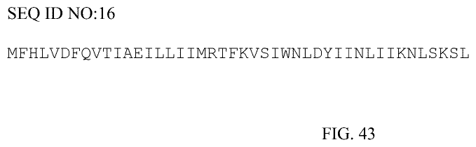

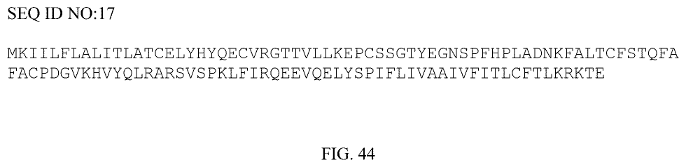

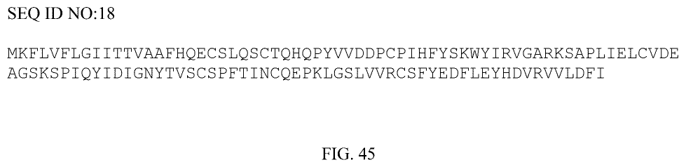

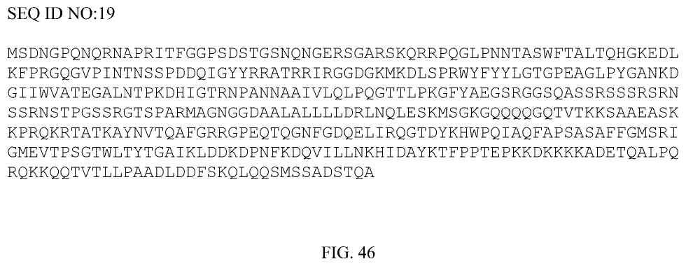

An immunogen can be a full-length immunogenic polypeptide or a portion thereof (e.g., can be derived from an immunogenic polypeptide). For example, a nucleic acid sequence encoding an immunogenic polypeptide can be modified to remove portions of nucleic acid such that the encoded polypeptide lacks any number of amino acids (e.g., 5, 10, 15, 20, 30 amino acids, or all amino acids of the immunogenic polypeptide). In some cases, portions of a nucleic acid sequence encoding an immunogenic polypeptide can be removed from anywhere along the length of the sequence. For example, portions of the nucleic acid sequence can be removed at the 5′ end, the 3′ end, or an internal region of the target nucleic acid. In some cases, an immunogen can be designed to be secreted from cells infected with the adenovirus vector encoding the immunogen. For example, a nucleic acid sequence encoding an ER retention sequence can be removed from a nucleic acid sequence encoding an immunogen (e.g., such that the encoded immunogen lacks an ER retention sequence). In some cases, an immunogen can be designed to extend from cells infected with the adenovirus vector encoding the immunogen into the extracellular space. For example, an immunogen can include an ectodomain of an immunogenic polypeptide. In some cases, an immunogen can bind (e.g., can be designed to bind) to viral receptor (e.g., an ACE2 polypeptide). For example, an immunogen can include a receptor binding domain of an immunogenic polypeptide. Examples of immunogens derived from immunogenic polypeptides that can be used as described herein include, without limitation, the amino acid sequence set forth in SEQ ID NO:2 (see, e.g., FIG. 29), the amino acid sequence set forth in SEQ ID NO:3 (see, e.g., FIG. 30), the amino acid sequence set forth in SEQ ID NO:4 (see, e.g., FIG. 31), the amino acid sequence set forth in SEQ ID NO:11 (see, e.g., FIG. 38), the amino acid sequence set forth in SEQ ID NO:12 (see, e.g., FIG. 39), the amino acid sequence set forth in SEQ ID NO:13 (see, e.g., FIG. 40), the amino acid sequence set forth in SEQ ID NO:14 (see, e.g., FIG. 41), the amino acid sequence set forth in SEQ ID NO:15 (see, e.g., FIG. 42), the amino acid sequence set forth in SEQ ID NO:16 (see, e.g., FIG. 43), the amino acid sequence set forth in SEQ ID NO:17 (see, e.g., FIG. 44), the amino acid sequence set forth in SEQ ID NO:18 (see, e.g., FIG. 45), and the amino acid sequence set forth in SEQ ID NO:19 (sec, e.g., FIG. 46).

In some cases, an immunogen described herein can be a variant of a wild-type immunogen. For example, a variant of a coronavirus Spike polypeptide (e.g., a SARS-CoV-2 Spike polypeptide) can comprise or consist essentially of an amino acid sequence set forth in any one of SEQ ID NOs:1-4 with one or more (e.g., one, two, three, four, five, six, seven, eight, nine, ten, or more) amino acid deletions, additions, substitutions, or combinations thereof.

In some cases, an immunogen described herein can have an amino acid sequence with at least 85% sequence identity (e.g., at least 88% sequence identity, at least 90% sequence identity, at least 93% sequence identity, at least 95% sequence identity, at least 97% sequence identity, at least 98% sequence identity, or at least 99% sequence identity) to the amino acid sequence set forth in any one of SEQ ID NOs:1-4.

The percent sequence identity between a particular amino acid sequence and a sequence referenced by a particular sequence identification number is determined as follows. First, an amino acid sequence is compared to the sequence set forth in a particular sequence identification number using the BLAST 2 Sequences (B12seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the B12seq program can be found in the readme file accompanying BLASTZ. B12seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt) ; -j is set to a file containing the second nucleic acid sequence to be compared (e.g., C: \seq2.txt) ; -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\B12seq -i c:\seq1.txt -j c:\seq2.txt -p blastn -o c:\output.txt -q -1 -r 2. To compare two amino acid sequences, the options of B12seq are set as follows: -i is set to a file containing the first amino acid sequence to be compared (e.g., C:\seq1.txt) ; -j is set to a file containing the second amino acid sequence to be compared (e.g., C:\seq2.txt) ; -p is set to blastp; -o is set to any desired file name (e.g., C:\output.txt); and all other options are left at their default setting. For example, the following command can be used to generate an output file containing a comparison between two amino acid sequences: C:\B12seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences. Once aligned, the number of matches is determined by counting the number of positions where an identical nucleotide or amino acid residue is presented in both sequences. A matched position refers to a position in which identical amino acid occur at the same position in aligned sequences. The percent sequence identity is determined by dividing the number of matches by the length of the sequence set forth in the identified sequence (e.g., SEQ ID NO:1, SEQ ID NO:2, SEQ ID NO:3, or SEQ ID NO:4), followed by multiplying the resulting value by 100. For example, an amino acid sequence that has 220 matches when aligned with the sequence set forth in SEQ ID NO:2 is 93.2 percent identical to the sequence set forth in SEQ ID NO:2 (i.e., 220÷236×100=93.2). It is noted that the percent sequence identity value is rounded to the nearest tenth. For example, 75.1, 75.2, 75.3, and 75.4 is rounded down to 75, while 75.5, 75.6, 75.7, 75.8, and 75.9 is rounded up to 76. It also is noted that the length value will always be an integer.

In some cases, a coronavirus Spike polypeptide variant can contain the entire amino acid sequence set forth in any one of SEQ ID NOs:1-4, except that the amino acid sequence contains from one to ten (e.g., one to nine, two to nine, one to eight, two to eight, one to seven, one to six, one to five, one to four, one to three, two, or one) amino acid additions, deletions, substitutions, or combinations thereof, provided that the coronavirus Spike polypeptide variant has the ability to induce an immune response against a coronavirus within a mammal (e.g., a human). In some cases, a coronavirus Spike polypeptide variant can consist essentially of the amino acid sequence set forth in any one of SEQ ID NOs:1-4 except that the amino acid sequence contains one, two, three, four, or five amino acid residues preceding the articulated sequence of the sequence identifier (e.g., SEQ ID NO:1), and/or has one, two, three, four, or five amino acid residues following the articulated sequence of the sequence identifier (e.g., SEQ ID NO:1), provided that the coronavirus Spike polypeptide has the ability to induce an immune response against a coronavirus within a mammal (e.g., a human).

In some cases, an adenovirus vector provided herein (e.g., an adenovirus vector encoding one or more immunogens) can include nucleic acid sequence encoding two or more (e.g., two, three, four, five, six, seven, eight, nine, ten, or more) immunogens from different immunogenic polypeptides. For example, an adenovirus vector provided herein can include nucleic acid sequence encoding an immunogen derived from a first pathogen (e.g., an immunogen derived from an immunogenic polypeptide expressed by SARS-CoV-2) and can include nucleic acid sequence encoding an immunogen derived from a second pathogen (e.g., an immunogen derived from an immunogenic polypeptide expressed by a pathogen other than SARS-CoV-2). In some cases, an adenovirus vector provided herein that includes a nucleic acid sequence encoding two or more immunogens derived from an immunogenic polypeptide expressed by different pathogens can include nucleic acid sequence encoding a polypeptide comprising, consisting of, or consisting essentially of the amino acid sequence set forth in any one of SEQ ID NOs:1-4 and can include nucleic acid sequence encoding a polypeptide comprising, consisting of, or consisting essentially of the amino acid sequence set forth in any one of SEQ ID NOs:20-21. When an adenovirus vector provided herein includes nucleic acid sequence encoding two or more immunogens from immunogenic polypeptides expressed by different pathogens, the adenovirus vector can be used to induce an immune response against two or more pathogens.

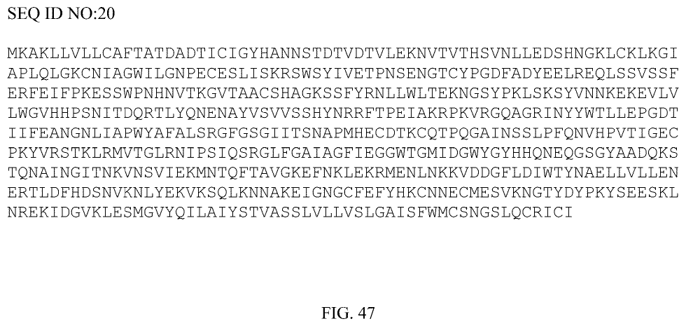

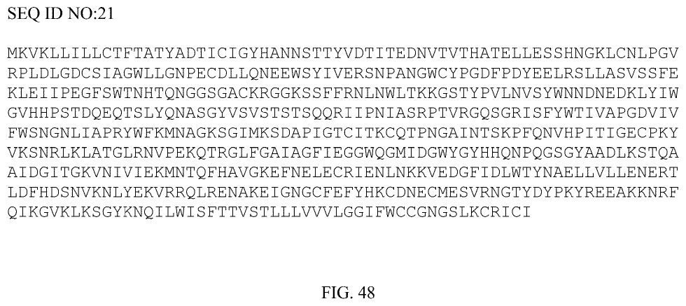

In some cases, an adenovirus vector provided herein (e.g., an adenovirus vector encoding one or more immunogens) can include nucleic acid sequence encoding two or more (e.g., two, three, four, five, six, seven, eight, nine, ten, or more) immunogens from the same immunogenic polypeptide and/or the same pathogen. For example, an adenovirus vector provided herein can include nucleic acid sequence encoding two or more immunogens derived from the same pathogen. In some cases, an adenovirus vector provided herein that includes a nucleic acid sequence encoding two or more immunogens derived from an immunogenic polypeptide expressed by influenza can include nucleic acid sequence encoding a polypeptide comprising, consisting of, or consisting essentially of the amino acid sequence set forth in SEQ ID NO:20 (see, e.g., FIG. 47) and can include nucleic acid sequence encoding a polypeptide comprising, consisting of, or consisting essentially of the amino acid sequence set forth in SEQ ID NO:21 (see, e.g., FIG. 48).

In some cases, an adenovirus vector provided herein (e.g., an adenovirus vector encoding one or more immunogens) that includes a nucleic acid sequence encoding one or more (e.g., one, two, three, four, five, six, seven, eight, nine, ten, or more) immunogens also can include one or more regulatory sequences (e.g., an enhancer or a promoter sequence such as a constitutive, inducible, and/or tissue-specific promoter sequence) to drive transcription of the immunogen(s). Examples of enhancers and promoters that can be used to drive expression of a nucleic acid sequence encoding one or more immunogens of an adenovirus provided herein include, without limitation, a CMV enhancer sequence, a CMV promoter sequence, a CAG enhancer sequence, a CAG promoter sequence, a RSV enhancer sequence, a RSV promoter sequence, a Ef1alpha enhancer sequence, a Ef1alpha promoter sequence, a ubiquitin enhancer sequence, a ubiquitin promoter sequence, adenovirus enhancer sequences, and adenovirus promoter sequences. Any appropriate method can be used to detect expression of an immunogen from adenovirus vector infected cells. For example, antibodies that recognize an immunogen can be used to detect the presence or absence of the immunogen.

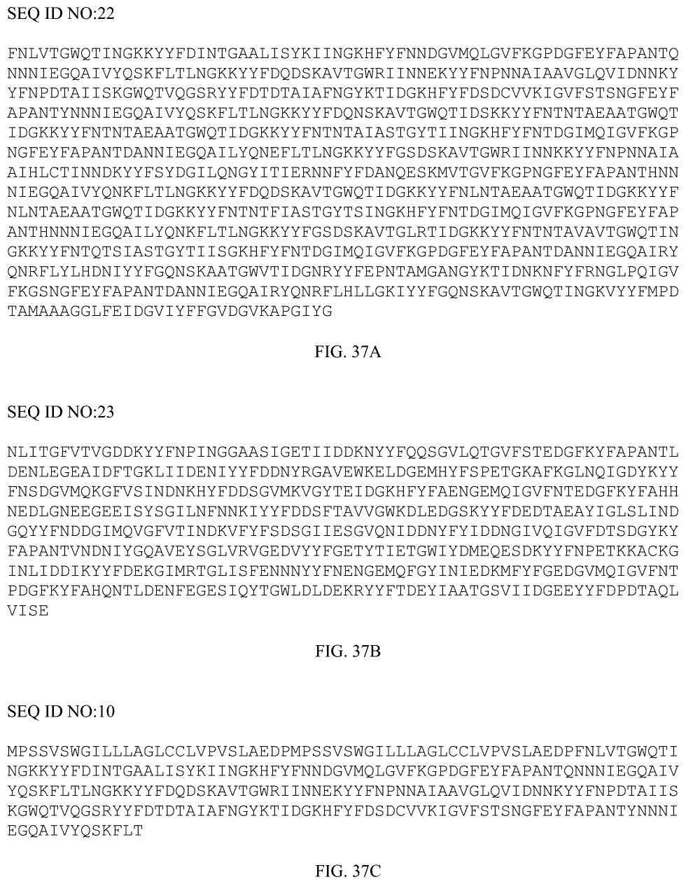

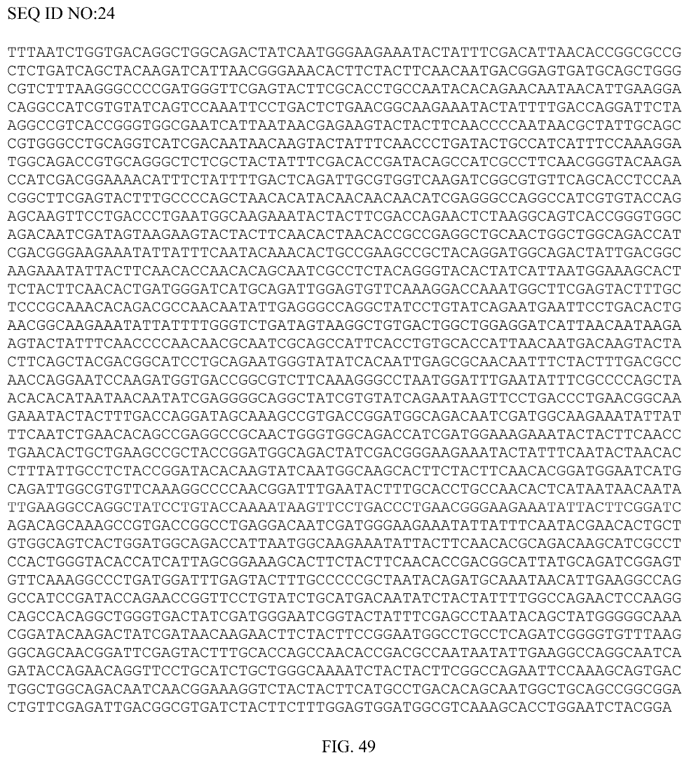

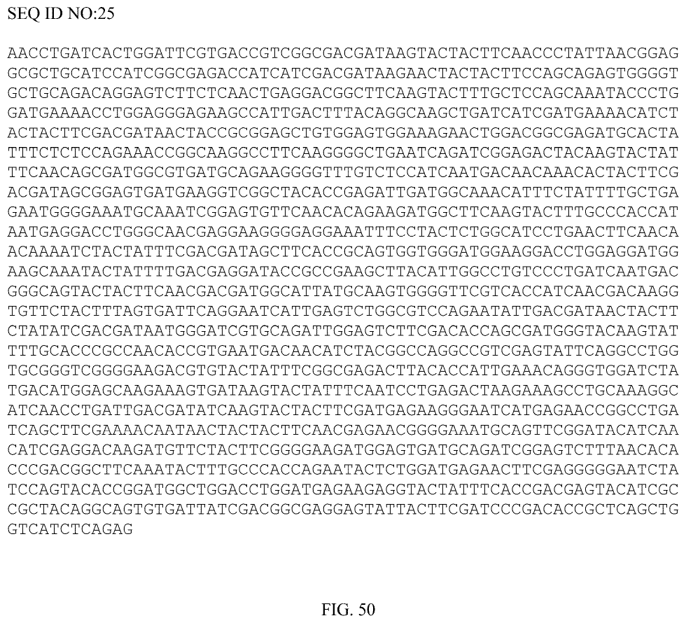

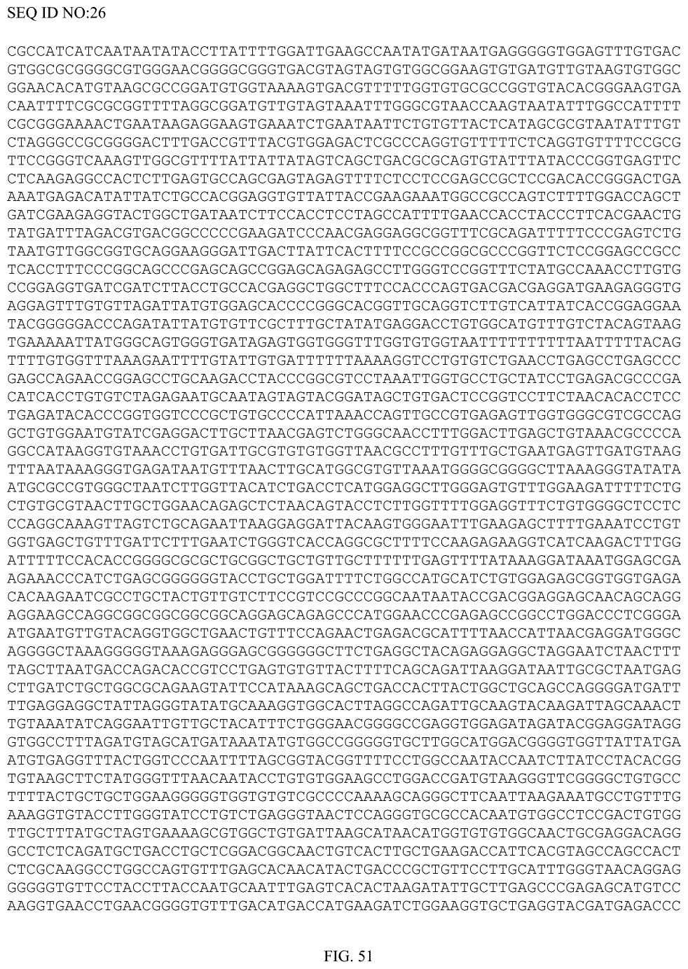

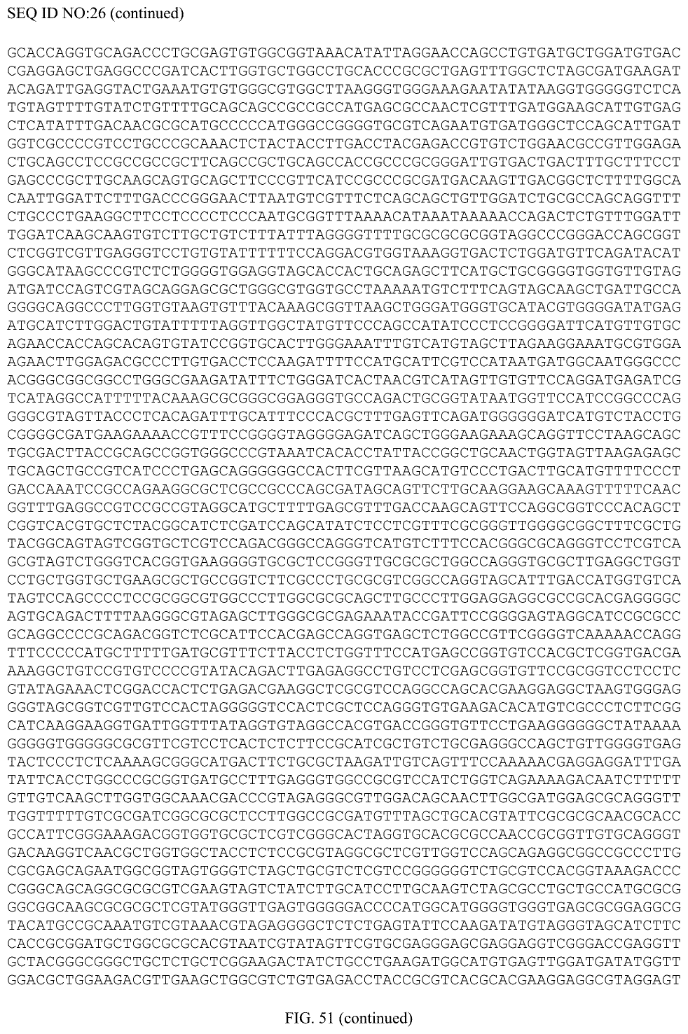

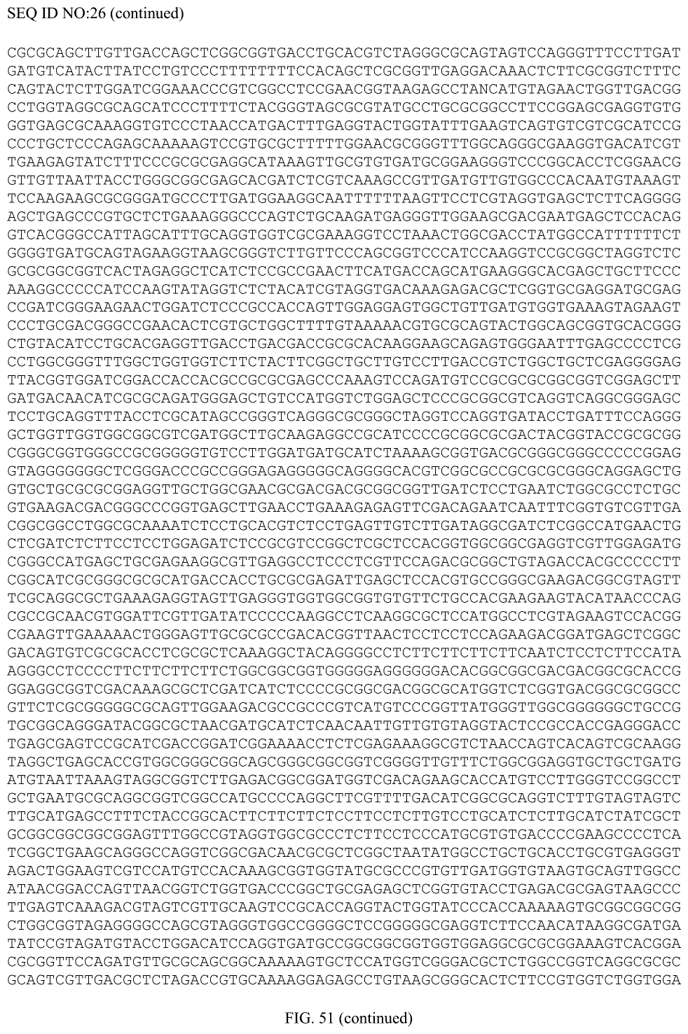

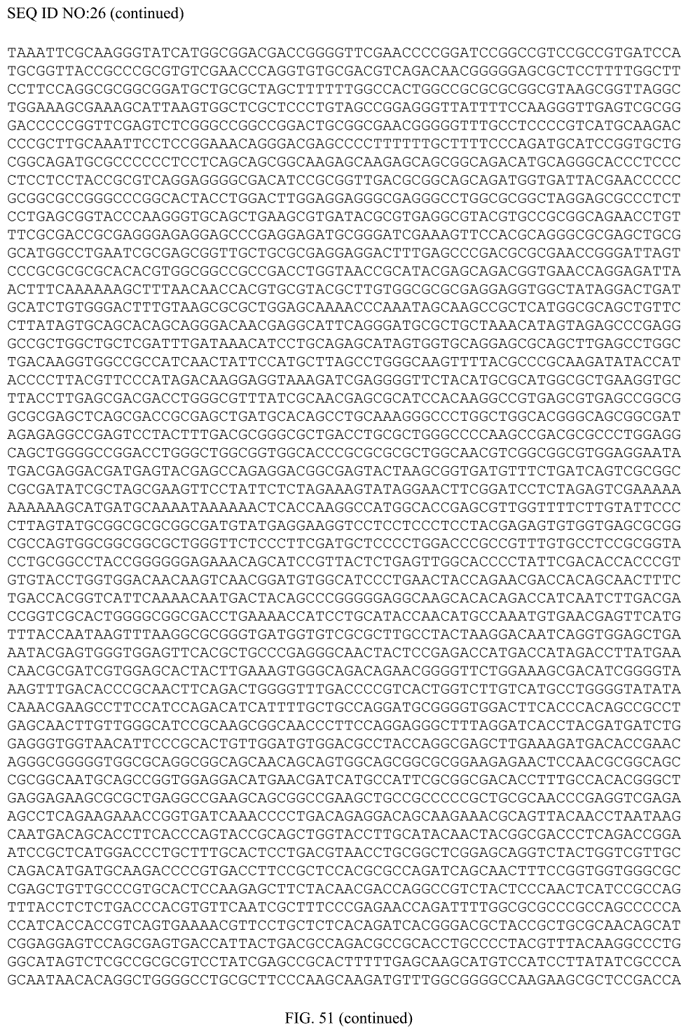

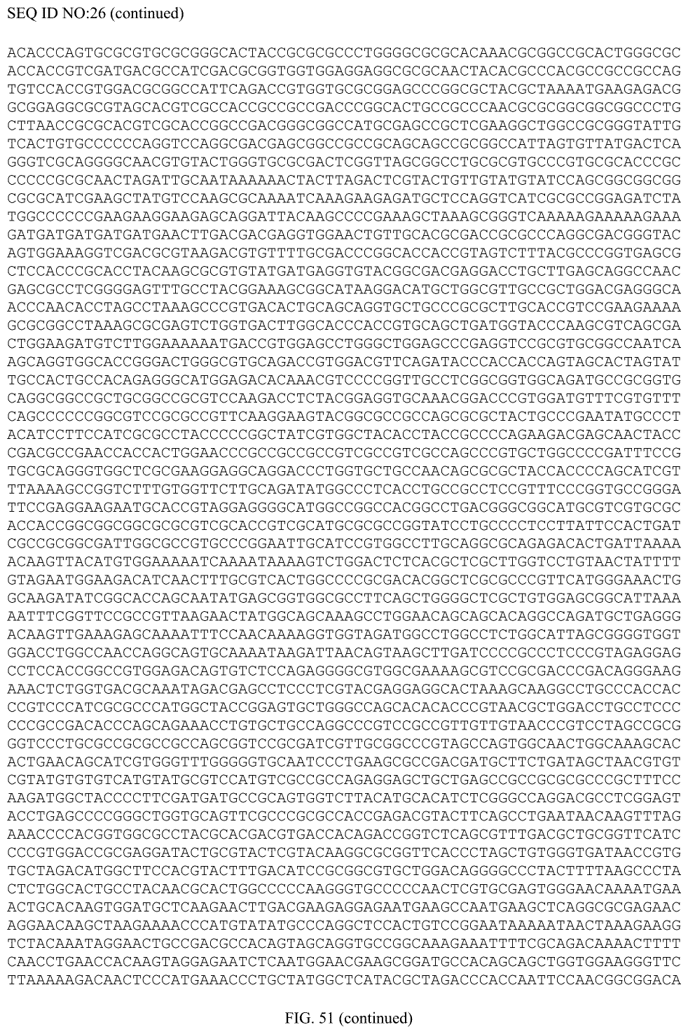

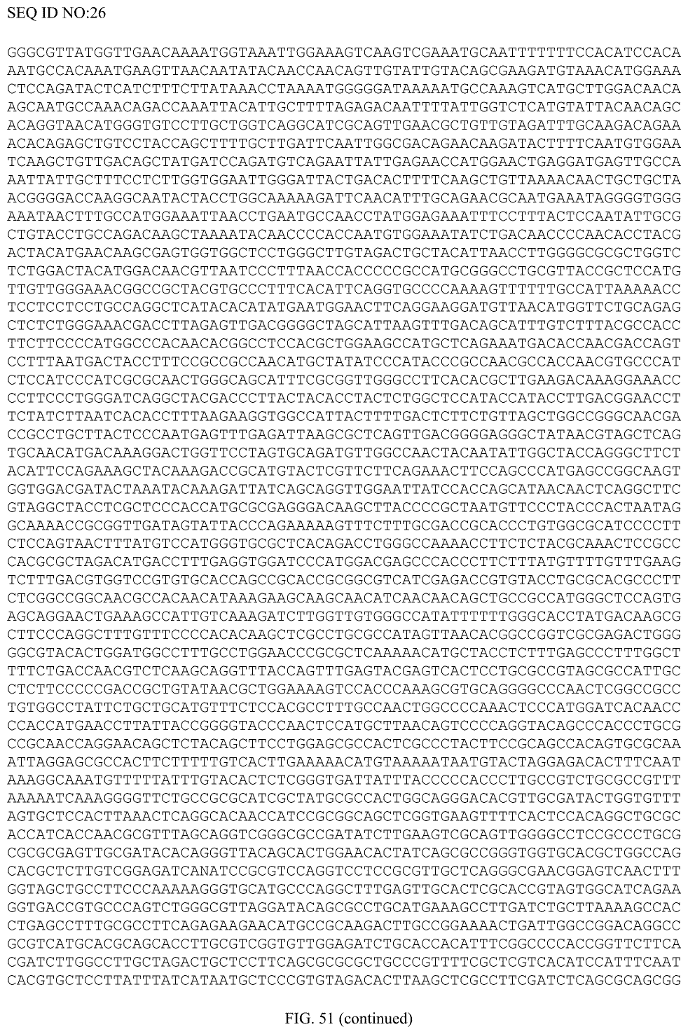

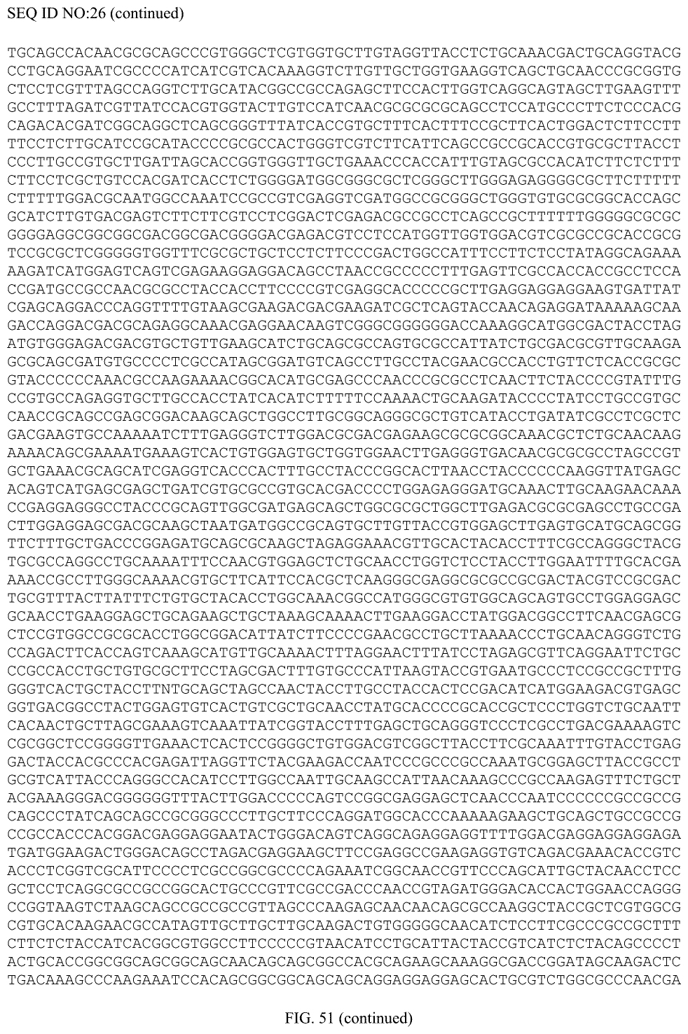

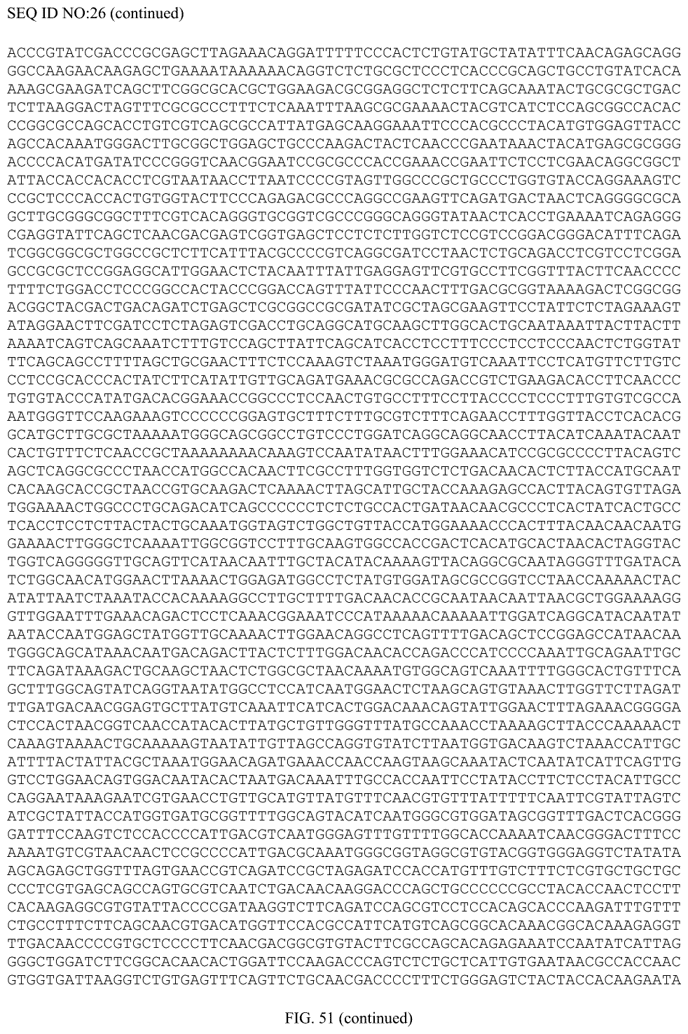

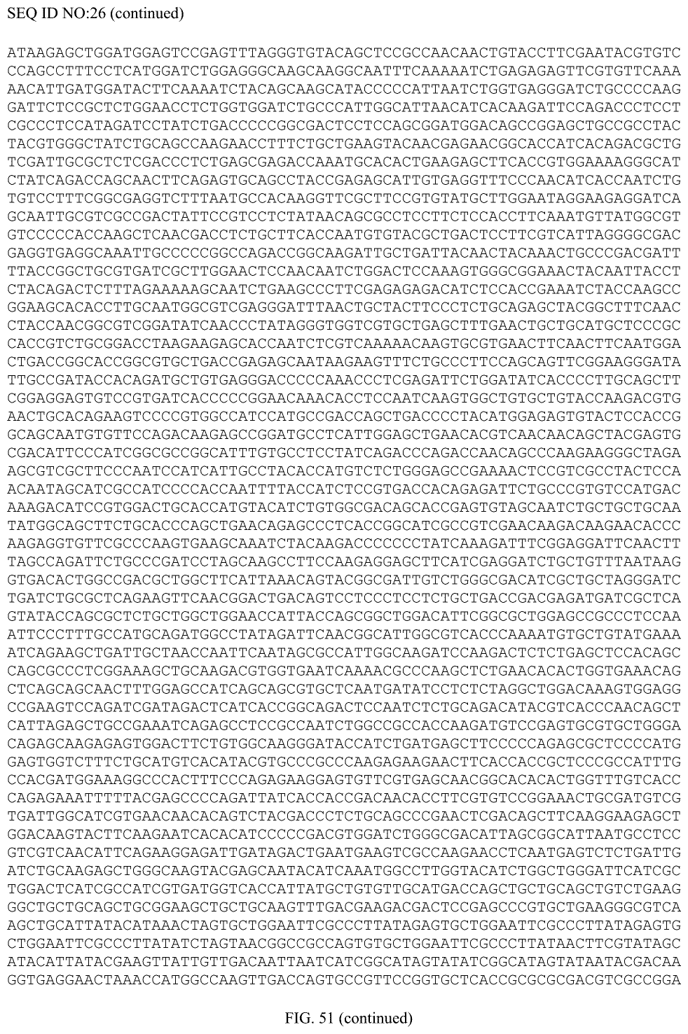

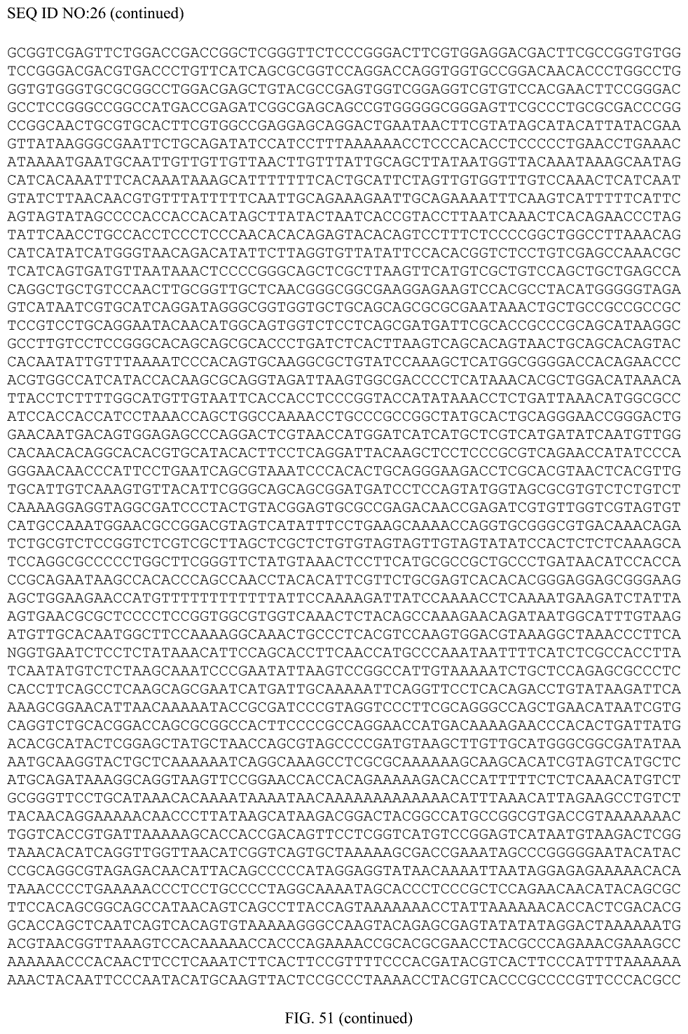

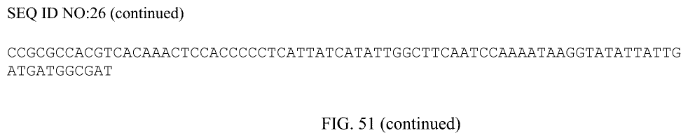

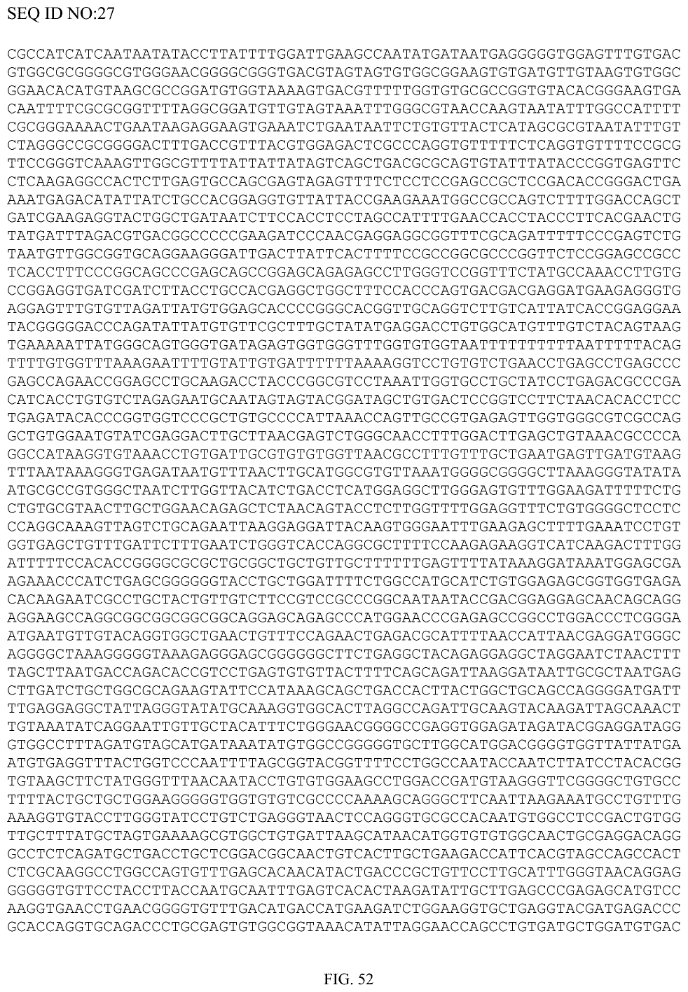

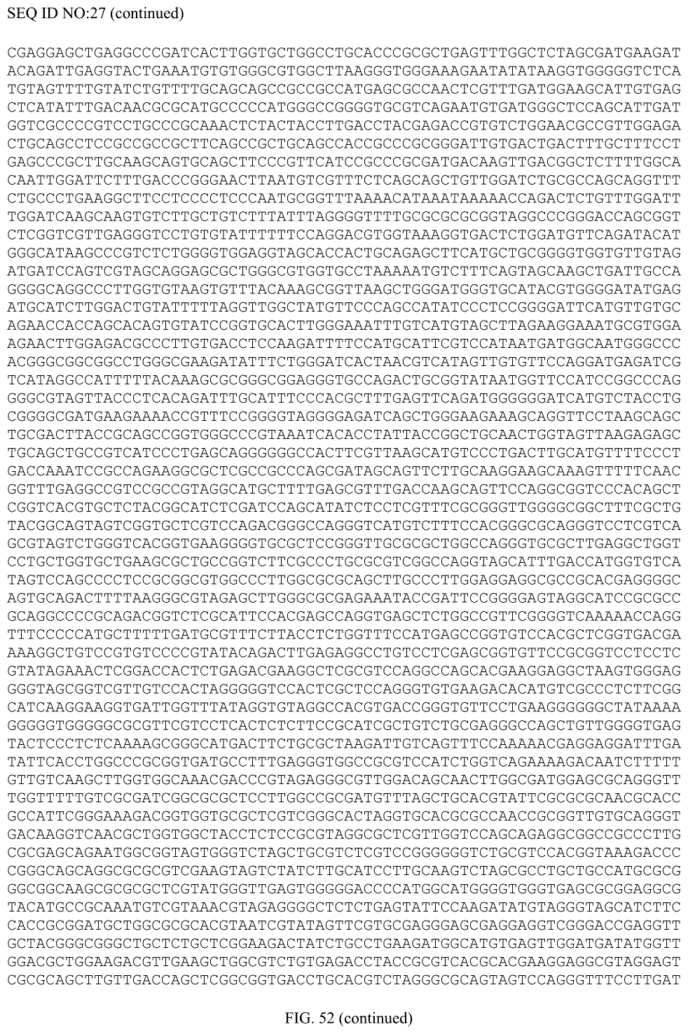

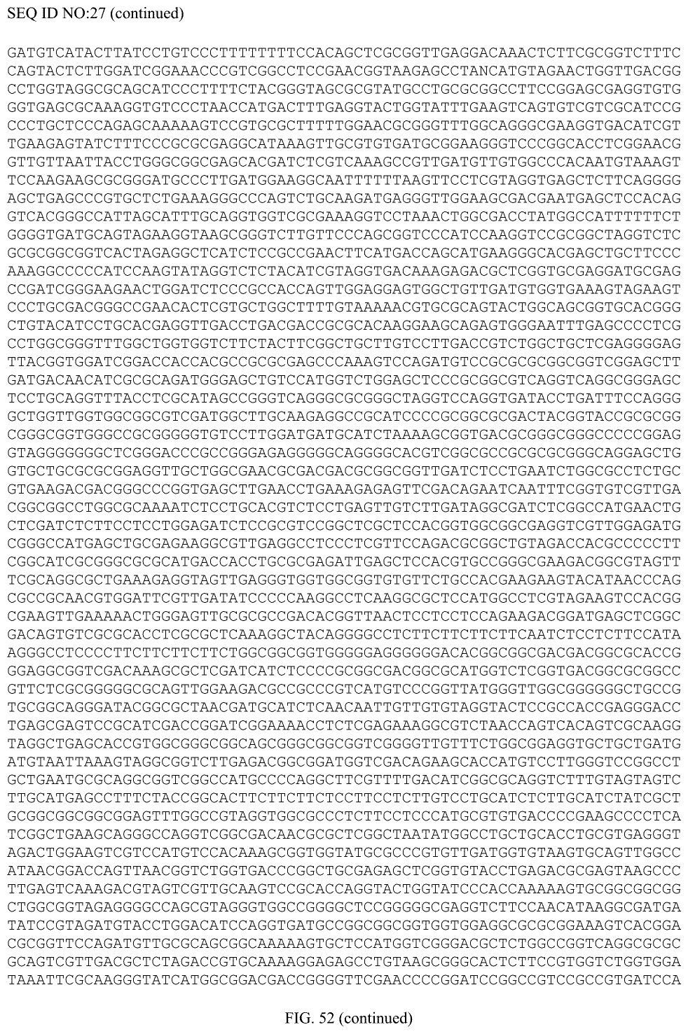

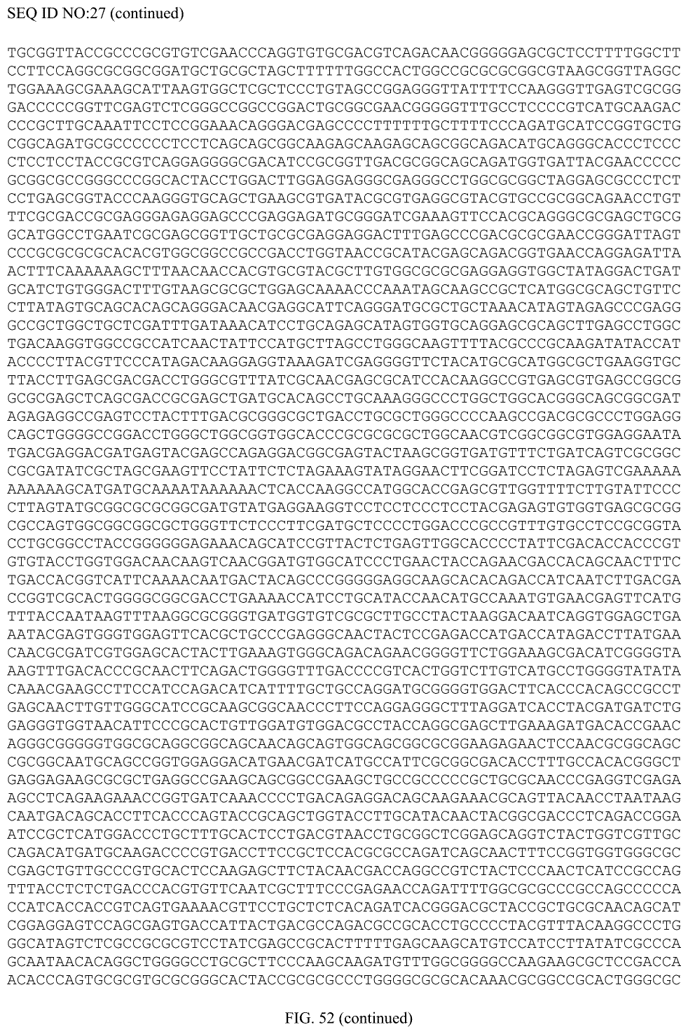

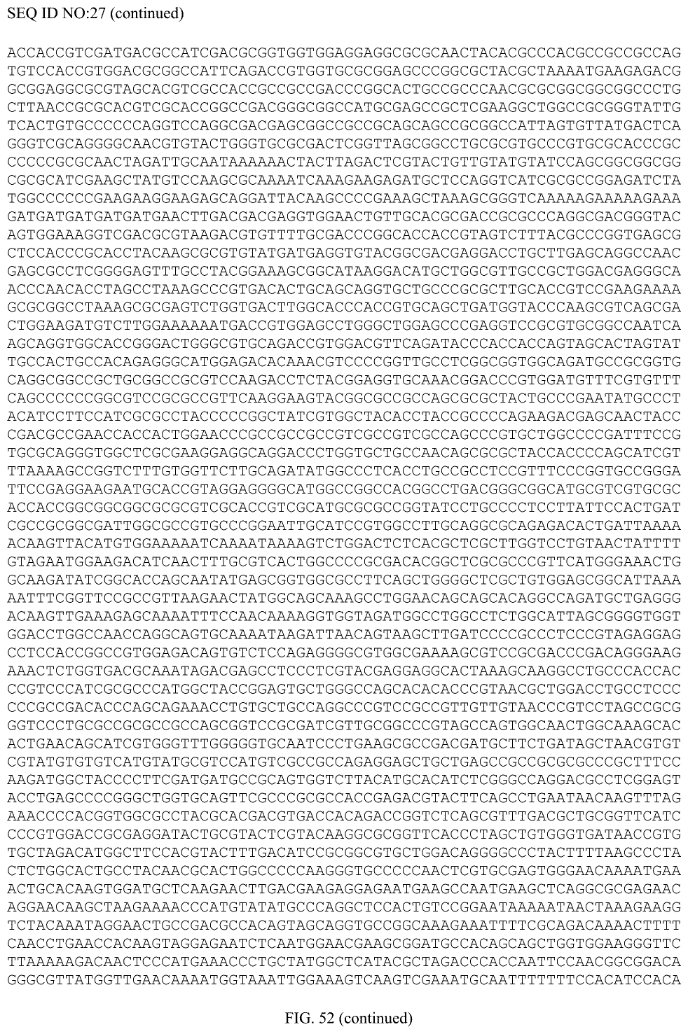

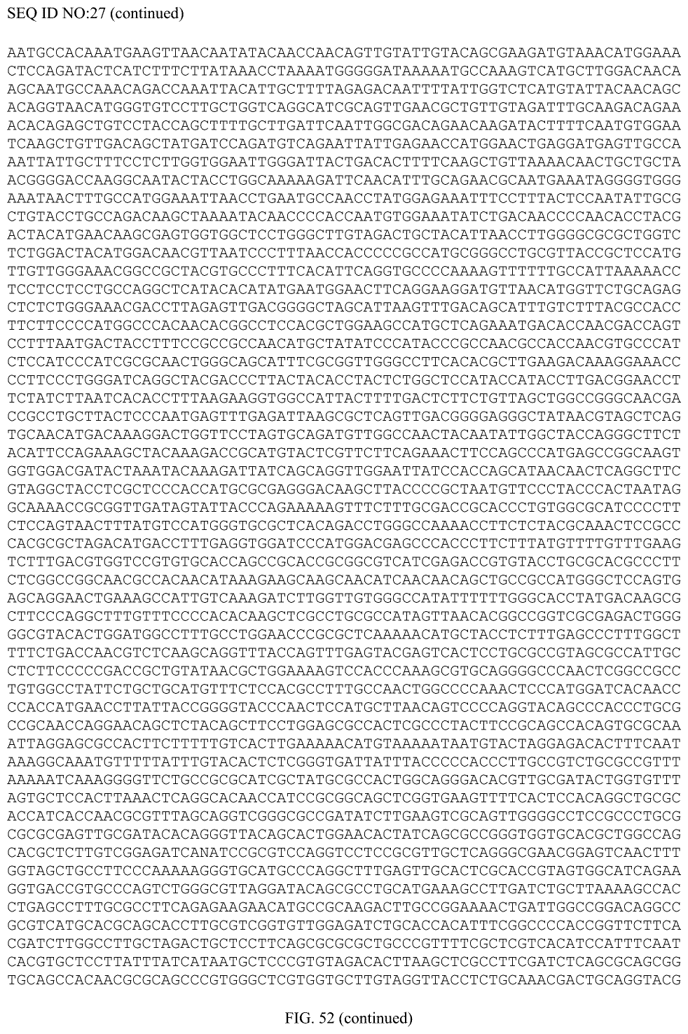

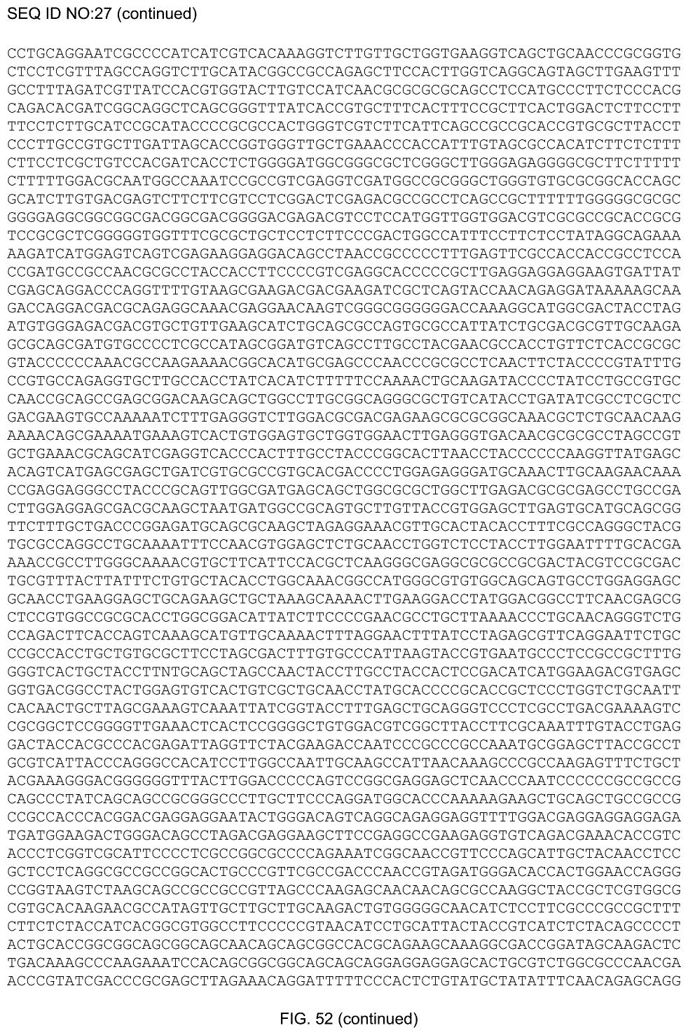

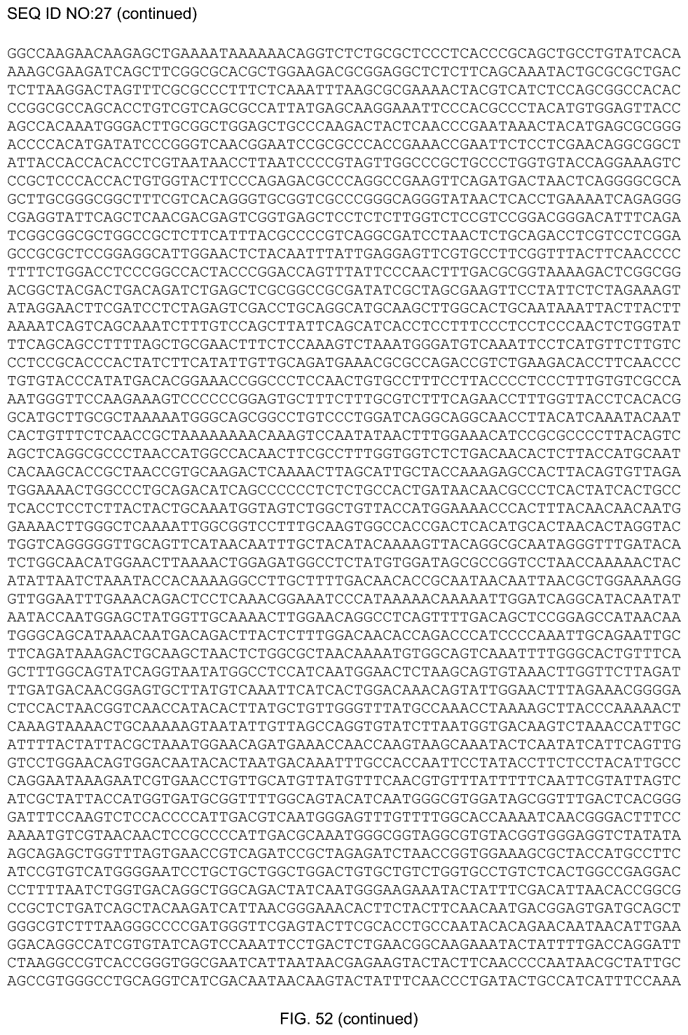

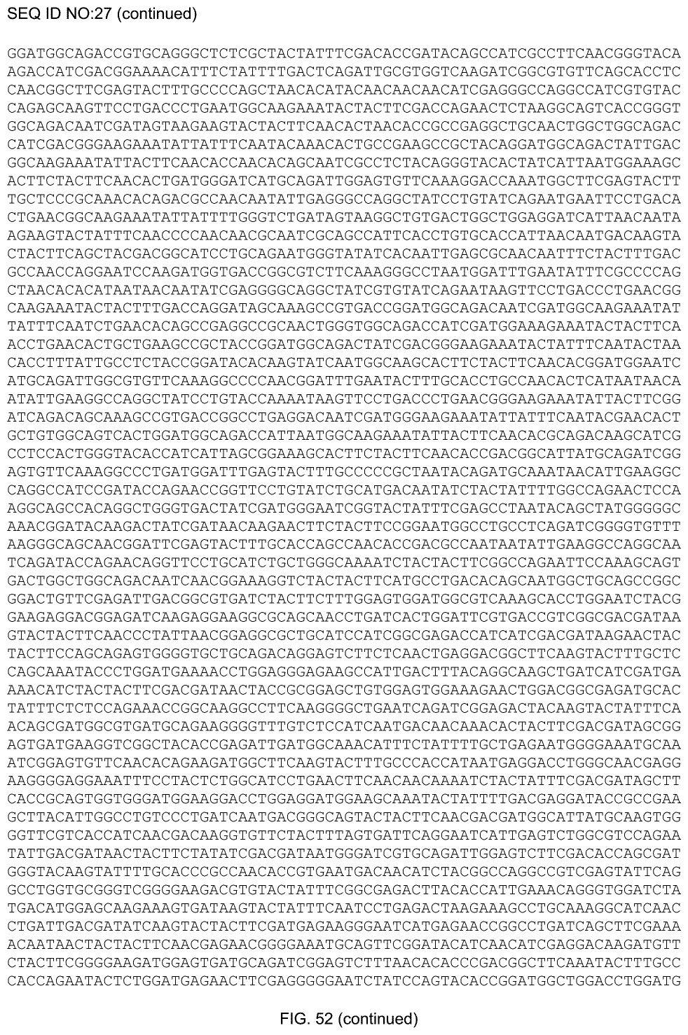

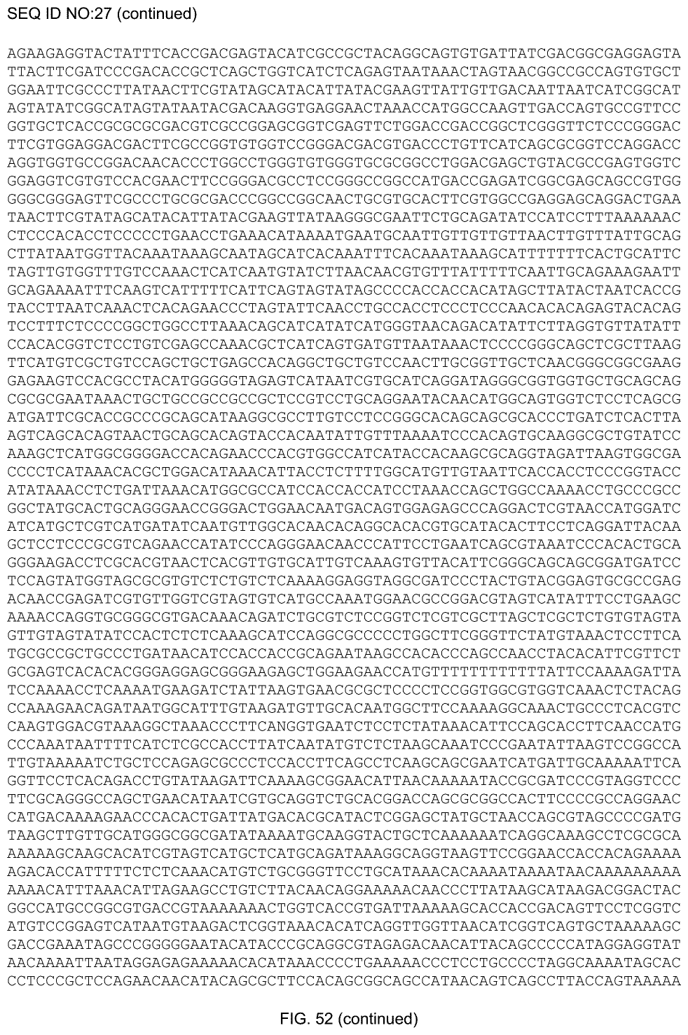

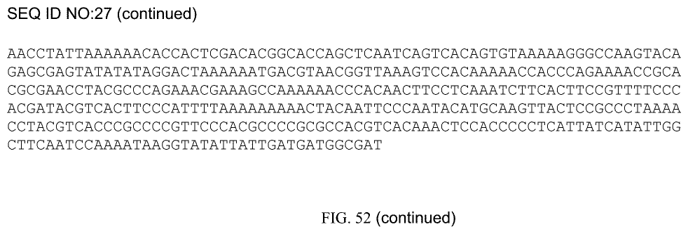

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) adjuvant polypeptides (e.g., nucleic acid that drives expression of one or more adjuvant polypeptides). For example, an adenovirus vector can include a nucleic acid sequence encoding one or more polypeptides that can enhance an immune response within a mammal. In some cases, an adjuvant polypeptide can be a cytokine. In some cases, an adjuvant polypeptide can be an immune stimulator. In some cases, an adjuvant polypeptide can be a toxin. In some cases, an adjuvant polypeptide can accelerate a systemic T cell response against a pathogen present within a mammal. In some cases, an adjuvant polypeptide can increase a concentration of antibodies against a pathogen at a site where the pathogen can enter a mammal's body. For example, an adjuvant polypeptide can increase a concentration of antibodies against a virus (e.g., a coronavirus) at a mucosal site where the virus can enter a mammal's body. Examples of adjuvant polypeptides that can be encoded by an adenovirus vectors encoding one or more immunogens described herein include, without limitation, granulocyte-macrophage colony-stimulating factor (GM-CSF) polypeptides, interleukin 4 (IL-4) polypeptides, interleukin 21 (IL-21) polypeptides, CD40 ligand (CD40L) polypeptides, 4-1BB ligand (4-1BBL) polypeptides, transforming growth factor beta (TGF-β) polypeptides, C. difficile toxin polypeptides (e.g., C. difficile TcdA polypeptides (see, e.g., SEQ ID NO:22), C. difficile TcdB polypeptides (see, e.g., SEQ ID NO:23), and/or the amino acid sequence set forth in SEQ ID NO:10 (see, e.g., FIG. 37)), and influenza polypeptides (e.g. N polypeptides, H polypeptides, M polypeptides, the amino acid sequence set forth in SEQ ID NO:20 (see, e.g., FIG. 47), and/or the amino acid sequence set forth in SEQ ID NO:21 (see, e.g., FIG. 48)). In some cases, an adjuvant polypeptide (e.g., SEQ ID NO:22) can be preceded by an AAT secretory sequence (e.g., MPSSVSWGILLLAGLCCLVPVSLAEDP; SEQ ID NO:28). In some cases, a nucleic acid sequence encoding an adjuvant polypeptide (e.g., SEQ ID NO:23) can be preceded by a cleavage site such as a synthetic furin cleavage site (e.g., RGRRSRGRRS; SEQ ID NO:29). Examples of nucleic acid sequences that can encoding an adjuvant polypeptide described herein include, without limitation, the nucleic acid sequence set forth in SEQ ID NO:24 (see, e.g., FIG. 49) and the nucleic acid sequence set forth in SEQ ID NO:25 (see, e.g., FIG. 50). In some cases, a nucleic acid sequence encoding an adjuvant polypeptide (e.g., SEQ ID NO:24) can be preceded by an AAT secretory sequence (e.g., ATGCCTTCATCCGTGTCATGGGGAATCCTGCTGCTGGCTGGACTGTGCTGTCTGGT GCCTGTCTCACTGGCCGAGGACCCT; SEQ ID NO:30). In some cases, a nucleic acid sequence encoding an adjuvant polypeptide (e.g., SEQ ID NO:25) can be preceded by a cleavage site such as a synthetic furin cleavage site (e.g., AGAGGACGGAGATCAAGAGGAAGGCGCAGC; SEQ ID NO:31). An adjuvant polypeptide can be a full-length polypeptide or a fragment of an adjuvant polypeptide described herein provided that the fragment has the ability to enhance an immune response (e.g., a biologically active fragment). In some cases, an adjuvant polypeptide can be as described elsewhere (see, e.g., Matchett et al., Vaccines, 8(1):64 (2020)).

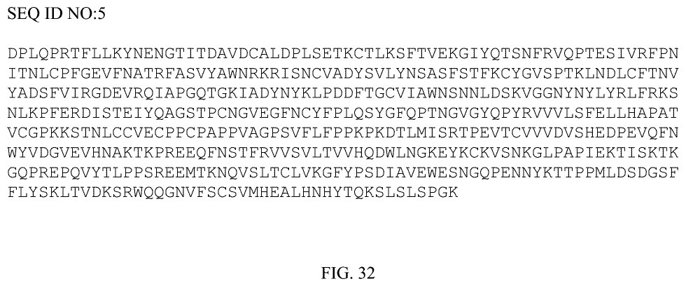

In some cases, an adenovirus vector provided herein (e.g., an adenovirus vector encoding one or more immunogens) can encode (e.g., can be designed to encode) a polypeptide that includes an immunogen fused to an adjuvant polypeptide. For example, a nucleic acid sequence encoding an immunogen can be fused to a nucleic acid sequence encoding an adjuvant polypeptide (e.g., such that the encoded immunogen is fused to the encoded adjuvant polypeptide). An example of an immunogen fused to an adjuvant polypeptide that can be encoded by an adenovirus vectors encoding one or more immunogens described herein include, without limitation, the amino acid sequence set forth in SEQ ID NO:5 (see, e.g., FIG. 32).

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) chaff polypeptides (e.g., nucleic acid that drives expression of one or more chaff polypeptides). A chaff polypeptide can be a full-length polypeptide or a fragment thereof provided that it reduces the rate of entry or inhibits entry of a pathogen into a cell within a mammal. In some cases, a chaff polypeptide can be a soluble polypeptide. For example, a soluble chaff polypeptide can be a full-length chaff polypeptide or a fragment of a chaff polypeptide that lacks a transmembrane domain. For example, a soluble chaff polypeptide can include an ectodomain of a chaff polypeptide. In some cases, a chaff polypeptide can target (e.g., target and bind to) a particular pathogen (e.g., a virus such as a coronavirus) to reduce the rate of entry or inhibit entry of the pathogen into a cell within a mammal. In some cases, a chaff polypeptide can target (e.g., target and bind to) two, three, four, five, six, or more different pathogens. In some cases, a chaff polypeptide can include one or more mutations (e.g., inactivating mutations). Examples of chaff polypeptides that can be encoded by an adenovirus vectors encoding one or more immunogens described herein include, without limitation, full-length ACE2 polypeptides and fragments thereof, full-length CD13 polypeptides and fragments thereof, full-length CEACAM1 polypeptides and fragments thereof, full-length sialydated polypeptides and fragments thereof, full-length CD46 polypeptides and fragments thereof, full-length nestin polypeptides and fragments thereof, the amino acid sequence set forth in SEQ ID NO:8 (see, e.g., FIG. 37), and the amino acid sequence set forth in SEQ ID NO:9 (see, e.g., FIG. 38).

In some cases, an adenovirus vector provided herein (e.g., an adenovirus vector encoding one or more immunogens) can encode (e.g., can be designed to encode) a polypeptide that includes an immunogen fused to a chaff polypeptide. For example, a nucleic acid sequence encoding an immunogen can include a nucleic acid sequence encoding a chaff polypeptide (e.g., such that the encoded immunogen is fused to the encoded chaff polypeptide).

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) marker polypeptides (e.g., nucleic acid that drives expression of one or more marker polypeptides). Examples of marker polypeptides that can be encoded by an adenovirus vector encoding one or more immunogens described herein include, without limitation, fluorescent polypeptides (e.g., GFP, RFP, CFP, and YFP), streptavidin polypeptides, Cre recombinase polypeptides, Cas polypeptides, luciferase polypeptides, betagalactosidase polypeptides, and sodium iodide symporter polypeptides.

In some cases, an adenovirus vector provided herein (e.g., an adenovirus vector encoding one or more immunogens) can include a nucleic acid sequence encoding one or more (e.g., one, two, three, four, five, six, seven, eight, nine, ten, or more) polypeptides that can form a multimer (e.g., nucleic acid that drives expression of one or more polypeptides that can form a multimer). Examples of polypeptides that can form a multimer that can be encoded by an adenovirus vectors encoding one or more immunogens described herein include, without limitation, immunoglobulin constant region polypeptides (e.g., an Ig polypeptide), streptavidin polypeptides, and sigma coil polypeptides.

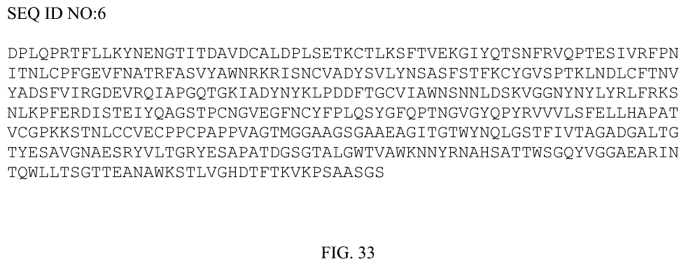

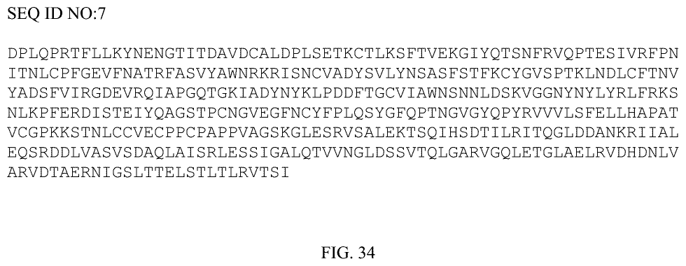

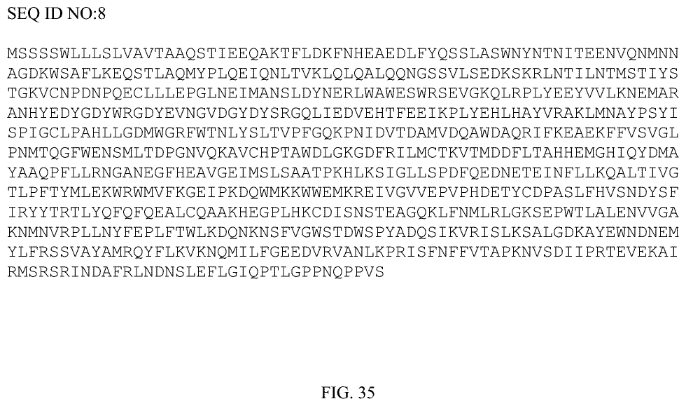

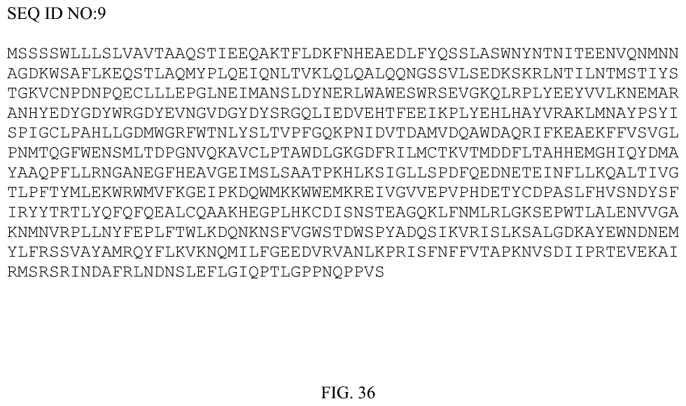

In some cases, an adenovirus vector provided herein (e.g., an adenovirus vector encoding one or more immunogens) can encode (e.g., can be designed to encode) a polypeptide that includes an immunogen fused to a polypeptide that can form a multimer. For example, a nucleic acid sequence encoding an immunogen can include a nucleic acid sequence encoding a polypeptide that can form a multimer (e.g., such that the encoded immunogen is fused to the encoded polypeptide that can form a multimer). Examples of immunogens fused to a polypeptide that can form a multimer that can be encoded by an adenovirus vectors encoding one or more immunogens described herein include, without limitation, the amino acid sequence set forth in SEQ ID NO:5 (see, e.g., FIG. 32), the amino acid sequence set forth in SEQ ID NO:6 (see, e.g., FIG. 33), and the amino acid sequence set forth in SEQ ID NO:7 (sec, e.g., FIG. 34).

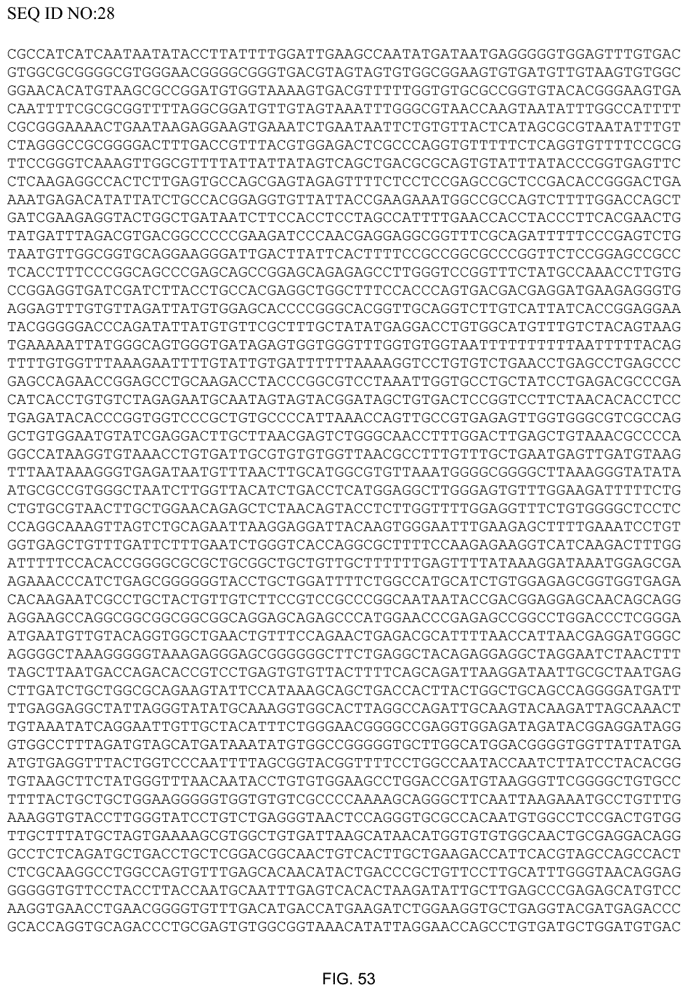

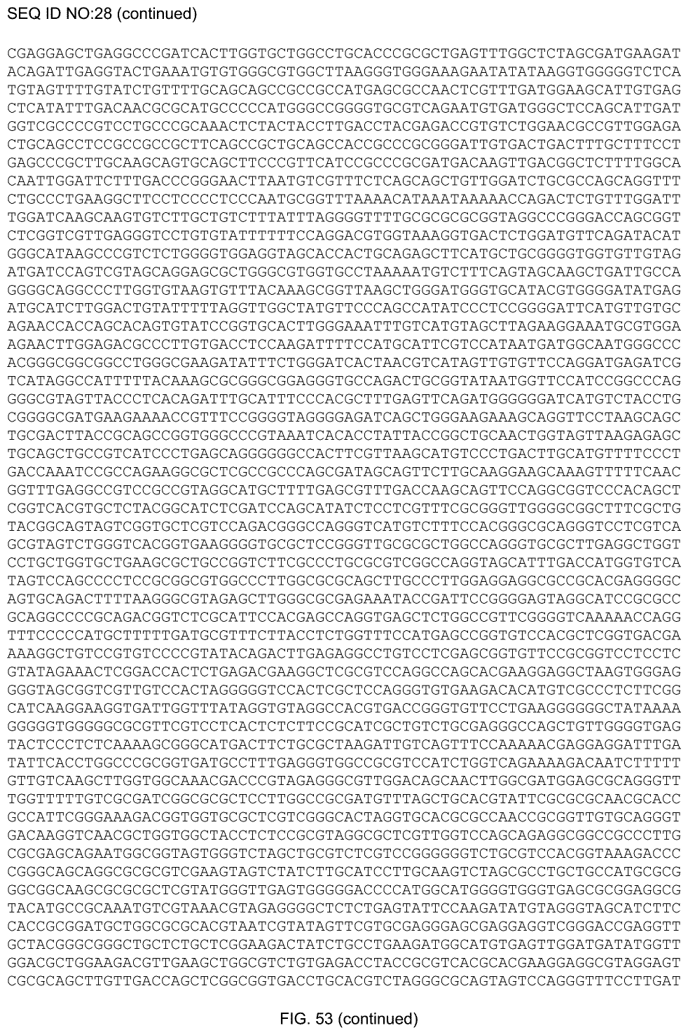

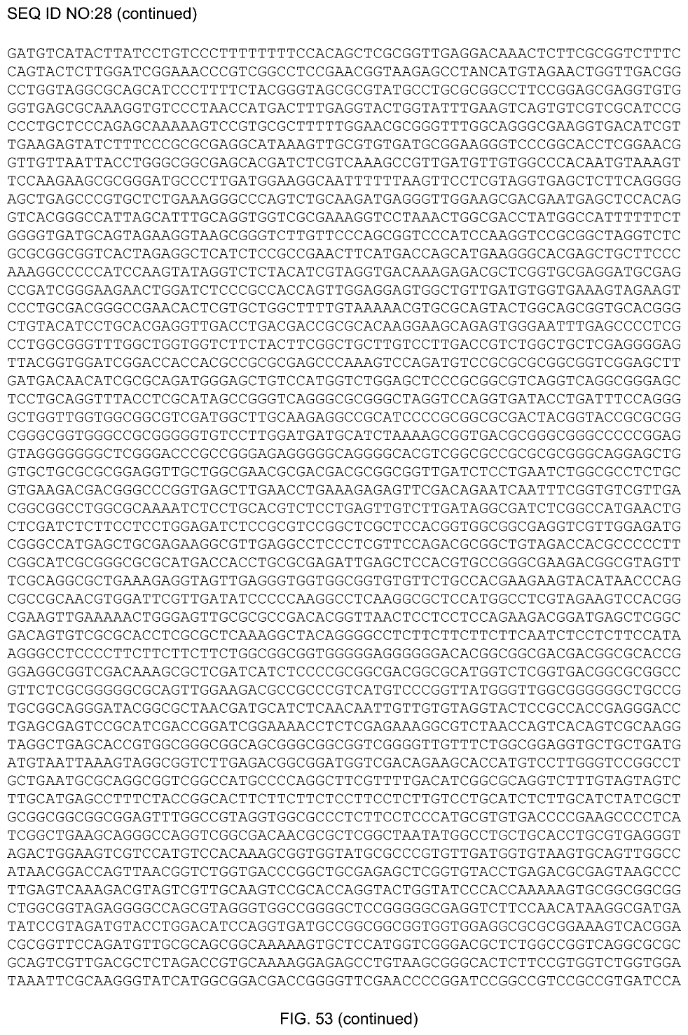

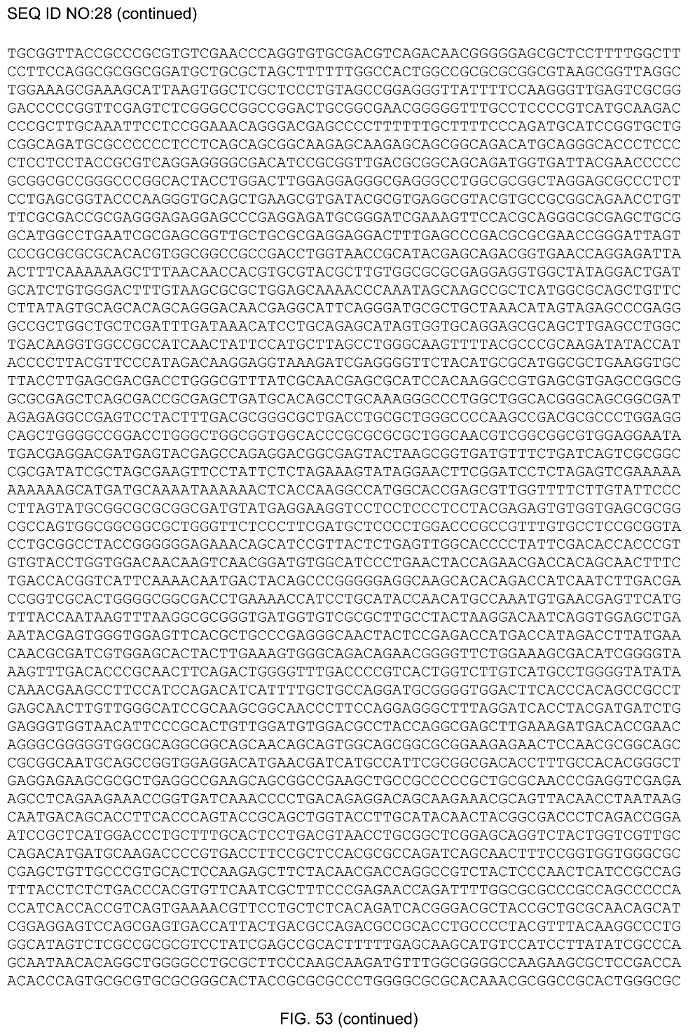

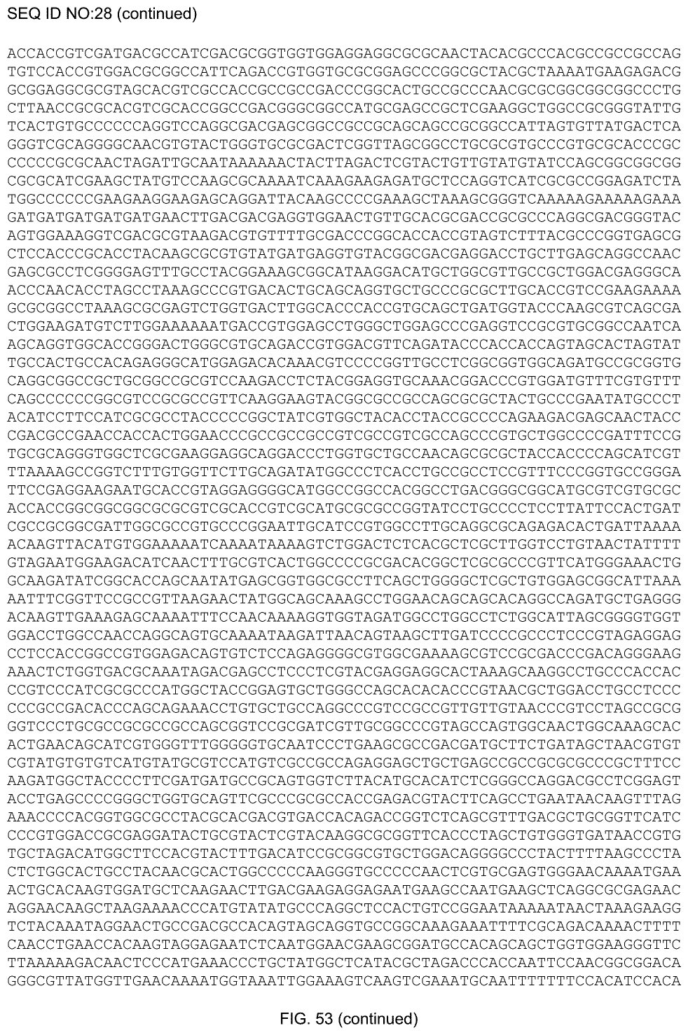

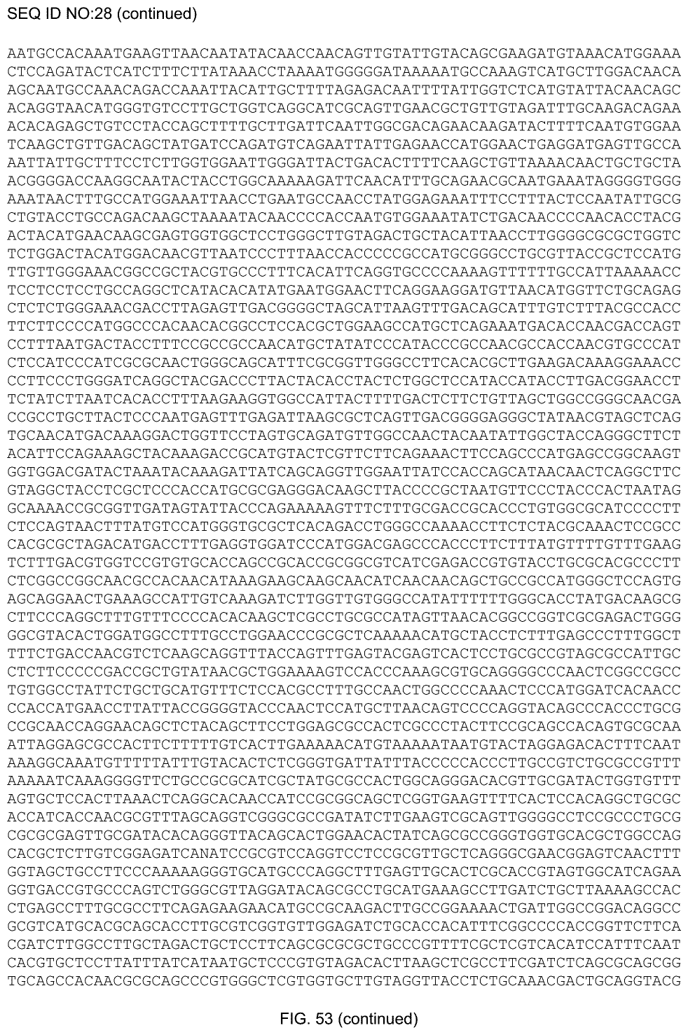

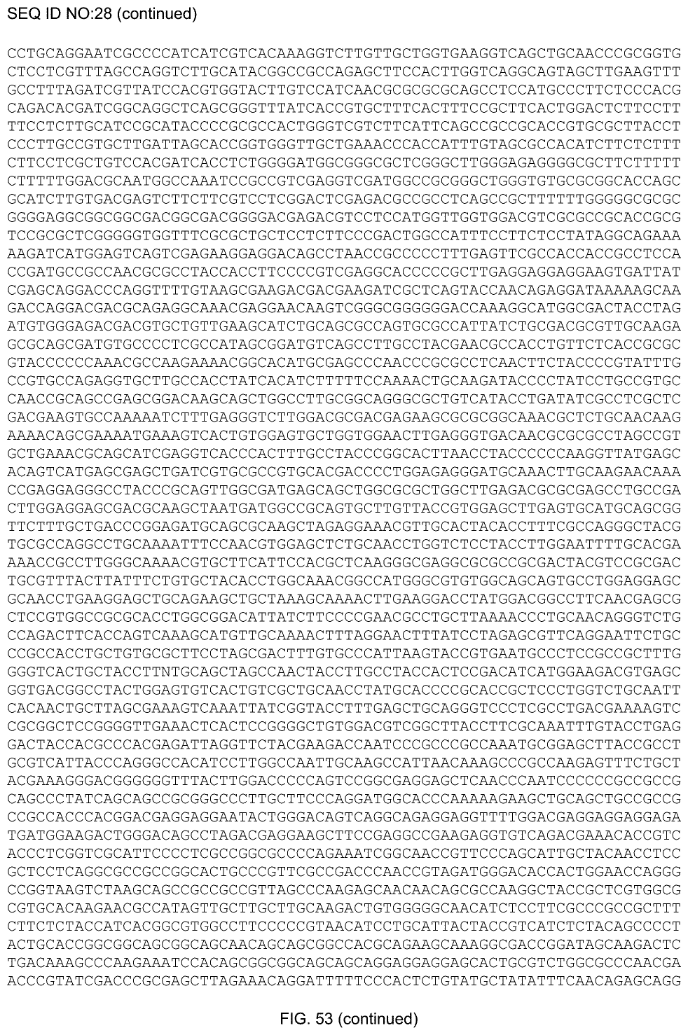

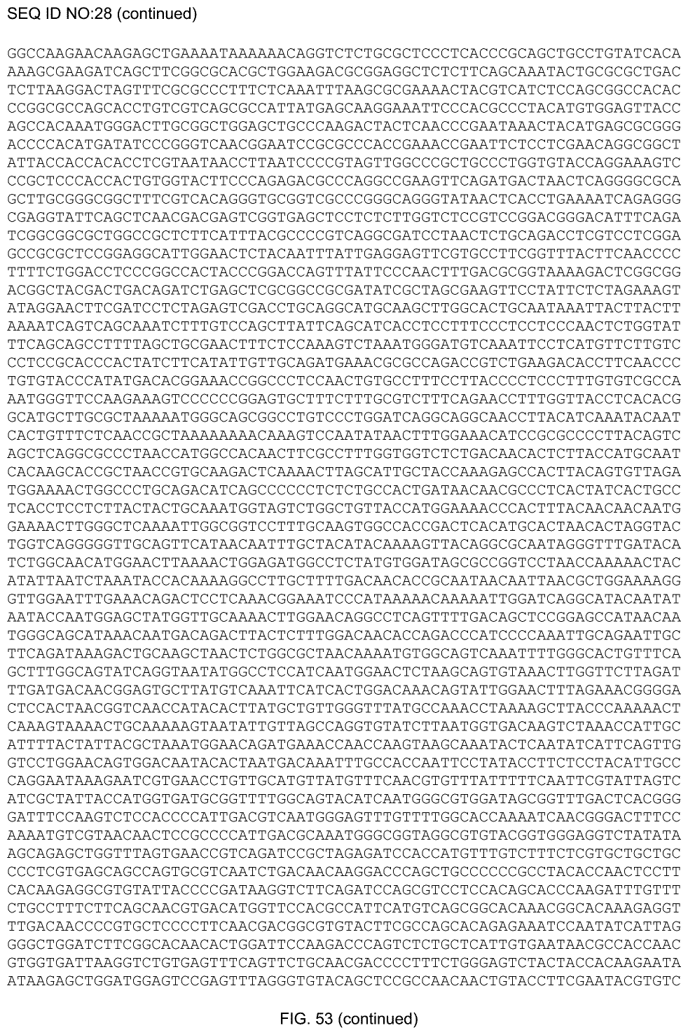

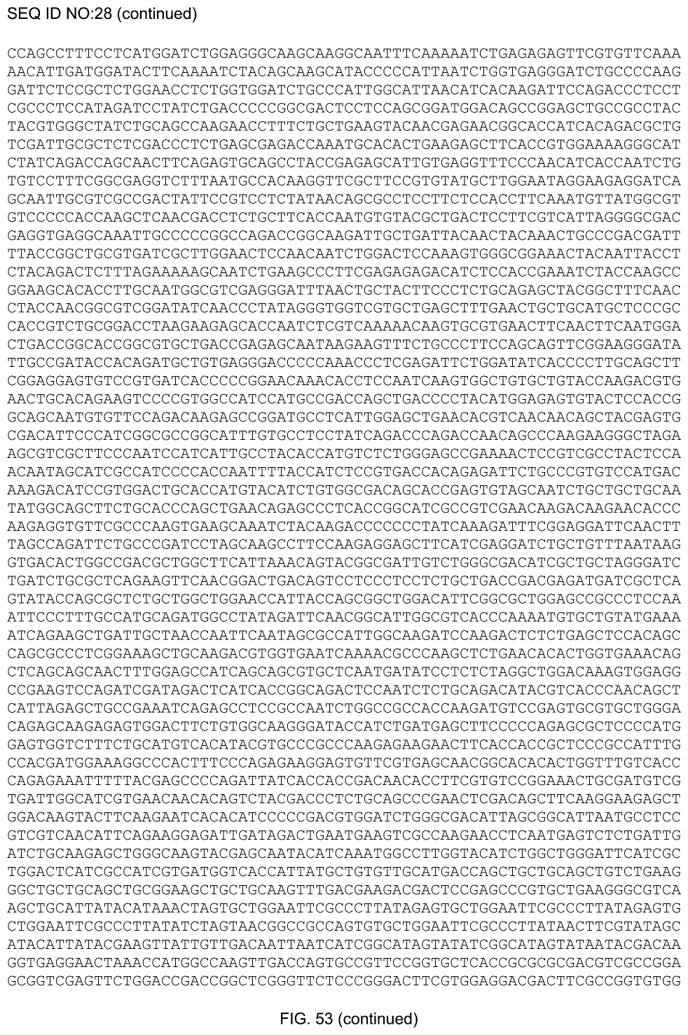

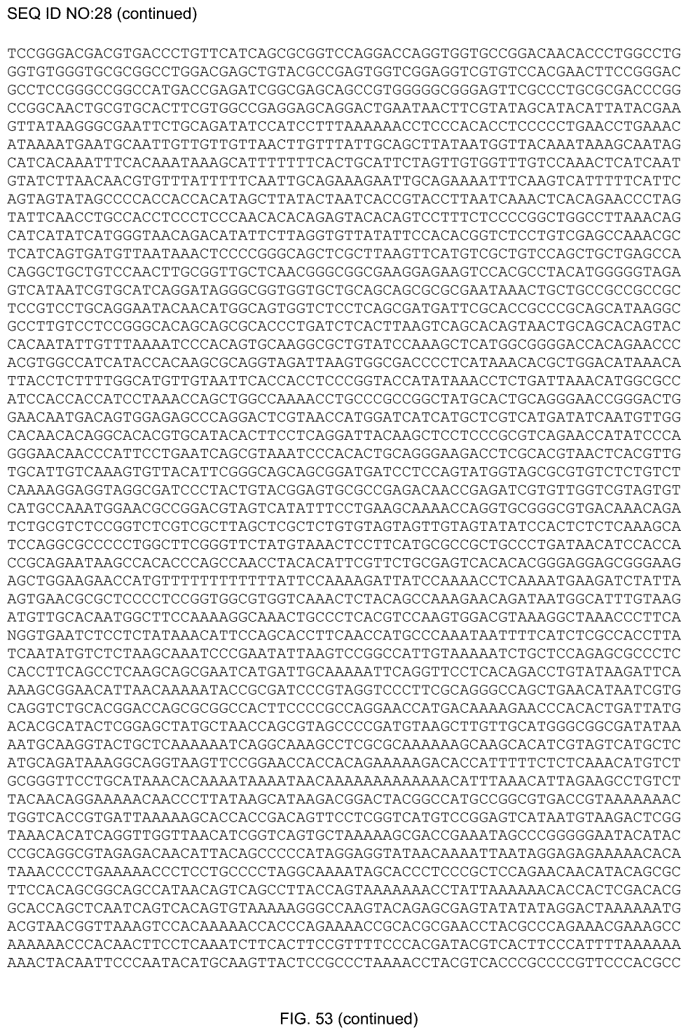

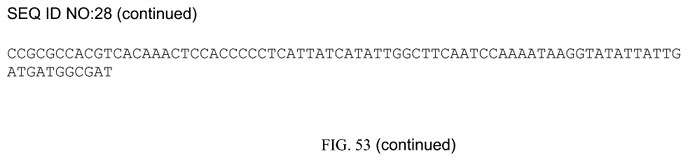

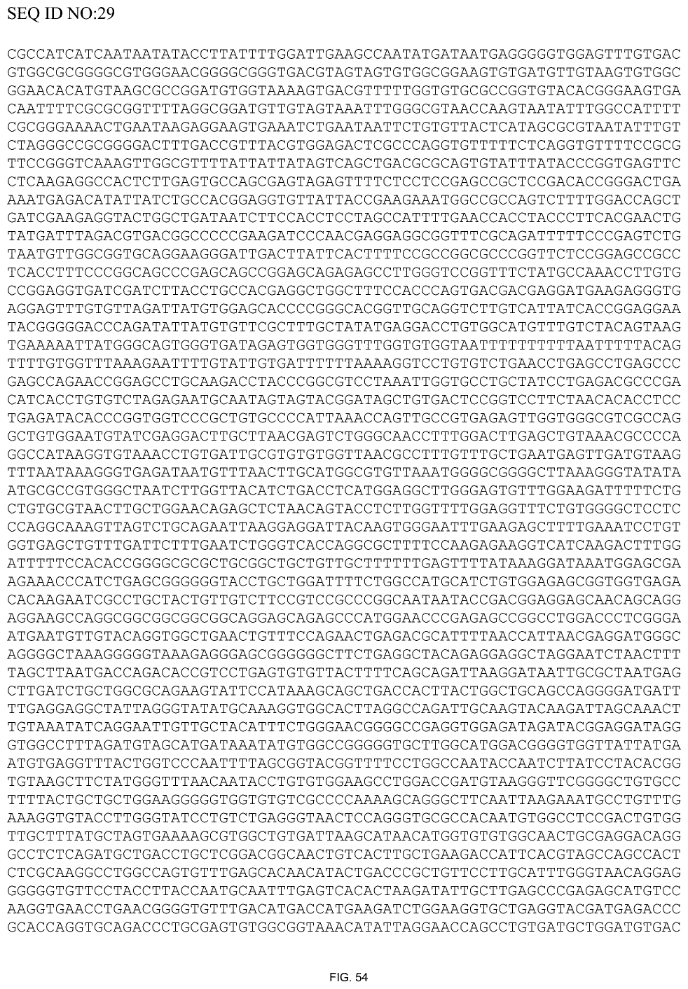

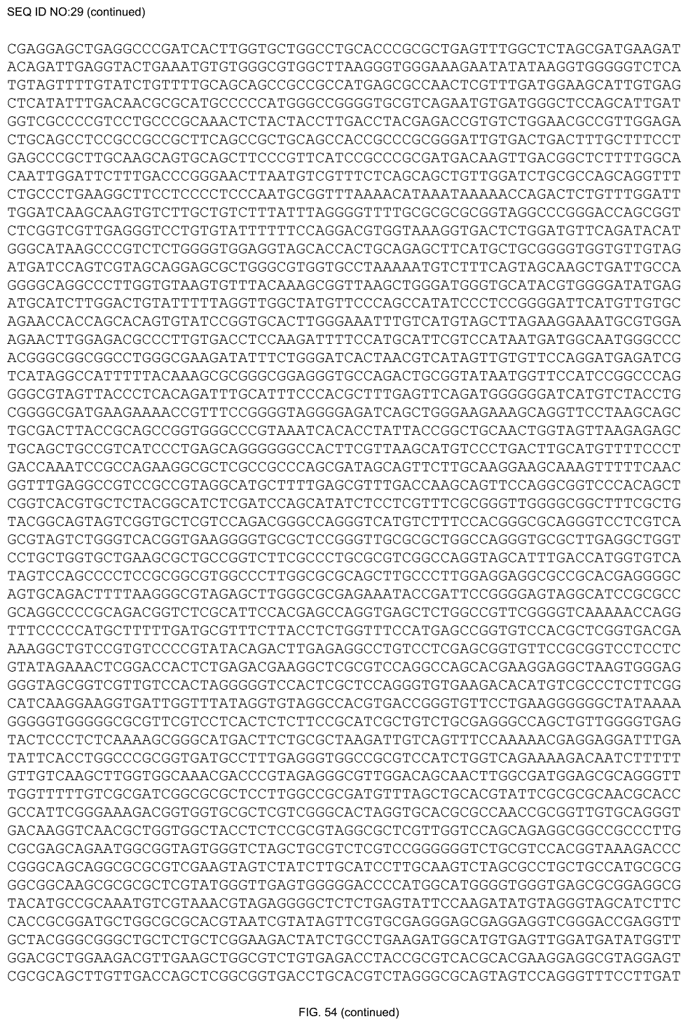

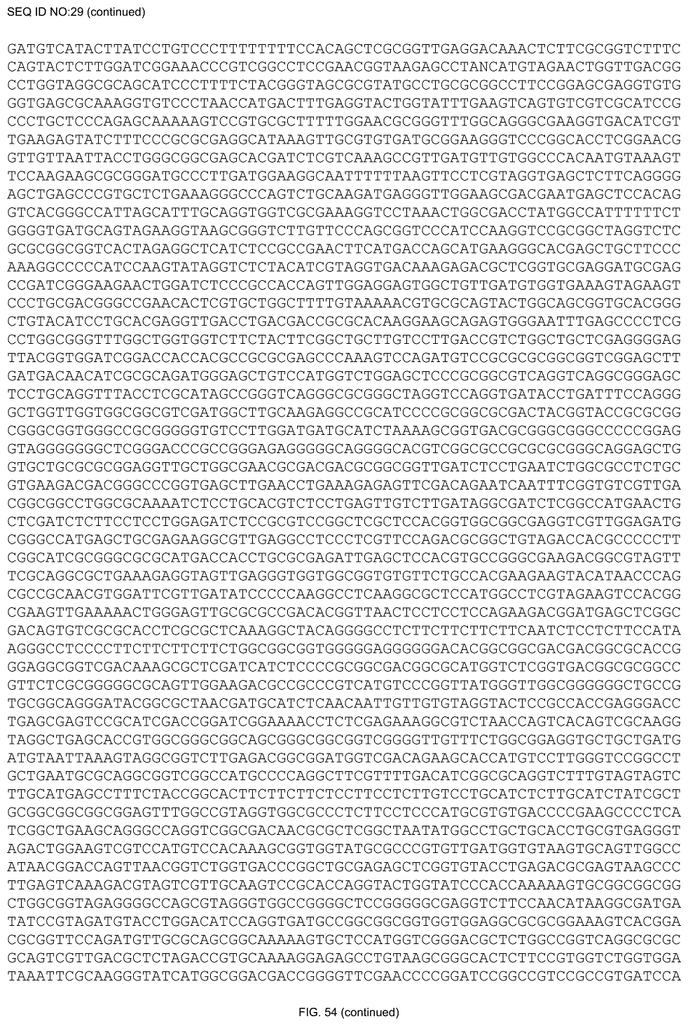

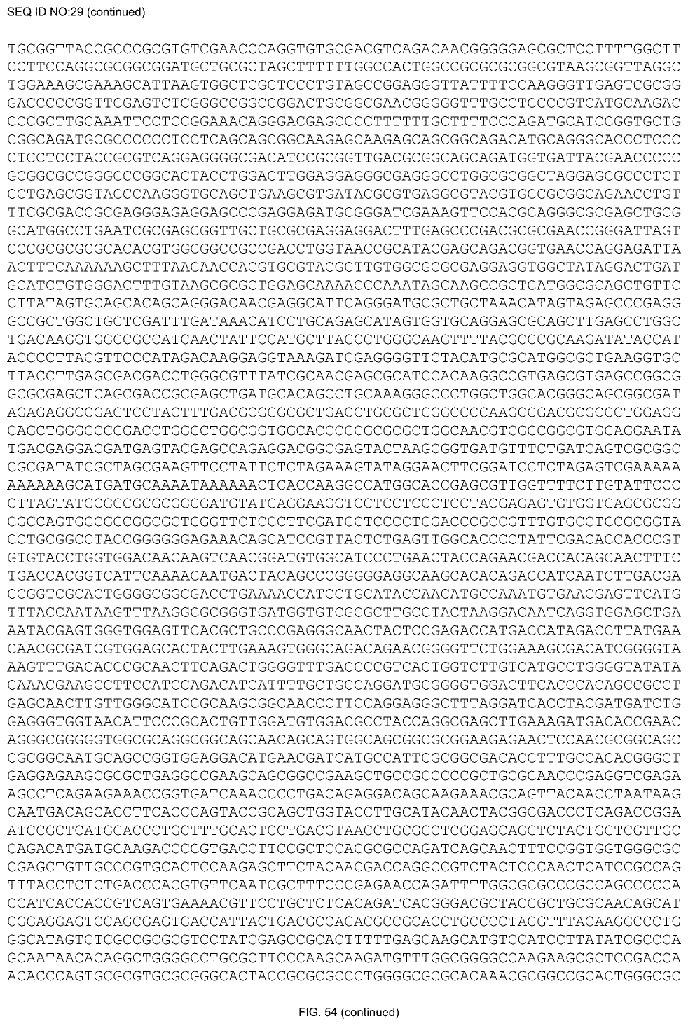

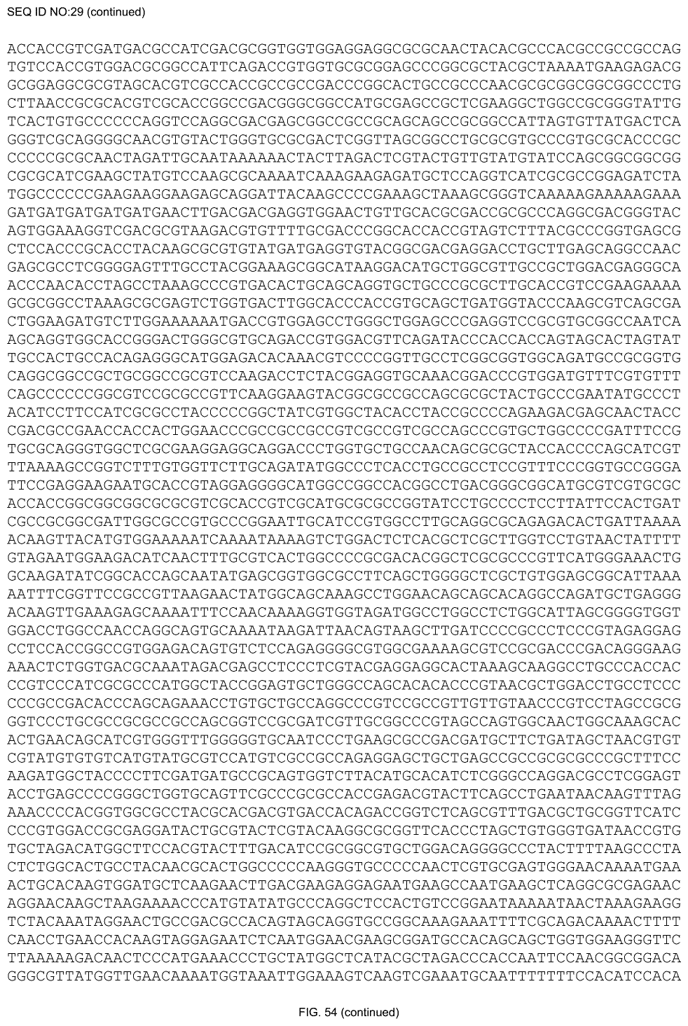

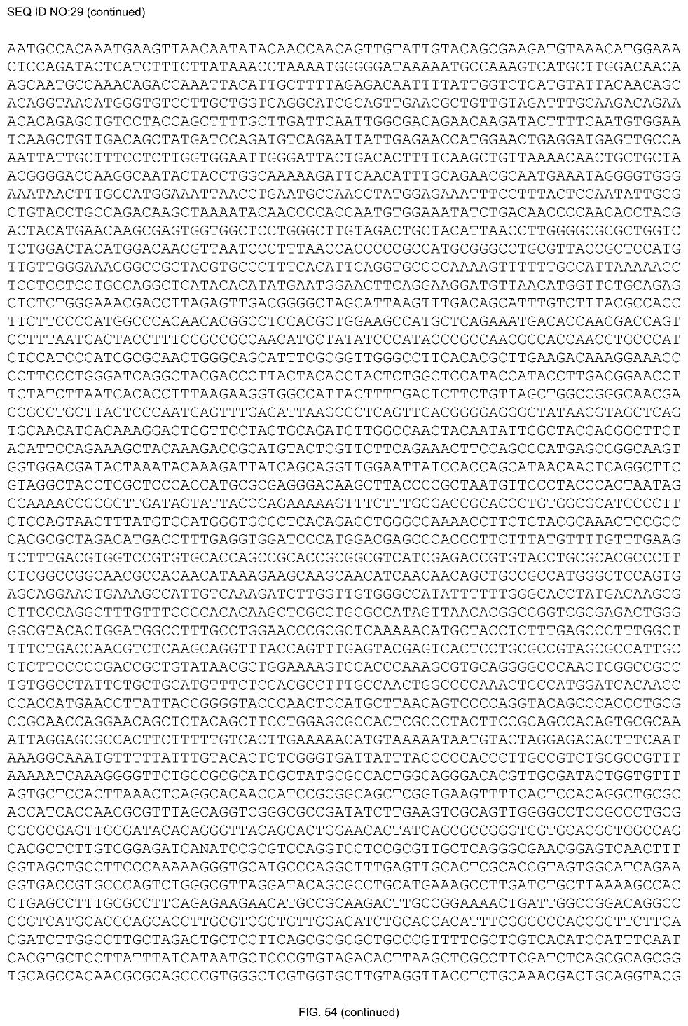

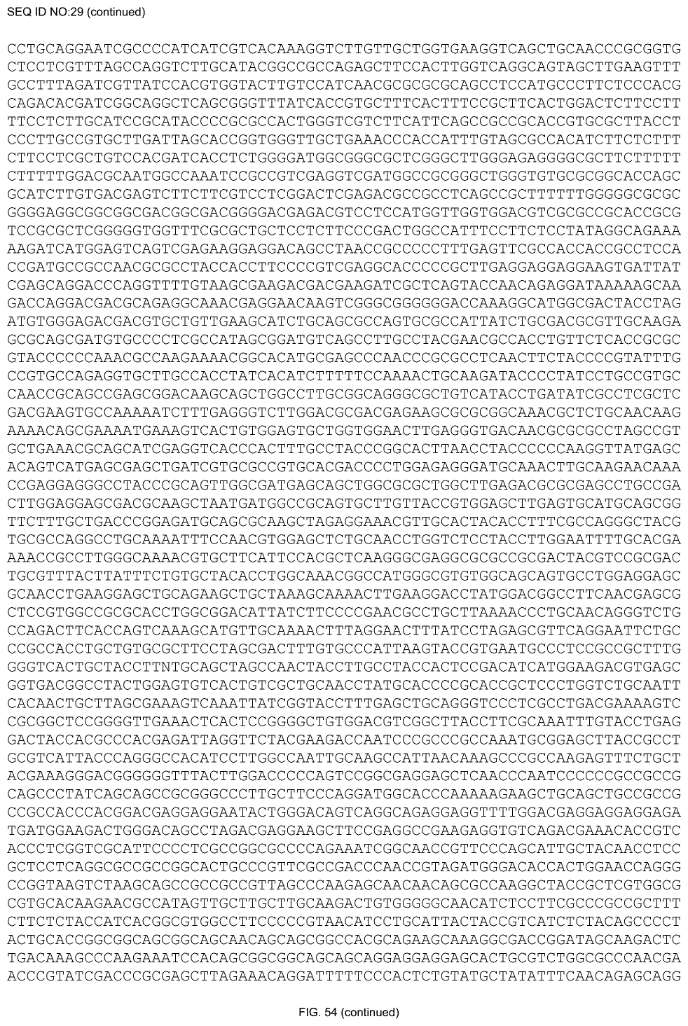

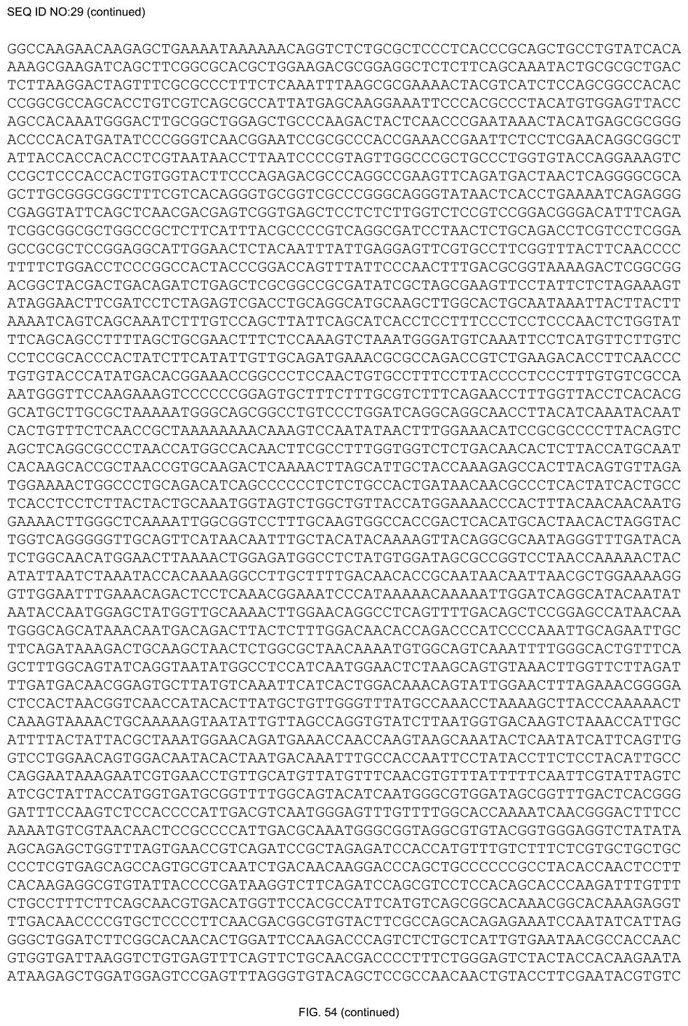

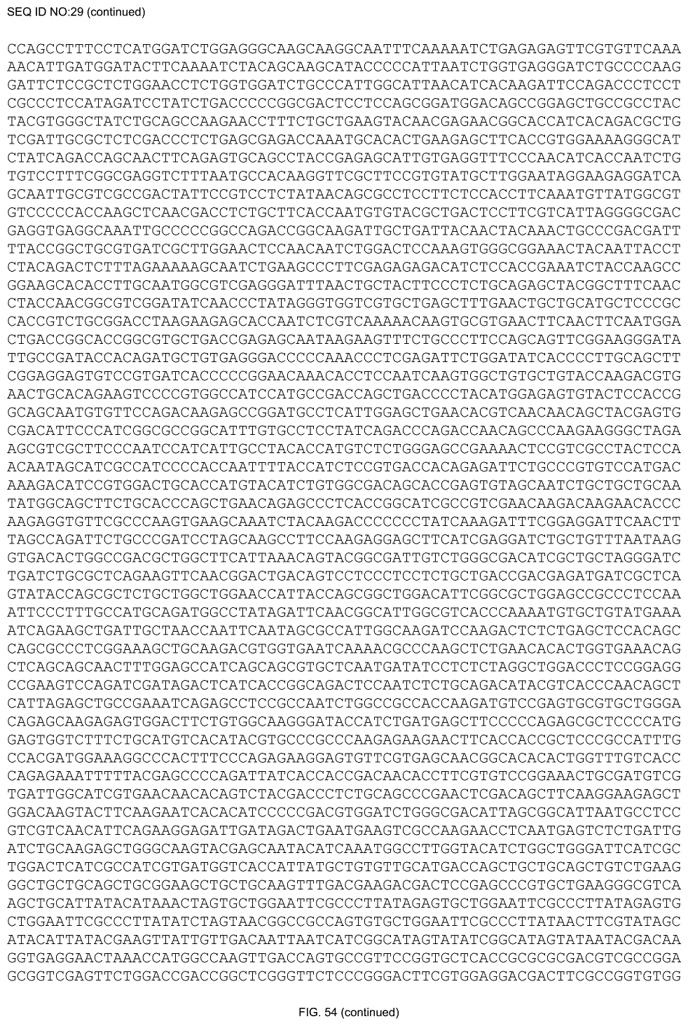

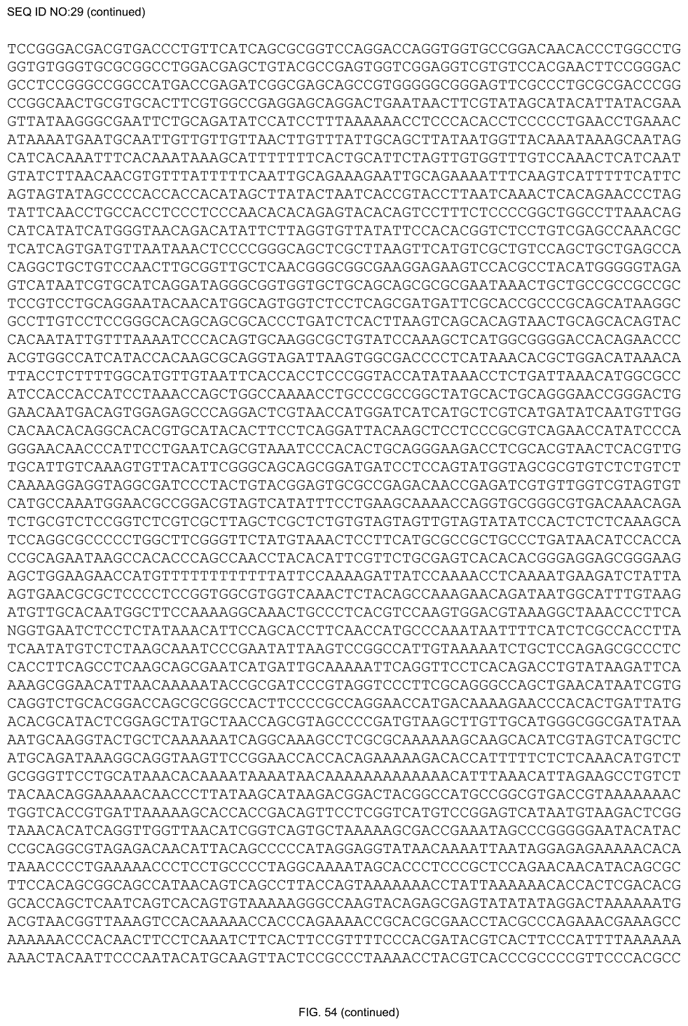

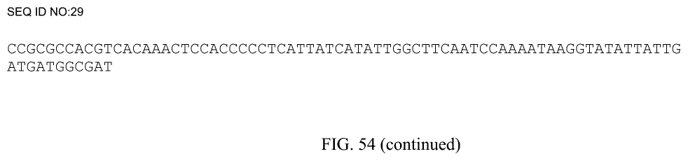

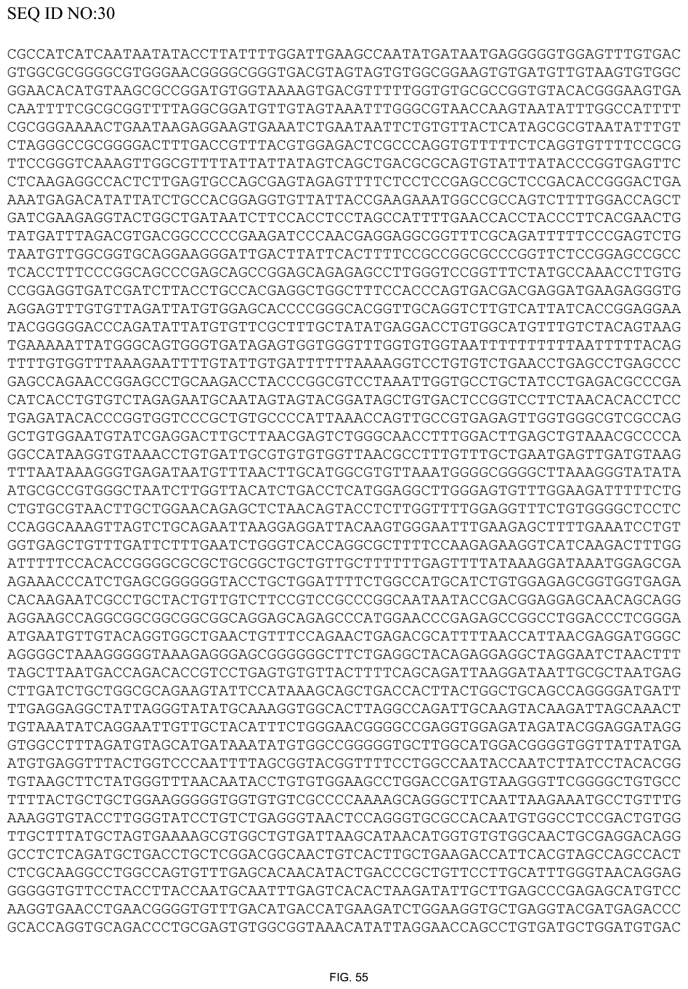

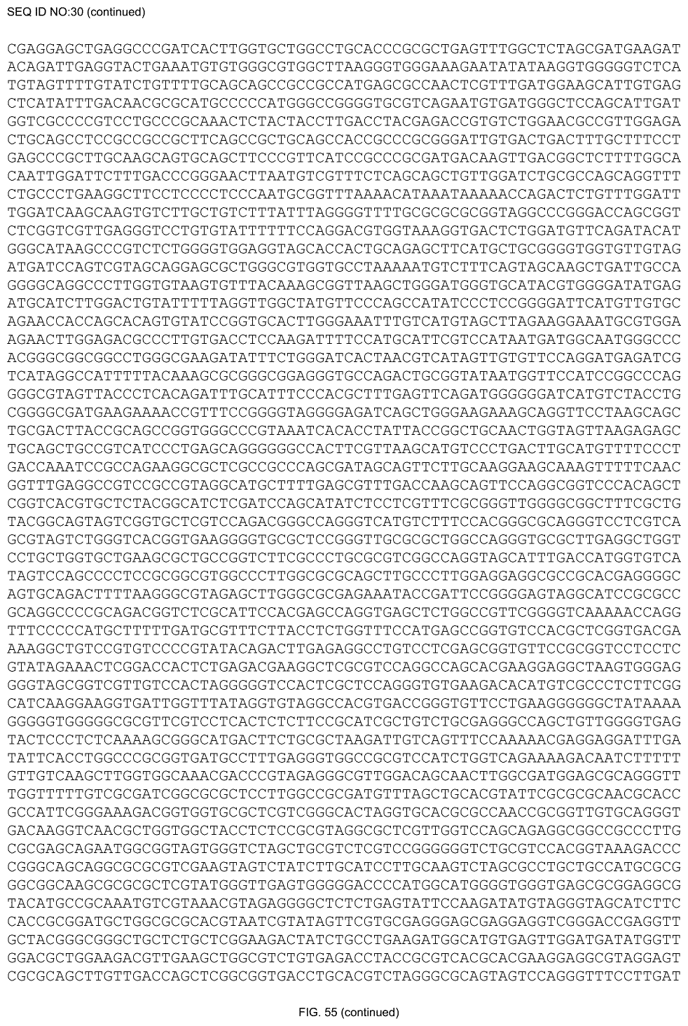

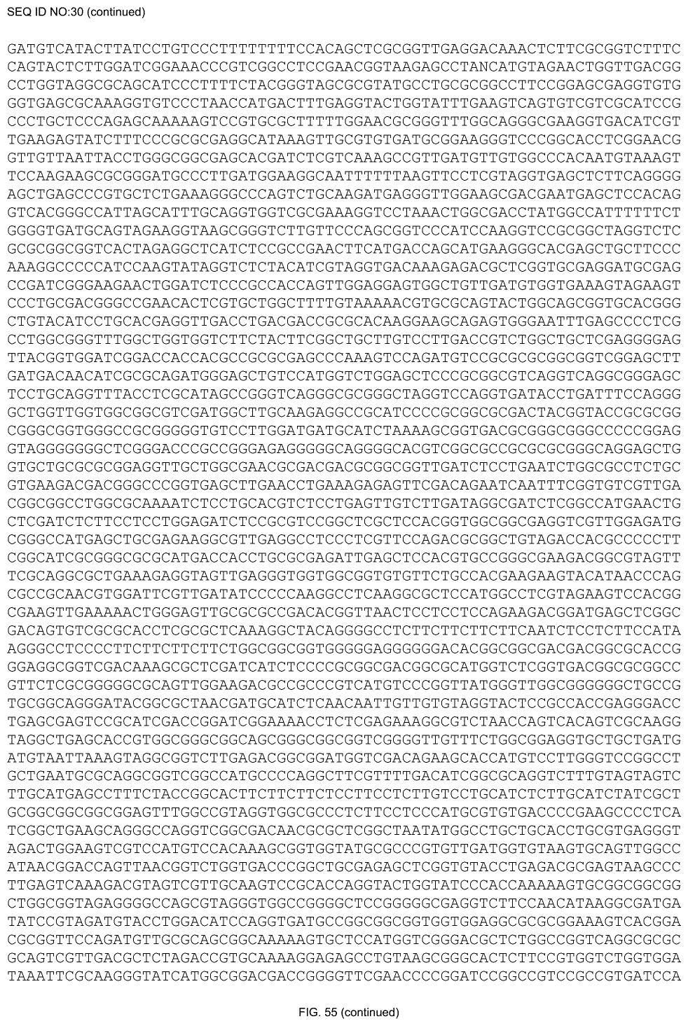

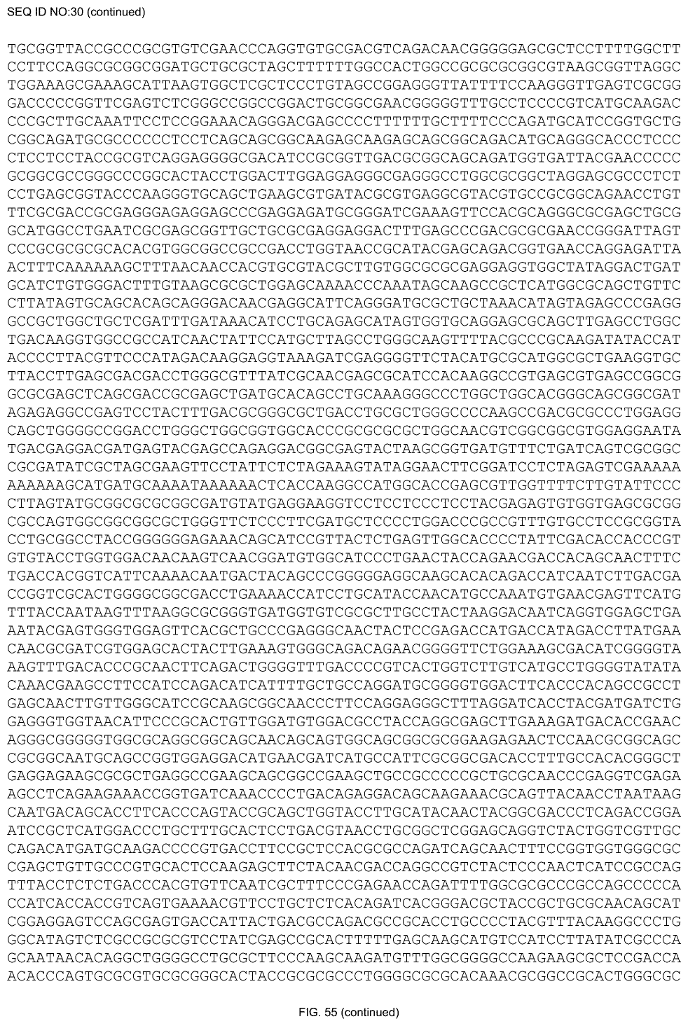

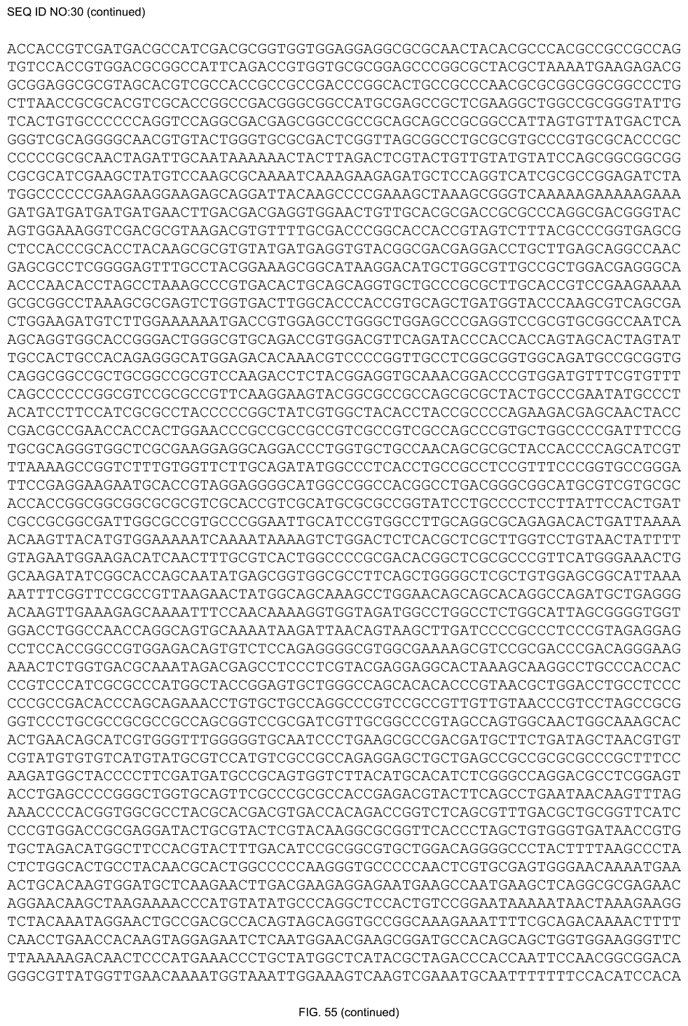

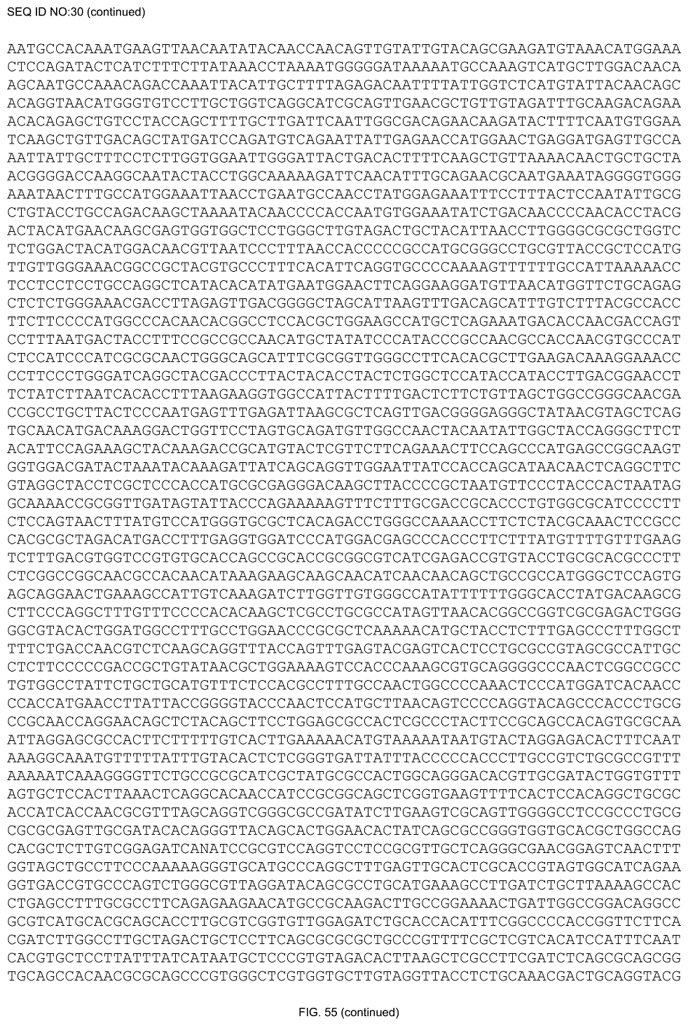

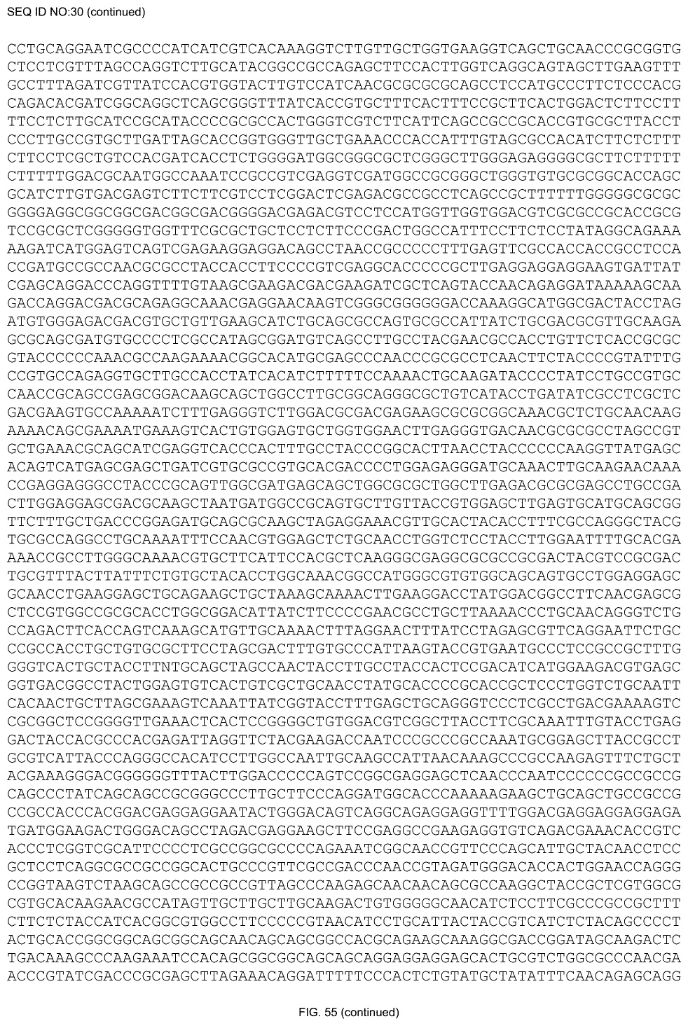

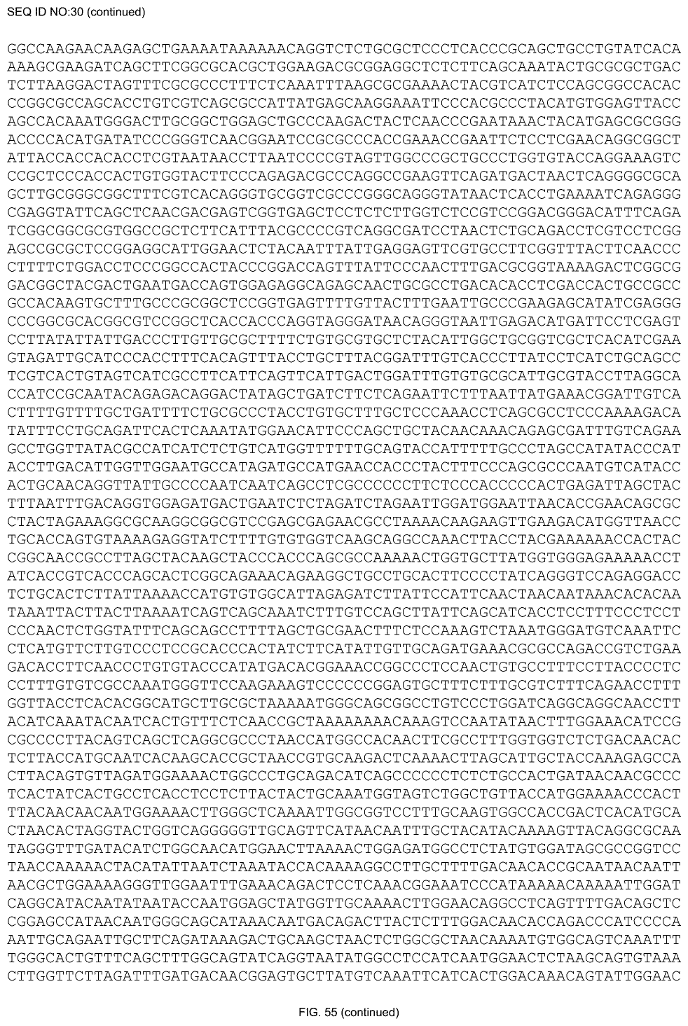

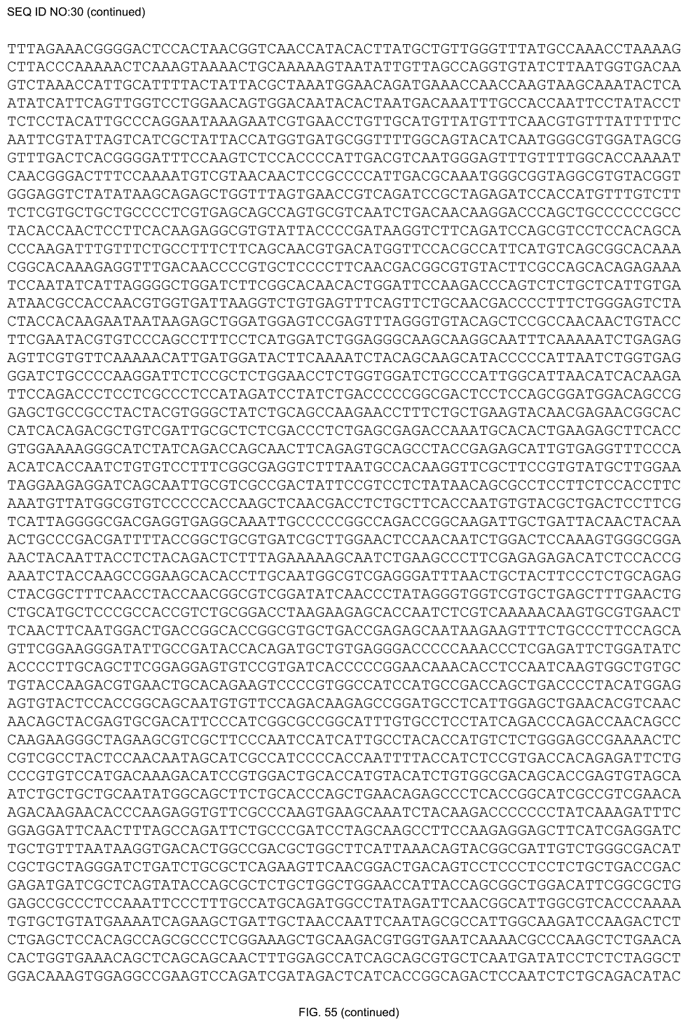

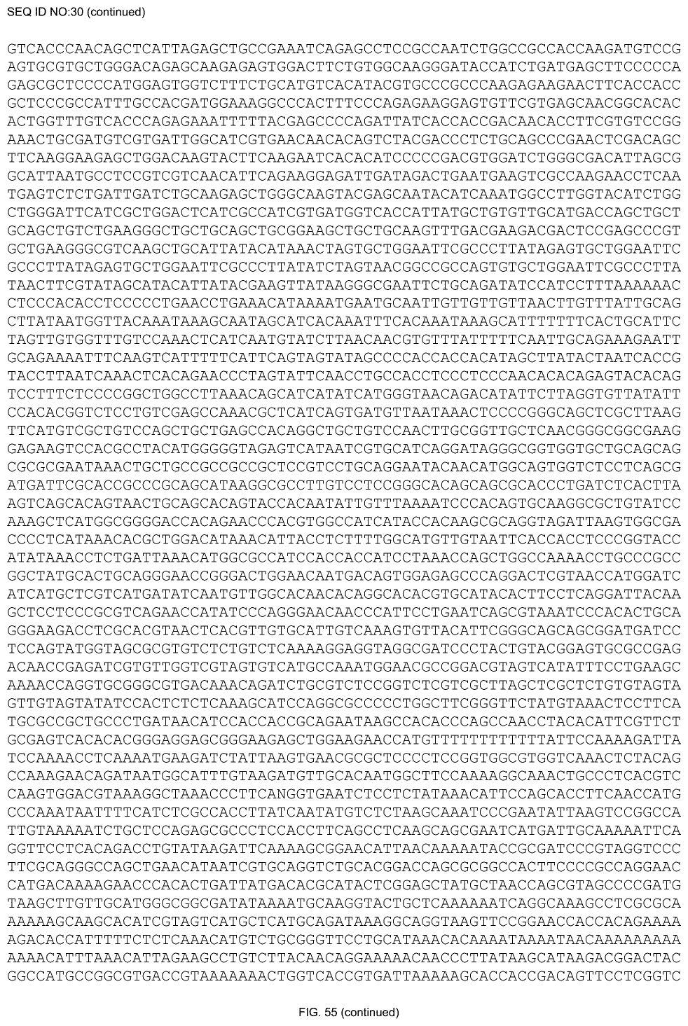

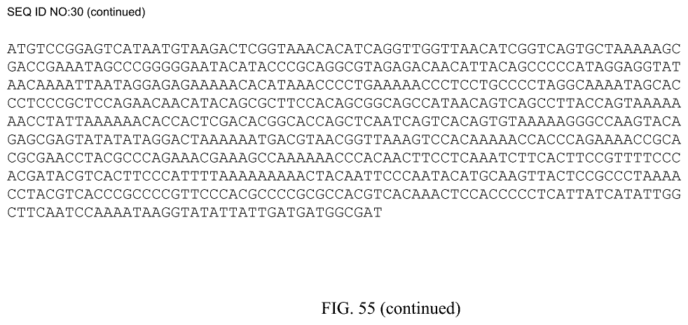

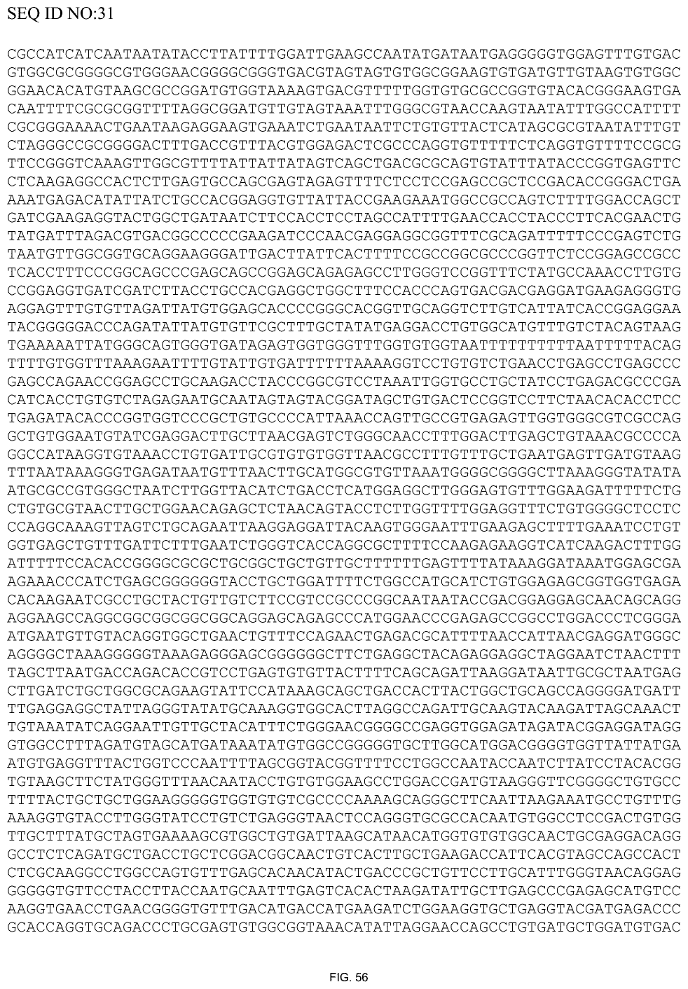

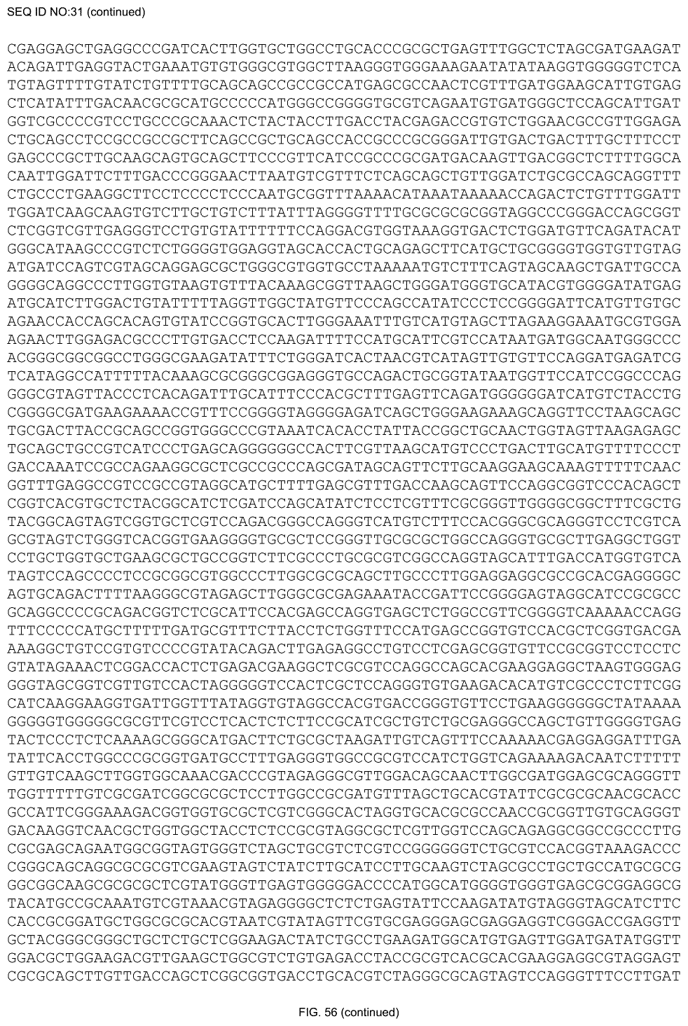

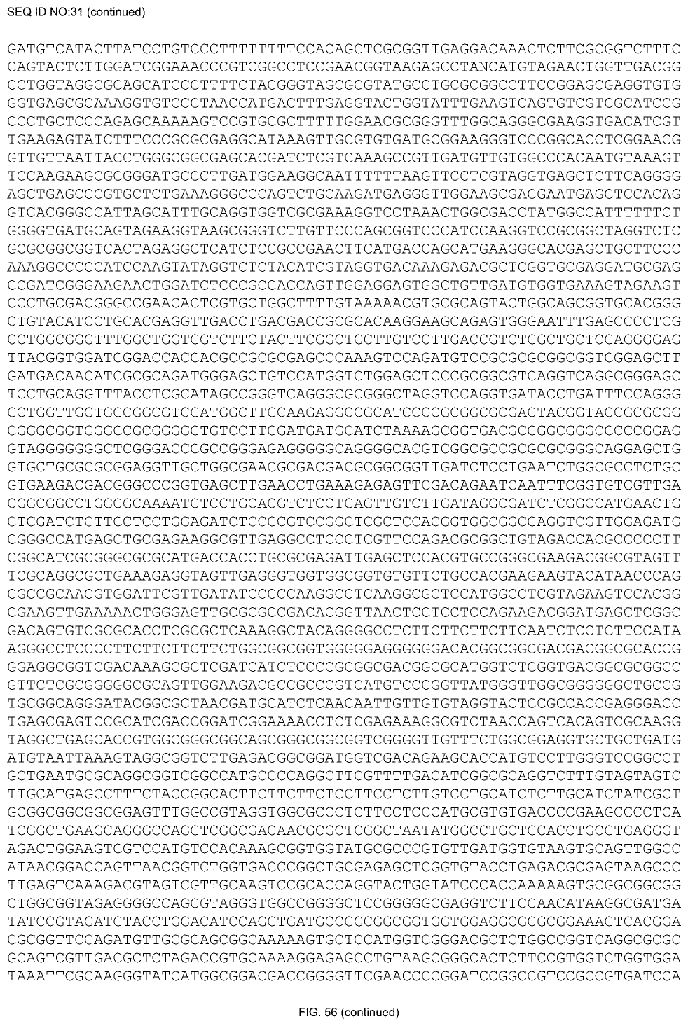

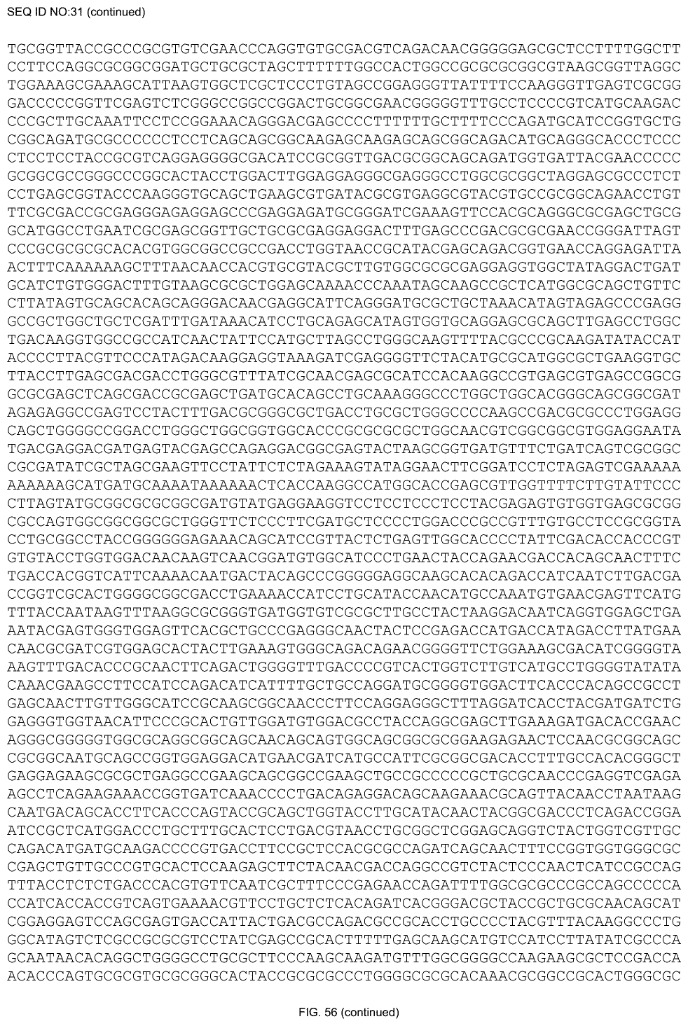

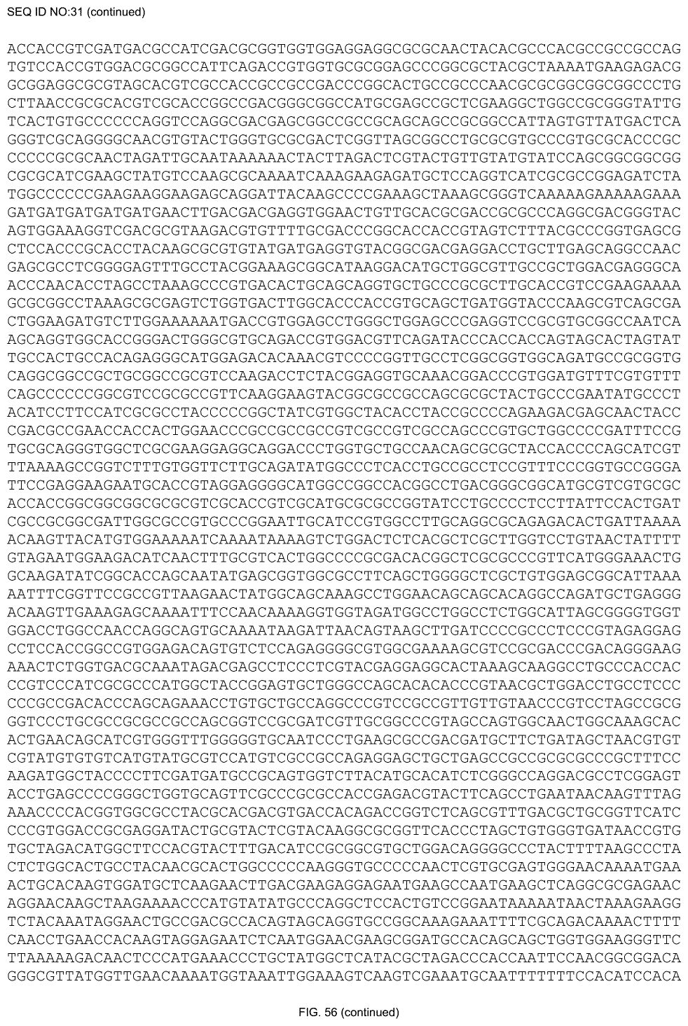

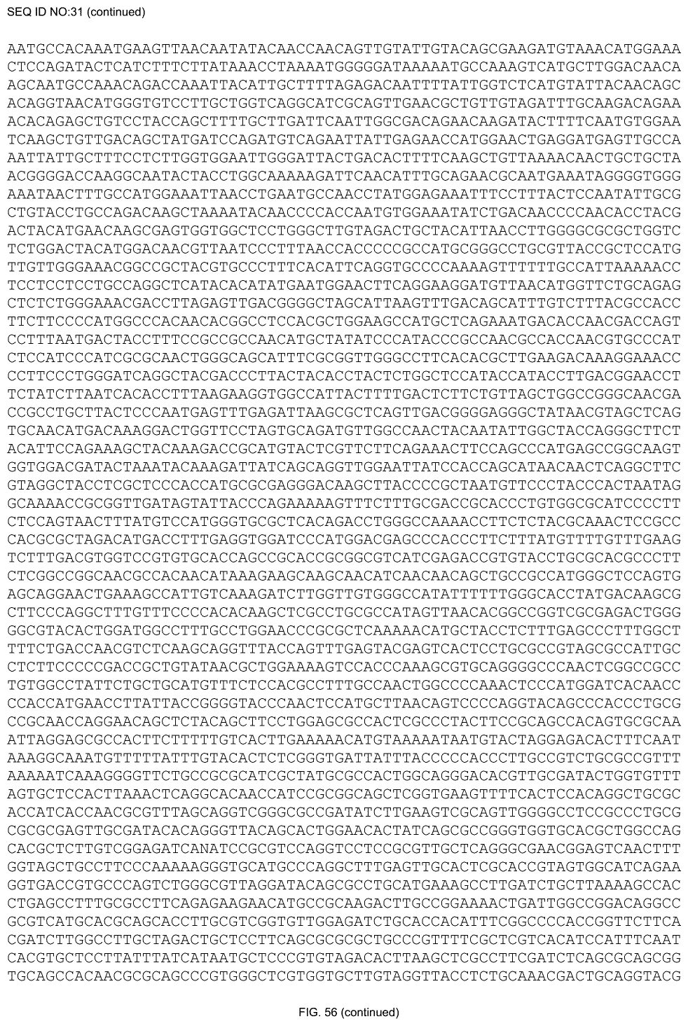

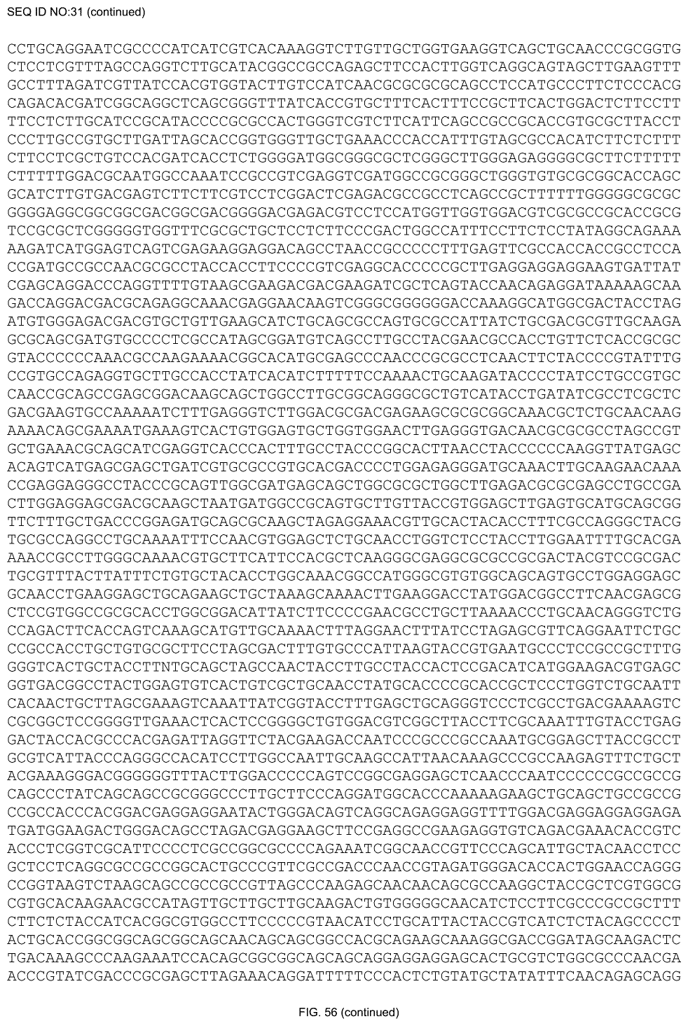

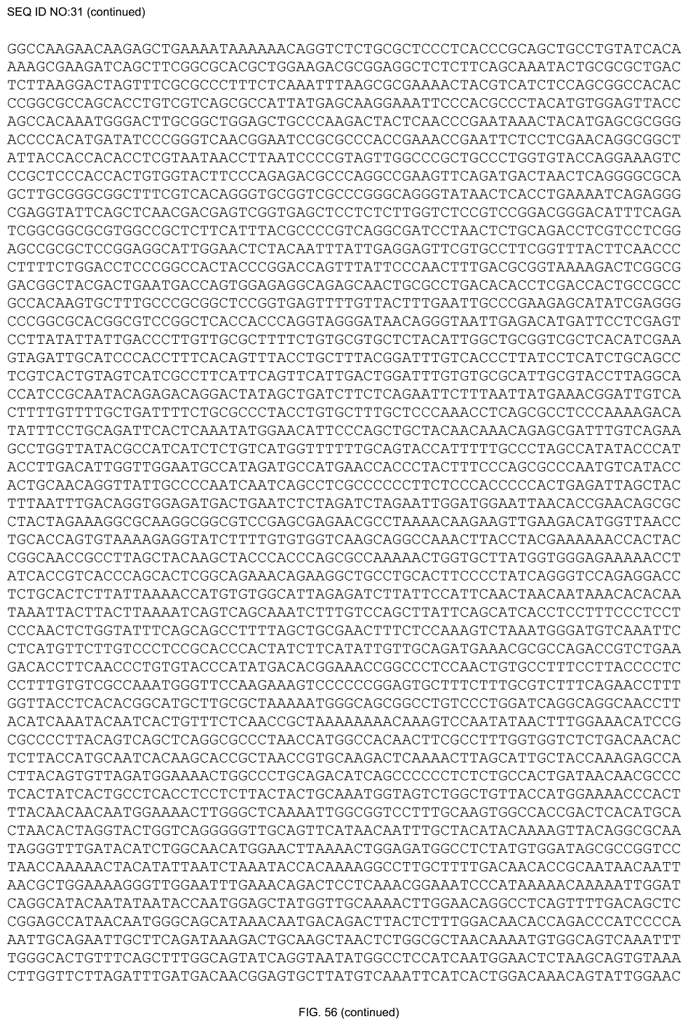

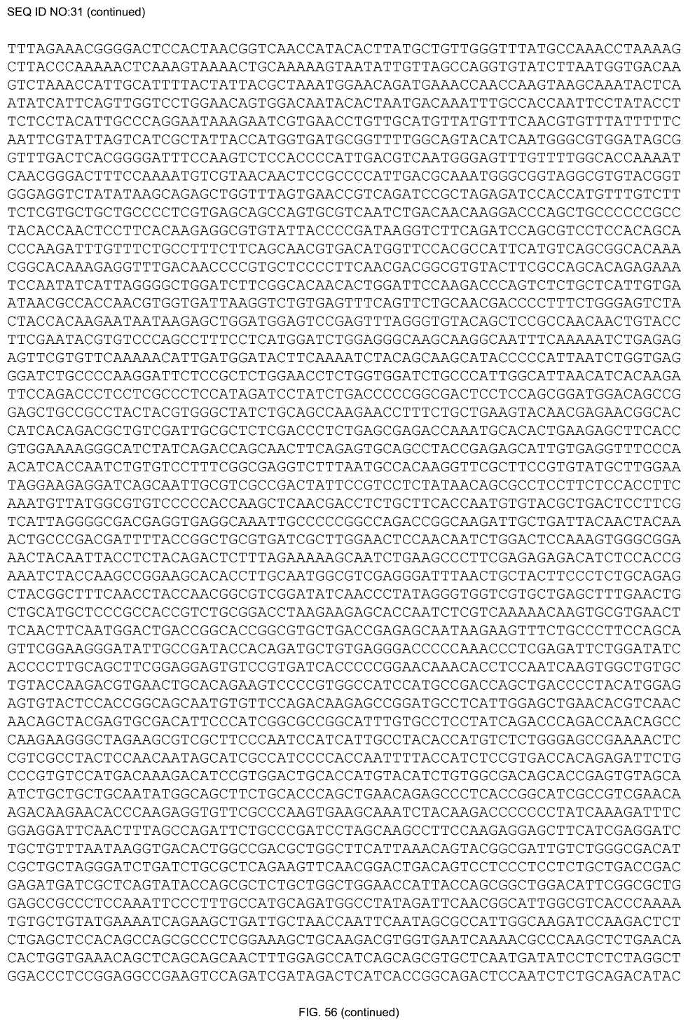

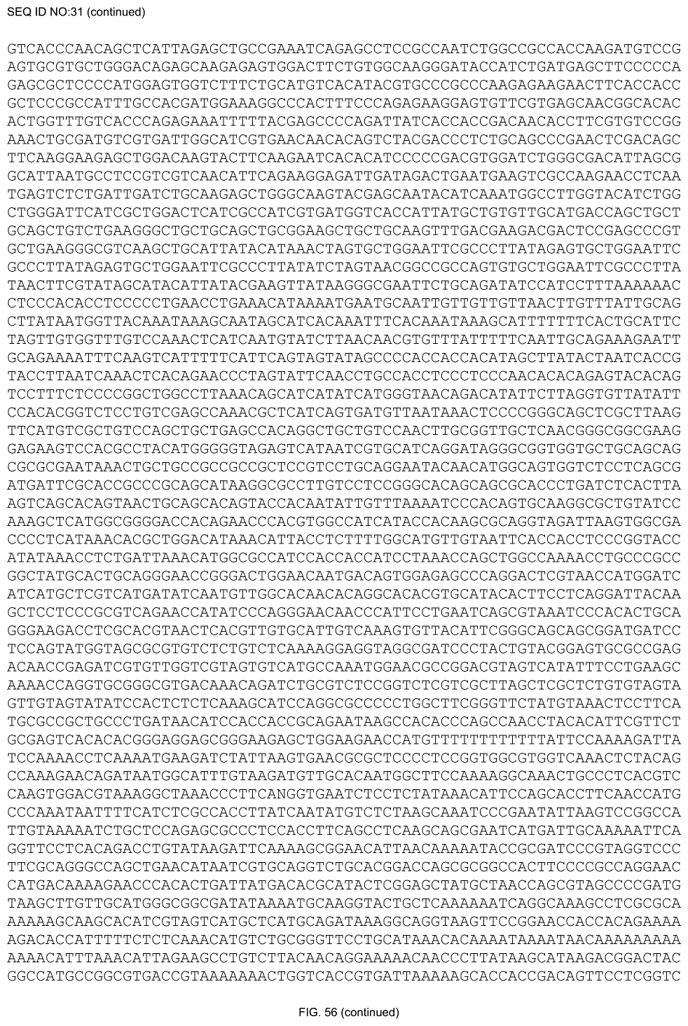

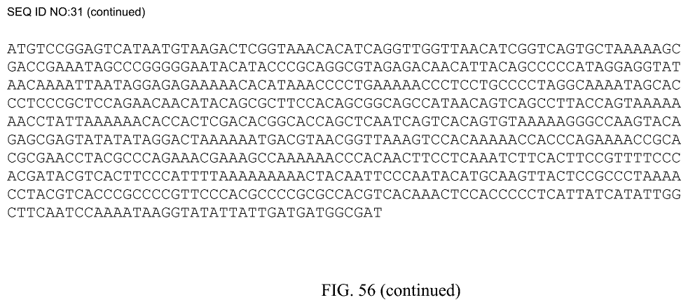

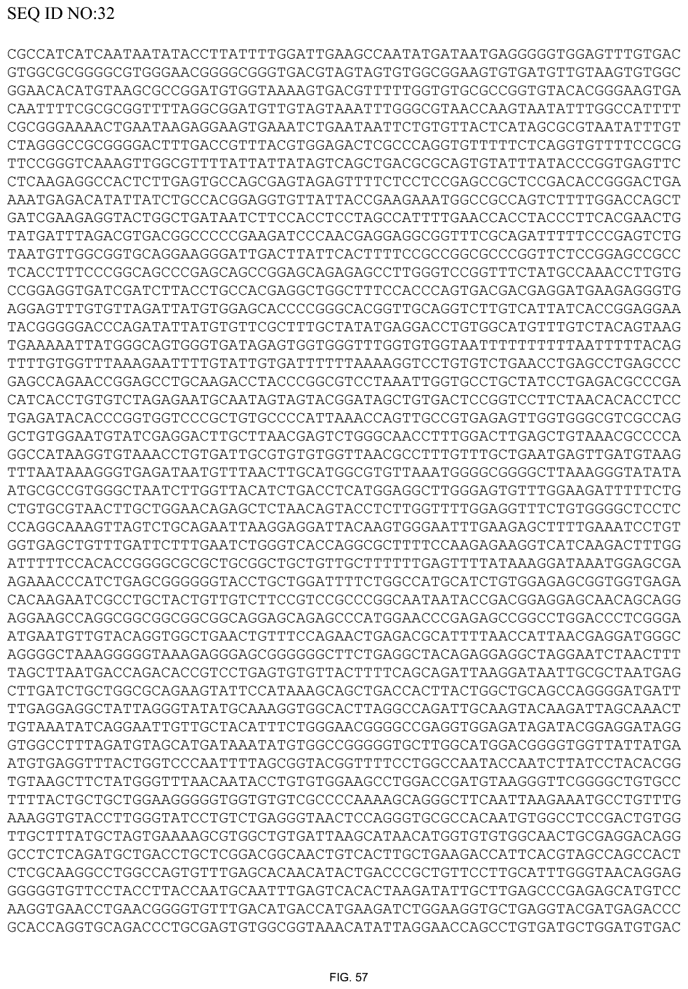

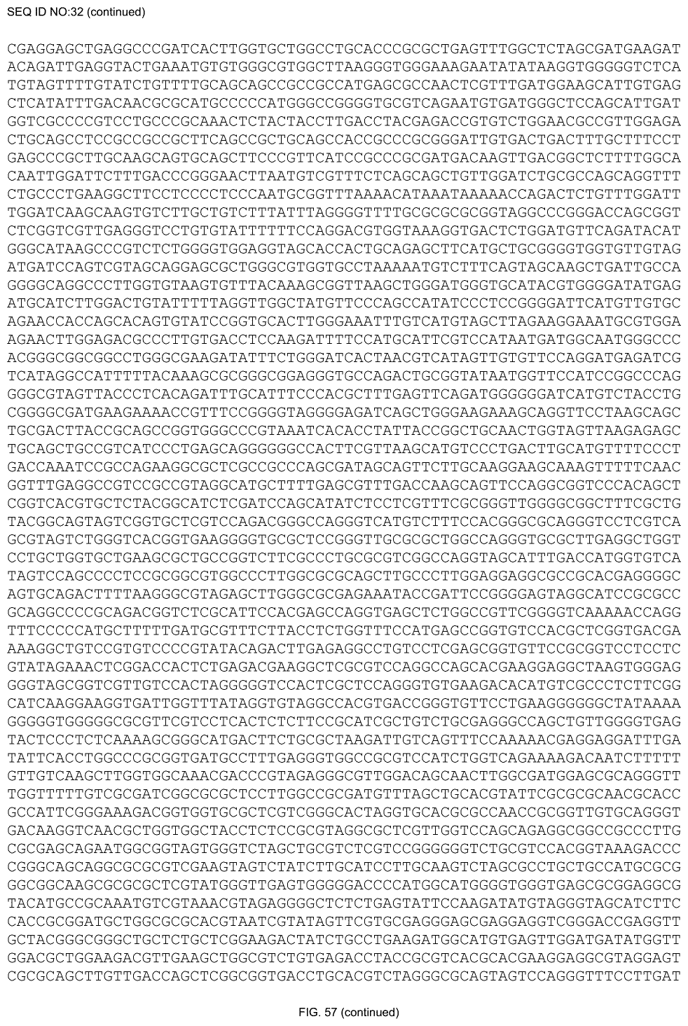

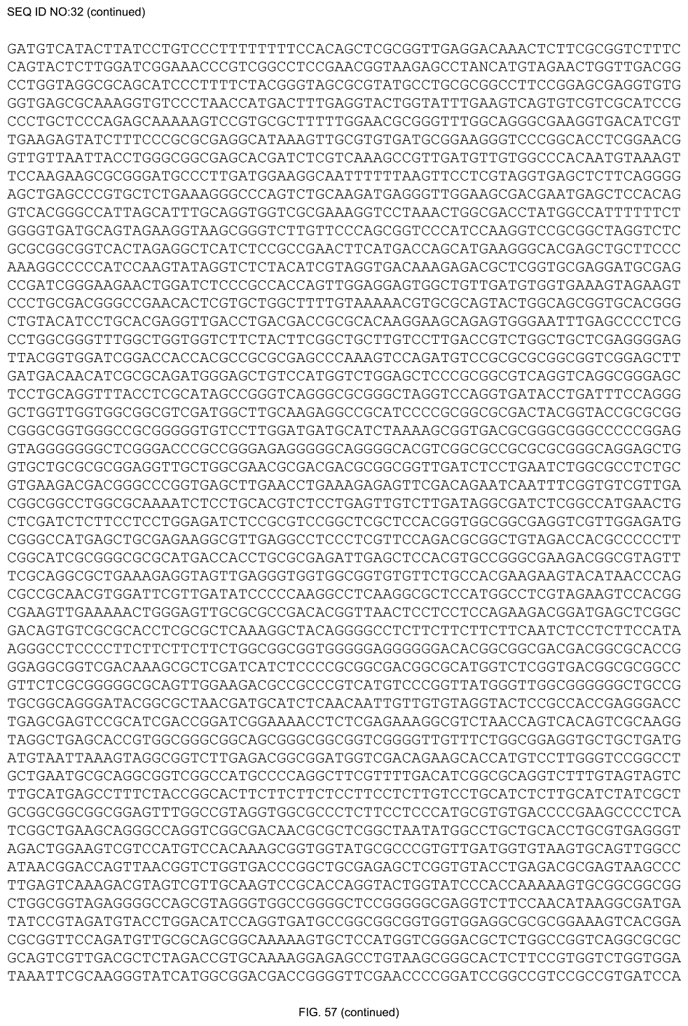

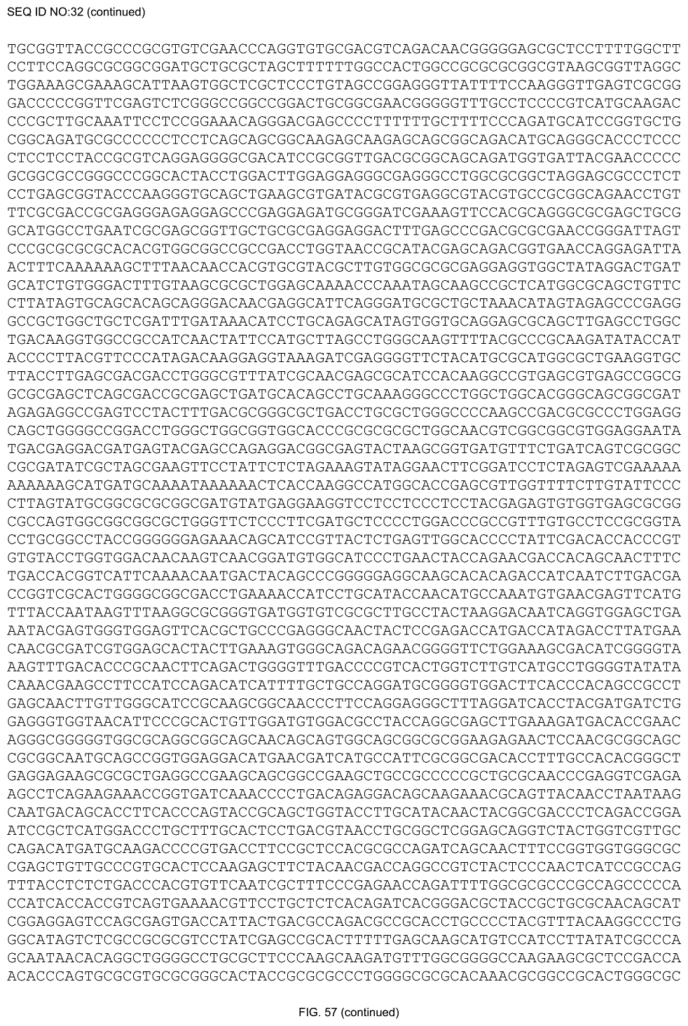

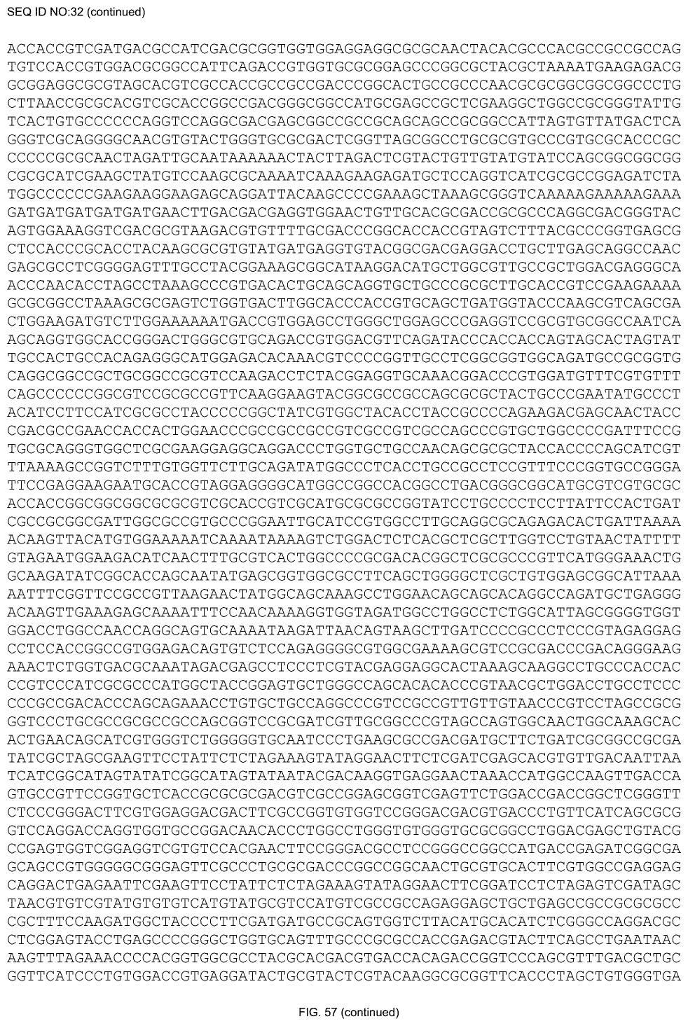

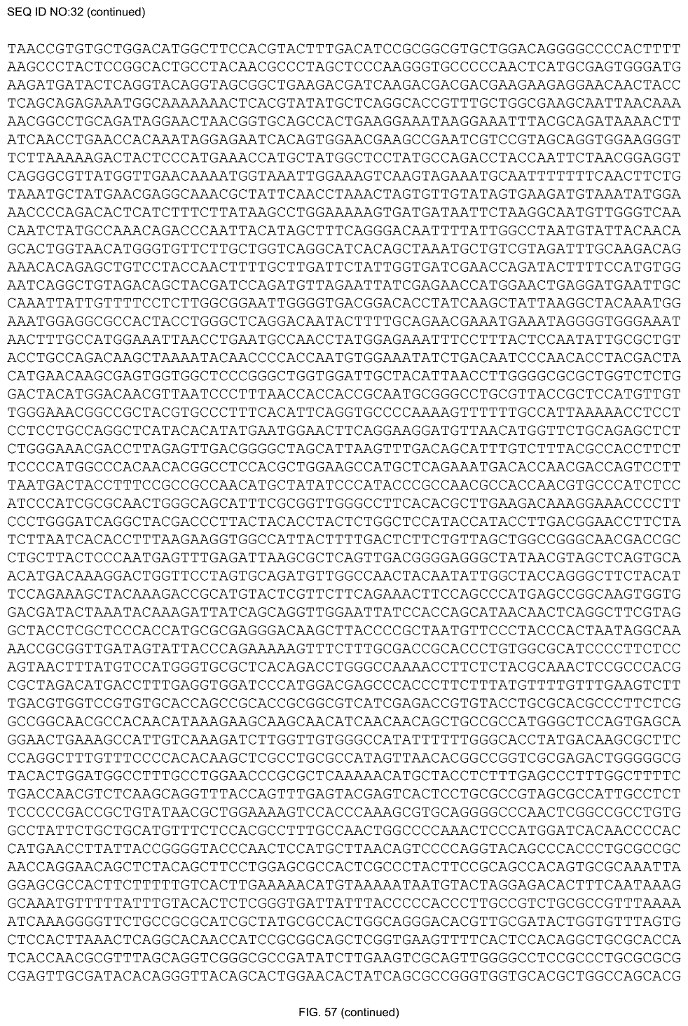

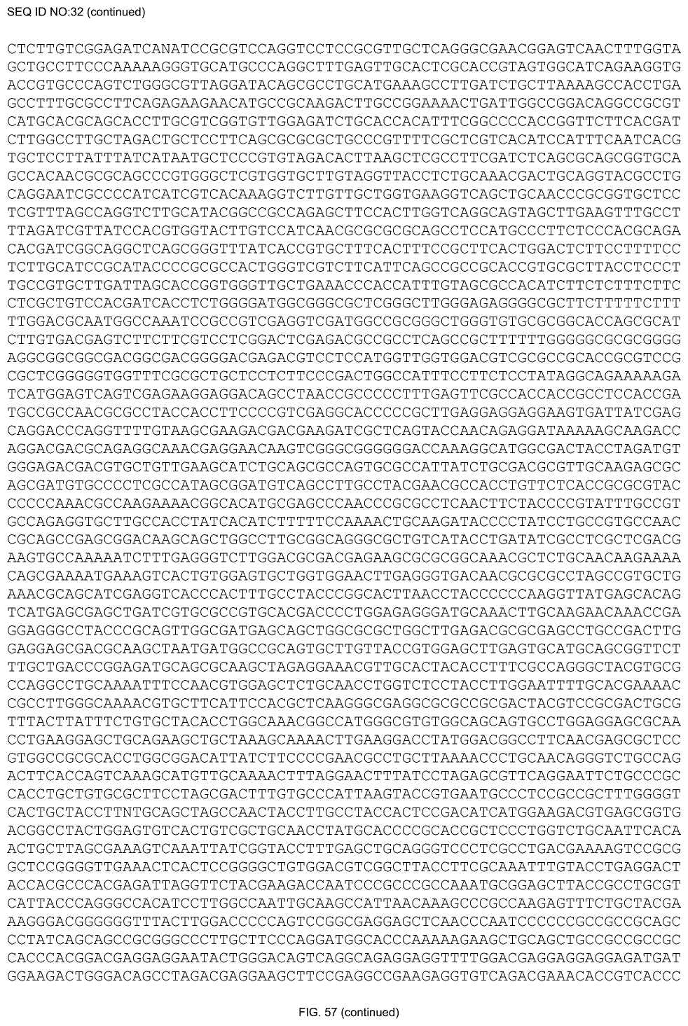

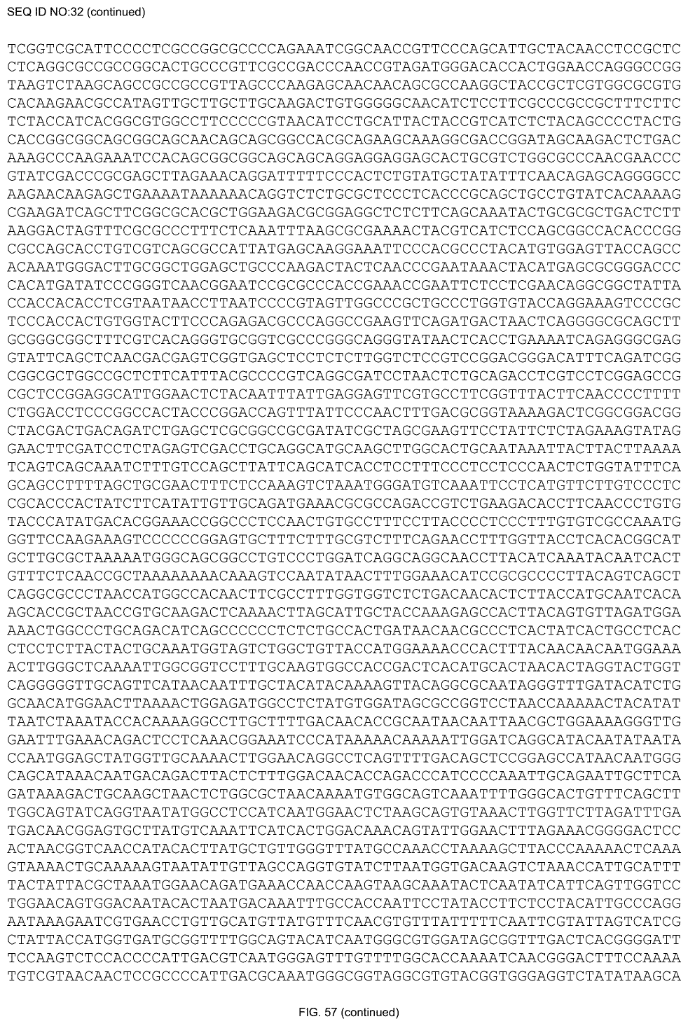

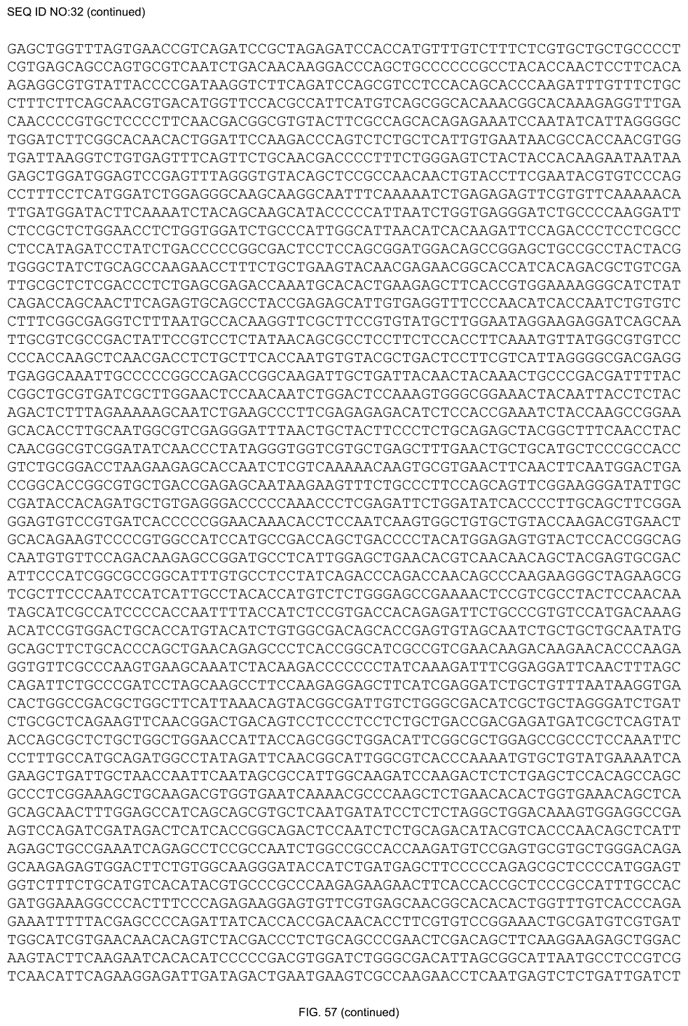

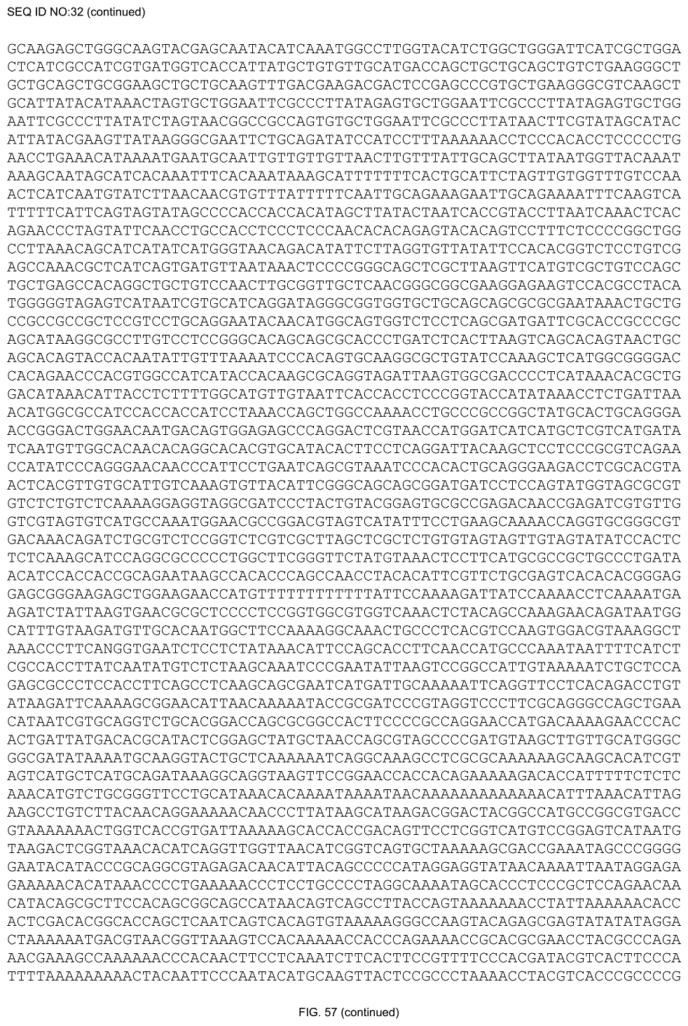

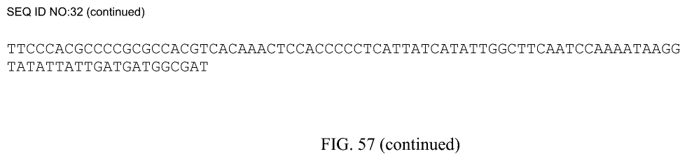

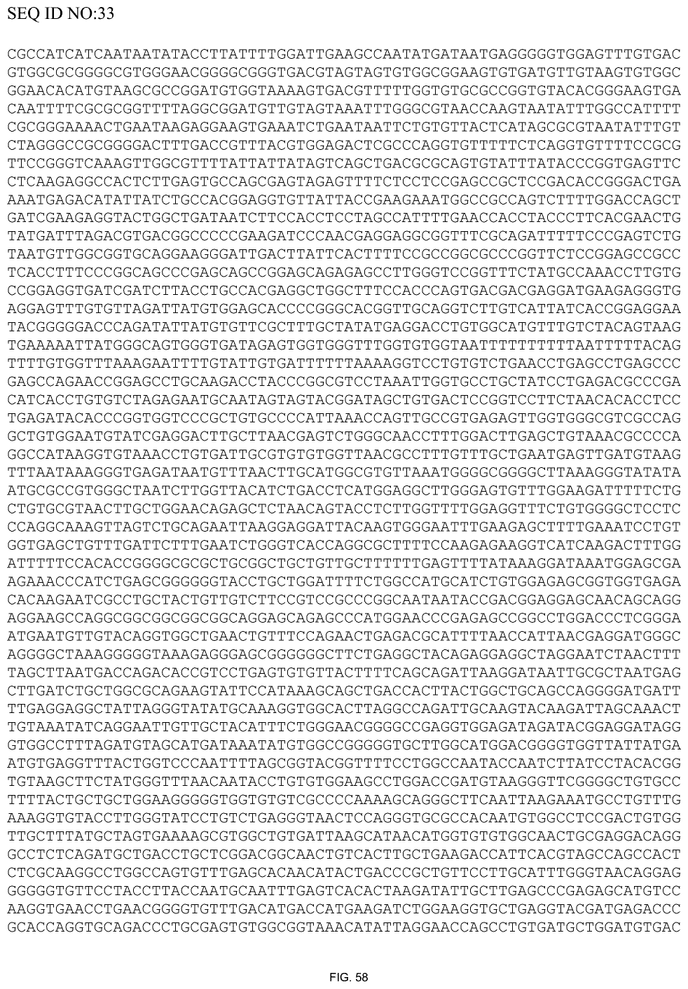

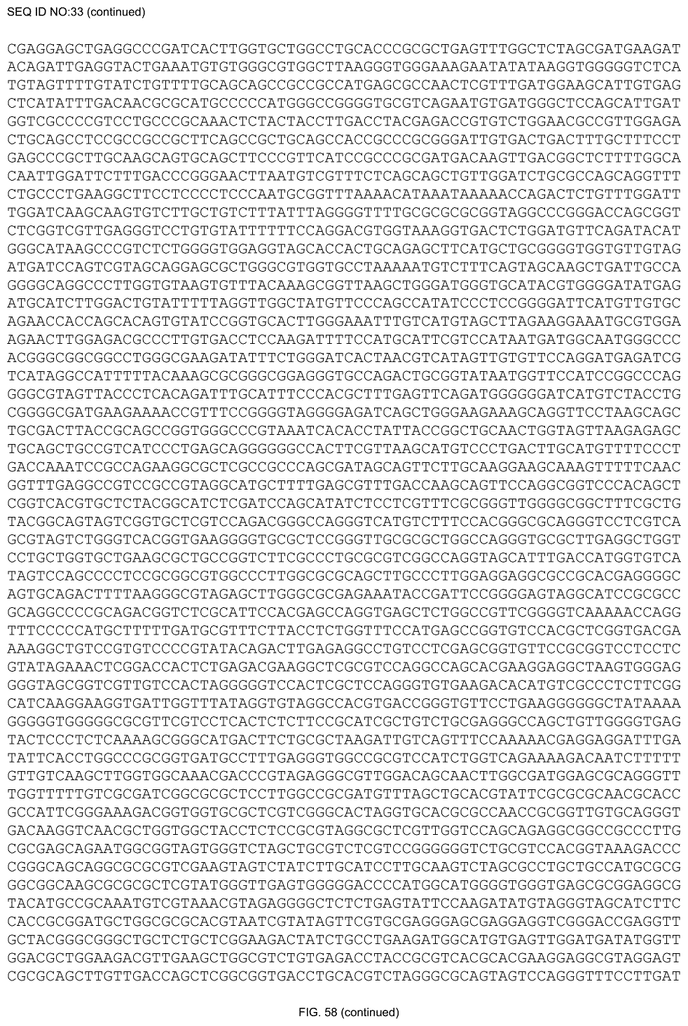

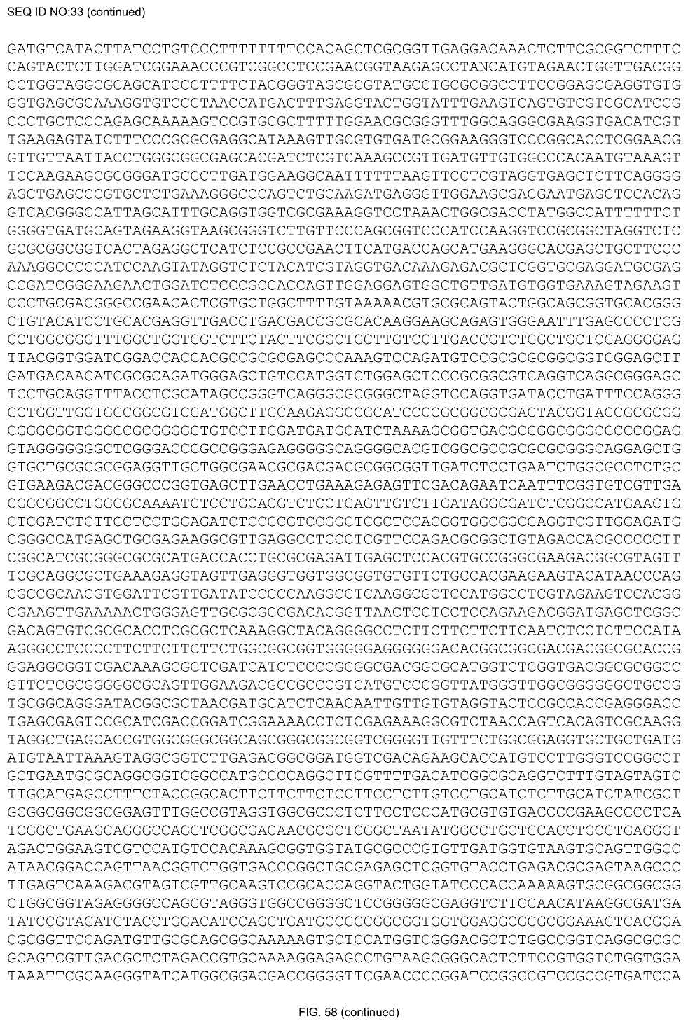

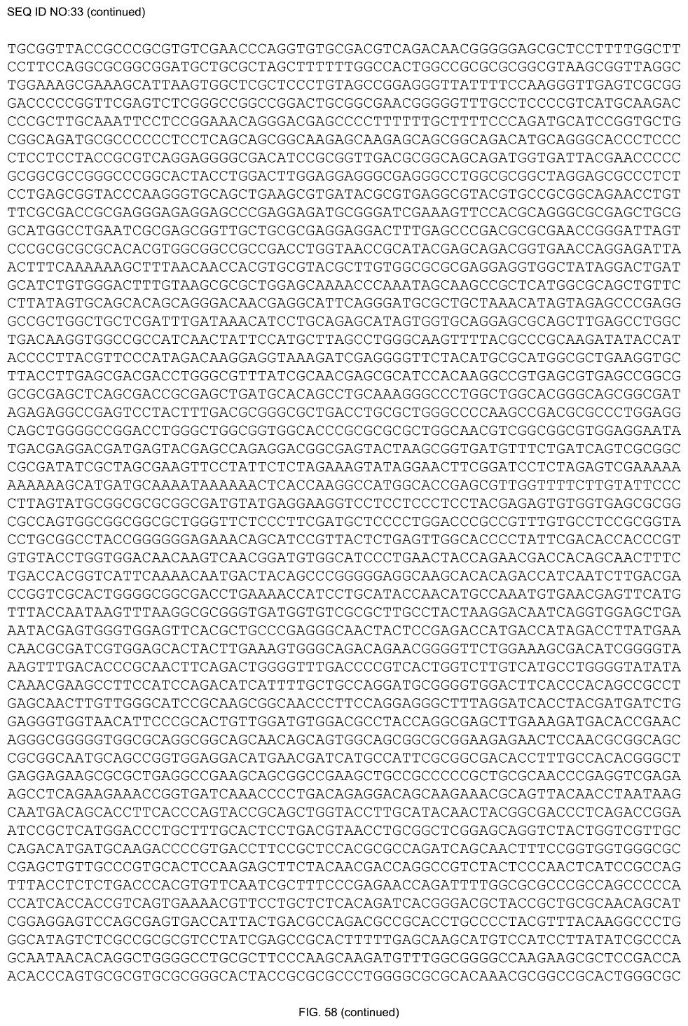

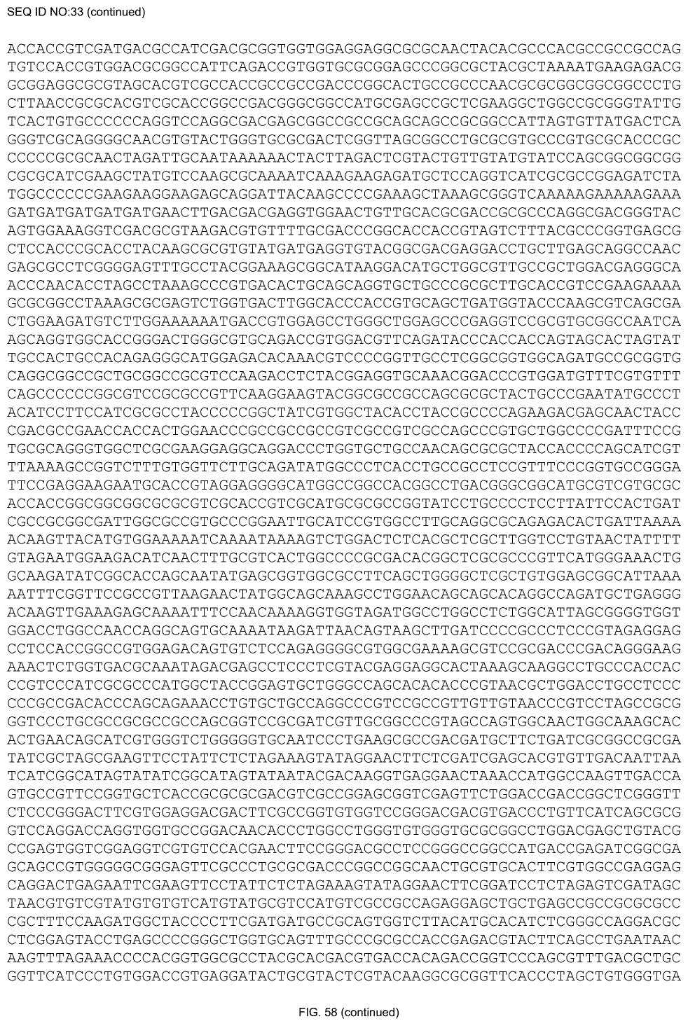

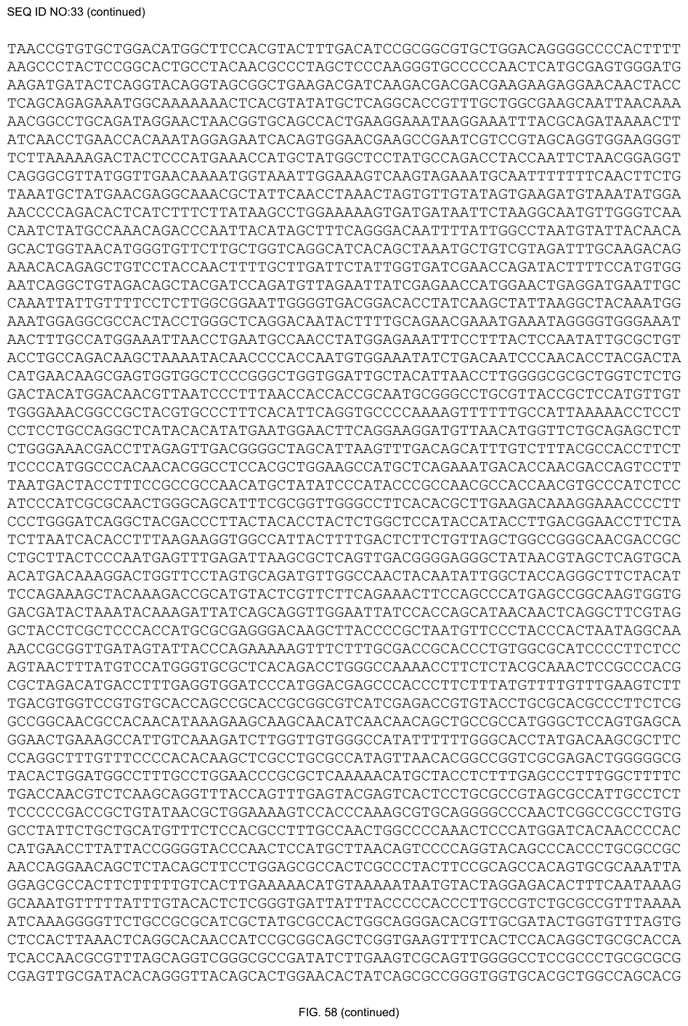

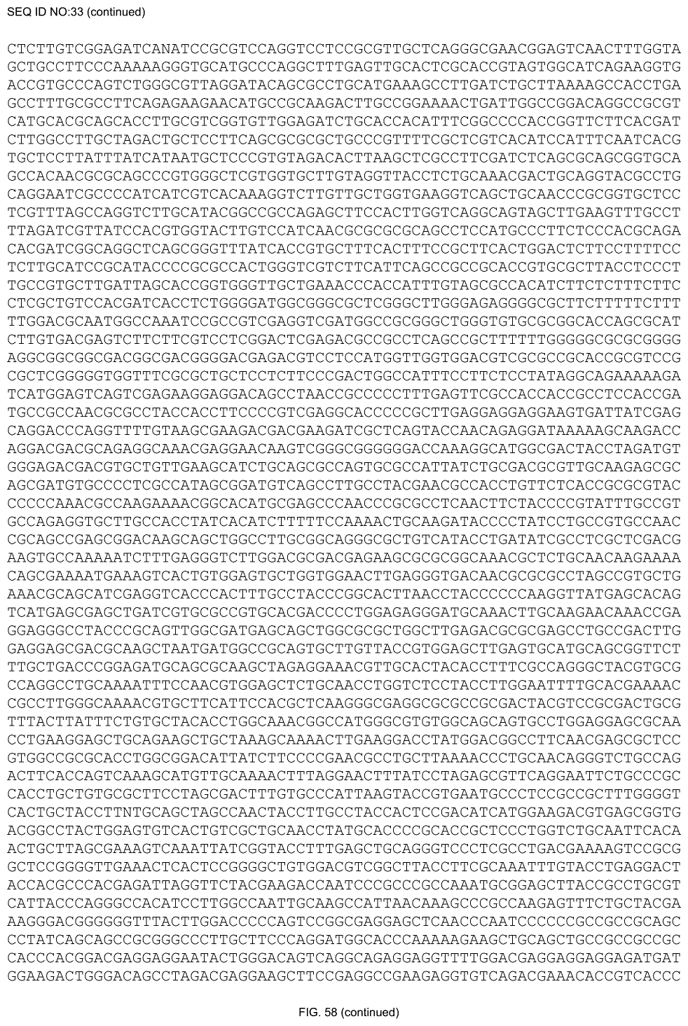

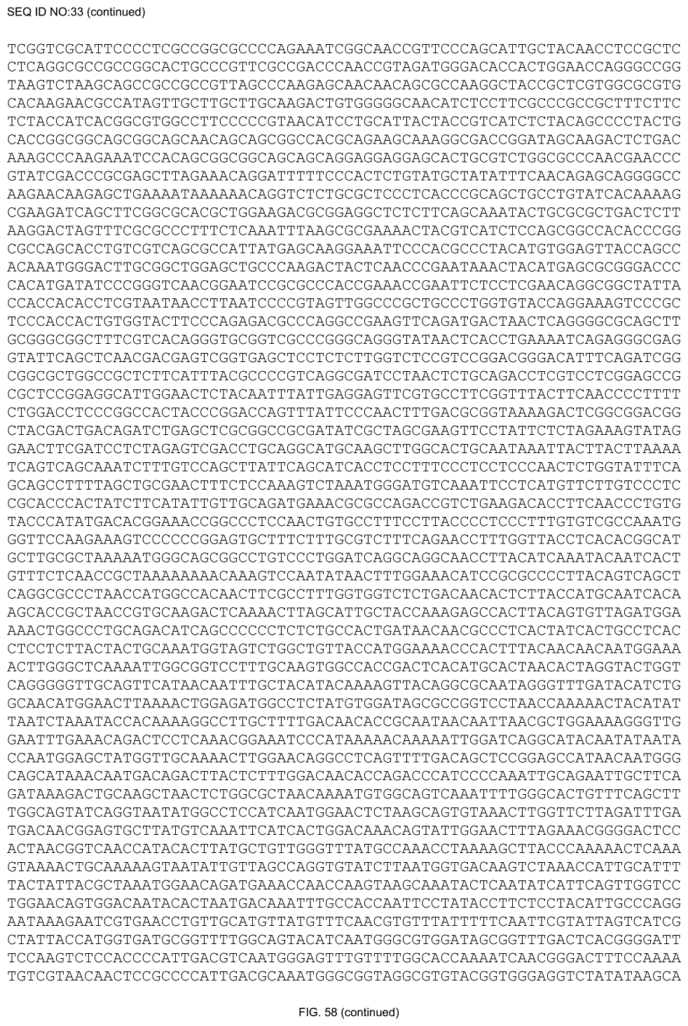

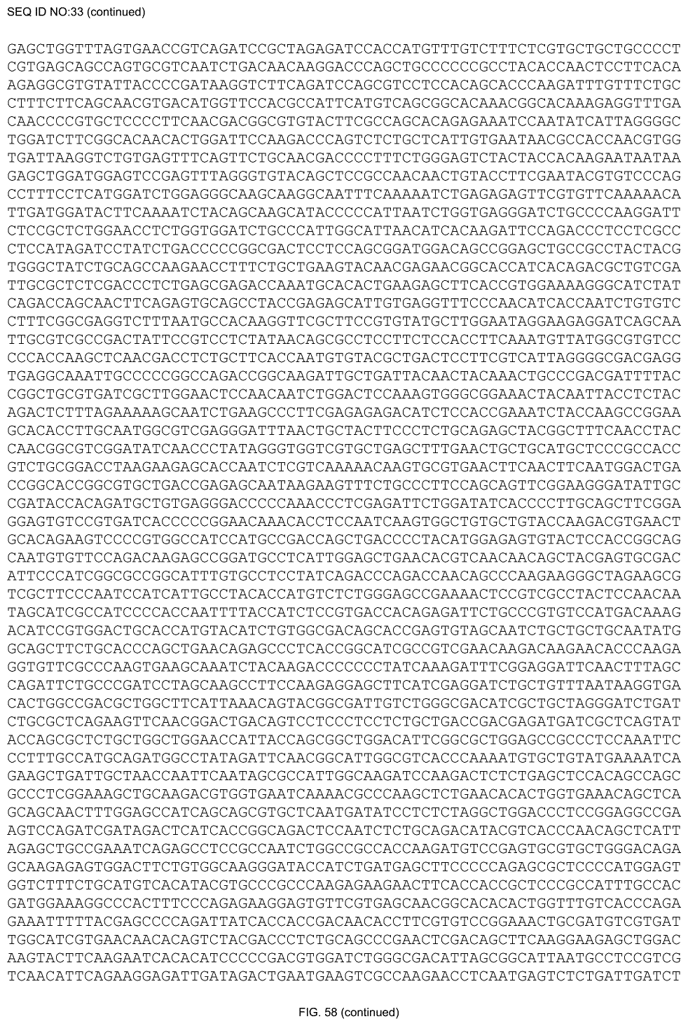

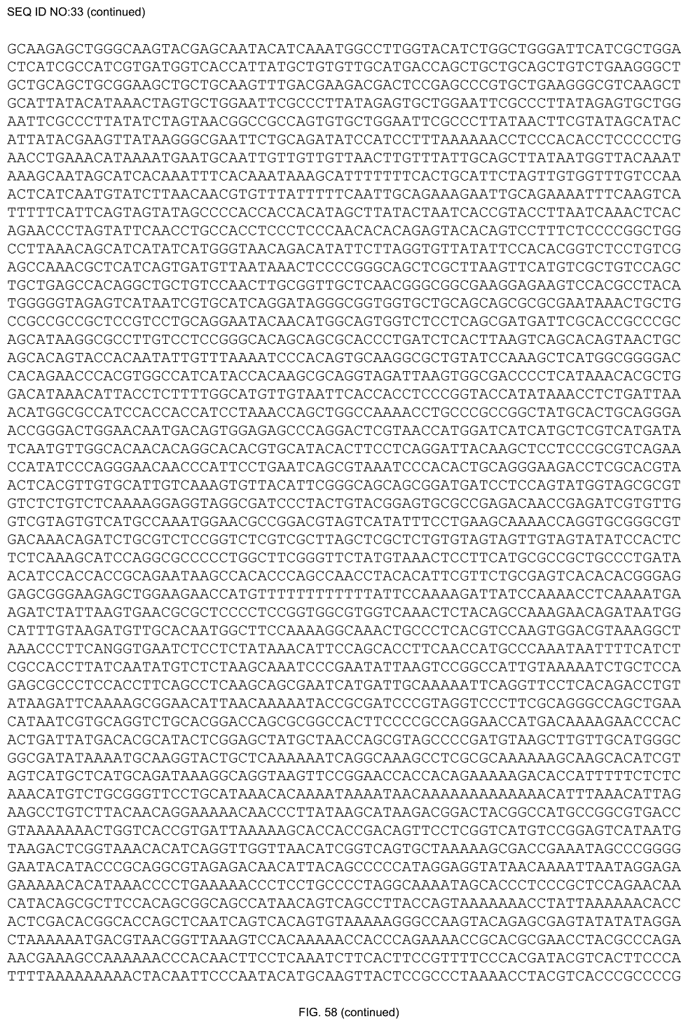

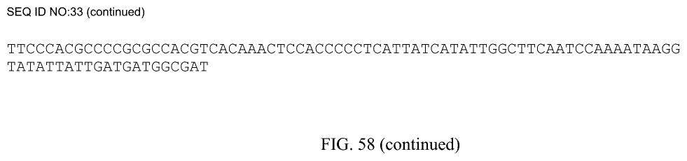

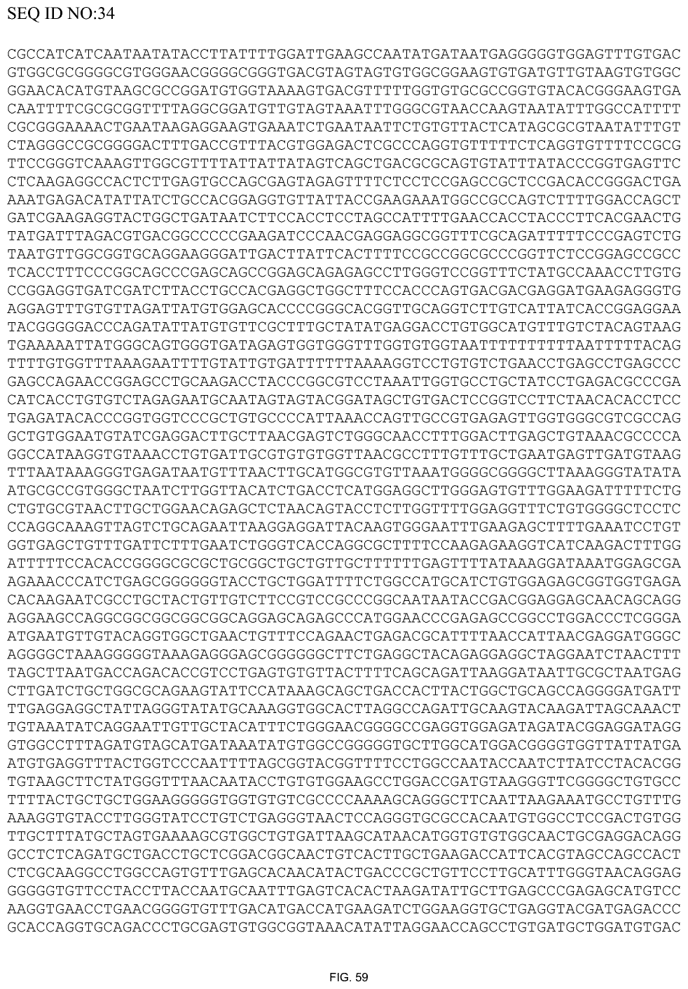

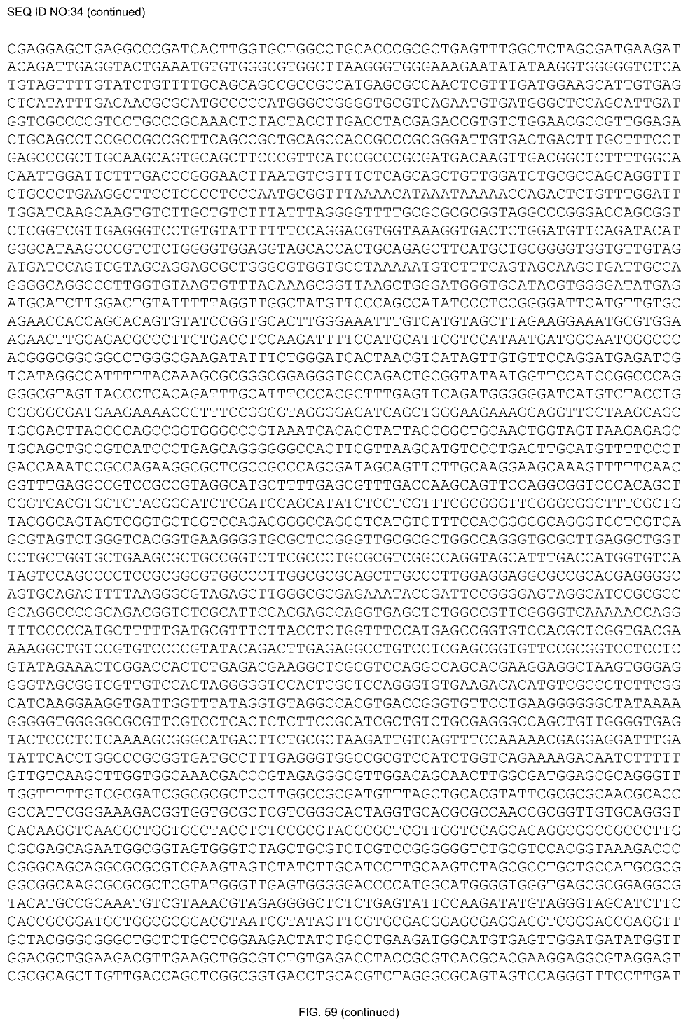

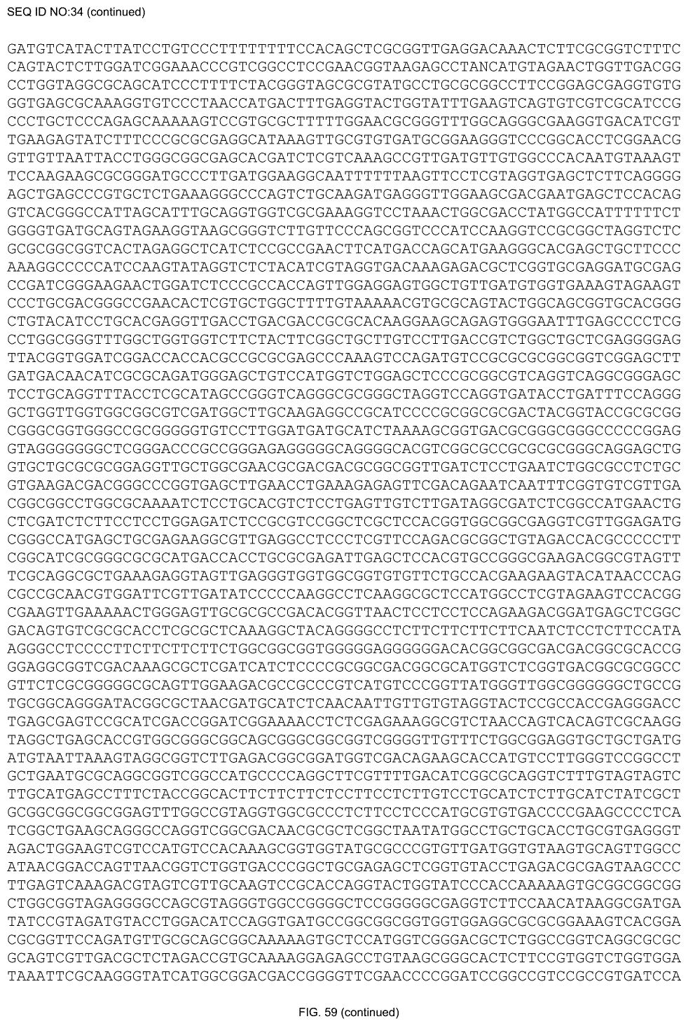

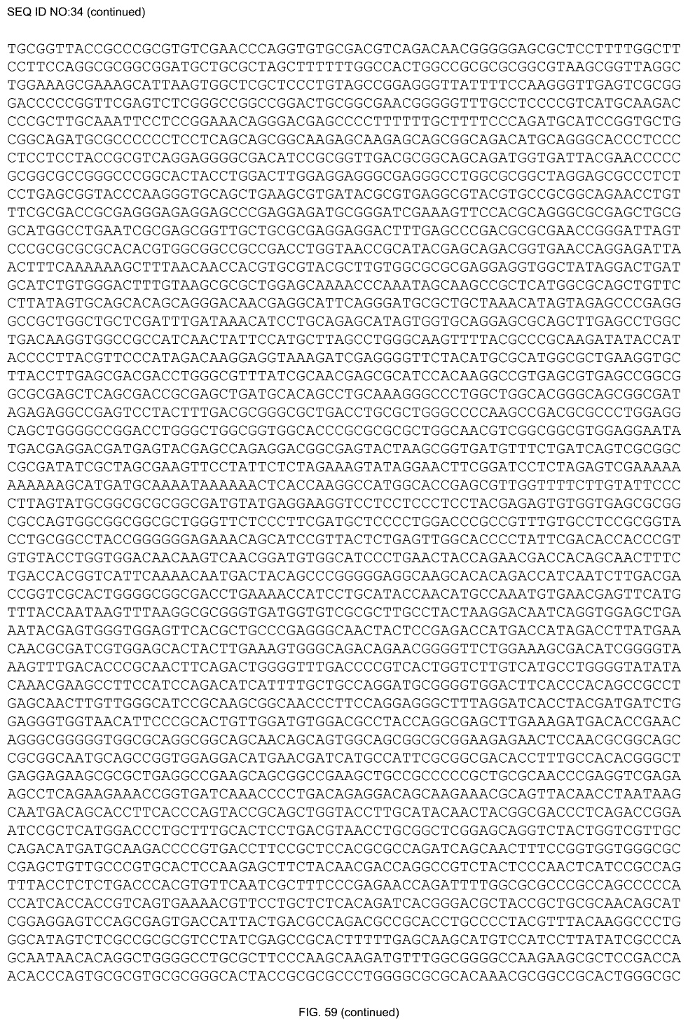

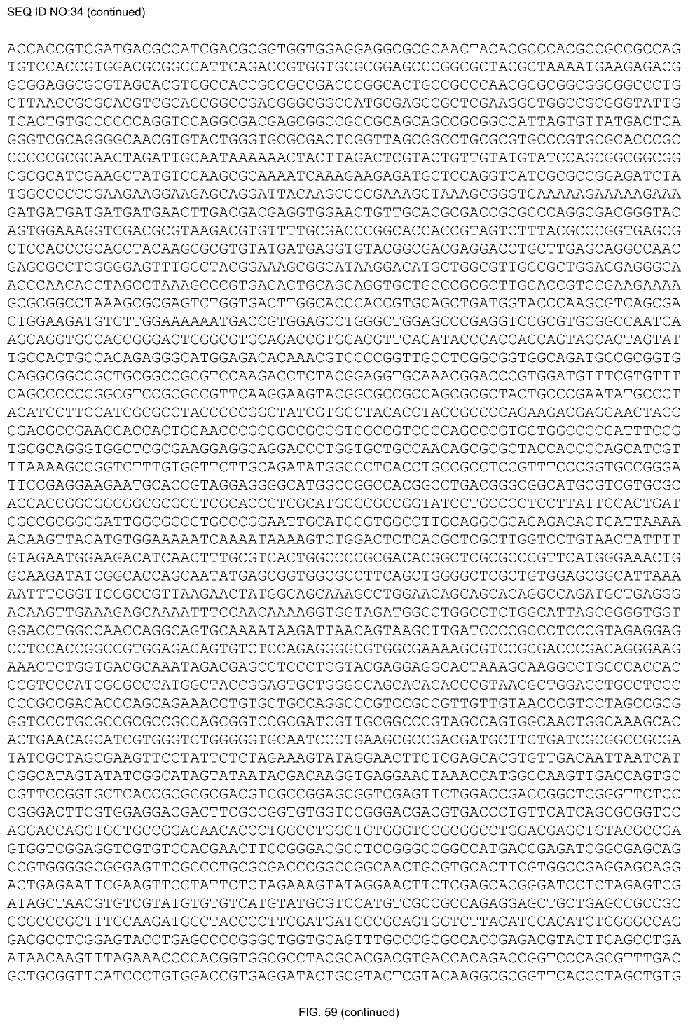

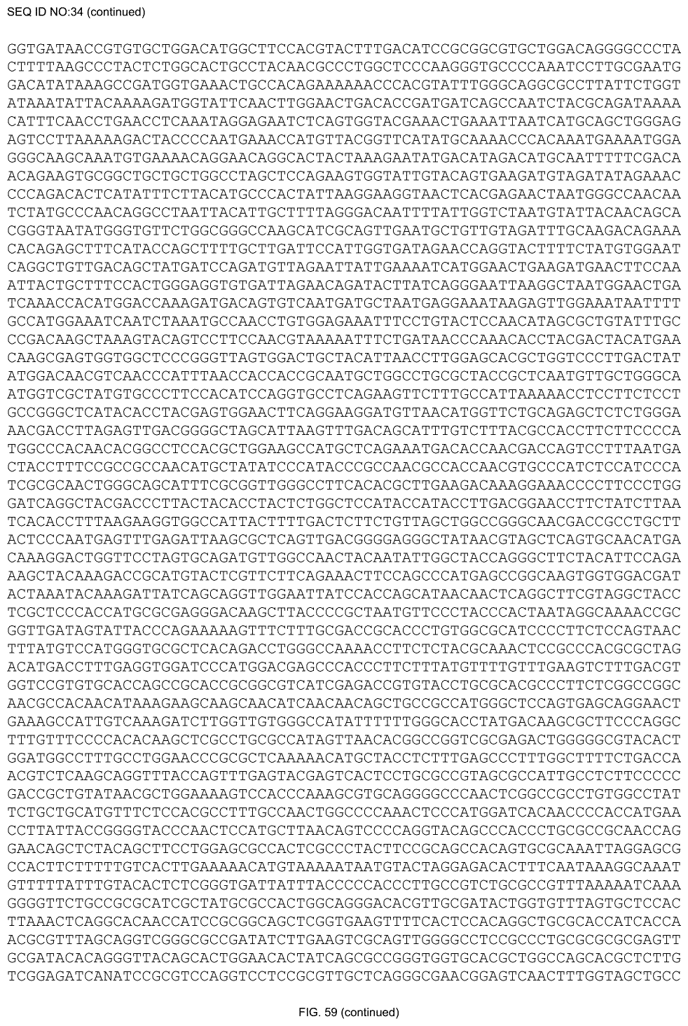

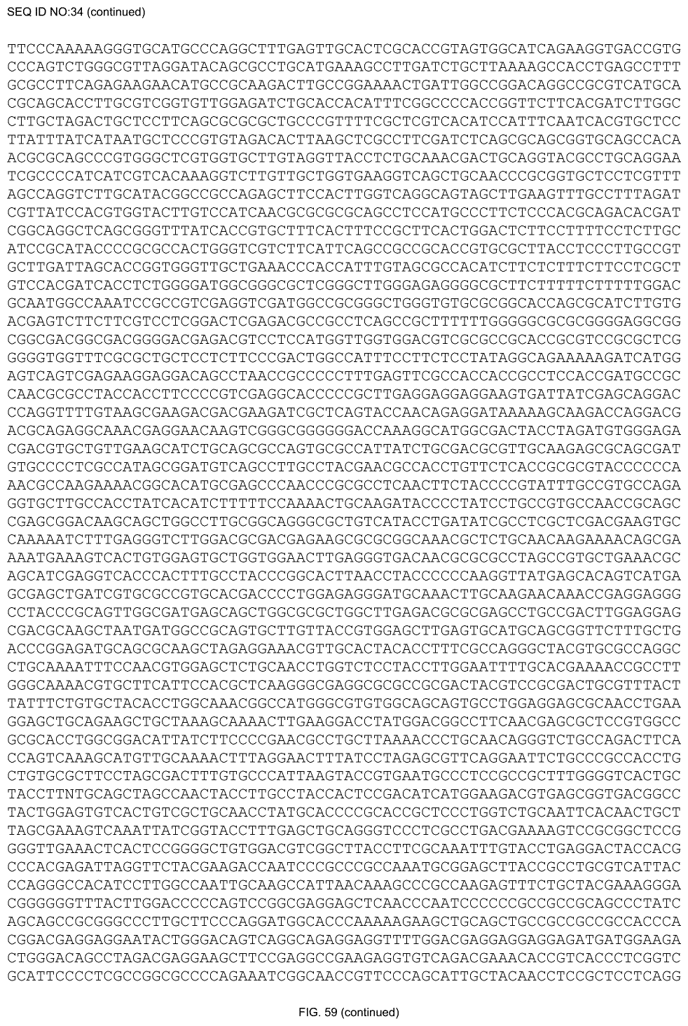

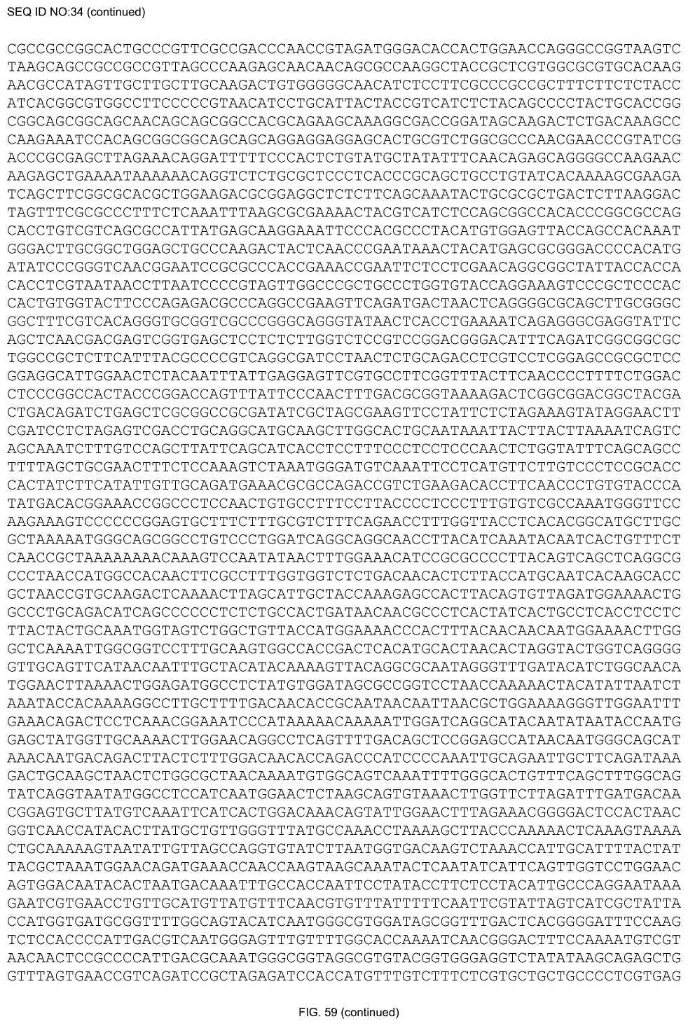

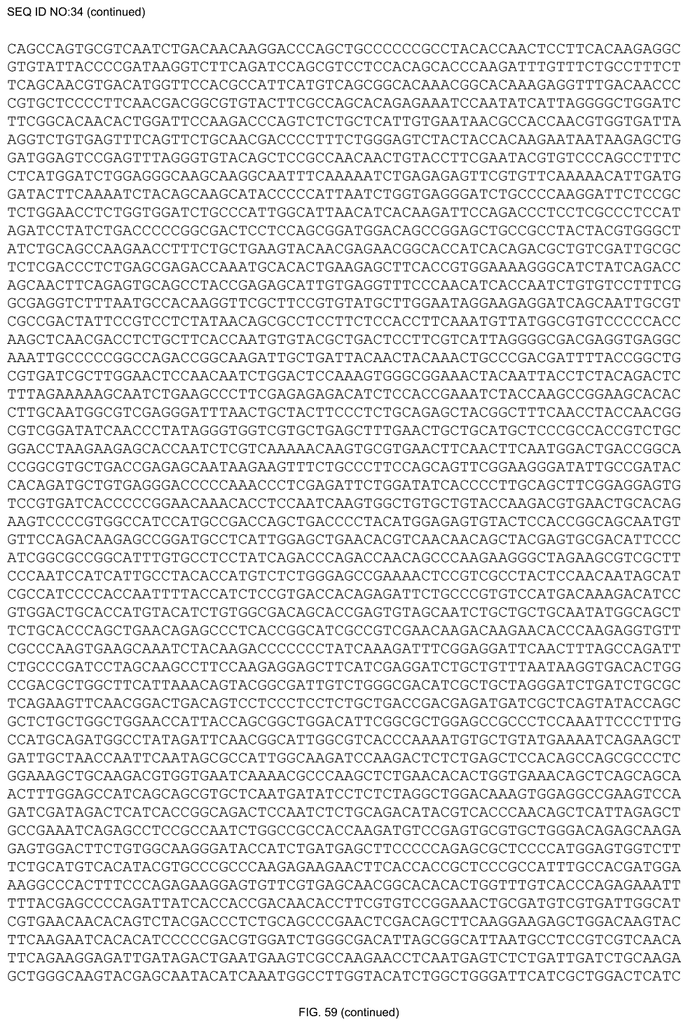

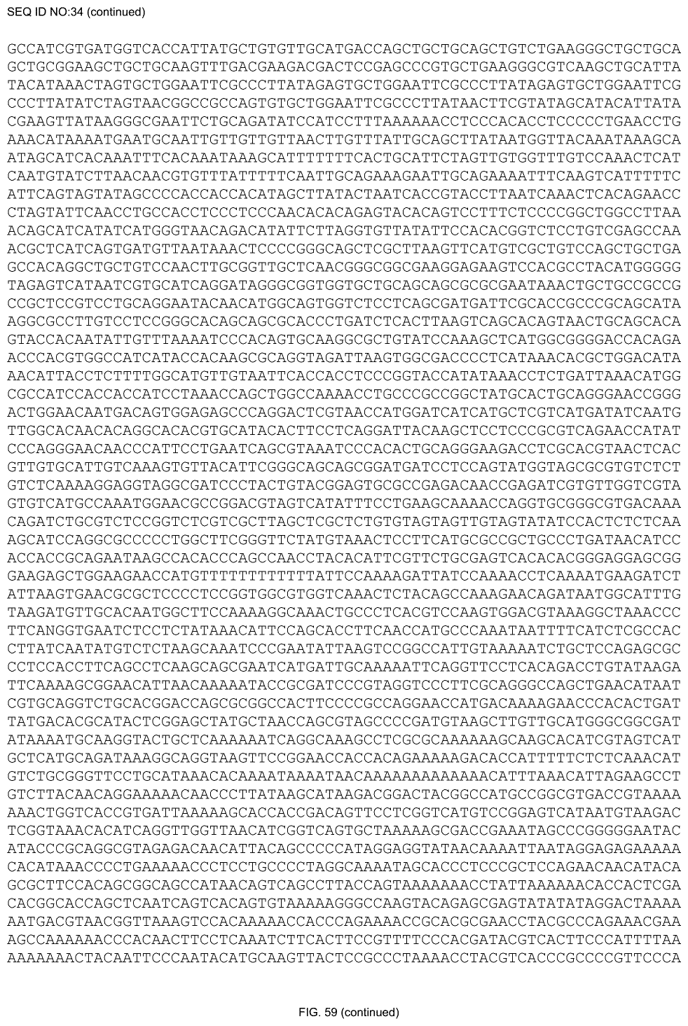

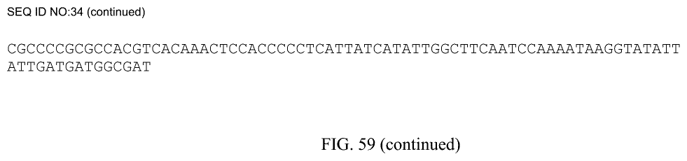

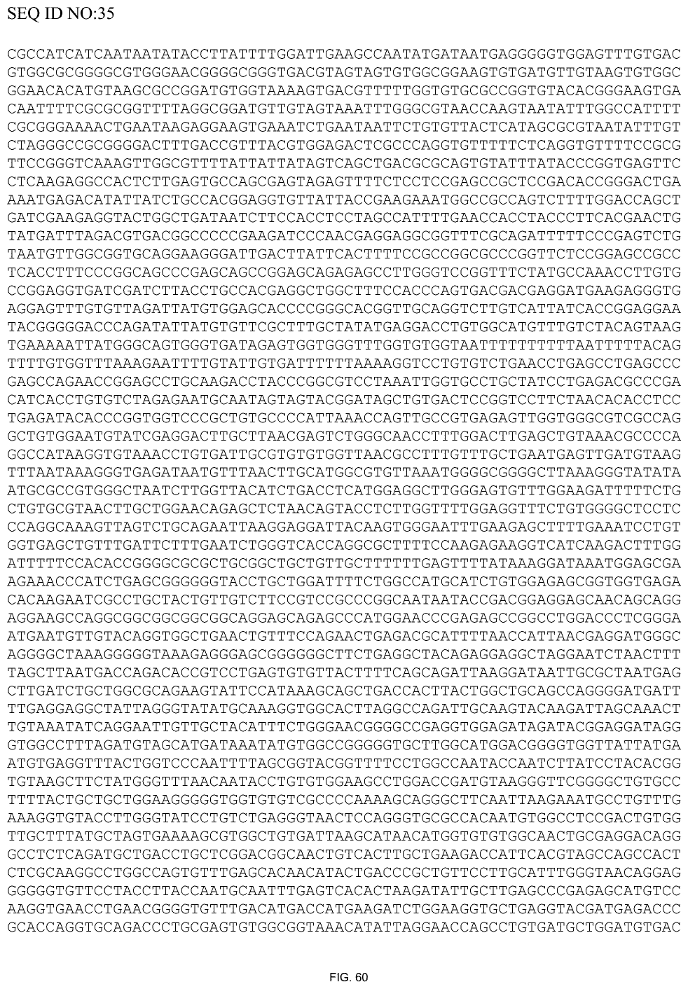

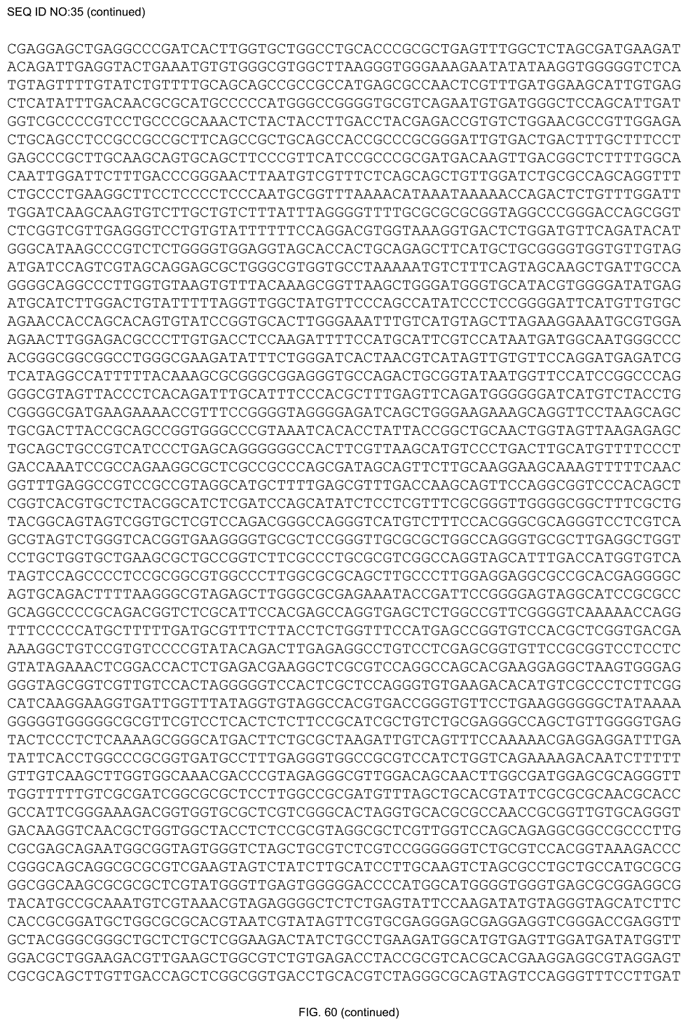

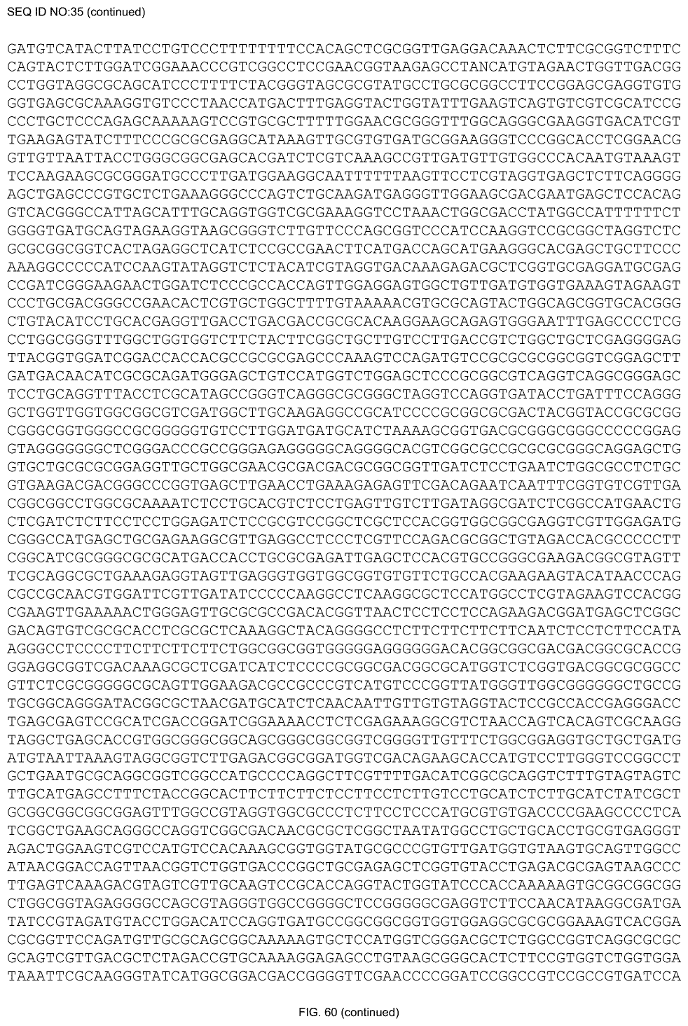

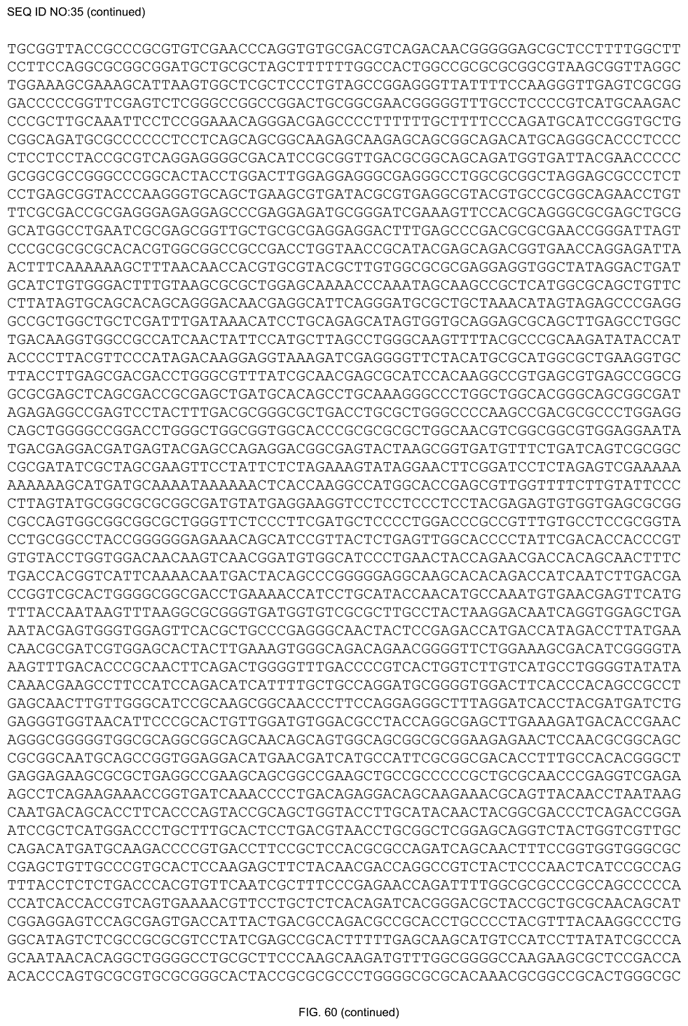

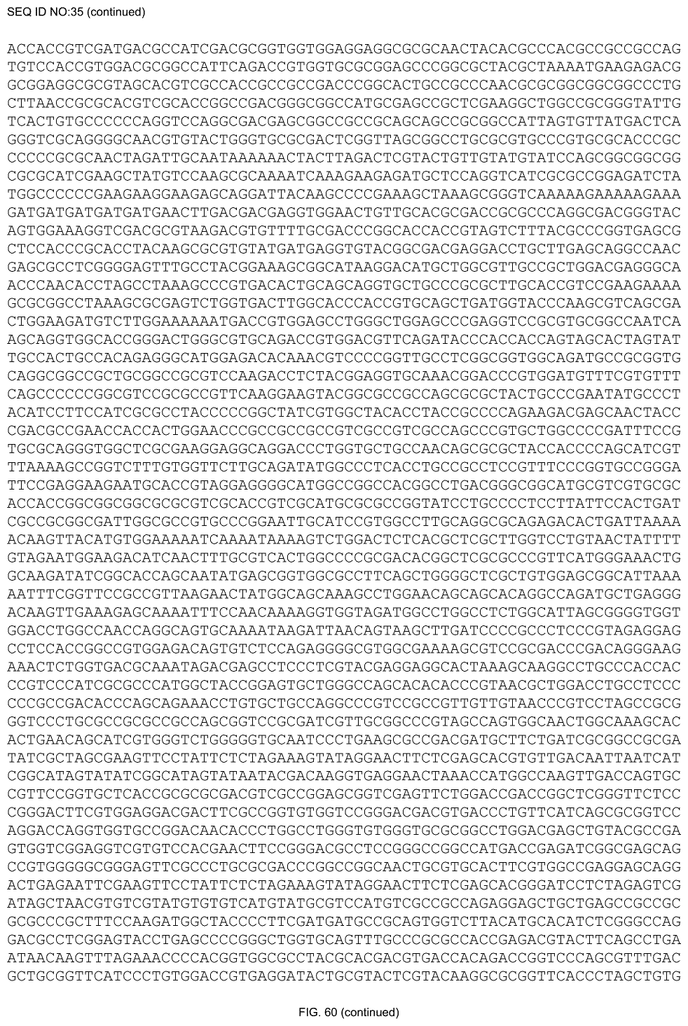

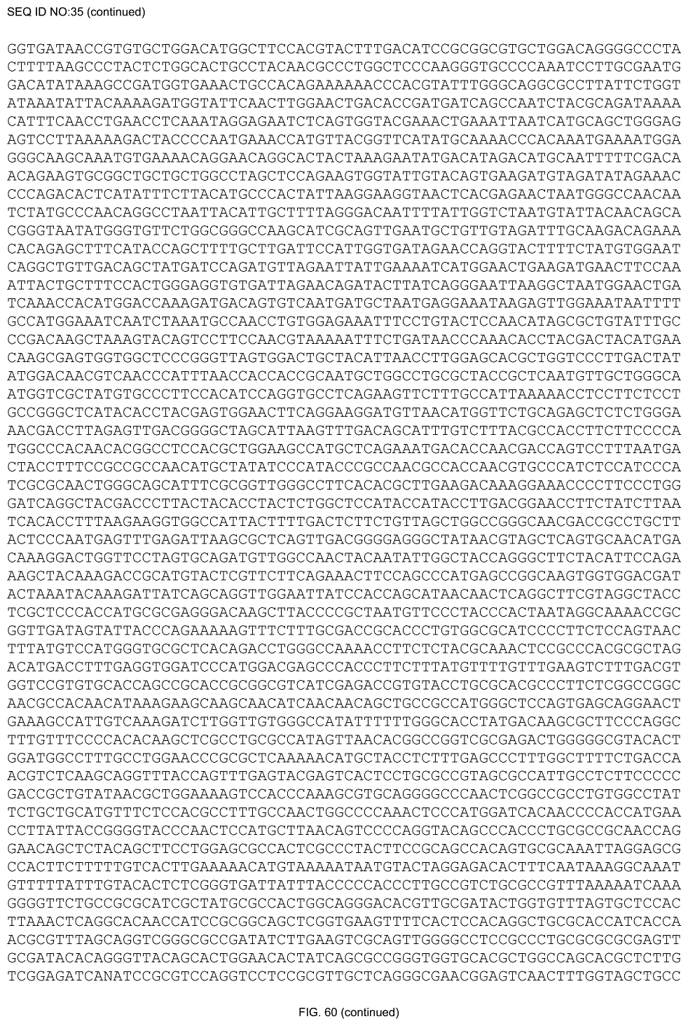

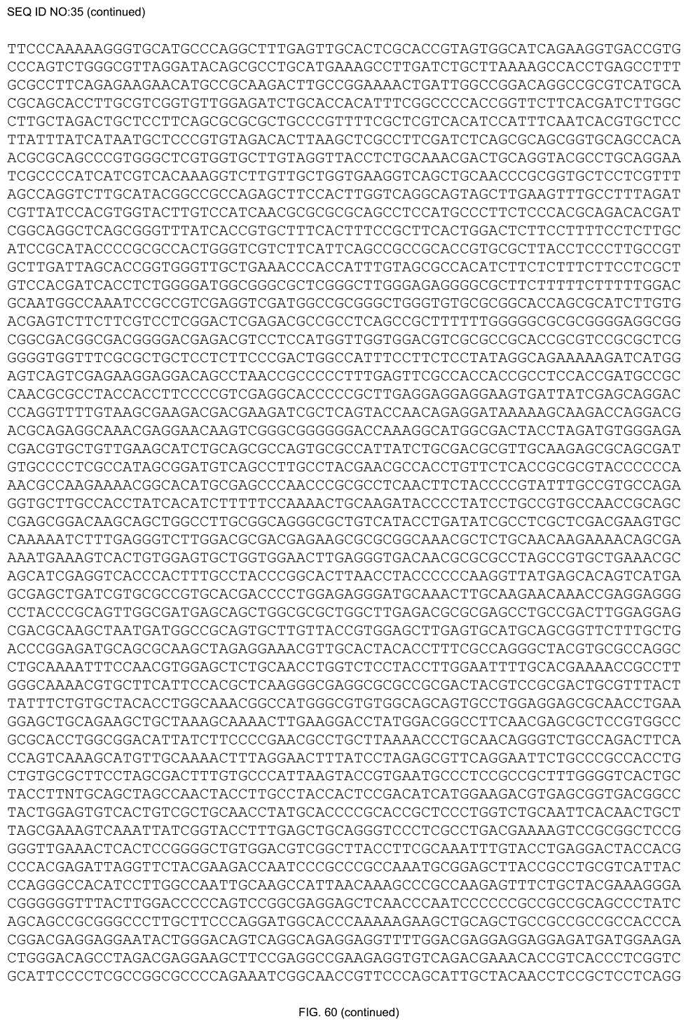

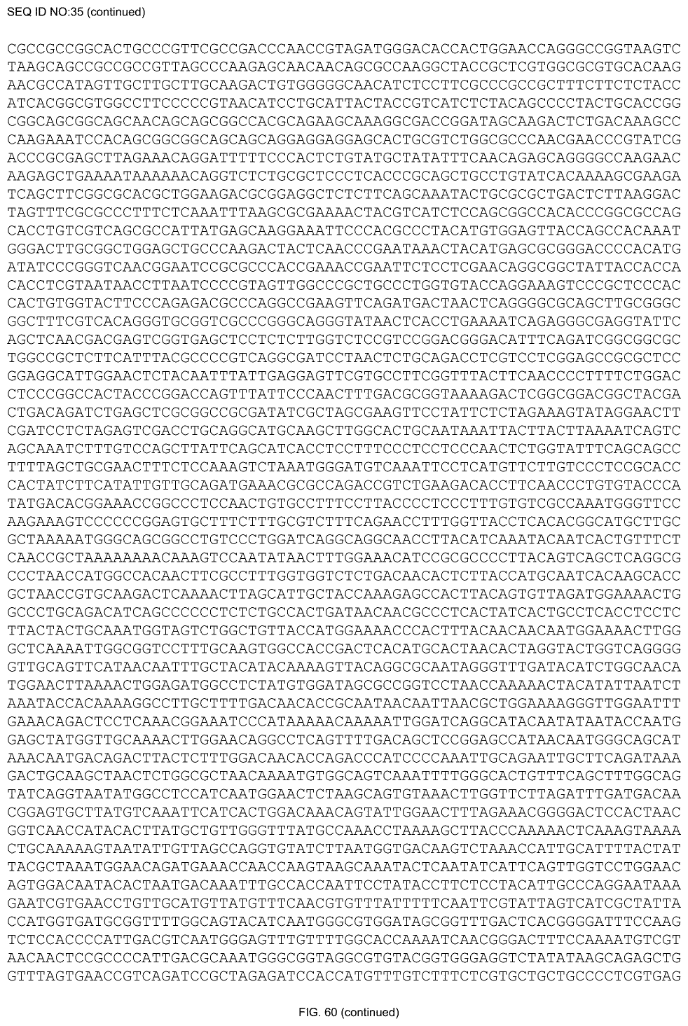

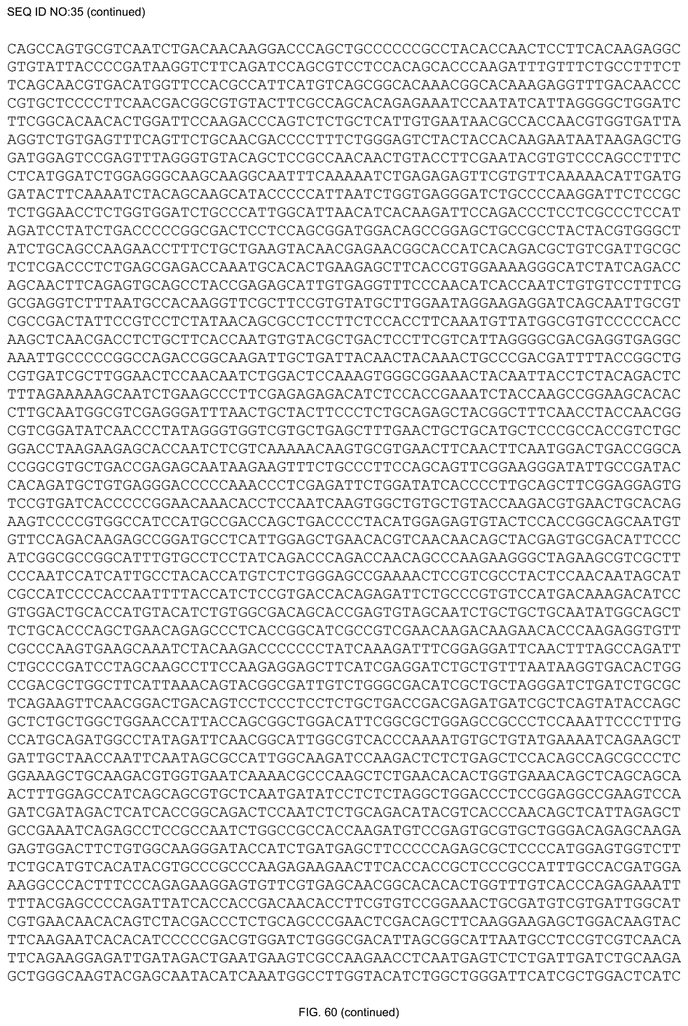

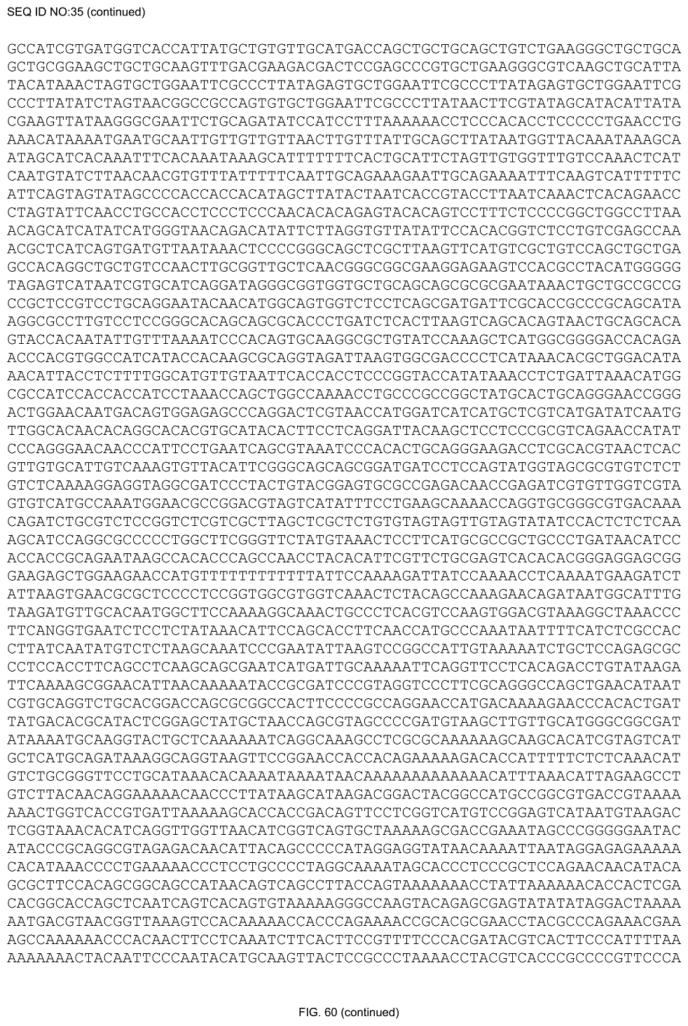

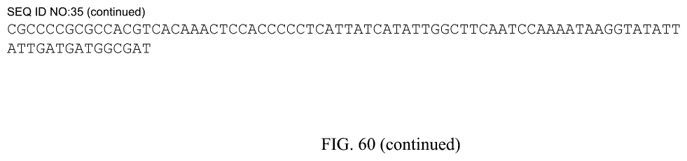

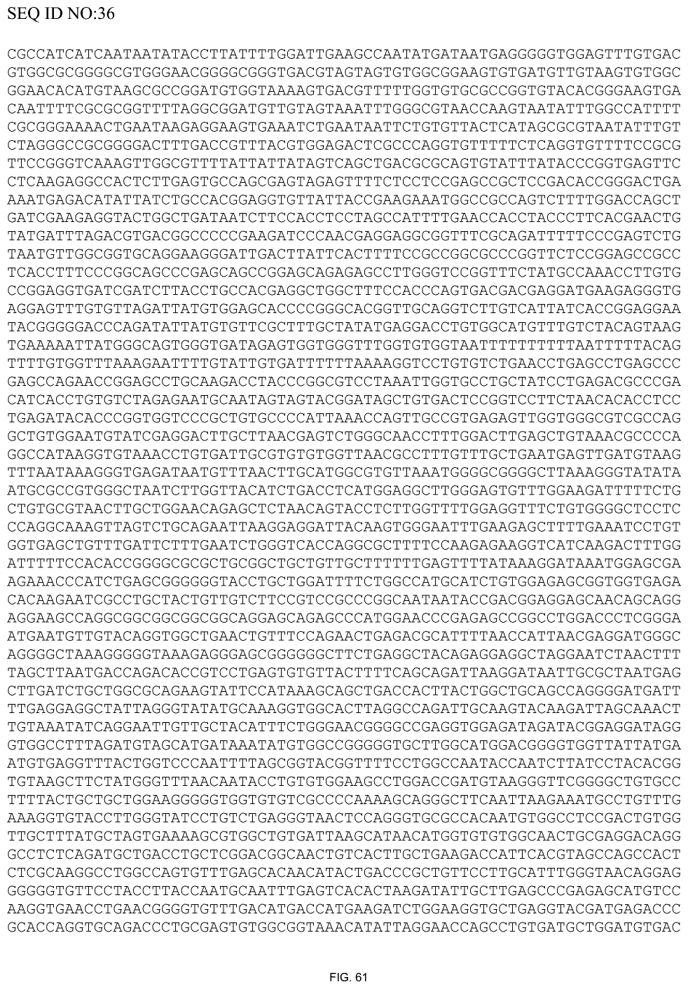

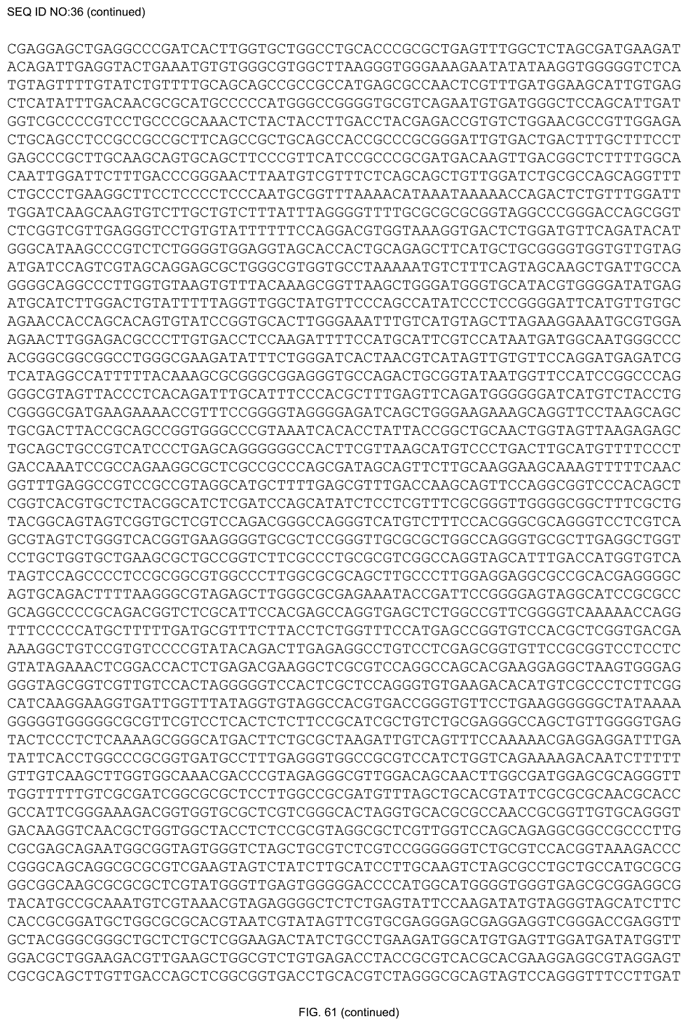

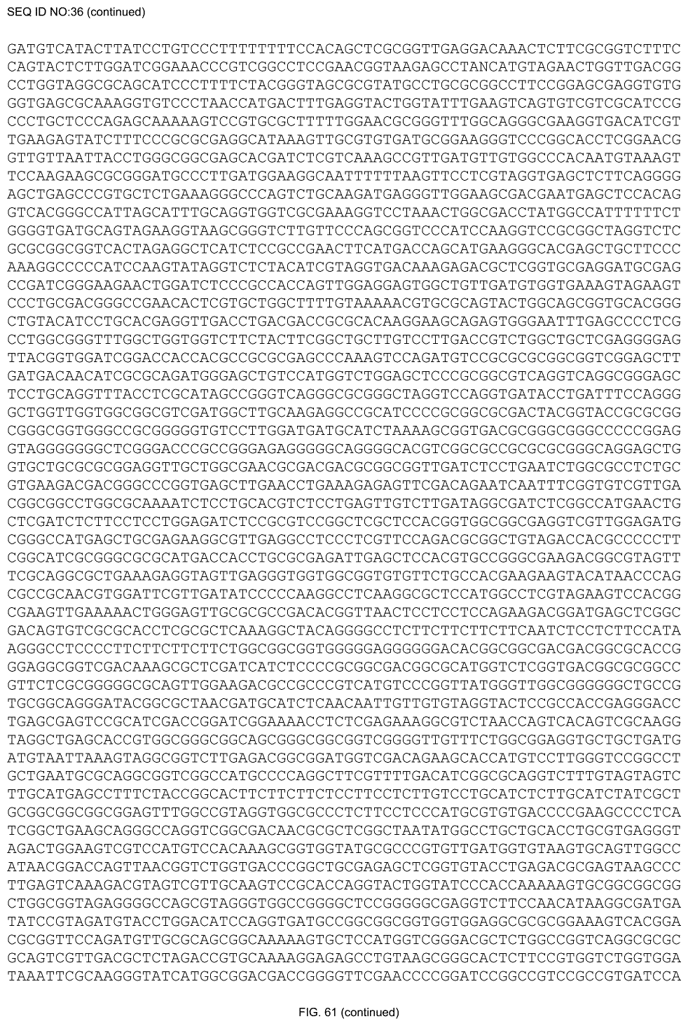

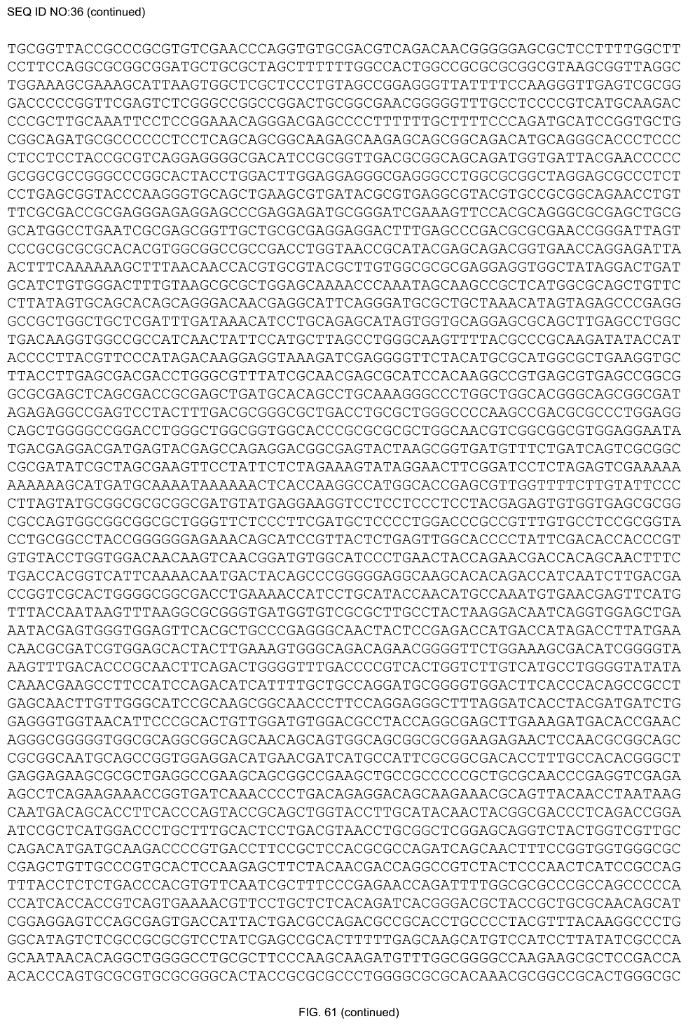

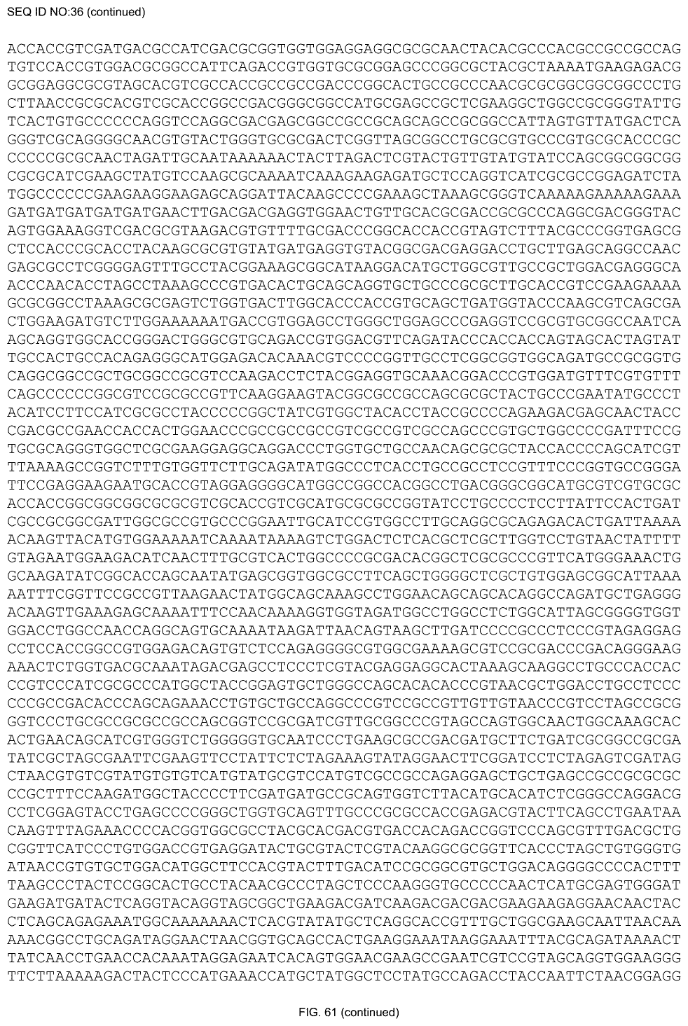

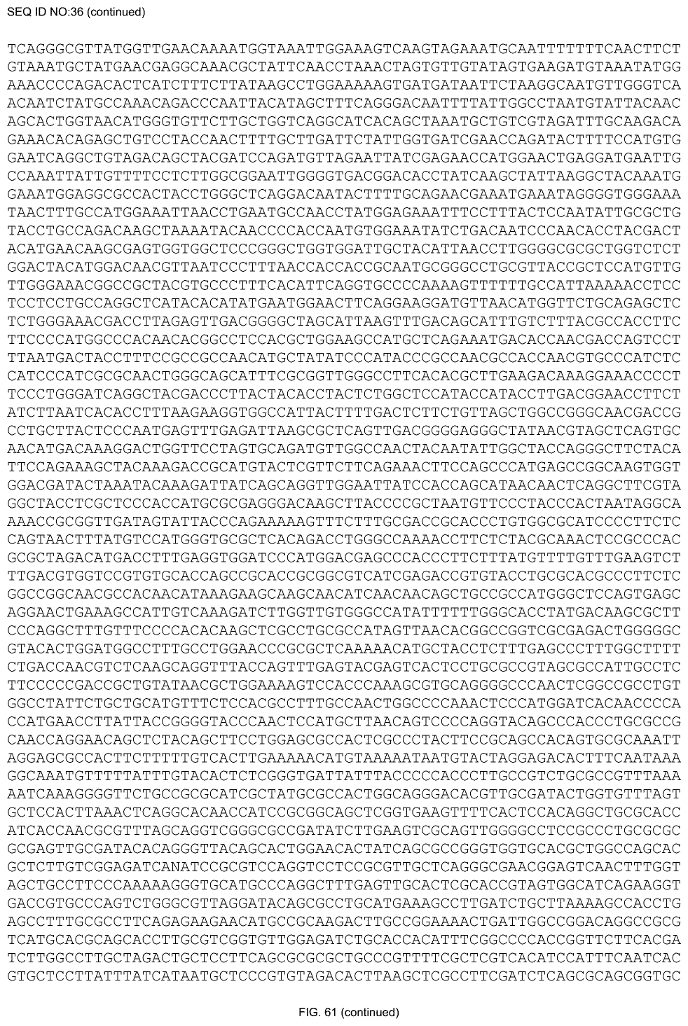

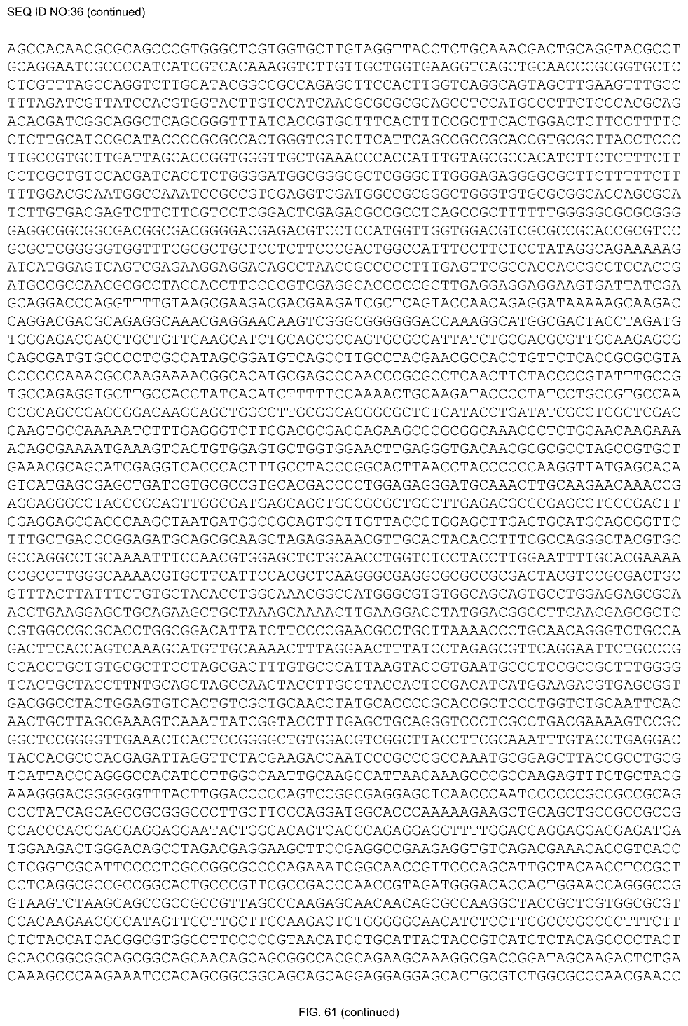

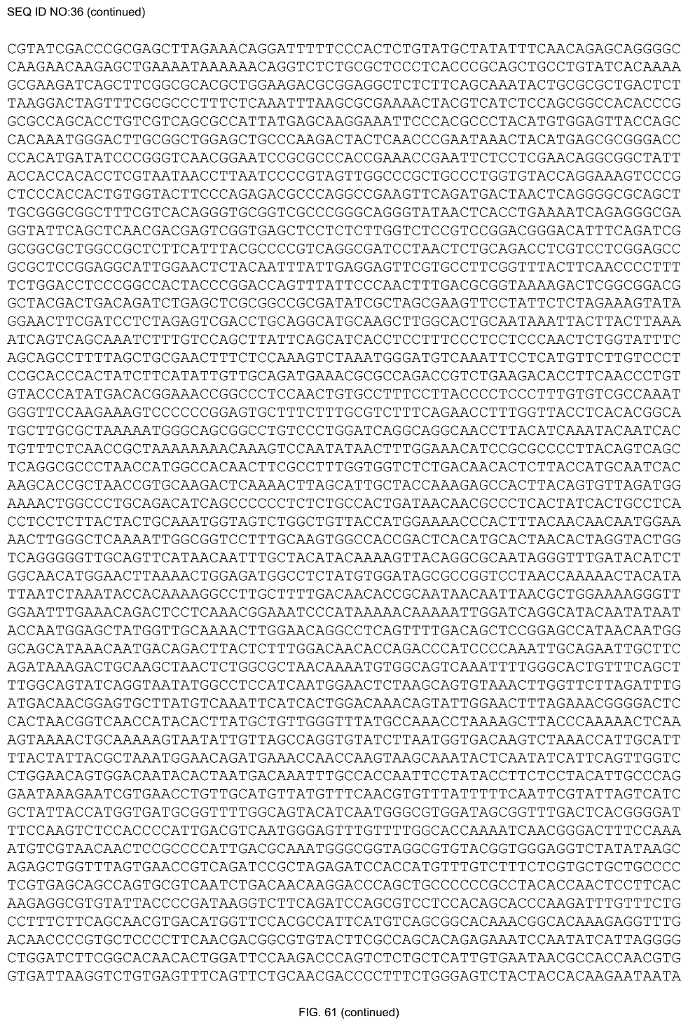

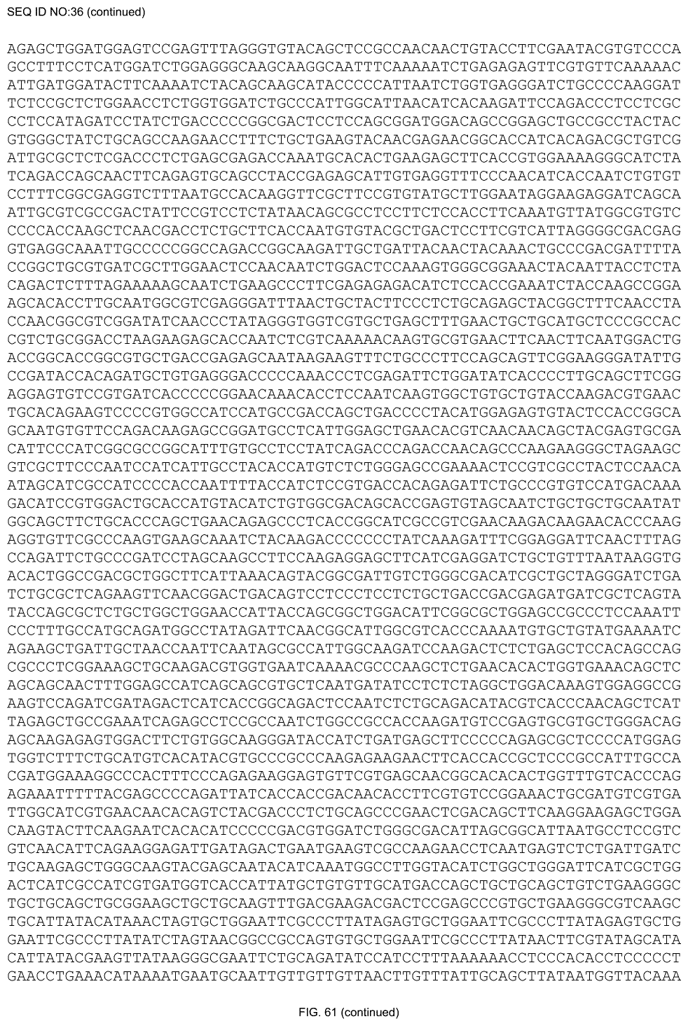

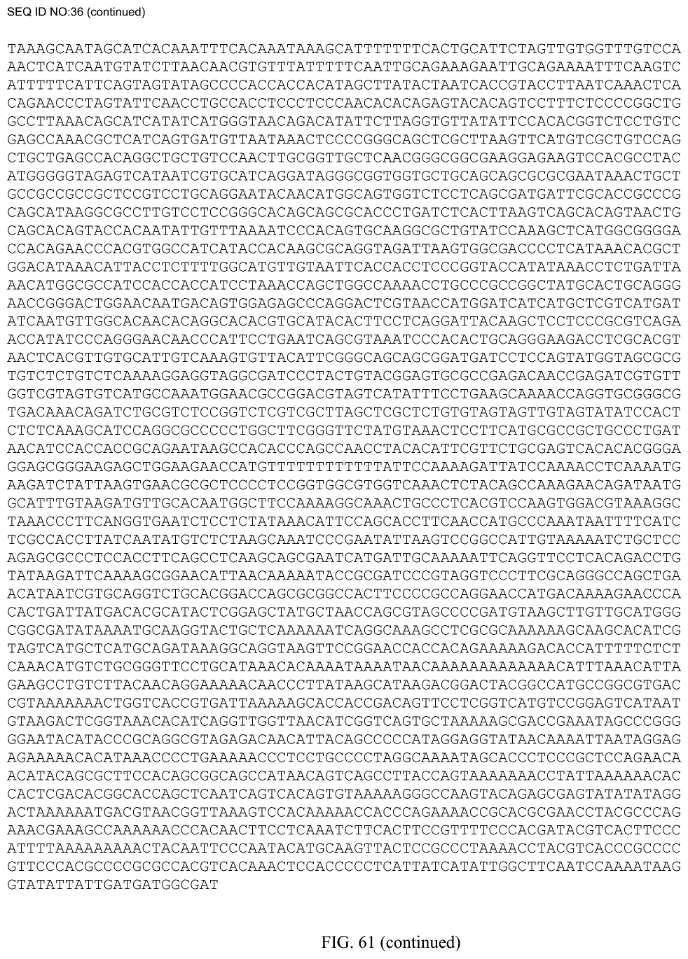

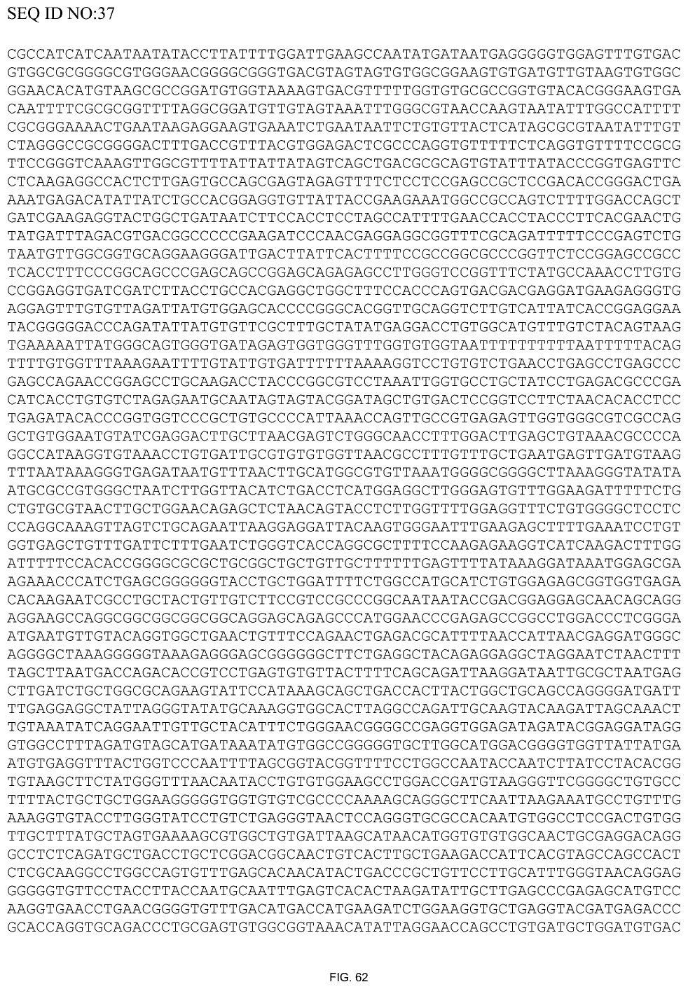

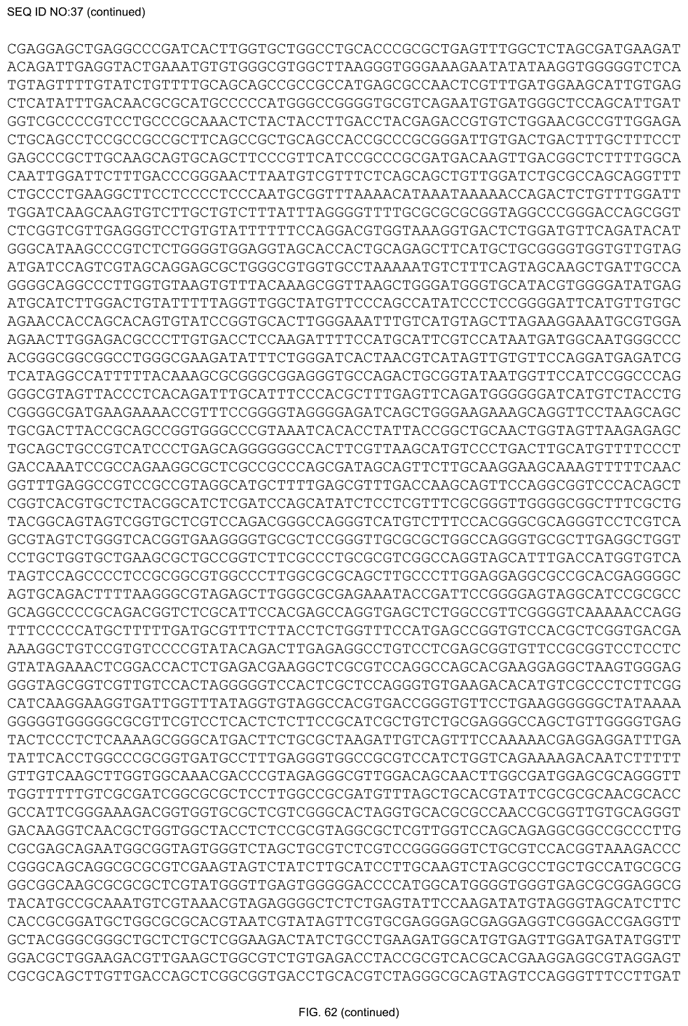

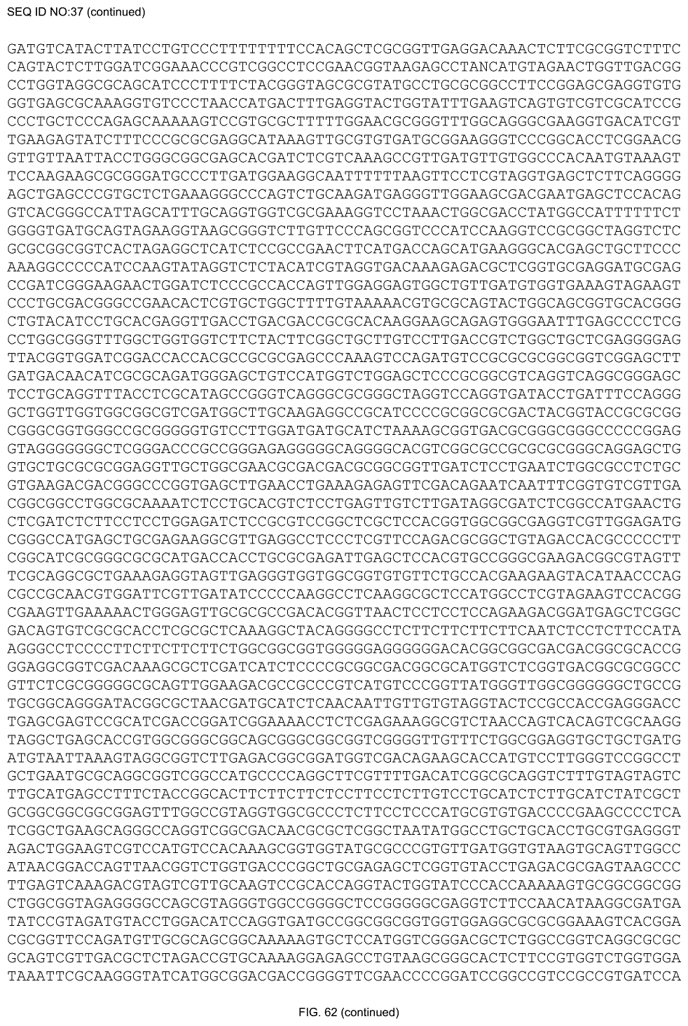

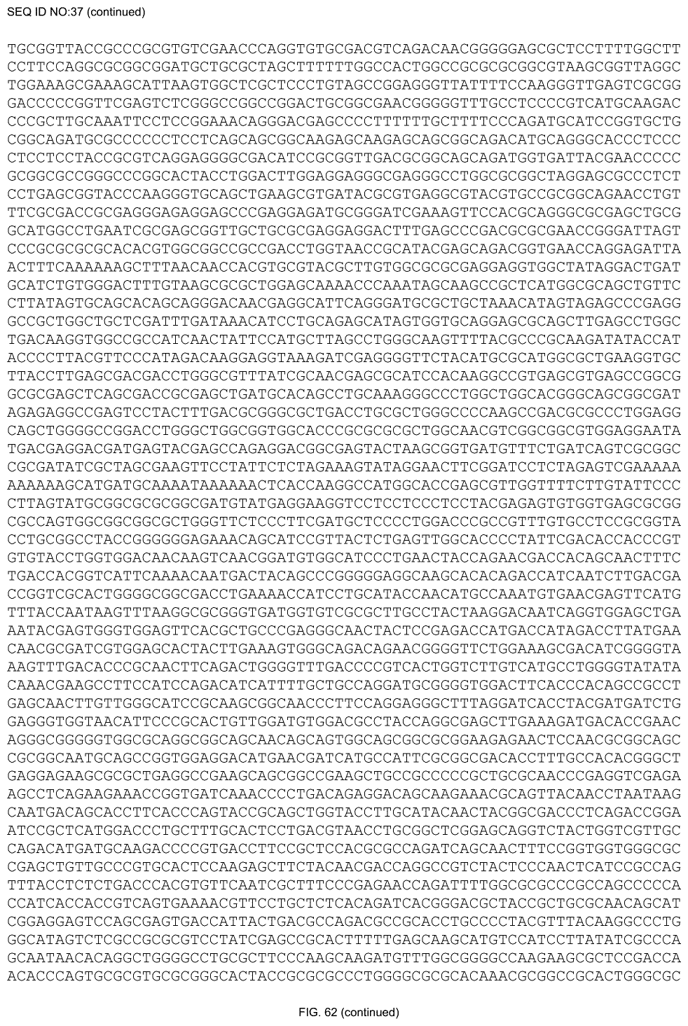

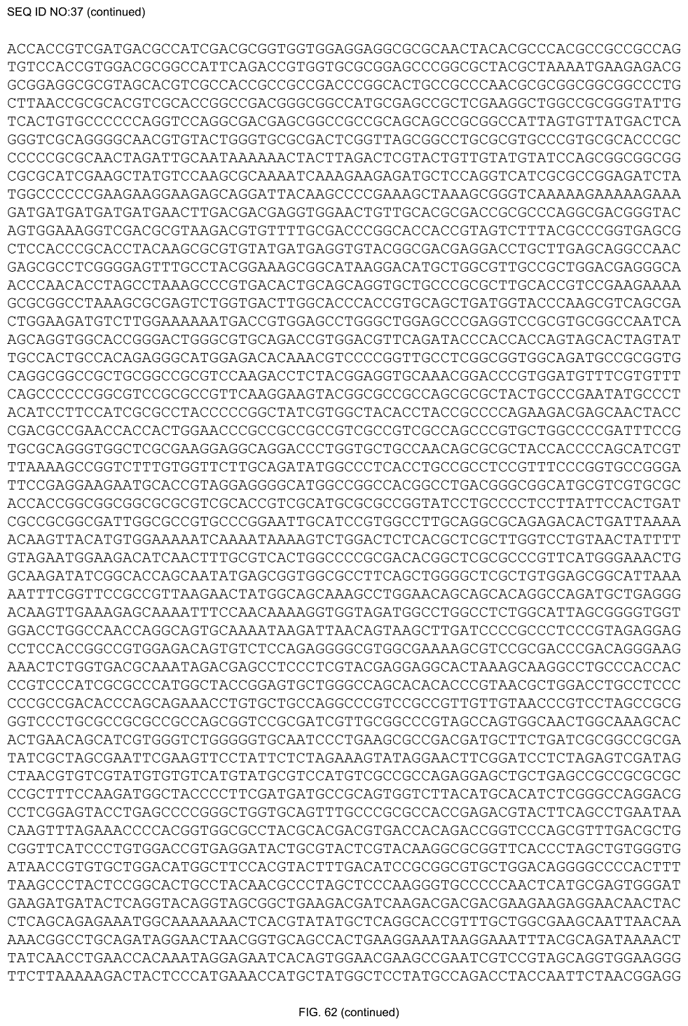

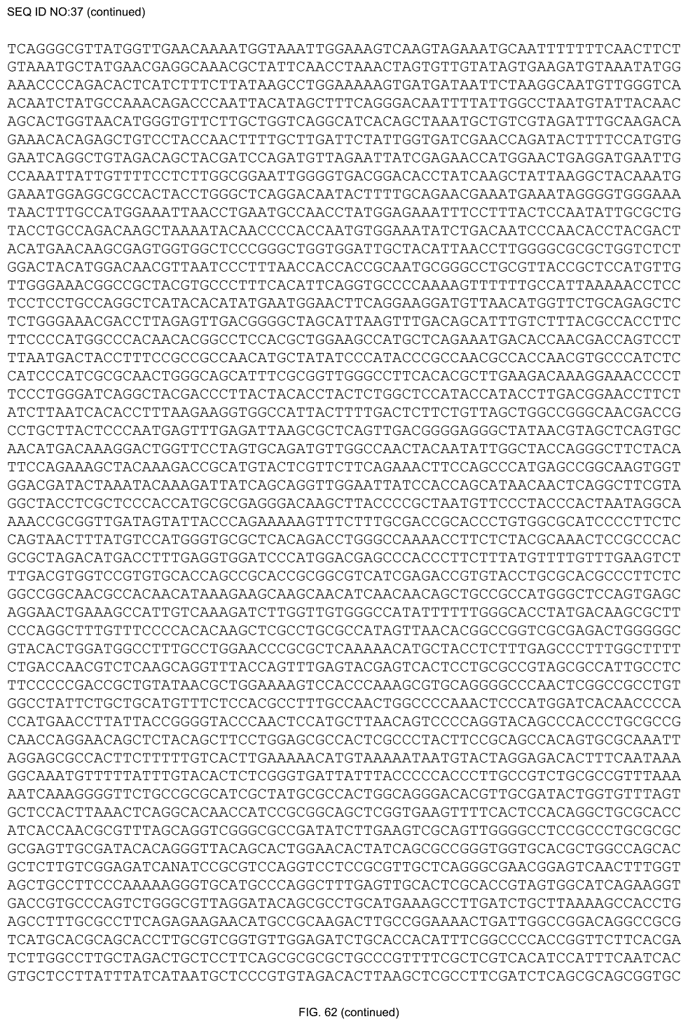

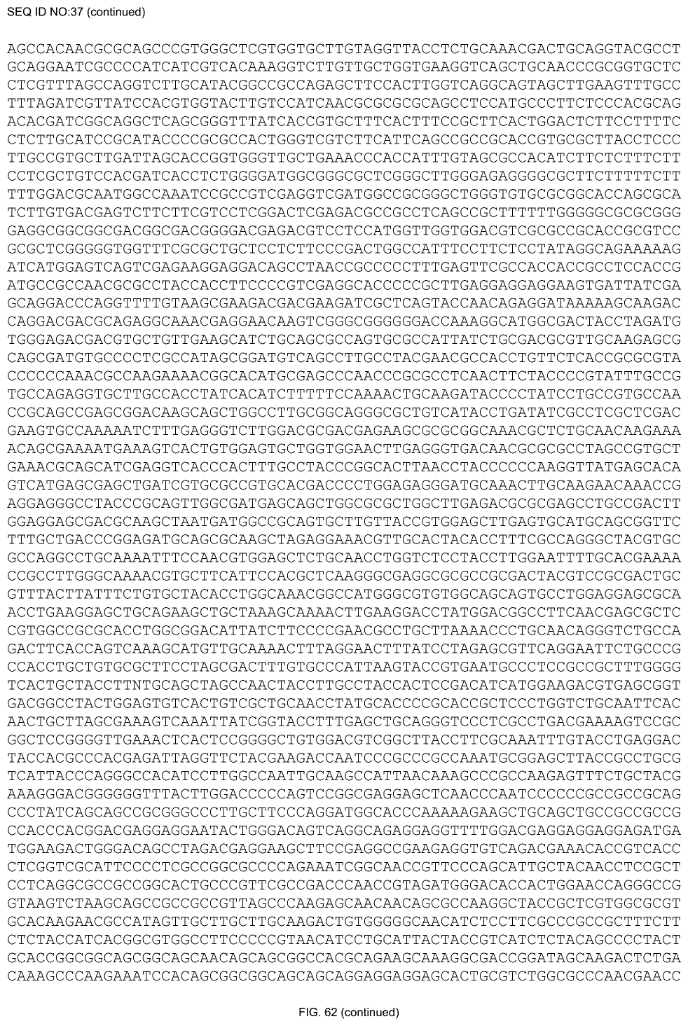

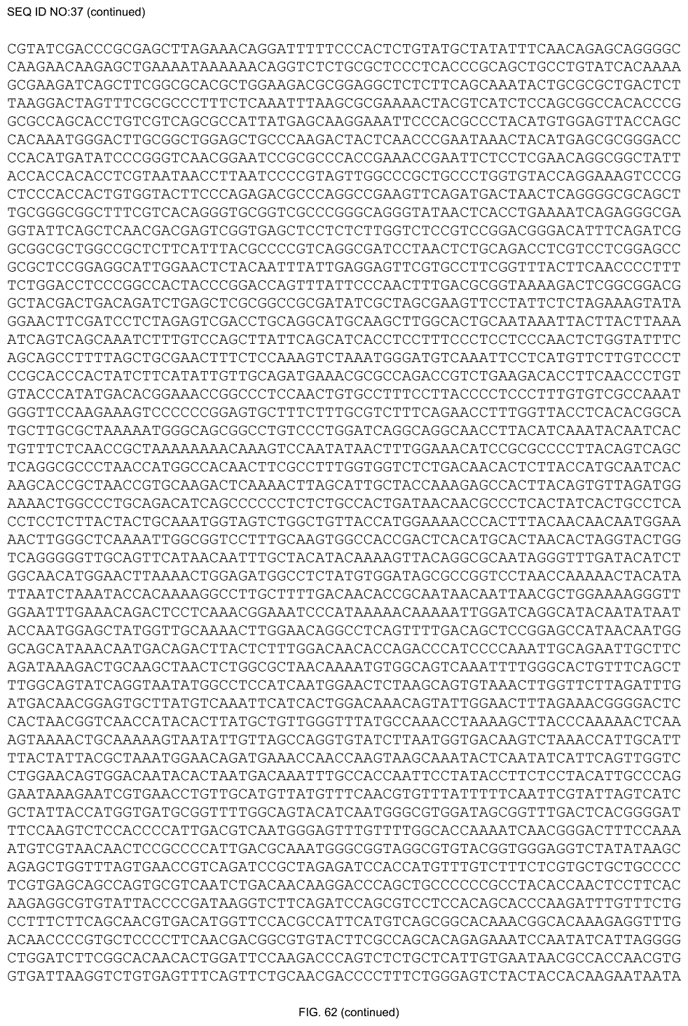

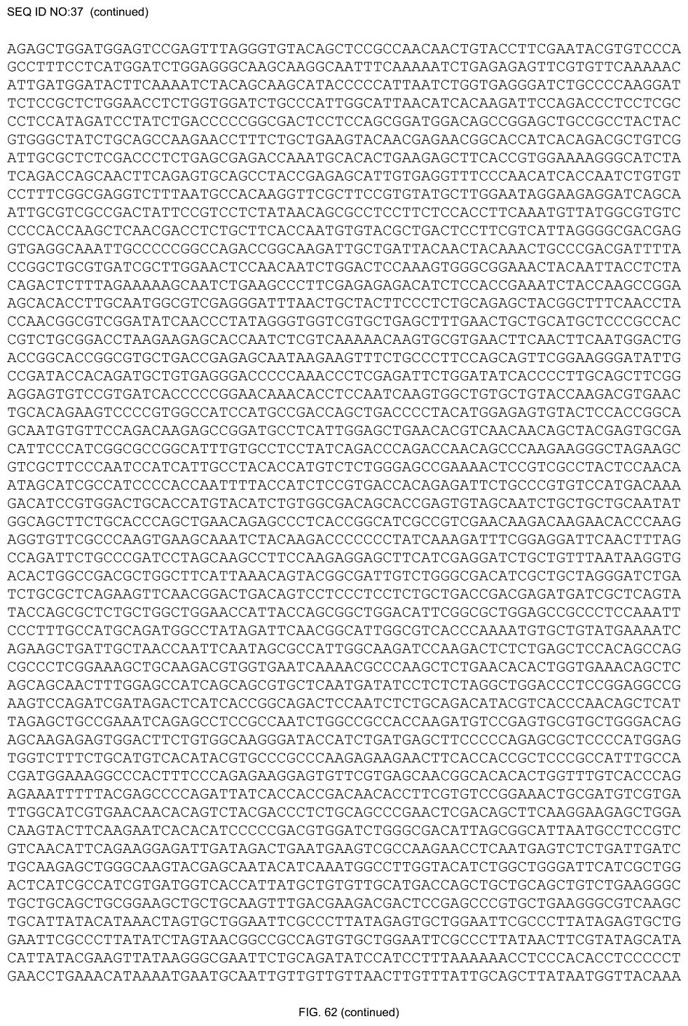

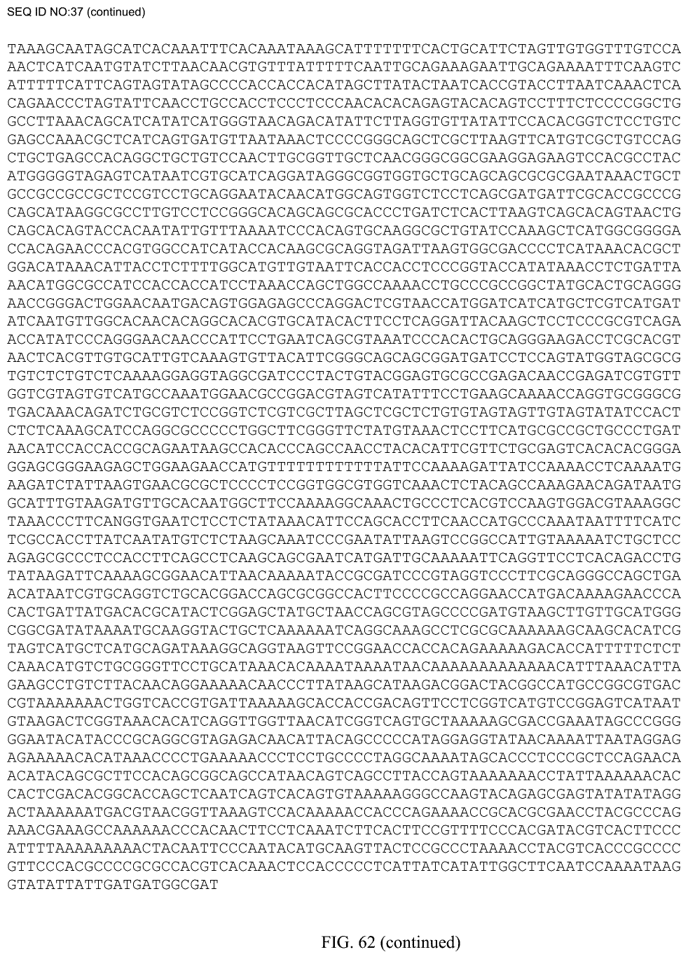

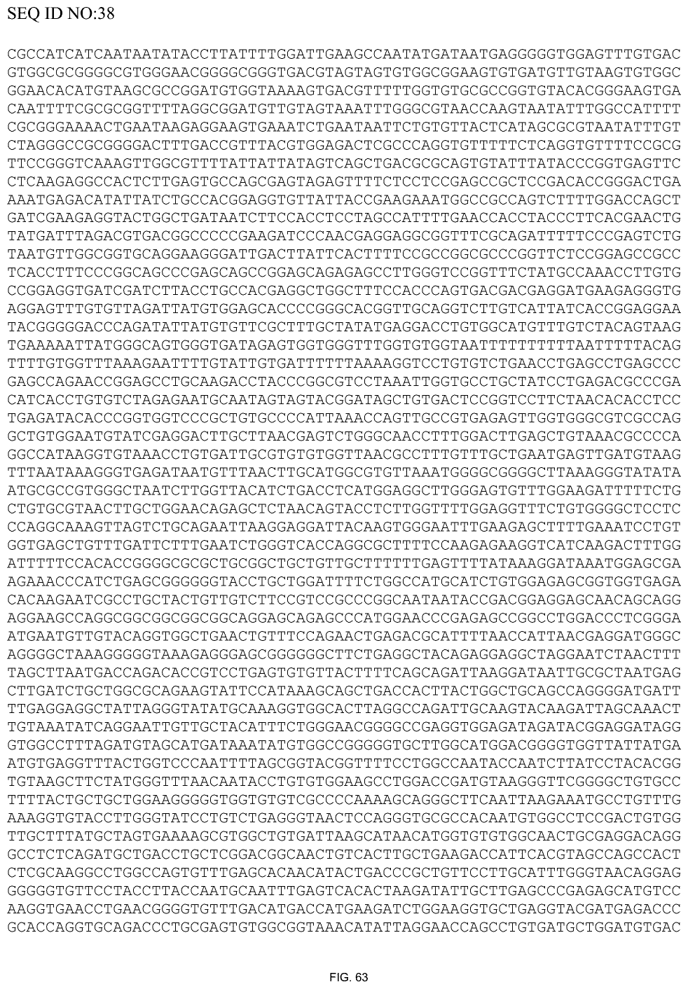

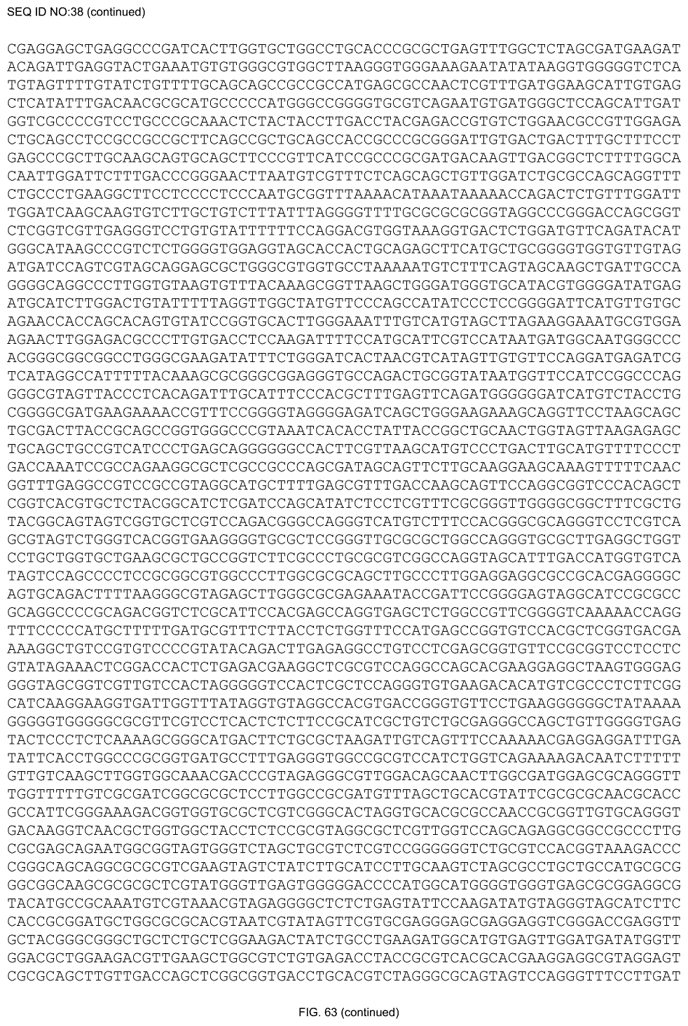

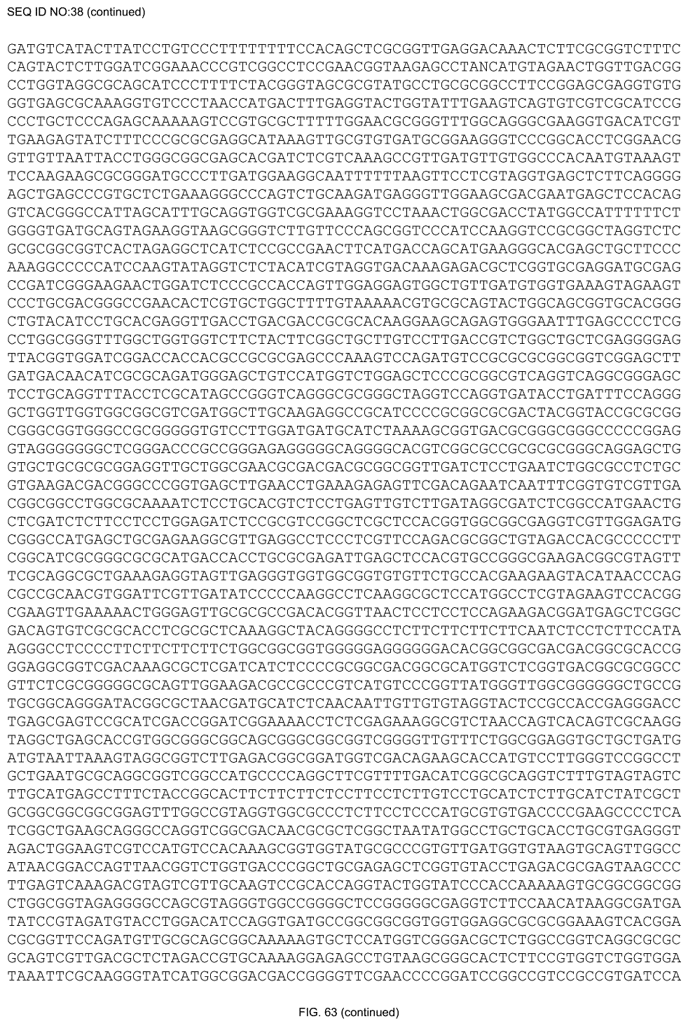

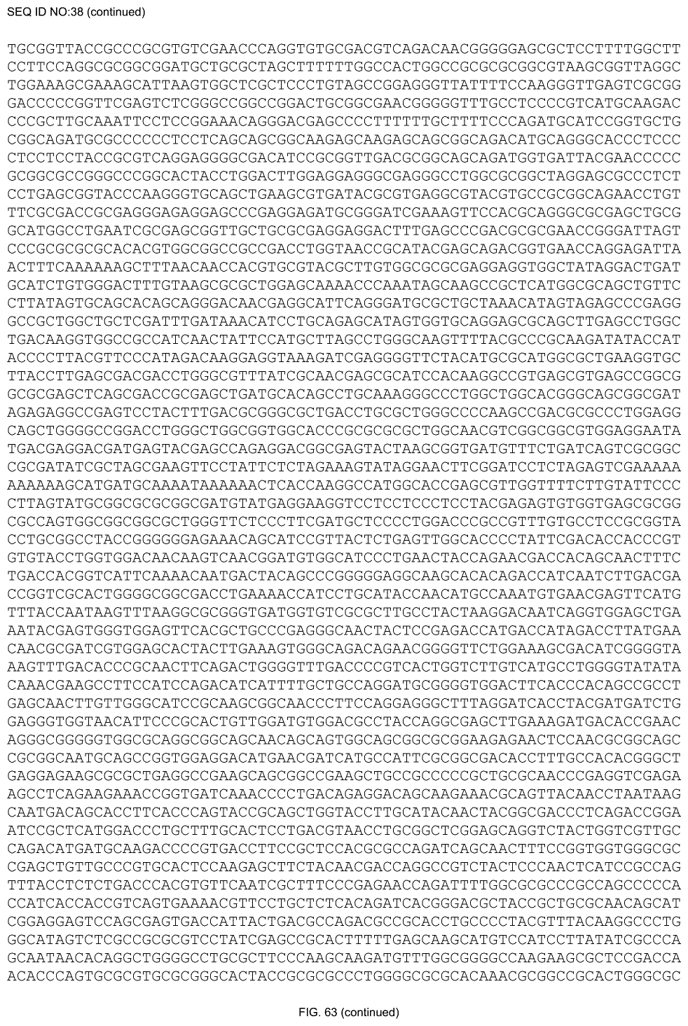

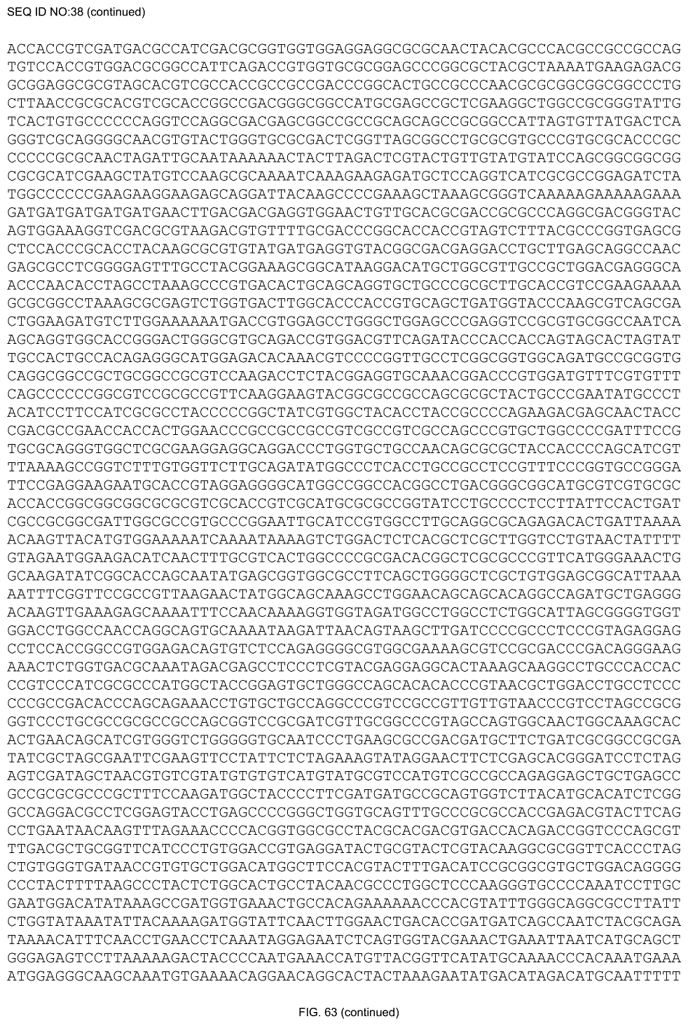

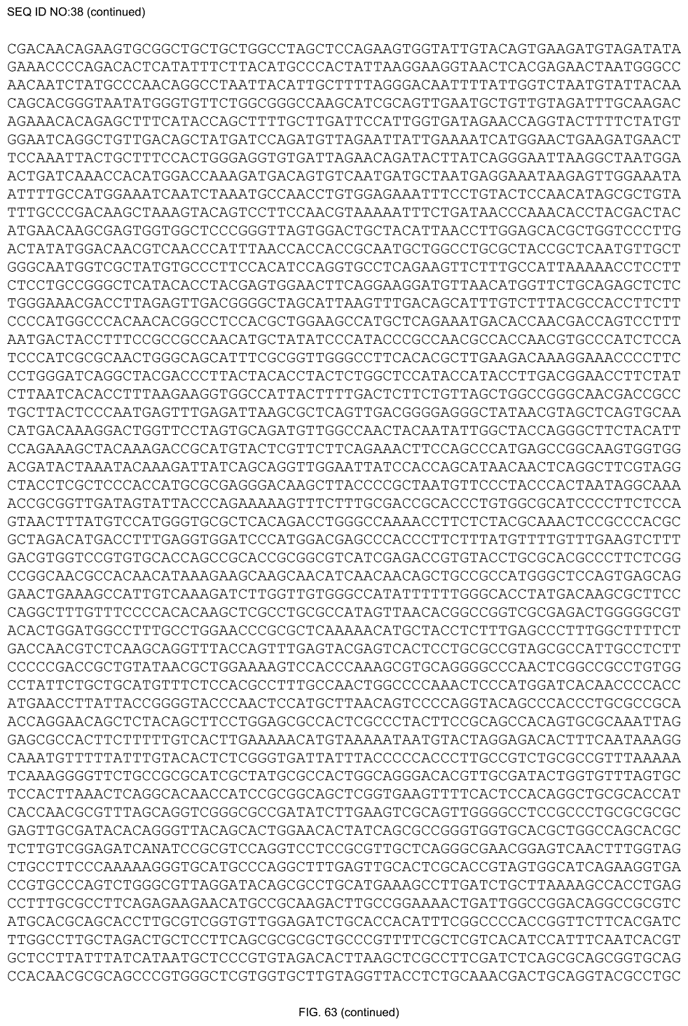

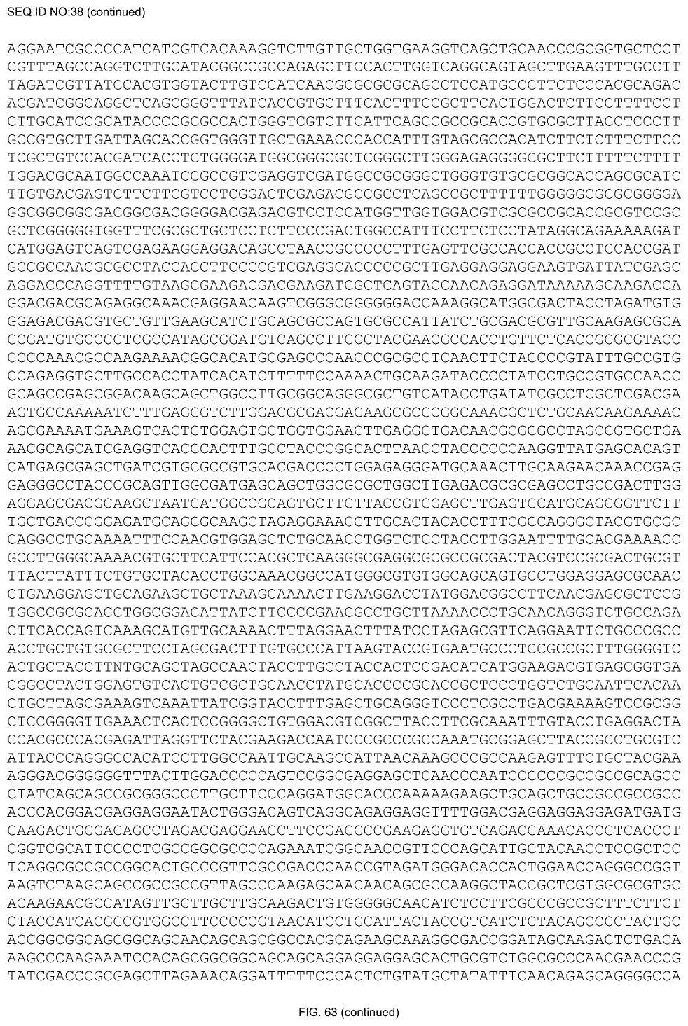

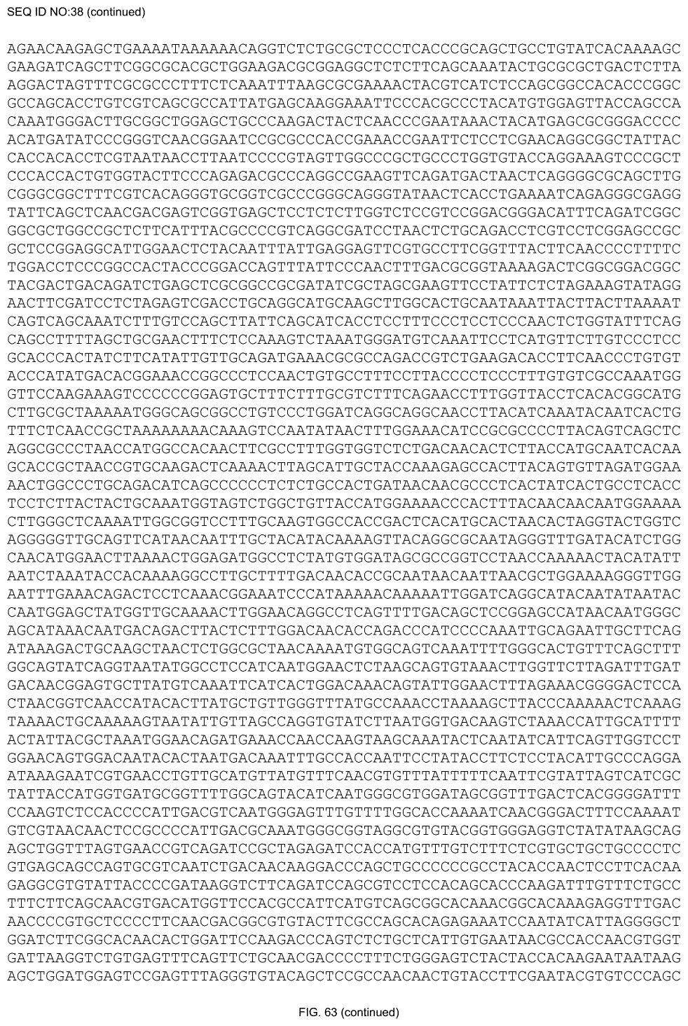

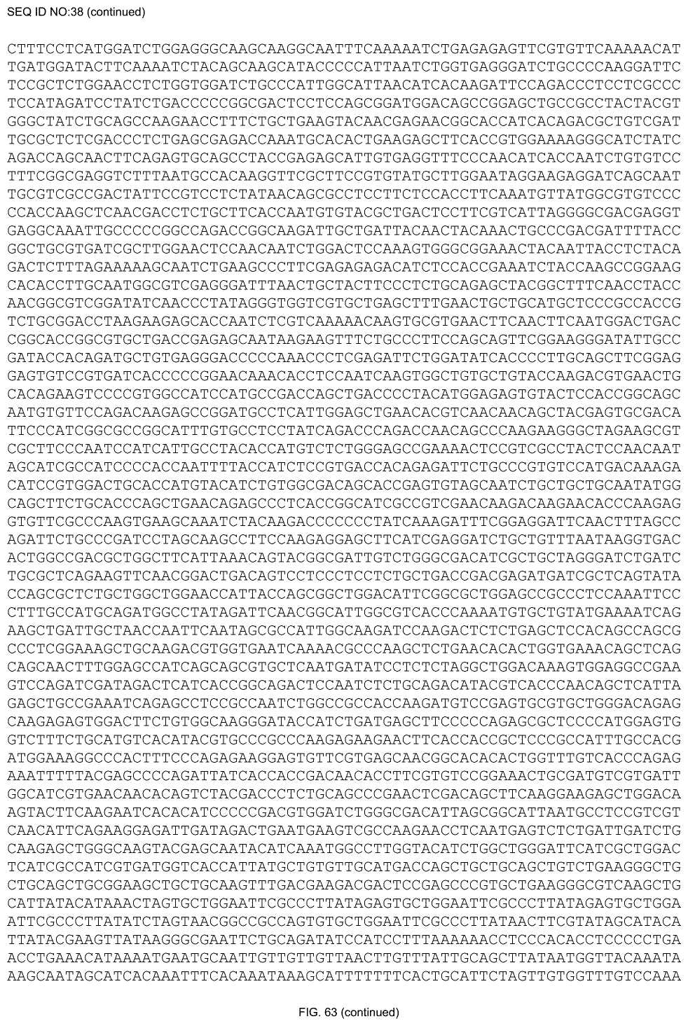

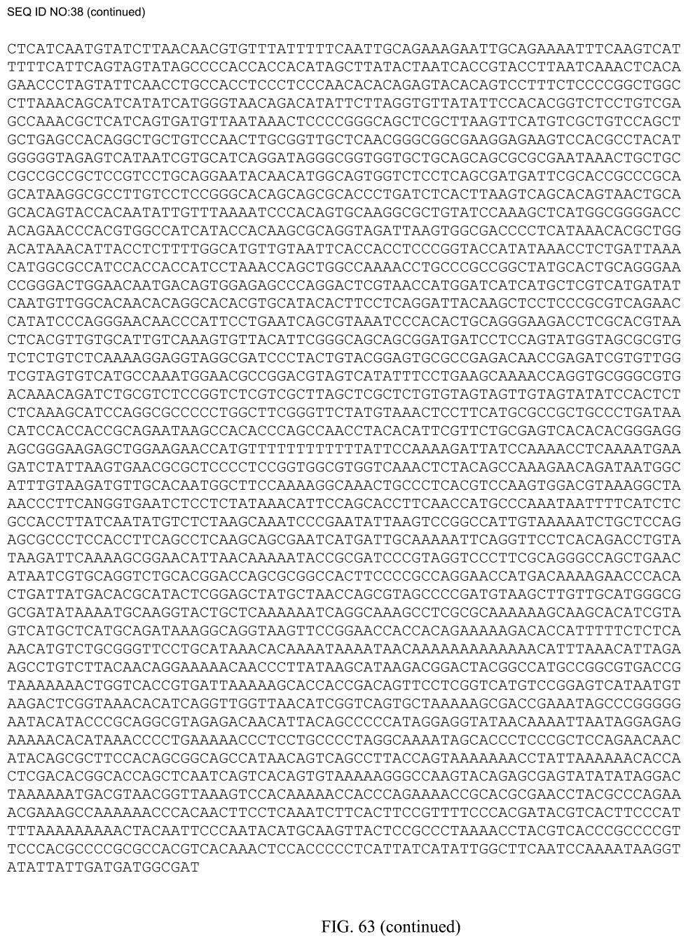

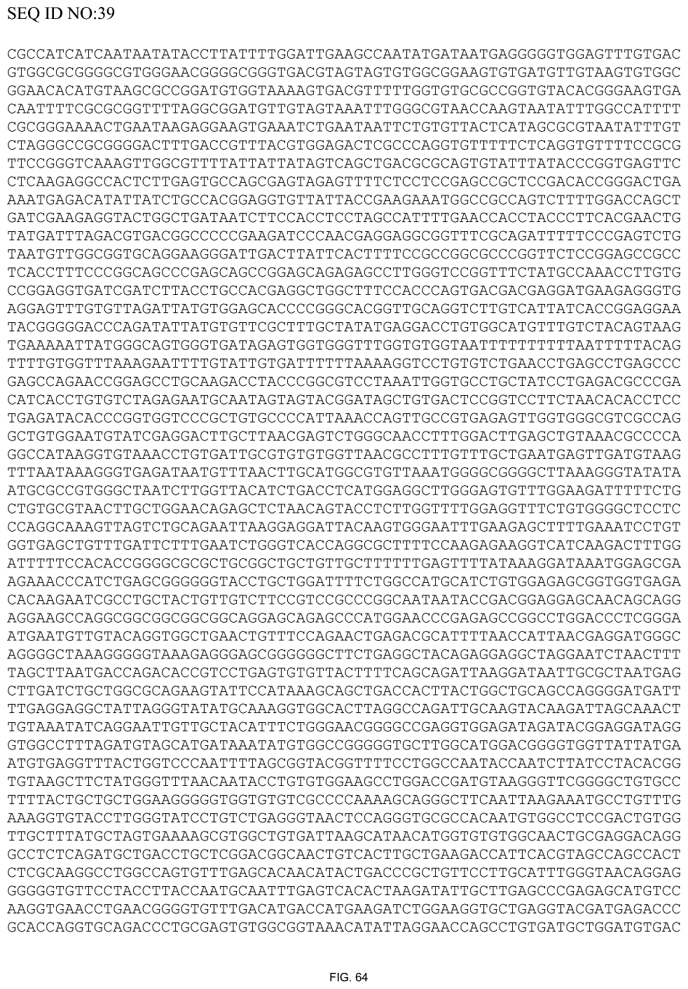

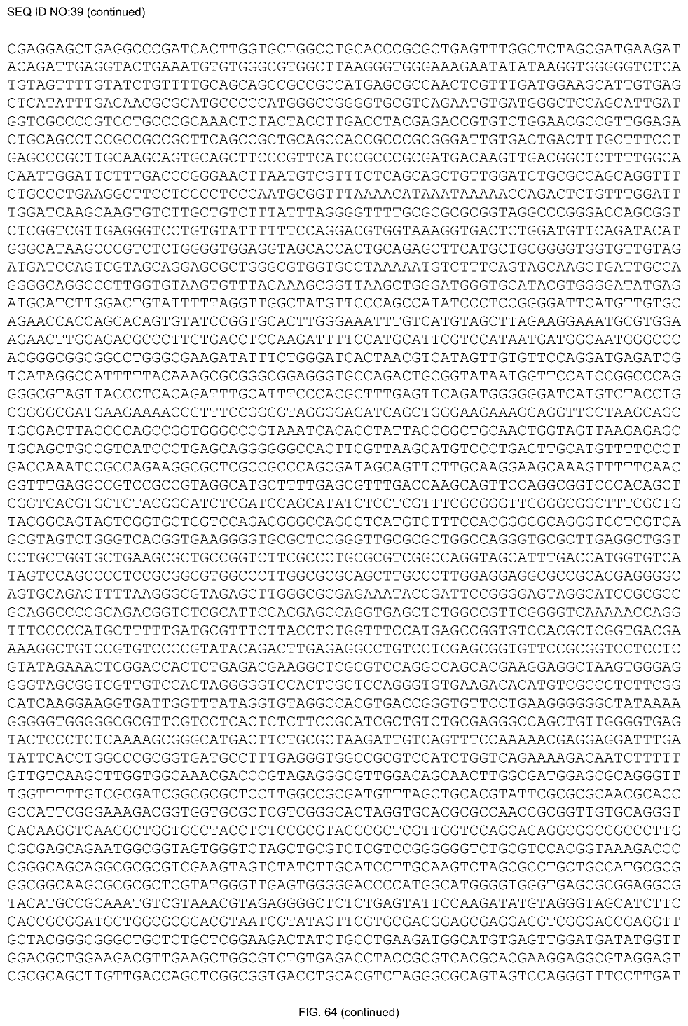

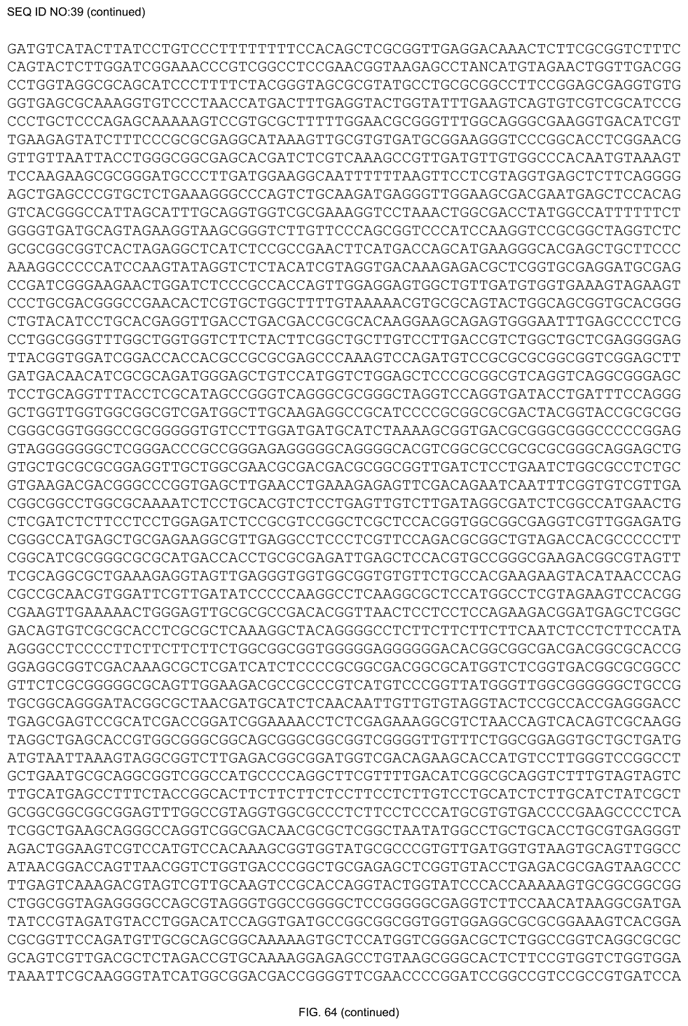

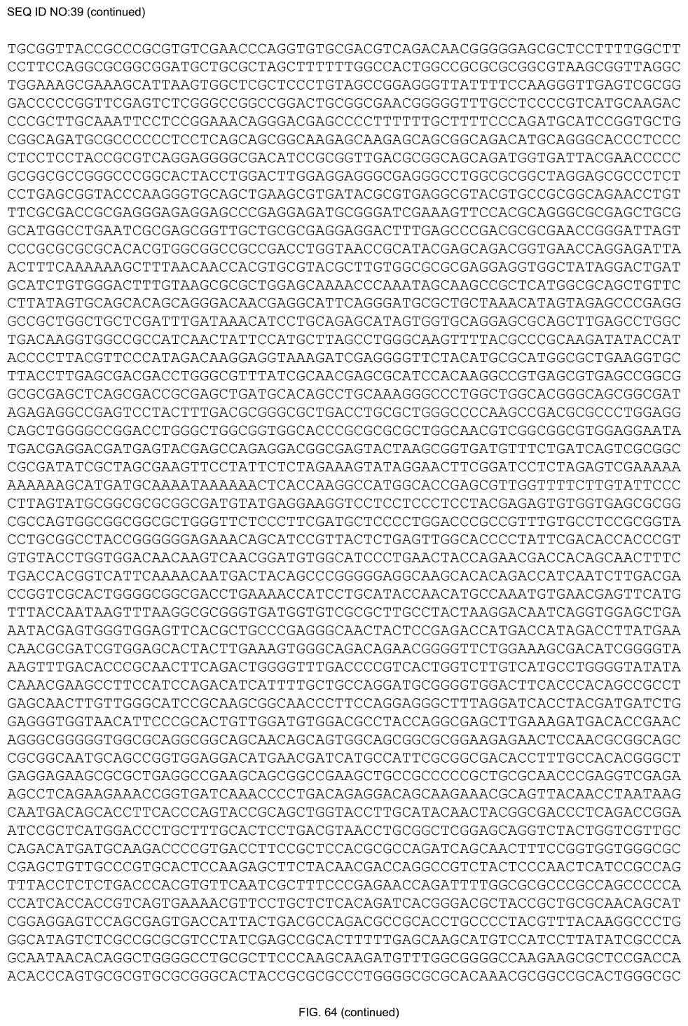

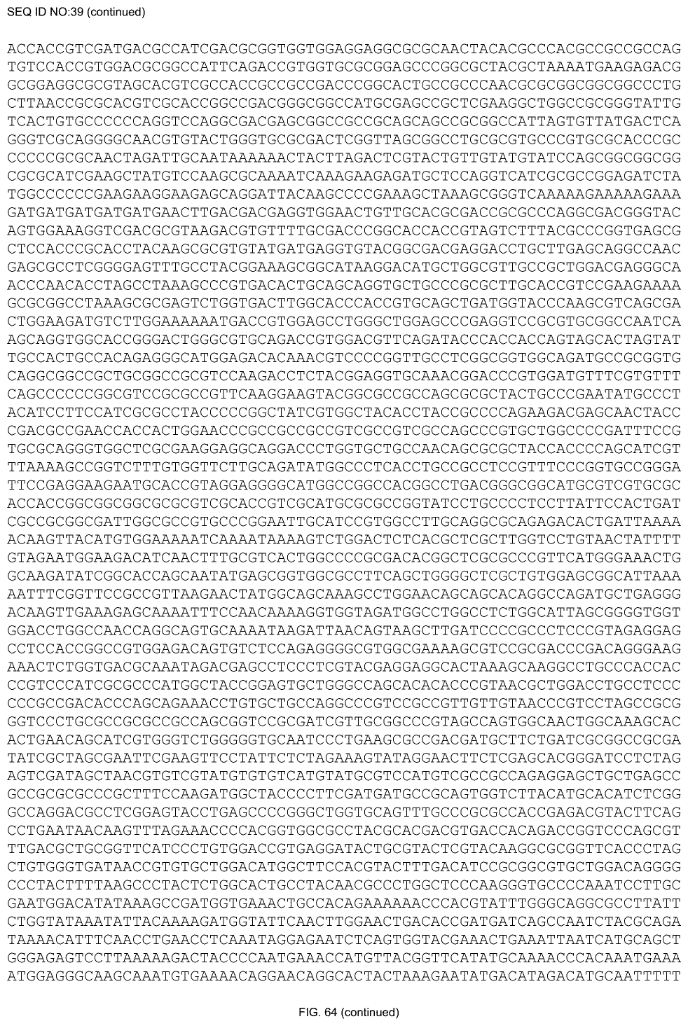

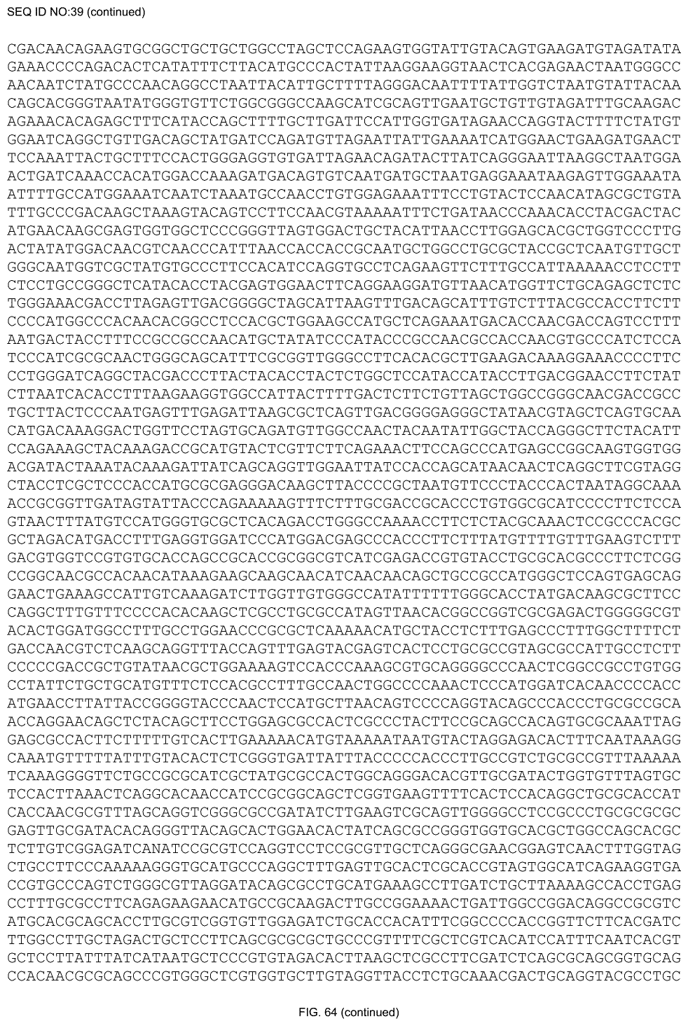

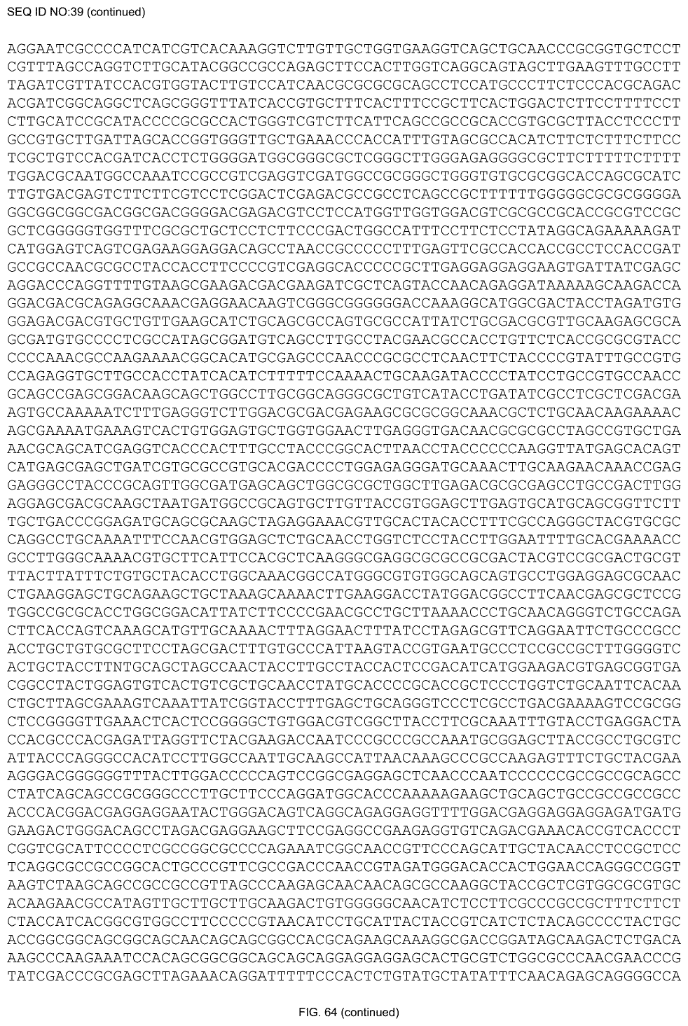

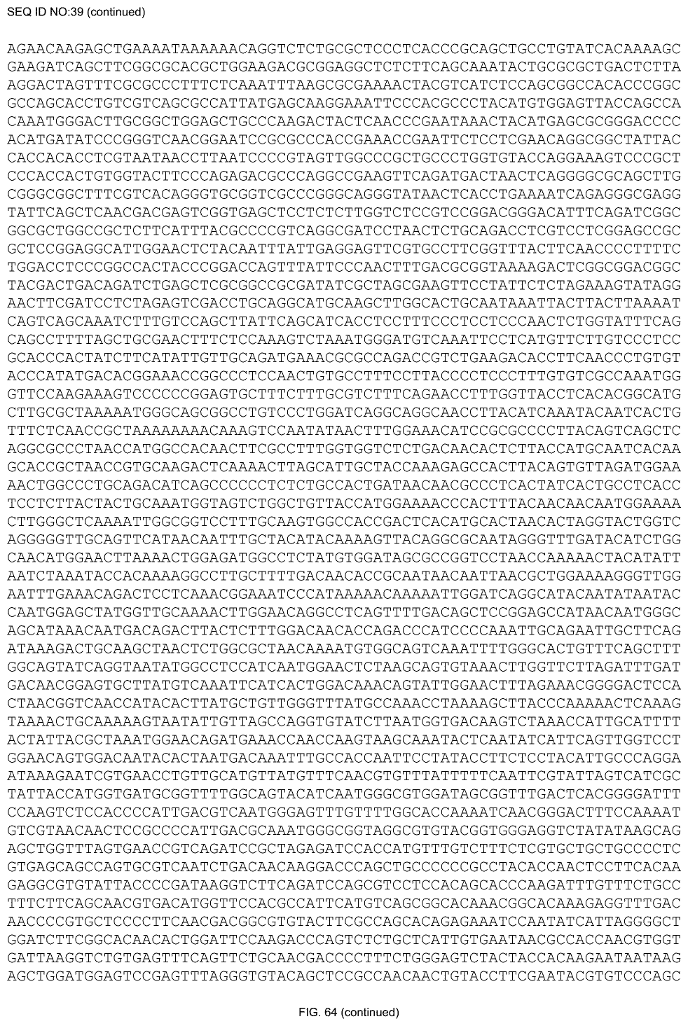

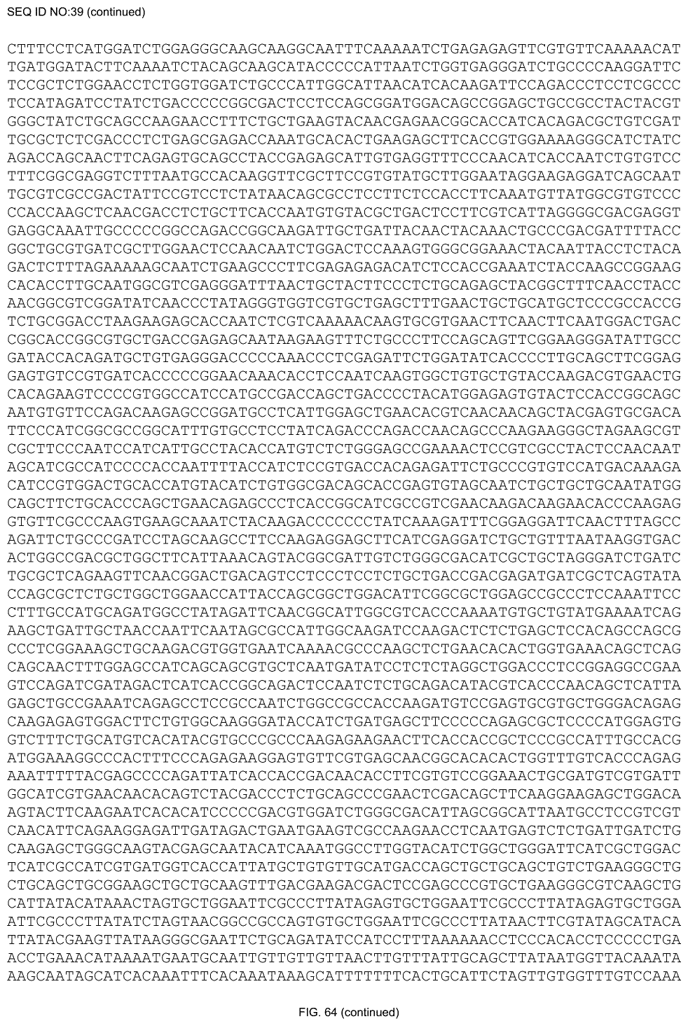

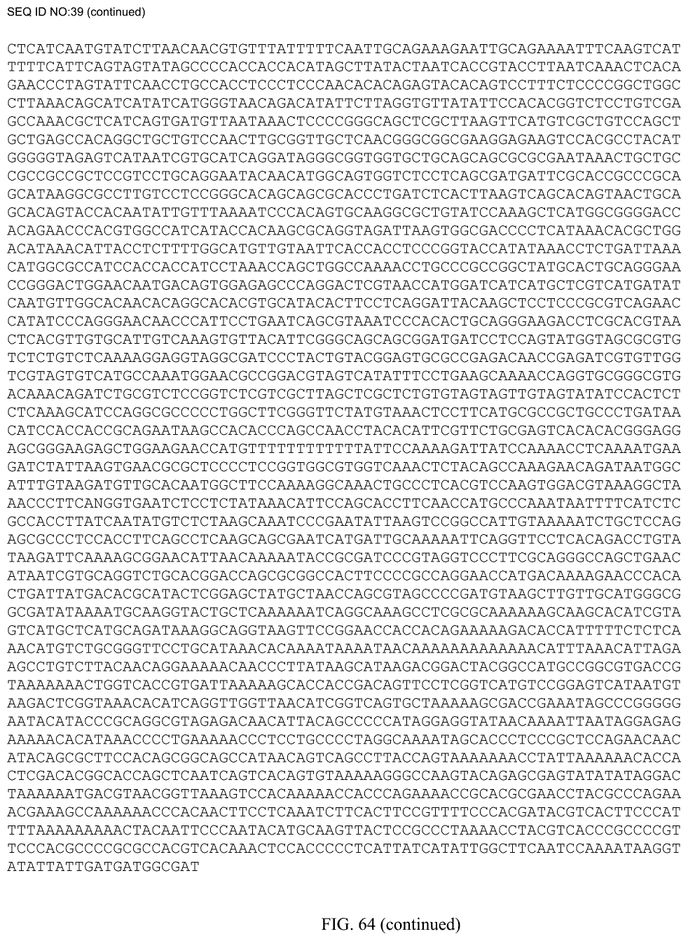

In some cases, an adenovirus vector provided herein (e.g., an adenovirus vector encoding one or more immunogens) can have a genome that is at least 85% percent identical (e.g., at least 88% sequence identical, at least 90% sequence identical, at least 93% sequence identical, at least 95% sequence identical, at least 97% sequence identical, at least 98% sequence identical, or at least 99% sequence identical) to a sequence set forth in any one of SEQ ID NOs:28-39. For example, an adenovirus vector provided herein can have a genome that comprising, consisting of, or consisting essentially of the nucleic acid sequence set forth in SEQ ID NO:28 (see, e.g., FIG. 53). For example, an adenovirus vector provided herein can have a genome that comprising, consisting of, or consisting essentially of the nucleic acid sequence set forth in SEQ ID NO:29 (see, e.g., FIG. 54). For example, an adenovirus vector provided herein can have a genome that comprising, consisting of, or consisting essentially of the nucleic acid sequence set forth in SEQ ID NO:30 (see, e.g., FIG. 55). For example, an adenovirus vector provided herein can have a genome that comprising, consisting of, or consisting essentially of the nucleic acid sequence set forth in SEQ ID NO:31 (see, e.g., FIG. 56). For example, an adenovirus vector provided herein can have a genome that comprising, consisting of, or consisting essentially of the nucleic acid sequence set forth in SEQ ID NO:32 (see, e.g., FIG. 57). For example, an adenovirus vector provided herein can have a genome that comprising, consisting of, or consisting essentially of the nucleic acid sequence set forth in SEQ ID NO:33 (see, e.g., FIG. 58). For example, an adenovirus vector provided herein can have a genome that comprising, consisting of, or consisting essentially of the nucleic acid sequence set forth in SEQ ID NO:34 (see, e.g., FIG. 59). For example, an adenovirus vector provided herein can have a genome that comprising, consisting of, or consisting essentially of the nucleic acid sequence set forth in SEQ ID NO:35 (see, e.g., FIG. 60). For example, an adenovirus vector provided herein can have a genome that comprising, consisting of, or consisting essentially of the nucleic acid sequence set forth in SEQ ID NO:36 (see, e.g., FIG. 61). For example, an adenovirus vector provided herein can have a genome that comprising, consisting of, or consisting essentially of the nucleic acid sequence set forth in SEQ ID NO:37 (see, e.g., FIG. 62). For example, an adenovirus vector provided herein can have a genome that comprising, consisting of, or consisting essentially of the nucleic acid sequence set forth in SEQ ID NO:38 (see, e.g., FIG. 63). For example, an adenovirus vector provided herein can have a genome that comprising, consisting of, or consisting essentially of the nucleic acid sequence set forth in SEQ ID NO:39 (see, e.g., FIG. 64).

In some cases, an adenovirus vector provided herein (e.g., an adenovirus vector encoding one or more immunogens) can have a genome that includes one or more of the coding regions set forth in any one of SEQ ID NOs:28-39. For example, a SC-Ad can be designed to have a genome where all the encoded polypeptides of the SC-Ad have the same amino acid sequence as those polypeptides encoded by the nucleic acid set forth in any one of SEQ ID NOs:28-39.

This document also provides nucleic acid molecules that can encode an adenovirus vector provided herein (e.g., an adenovirus vector encoding one or more immunogens such as a SC-Ad described herein). The term “nucleic acid” as used herein encompasses both RNA and DNA, including cDNA, genomic DNA, and synthetic (e.g., chemically synthesized) DNA. A nucleic acid can be double-stranded or single-stranded. A single-stranded nucleic acid can be the sense strand or the antisense strand. In addition, a nucleic acid can be circular or linear.

This document also provides cells (e.g., cell lines) containing adenovirus vectors described herein (e.g., adenovirus vectors encoding one or more immunogens such as SC-Ads described herein). In cases where an adenovirus vector lacks all or a portion of at least of one adenovirus sequences, the cells containing the adenovirus vector can provide the missing adenovirus polypeptide. For example, when the adenovirus is designed to lack nucleic acid encoding the adenovirus fiber polypeptide, an adenovirus fiber polypeptide-expressing cell line can be used to generate the adenovirus such that the adenovirus contains the fiber polypeptide (e.g., the wild-type fiber polypeptide) while lacking the nucleic acid that encodes the fiber polypeptide (e.g., the wild-type fiber polypeptide). For example, when the adenovirus is designed to lack nucleic acid encoding the V polypeptide, an adenovirus V polypeptide-expressing cell line can be used to generate the adenovirus such that the adenovirus contains the V polypeptide (e.g., the wild-type V polypeptide) while lacking the nucleic acid that encodes the V polypeptide (e.g., the wild-type V polypeptide). For example, when the adenovirus is designed to lack nucleic acid encoding the pIIIa polypeptide, an adenovirus pIIIa polypeptide-expressing cell line can be used to generate the adenovirus such that the adenovirus contains the pIIIa polypeptide (e.g., the wild-type pIIIa polypeptide) while lacking the nucleic acid that encodes the pIIIa polypeptide (e.g., the wild-type pIIIa polypeptide). In some cases, cells containing adenovirus vectors described herein can increase the available number of copies of that virus by at least 100-fold (e.g., by 100-fold to 15,000-fold, by 500- to 10,000-fold, by 5,000- to 10,000-fold, or by 5,000- to 15,000-fold). A virus can be expanded until a desired concentration is obtained in standard cell culture media (e.g., DMEM or RPMI-1640 supplemented with 5-10% fetal bovine serum at 37° C. in 5% CO₂). A viral titer typically is assayed by inoculating cells (e.g., A549 or 293 cells) in culture or by quantitating viral genomes by optical density or real-time PCR. In some cases, cells containing an adenovirus vector provided herein can be used to propagate the adenovirus vector (e.g., to establish a stock of the adenovirus vector). For example, a stock of the adenovirus vector can be produced by growth in mammalian cells. In some cases, a stock of the adenovirus vector can be aliquoted and frozen, and can be stored at −70° C. to −80° C. (e.g., at concentrations higher than the therapeutically effective dose). In some cases, a stock of the adenovirus vector can be stored in a stabilizing solution. Examples of stabilizing solutions include, without limitation, sugars (e.g., trehalose, dextrose, and glucose), amino acids, glycerol, gelatin, monosodium glutamate, Ca²⁺, and Mg²⁺.

In some cases, adenovirus vectors described herein encoding one or more immunogens (and/or nucleic acid molecules that can encode an adenovirus vector described herein encoding one or more immunogens) can be formulated into a composition (e.g., a pharmaceutical composition such as a vaccine composition) for administration to a mammal. For example, adenovirus vectors described herein encoding one or more immunogens (and/or nucleic acid molecules that can encode an adenovirus vector described herein encoding one or more immunogens) can be formulated together with one or more pharmaceutically acceptable carriers (additives), excipients, and/or diluents. Examples of pharmaceutically acceptable carriers, excipients, and diluents that can be used in a composition described herein include, without limitation, sucrose, lactose, starch (e.g., starch glycolate), cellulose, cellulose derivatives (e.g., modified celluloses such as microcrystalline cellulose, and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), polyethylene glycol (PEG), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum proteins (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, lecithin, and corn oil. Suitable pharmaceutical formulations depend in part upon the use and the route of administration. Such forms should not prevent the composition or formulation from reaching target cells or from exerting its effect. For example, pharmacological compositions injected into the blood stream should be soluble.

This document also provides methods for using adenovirus vectors described herein (e.g., adenovirus vectors encoding one or more immunogens) and/or nucleic acid molecules that can encode an adenovirus vector described herein. In some cases, adenovirus vectors described herein and/or nucleic acid molecules that can encode an adenovirus vector described herein can be administered to a mammal (e.g., a human) to increase an immune response (e.g., an increased antibody response and/or an increased T cell response) against a pathogen (e.g., a bacterial or a viral pathogen associated with an immunogen encoded by the adenovirus vectors). For example, adenovirus vectors described herein encoding one or more immunogens (and/or nucleic acid molecules that can encode an adenovirus vector described herein encoding one or more immunogens) can be administered to a mammal to provide the mammal with an immune response effective to reduce the severity of an infection caused by a pathogen associated with the immunogen(s) encoded by the adenovirus vectors. In some cases, adenovirus vectors described herein encoding one or more immunogens (and/or nucleic acid molecules that can encode an adenovirus vector encoding one or more immunogens) can be administered to a mammal as described herein to provide the mammal with an immune response effective to prevent the mammal from exhibiting symptoms of an infection caused by a pathogen associated with the immunogen(s) encoded by the adenovirus vectors. In some cases, adenovirus vectors described herein and/or nucleic acid molecules that can encode an adenovirus vector described herein can be administered to a mammal (e.g., a human) to increase a B cell response within the mammal. For example, adenovirus vectors described herein encoding one or more immunogens (and/or nucleic acid molecules that can encode an adenovirus vector described herein encoding one or more immunogens) can be administered to a mammal as described herein to increase the number of activated B cells (e.g., plasmablasts, plasma cells, and memory B cells) within the mammal by, for example, 10, 20, 30, 40, 50, 60, 70, 80, 90, 95, or more percent.

In some cases, adenovirus vectors described herein and/or nucleic acid molecules that can encode an adenovirus vector described herein can be administered to a mammal (e.g., a human) to increase a number of antibodies (e.g., antibodies against a pathogen such as a bacterial or a viral pathogen associated with the immunogen(s) encoded by the adenovirus vectors) within the mammal. For example, adenovirus vectors described herein encoding one or more immunogens (and/or nucleic acid molecules that can encode an adenovirus vector described herein encoding one or more immunogens) can be administered to a mammal as described herein to increase the number of antibodies within the mammal by, for example, 10, 20, 30, 40, 50, 60, 70, 80, 90, 95, or more percent. In some cases, adenovirus vectors described herein and/or nucleic acid molecules that can encode an adenovirus vector described herein can be administered to a mammal as described herein to produce antibodies against the immunogen(s) within the mammal for, for example, about one week (e.g., from about 1 to about 7 days, from about 1 to about 6 days, from about 1 to about 5 days, from about 1 to about 4 days, from about 1 to about 3 days, from about 2 to about 7 days, from about 3 to about 7 days, from about 4 to about 7 days, from about 5 to about 7 days, from about 2 to about 6 days, from about 3 to about 5 days, from about 2 to about 4 days, from about 3 to about 5 days, or from about 4 to about 6 days).

In some cases, adenovirus vectors described herein and/or nucleic acid molecules that can encode an adenovirus vector described herein can be administered to a mammal (e.g., a human) to increase a T cell response within the mammal. For example, adenovirus vectors described herein encoding one or more immunogens (and/or nucleic acid molecules that can encode an adenovirus vector described herein encoding one or more immunogens) can be administered to a mammal as described herein to increase the number of activated T cells (e.g., cytotoxic T cells and macrophages) within the mammal by, for example, 10, 20, 30, 40, 50, 60, 70, 80, 90, 95, or more percent.

Adenovirus vectors described herein (e.g., adenovirus vectors encoding one or more immunogens) and/or nucleic acid molecules that can encode an adenovirus vector described herein can be administered to any appropriate mammal (e.g., to increase an immune response against a pathogen such as a bacterial or a viral pathogen associated with the immunogen(s) encoded by the adenovirus vectors within that mammal). In some cases, the mammal can be a mammal that has not had a previous infection with a pathogen associated with an immunogen encoded by an adenovirus vector provided herein. In some cases, the mammal can be a mammal that has had a previous infection with a pathogen closely related (e.g., genetically related) to a pathogen associated with an immunogen encoded by an adenovirus vector provided herein. In some cases, the mammal can be a mammal that has an infection (e.g., an ongoing infection) with a pathogen associated with an immunogen encoded by an adenovirus vector provided herein. Examples of mammals that can be administered adenovirus vectors described herein encoding one or more immunogens (and/or nucleic acid molecules that can encode an adenovirus vector described herein encoding one or more immunogens) include, without limitation, humans, non-human primates such as monkeys, dogs, cats, horses, cows, pigs, sheep, mice, rats, rabbits, hamsters, bats, raccoons, and ferrets. In some cases, the methods and materials described herein can be applied to an avian species instead of a mammal. For example, the methods and materials described herein for treating a mammal can be applied to chickens and turkeys. In some cases, the methods and materials described herein can be applied to a species of reptile instead of a mammal. In some cases, the methods and materials described herein can be applied to a species amphibian of instead of a mammal. In some cases, the methods and materials described herein can be applied to a species of fish instead of a mammal. In some cases, a human can be administered one or more adenovirus vectors described herein and/or nucleic acid molecules that can encode an adenovirus vector described herein to increase an immune response against a pathogen (e.g., a bacterial or a viral pathogen associated with an immunogen encoded by the adenovirus vectors).

When administering adenovirus vectors described herein (e.g., adenovirus vectors encoding one or more immunogens) and/or nucleic acid molecules that can encode an adenovirus vector described herein (e.g., a composition such as a vaccine composition including adenovirus vectors described herein and/or nucleic acid molecules that can encode an adenovirus vector described herein), any appropriate route of administration can be used. For example, a composition (e.g., a vaccine composition) provided herein can be administered to a mammal (e.g., a human) orally (e.g., sublingually) or parenterally (including, without limitation, intranasally, subcutaneously, intramuscularly, intravenously, intradermally, intra-cerebrally, intrathecally, or intraperitoneally). In some cases, the route and/or mode of administration of a composition (e.g., a vaccine composition) provided herein can be adjusted for the mammal being treated. In some cases, adenovirus vectors described herein and/or nucleic acid molecules that can encode an adenovirus vector described herein can be administered to a mammal via mucosal delivery. In some cases, adenovirus vectors described herein and/or nucleic acid molecules that can encode an adenovirus vector described herein can be administered to a mammal as described elsewhere (see, e.g., Weaver et al. PLOS ONE, 8(7):e67574 (2013)).

Adenovirus vectors described herein (e.g., adenovirus vectors encoding one or more immunogens) and/or nucleic acid molecules that can encode an adenovirus vector described herein (e.g., a composition such as a vaccine composition including adenovirus vectors provided herein and/or nucleic acid molecules that can encode an adenovirus vector provided herein) can be administered to a mammal (e.g., a human) in any appropriate amount (e.g., any appropriate dose). Effective amounts can vary depending on the route of administration, the age and general health condition of the subject, excipient usage, the possibility of co-usage with other therapeutic treatments such as use of other agents, and the judgment of the treating physician. An effective amount of a composition containing adenovirus vectors encoding one or more immunogens (and/or nucleic acid molecules that can encode an adenovirus vector encoding one or more immunogens) can be any amount that can induce an immune response in a mammal as described herein without producing significant toxicity to the mammal. For example, an effective amount adenovirus vectors encoding one or more immunogens can be, for example, from about 10⁸ viral particles (vp) to about 10¹⁴ vp (e.g., from about 10⁸ vp to about 10¹³ vp, from about 10⁸ vp to about 10¹² vp, from about 10⁸ vp to about 10¹¹ vp, from about 10⁸ vp to about 10¹⁰ vp, from about 10⁸ vp to about 10⁹ vp, from about 10⁹ vp to about 10¹⁴ vp, from about 10¹⁰ vp to about 10¹⁴ vp, from about 10¹¹ vp to about 10¹⁴ vp, from about 10¹² vp to about 10¹⁴ vp, from about 10¹³ vp to about 10¹⁴ vp, from about 10⁹ vp to about 10¹³ vp, from about 10¹⁰ vp to about 10¹² vp, from about 10⁹ vp to about 10¹¹ vp, from about 10¹⁰ vp to about 10¹² vp, or from about 10¹ vp to about 10¹³ vp). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, and/or route of administration may require an increase or decrease in the actual effective amount administered.

Adenovirus vectors provided herein (e.g., adenovirus vectors encoding one or more immunogens) and/or nucleic acid molecules that can encode an adenovirus vector provided herein (e.g., a composition such as a vaccine composition including adenovirus vectors provided herein and/or nucleic acid molecules that can encode an adenovirus vector provided herein) can be administered to a mammal (e.g., a human) in any appropriate frequency. The frequency of administration can be any frequency that can induce an immune response in a mammal without producing significant toxicity to the mammal. In some cases, adenovirus vectors provided herein and/or nucleic acid molecules that can encode an adenovirus vector provided herein can be administered to a mammal once (e.g., in a single administration). In some cases, adenovirus vectors provided herein and/or nucleic acid molecules that can encode an adenovirus vector provided herein can be administered to a mammal several times (e.g., as several administrations). For example, the frequency of administration can be from about once a day to about every three days, from about once a day to about once a week, from about once a week to about every 3 weeks, or from about once a week to about every 6 weeks. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

Adenovirus vectors provided herein (e.g., adenovirus vectors encoding one or more immunogens) and/or nucleic acid molecules that can encode an adenovirus vector provided herein (e.g., a composition such as a vaccine composition including adenovirus vectors provided herein and/or nucleic acid molecules that can encode an adenovirus vector provided herein) can be administered to a mammal (e.g., a human) for any appropriate duration. An effective duration for administering or using a composition containing adenovirus vectors encoding one or more immunogens (and/or nucleic acid molecules that can encode an adenovirus vector encoding one or more immunogens) can be any duration that can induce an immune response in a mammal without producing significant toxicity to the mammal. For example, the effective duration can vary from a couple of days to one week, from several days to several weeks, or from a few days to a month. Multiple factors can influence the actual effective duration used for a particular treatment. For example, an effective duration can vary with the frequency of administration, effective amount, use of multiple treatment agents, and/or route of administration.

In some cases, an adenovirus vector provided herein (e.g., an adenovirus vector encoding one or more immunogens) and/or a nucleic acid molecule that can encode an adenovirus vector provided herein (e.g., a composition such as a vaccine composition including adenovirus vectors provided herein and/or nucleic acid molecules that can encode an adenovirus vector provided herein) can be administered to a mammal (e.g., a human) at an effective amount one, two, or three times with one week to four weeks between each administration when more than one administration is used.

The invention will be further described in the following examples, which do not limit the scope of the invention described in the claims.

### EXAMPLES

### Example 1: A Replicating Single-Cycle Adenovirus Vaccine Against Clostridium difficile

Clostridium difficile causes nearly 500,000 infections and nearly 30,000 deaths each year in the U.S. and costs up to $4.8 billion per year. C. difficile infection (CD1) arises from bacteria colonizing the large intestine and releasing its two toxins, Toxin A (TcdA) and Toxin B (TcdB). This example describes a SC-Ad gene-based vaccine against C. difficile.

### Results

### Single-Cycle Adenovirus Expressing C. difficile Toxins A and B Fusion Protein

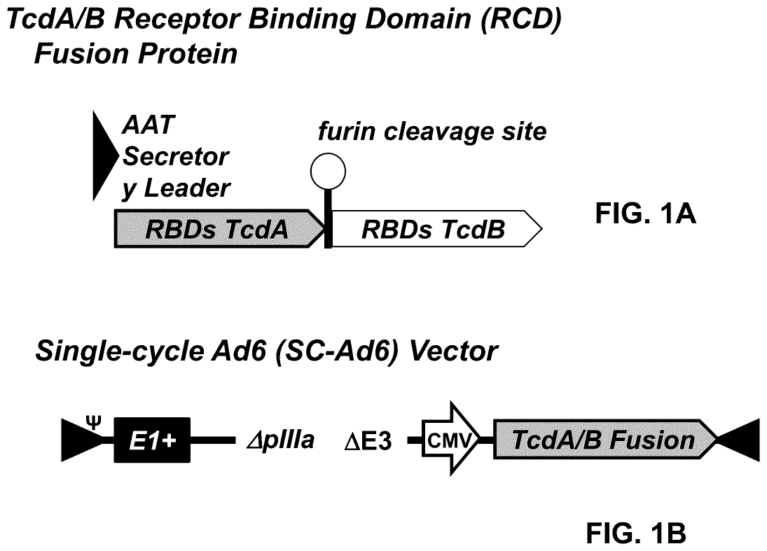

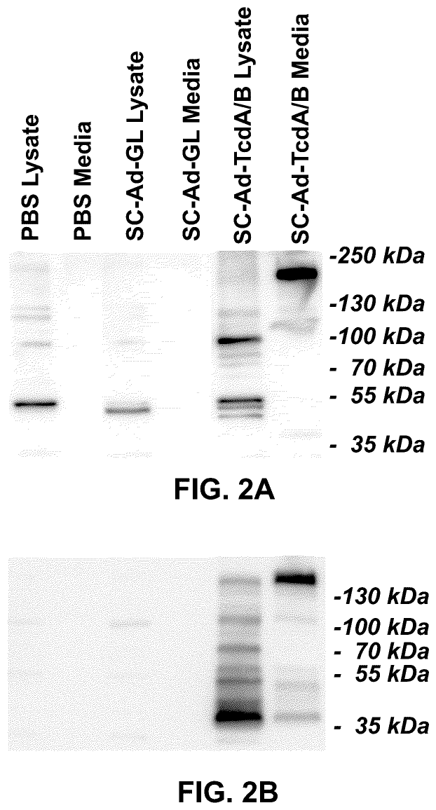

A SC-Ad-TcdA/B vector was generated using adenovirus type 6 (Ad6) (FIG. 1). This vector carries a mammalian codon-optimized cDNA that expresses a fusion protein consisting of a secretory leader and the RBDs of TedA and B separated by two furin cleavage sites. The toxin RBDs were derived from the strain VPI 10463 (toxinotype 0) with glutamine for the asparagine substitutions in putative n-linked glycosylation sites. This resulted in a total of eight substitutions in the TedA RBD and three alterations within the TedB RBD sequences. Human lung A549 cells were infected with SC-Ad6-TcdA/B and cell supernatants and lysates were analyzed by western blot using antibodies specific for TcdA and TcdB. The fusion protein was predicted to be 160 kDa with the RBDs of TedA and TcdB expected to be 100 and 60 kDa, respectively. Under these conditions, both RBDs and the fusion protein were observed in cells and in concentrated cell supernatants (FIG. 2).

### Single Intramuscular Vaccination with SC-Ad6-TcdA/B Induces Immune Responses in Mice

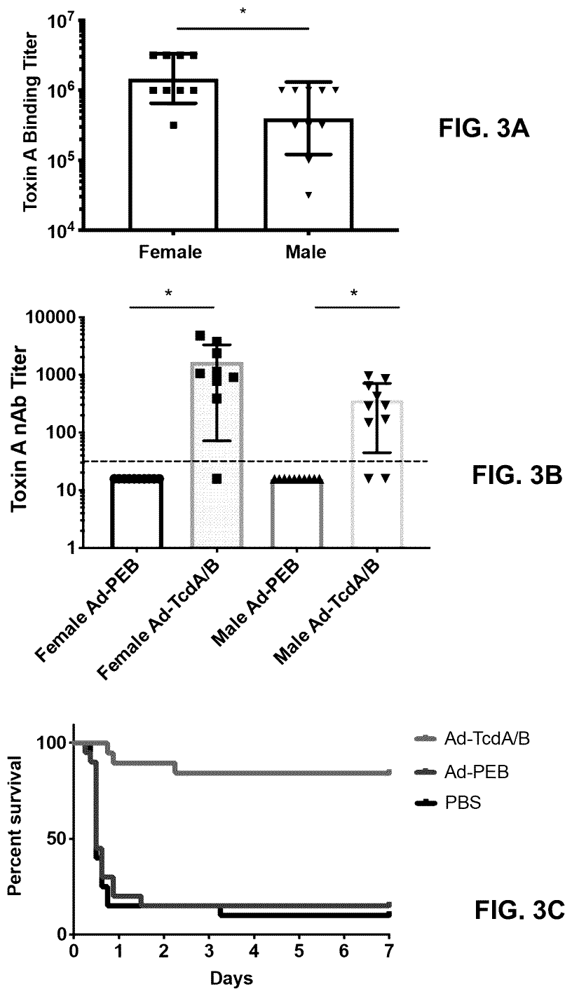

Groups of 10 male and female outbred CD-1 mice were immunized i.m. a single time with PBS or 10¹⁰ virus particles of SC-Ad6-TcdA/B or a negative control vector SC-Ad6-PEB1 that expresses a mismatched protein from another bacterium, Campylobacter jejuni. Serum was harvested 6 weeks after immunization and anti-toxin antibody responses were evaluated by ELISA and in vitro toxin neutralization assays. Single immunization generated significant antibodies against toxin A in the majority of the SC-Ad6-TcdA/B vaccinated animals (FIGS. 3A and 3B). Reciprocal titers were defined as those being significantly higher than levels in PBS control mice; therefore, antibody levels are not shown for the PBS group. Both animal sexes generated antibody responses that were significantly higher than in control mice. Female mice had a geometric mean reciprocal toxin A binding titer of 1,467,329 while males had titers of 397,964 (FIG. 3A). Female binding titers were significantly higher than male titers (p<0.0066 by Mann-Whitney). A similar pattern was observed in the TedA neutralizing (nAb) titers from the two sexes, with mean titers of 1682 in females and 379 in males (FIG. 3B). However, these differences were not significant (p=0.8004 by Dunn's). When these were compared to animals immunized with SC-Ad6-PEB1, those receiving SC-Ad6-TcdA/B had nAb titers that were significantly higher than their sex matched controls.

### Single Intramuscular Vaccination with SC-Ad6-TcdA/B Provides Protection Against Toxin Challenge

8 weeks after single immunization, the mice were challenged with 300 ng (6× LD50) of purified TcdA from List Labs. The recombinant toxin was derived from a Ribotype 087 and Toxinotype 0 strain similar to VPI 10463 antigens in the SC-Ad vaccine. Eight out of ten PBS and PEB1 mice succumbed to the toxin within 24 hours of challenge (FIG. 3C). One additional PBS mouse met sacrifice criteria 3 days later. Seventeen out of the twenty SC-Ad6-TcdA/B vaccinated mice survived the challenge. Log-rank comparison of the Kaplan-Meier survival curves demonstrated that SC-Ad6-TcdA/B vaccinated animals survived significantly better than PBS or PEB1 control animals. The three SC-Ad6-TcdA/B vaccinated mice that did not survive had reduced TedA binding titers and a complete absence of nAbs (FIGS. 3A and 3B).

### SC-Ad6-TcdA/B Provides Protection Against Toxin Challenge 38 Weeks After Single Immunization

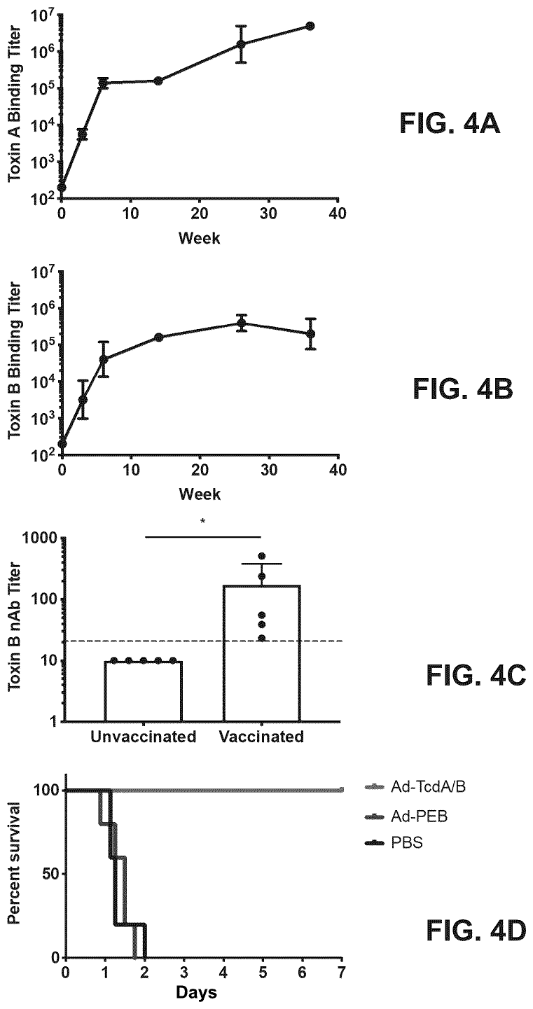

A second set of female CD1 mice were immunized i.m. a single time with 10¹⁰ virus particles of SC-Ad6-TcdA/B, SC-Ad6-PEB1, or PBS (n=5 per group). This single vaccination of SC-Ad6-TcdA/B generated strong antibody responses with reciprocal endpoint binding titers for TcdA and TedB that climbed above 100,000 over 26 weeks (FIGS. 4A and 4B). At week 26, the average reciprocal TcdB nAb titer in the SC-Ad-TcdA/B group reached 174 (FIG. 4C). At week 38, the mice were challenged with 300 ng (6× LD50) of TcdA. All PBS and control SC-Ad-PEB1 vaccinated animals succumbed to the toxin within 48 hours (FIG. 4D). In contrast, all animals in the SC-Ad C. difficile vaccine group survived.

### Pilot Toxicology and Efficacy Testing in Hamsters

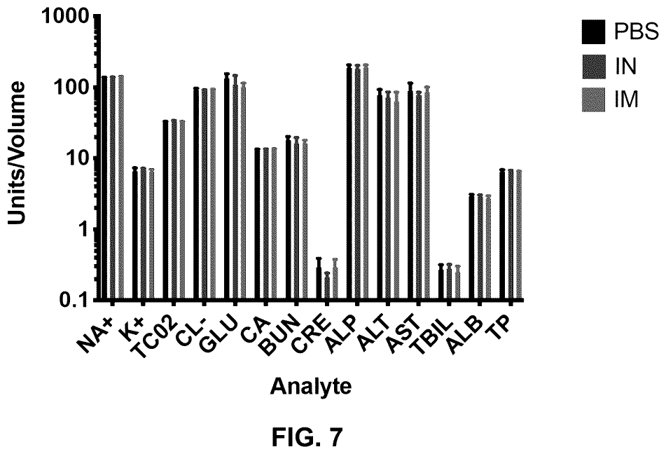

Groups of 10 Syrian hamsters were immunized a single time with 10¹¹ virus particles of SC-Ad6-TcdA/B by the i.n. or i.m. route. Control animals received i.n. PBS. Blood was collected 3 days after immunization for clinical chemistry. These revealed no significant differences in blood chemistry (FIG. 7).

### Single Intranasal or Intramuscular Vaccination with SC-Ad6-TcdA/B Induces Immune Responses in Hamsters

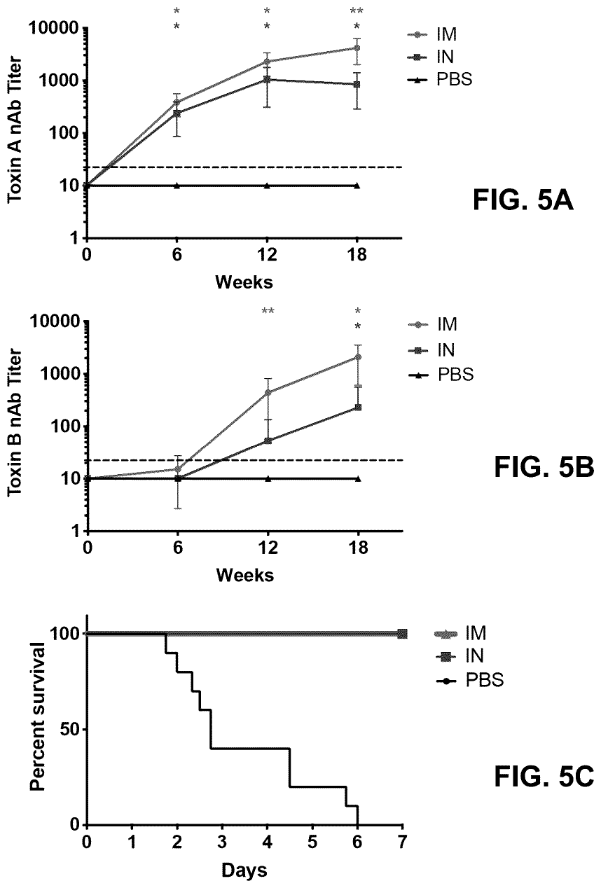

Serum was collected from the hamsters 6, 12, and 18 weeks after immunization and anti-toxin antibody responses were evaluated. Single immunization with SC-Ad6-TcdA/B generated significant serum nAbs against toxin A regardless of route. These antibody levels increased over the course of the study (FIG. 5A). While i.m. immunization produced higher mean nAbs against toxin A than the i.n. group, these were not significantly different until week 18 (p=0.0336 by Dunn's). Toxin B antibodies were detectable by ELISA at week 6. However, significant levels of nAbs against toxin B took longer to develop (FIG. 5B). All i.m. immunized animals and 6/10 of the i.n. immunized animals had significant toxin B nAb levels by week 12. At week 18, all i.m. and i.n. immunized animals had significant toxin B nAbs with mean reciprocal titers of 2084 and 229, respectively.

### Single Intramuscular Vaccination with SC-Ad6-TcdA/B Provides Protection against C. difficile Spore Challenge in Hamsters

CD1 can be induced in Syrian hamsters when sensitized with clindamycin. This model mimics the fecal-oral route of transmission by delivering purified C. difficile spores orogastrically producing symptoms similar to those observed in patients with CD1. Various toxin isoforms have been identified within clinical isolates of C. difficile. Recent studies report that the BI/NAP1/027 strain of C. difficile is the most prevalent cause of CD1 in North America. Given its clinical relevance and expression of heterologous toxin isoforms, vaccine efficacy was tested using spores from the UK1 (BI/NAP1/027) strain. Hamsters were administered clindamycin intraperitoneally 24 hours before receiving 10,000 spores 20-21 weeks after a single immunization. All of the PBS immunized animals succumbed to the spore challenge (FIG. 5C). Strikingly, all of the SC-Ad6-TcdA/B vaccinated hamsters survived to the end of the study regardless of vaccine administration route. Log-rank comparison of the Kaplan-Meier survival curves demonstrated that both i.n. and i.m. SC-Ad6-TcdA/B vaccinated animals had significantly better survival than PBS control animals. Weight loss was observed in all animals over the course of the experiment, but weight loss in SC-Ad6-TcdA/B animals either stabilized or began to reverse by day 7.

### SC-Ad6-TcdA/B Provides Protection Against Lethal Spore Challenge 45 Weeks After Single Immunization

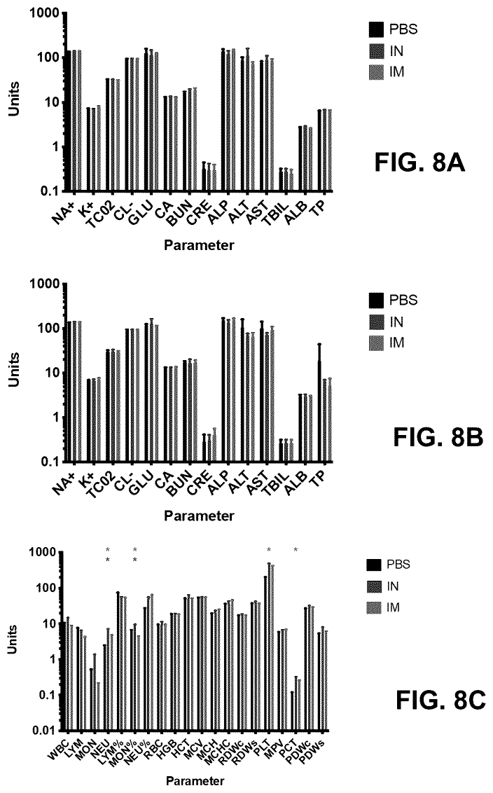

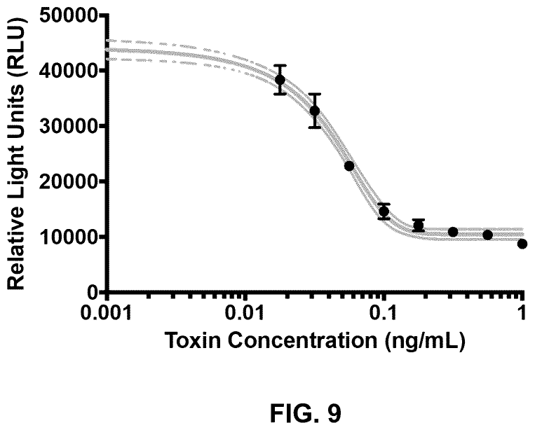

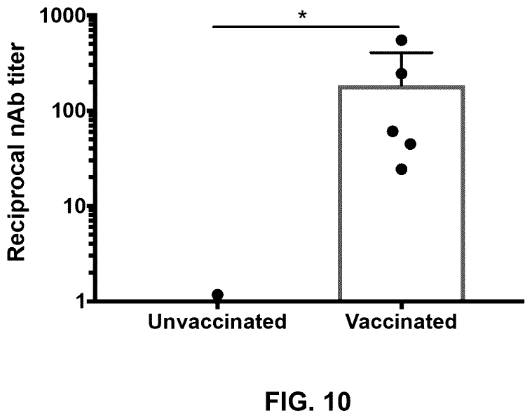

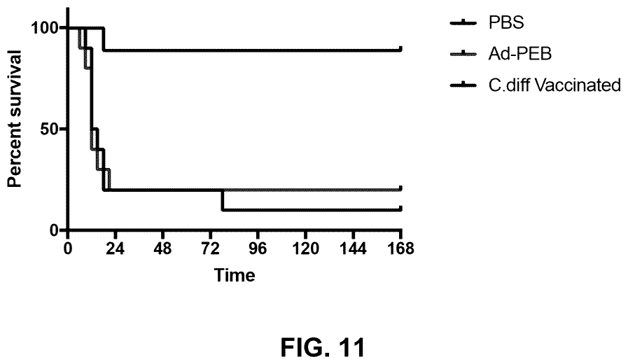

A second group of 10 female Syrian hamsters were immunized a single time with 10¹¹ virus particles of SC-Ad6-TcdA/B either i.n. or i.m. or with PBS. Blood was collected 3 or 4 days after immunization and tested using the same analyte panel as before. Similarly, no significant differences were observed between vaccinated and unvaccinated on days 3 or 4 (FIG. 8A and 8B). At day 4, CBCs were measured in half of the animals. There were significant increases in the percentage of neutrophils and a corresponding decrease in the percentage of lymphocytes in animals receiving vaccine compared to controls; however, comparison of the number of neutrophil and lymphocyte showed no differences (FIG. 8C). I.m. immunized animals saw increases in their platelets and plateletcrit, although these were still within normal ranges.

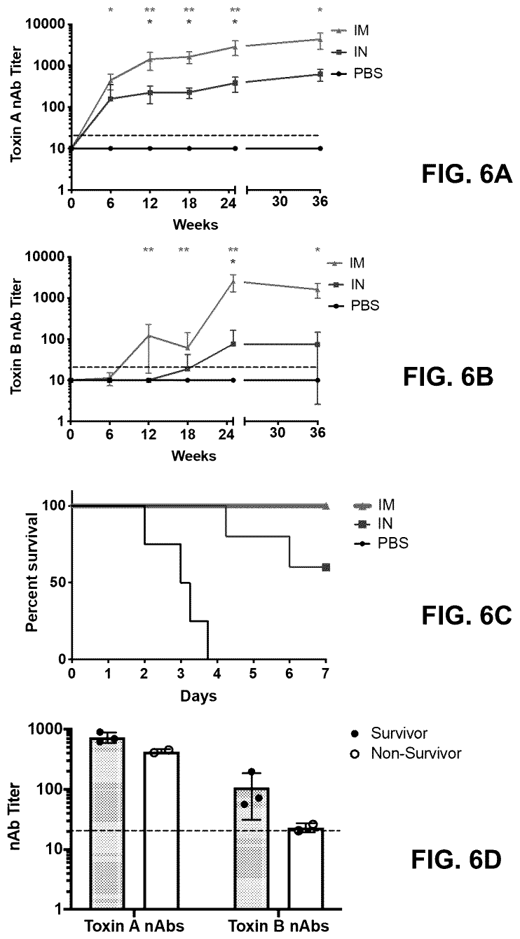

Serum collected at weeks 6, 12, and 18 showed increasing toxin A and B nAbs as in the first vaccination study (FIG. 6A and 6B). At week 24, half of the hamsters in each group were sensitized with clindamycin given orogastrically (30 mg/kg) and then were challenged with 200 spores of C. difficile 5 days later. This low dose challenge surprisingly induced no symptoms or indications of C. difficile infection in any hamsters including the PBS controls. Serum antibodies collected at the termination of this challenge revealed no increases in toxin A or B antibodies due to the pathogen challenge compared to unchallenged animals in the cohort. The unchallenged animals were followed for an additional 20 weeks. In this period, one PBS and one i.m. immunized animals became moribund and had to be euthanized at week 42 and 44, respectively.

The remaining animals were challenged at week 45 using the high dose 10,000 spore challenge. All PBS immunized animals met endpoint criteria and were euthanized (FIG. 6C). Two intranasally-immunized animals also succumbed to the challenge. All animals in the i.m. vaccine group survived spore challenge. Log-rank comparison of these survival curves showed significant differences in the survival of both i.n. and i.m. SC-Ad6-TcdA/B vaccinated animals compared to PBS. Toxin nAbs levels before challenge (week 36), correlated with survival in the groups (FIG. 6D). Animals in the i.n. group that survived spore challenge had high toxin nAbs prior to challenge. In contrast, animals in this group that did not survive had lower toxin nAbs before they were challenged. Protection against C. difficile challenge was observed 10 months after only a single immunization with SC-Ad vaccine.

### Materials and Methods

### Cell Culture

A549 cells and Vero cells were purchased from the American Type Culture Collection. The 293-IIIA cells were generated as described elsewhere (Crosby et al., Virology 462-463:158-165 (2014)). All cells were maintained at 37° C. in Dulbecco's Modified Eagle Medium (DMEM) supplemented with 10% heat inactivated fetal bovine serum (HI-FBS; HyClone) and penicillin/streptomycin at 100 U/mL (Invitrogen).

Single-Cycle Adenovirus Expressing C. difficile TcdA/B Fusion

A codon-optimized cDNA encoding a novel fusion of the receptor binding domains of C. difficile toxins A and B was synthesized by Genscript. This cDNA contains the secretory leader sequence from alpha-1 antitrypsin (AAT) to facilitate secretion of the fusion protein. The receptor binding domains are separated by two furin cleavage sites to liberate the two Teds from each other during secretion from mammalian cells. This cDNA was inserted into the shuttle plasmids pAd6-NdePf1 and was recombined into SC-Ad6 as described elsewhere (Crosby et al., Virology 462-463:158-165 (2014), Crosby et al., J. Virol. 91(2):e00720-16 (2017); and Crosby et al., J. Virol. 89:669-675 (2015)) to generate SC-Ad6-C. diff. Control SC-Ad6 viruses expressing GFP-Luciferase or Campylobacter jejuni PEB1 were also used. Viruses were rescued, amplified, and purified as described elsewhere (Crosby et al., Virology 462-463:158-165 (2014), Crosby et al., J. Virol. 91(2):e00720-16 (2017); and Crosby et al., J. Virol. 89:669-675 (2015)). Virus comparisons were based on virus particles.

### Western Blotting

A549 cells were infected with SC-Ad6-TcdA/B or SC-Ad-GL, which expresses GFP-Luciferase, with 10⁴ virus particles/cell. 24 hours after infection, the media was replaced with serum-free DMEM. 24 hours later, this media was collected and concentrated using Amicon Ultra-15 30 k (Millipore). The cells were harvested using Triton-X lysis buffer supplemented with Complete™ Protease Inhibitor Cocktail (Roche). Media concentrates and cell lysates were analyzed by western blot using antibodies against C. difficile toxin A or B (1:1000; List Biological Labs, Inc.) followed by a goat anti-chicken horseradish peroxidase secondary antibody (1:1000; Invitrogen). SuperSignal West Dura (Thermo Scientific) was added and blots were imaged on an In Vivo F Station (KODAK).

### Animals

Male and female outbred CD-1 mice (Charles River Laboratories) and female Golden Syrian hamsters (Envigo) were housed in the Mayo Clinic Animal Facility. All animal handling and experiments were carried out according to the provisions of the Animal Welfare Act, PHS Animal Welfare Policy, the principles of the NIH Guide for the Care and Use of Laboratory Animals, and the policies and procedures of the Institutional Animal Care and Use Committee at Mayo Clinic.

### Immunizations and Sample Collection

Mice were anesthetized with isoflurane and immunized by the intramuscular (i.m.) route with 10¹⁰ vp of the indicated vaccine. Hamsters were anesthetized with isoflurane and immunized intramuscularly (i.m.) or intranasally (i.n.) routes with 10¹¹ vp of the vaccine. In mice, serum was collected from the facial vein at the indicated time points. At various time points, hamsters were anesthetized and blood was collected from the jugular vein.

### Enzyme-Linked Immunosorbent Assay (ELISA)

Immulon 4 HBX plates (Thermo) were coated with 100 ng/well of either C. difficile A or B toxoids (List Biological Labs, Inc.) in 1× phosphate-buffered saline (PBS) overnight. Wells were washed and blocked with 5% milk in Tris-buffered saline with 0.1% Tween 20 (TBST) at room temperature (RT) for 2 hours. After washing with TBST, half log dilutions of each serum sample were plated in triplicate and incubated for 3 hours at RT. Wells were washed and 100 μL of 1:10,000 goat anti-mouse IgG-horseradish peroxidase (Thermo Fisher Scientific Inc.) was added to each well. Plates were incubated for 2 hours at RT. Wells were washed and 50 μL of 1 step Ultra TMB ELISA (Thermo Fisher Scientific Inc.) were added to each well. When color developed, 50 μL of 2M H₂SO₄ were added. OD450 was determined with a BioTek Synergy H1 Hybrid Multi-Mode Reader. Reciprocal titers were statistically defined based on 95% confidence interval.

### Cytotoxicity and Neutralization Assays

Cytotoxicity of C. difficile toxin A and toxin B were determined on Vero cells using the method described elsewhere (Donald et al., Microbiology 159:1254-1266 (2013)). Vero cells were used in place of IMR-90 as they have similar sensitivity to Toxin B compared to IMR-90 cells, but have an increased sensitivity to toxin A. Vero cells were plated at 10⁴ cells per well in a 96 well plate. C. difficile toxin A or toxin B (List Biological Laboratories, Inc) was serially diluted in DMEM supplemented with 10% HI-FBS and added to cells 24 hours after plating. Three days later, cell viability was determined using the bioluminescent CellTiter-Glo reagent (Promega). An EC50 was determined to be equivalent to the amount of toxin causing 50% reduction in luminescence by fitting the data with a four-parameter equation. Toxin neutralization was determined using serial dilutions of mouse or hamster serum mixed with eight times the EC50 value determined in the cytotoxicity assay. The mixtures were incubated at 37° C. for 90 minutes in a humidified incubator (5% CO₂) before being added to Vero cells on 96 well-plates. Three days later, cell viability was determined with Celltiter-Glo. A four-parameter regression response was fitted to the luciferase relative light units (RLU) values derived from the serum dilutions. Neutralizing antibody (nAb) titers were expressed as the derived sample dilution that exhibited a 50% reduction in cytotoxicity. If a serum titration failed to generate 50% inhibition within the range of concentrations tested, a titer value of ½ of the highest serum concentration tested was ascribed to it.

### Challenge With Recombinant C. difficile Toxin A in Mice

Immunized mice were challenged intraperitoneally (i.p.) with 300 ng of C. difficile toxin A (List Biological Laboratories, Inc). Following toxin challenge, mice were monitored every 3 hours for the first 30 hours, followed by monitoring at 6-hour intervals for 72 hours, and then every 12 hours from day 3 through day 7 (168 hours). Mice were monitored for clinical signs. Briefly, animals' symptoms were scored as normal, lethargic, abnormal or moribund. Moribund animals were euthanized immediately and recorded. The survival rate was determined for each treatment group.

### Hematology and Clinical Chemistry in Hamsters

Blood was collected for clinical chemistry analysis (200 μL into lithium heparin tubes; Greiner Bio-One) and for complete blood count (CBC, 100 μl in K₂ EDTA tubes; Greiner Bio-One). Blood chemistry was analyzed with a Piccolo Xpress Analyzer (Abaxis) and CBCs were determined with VetScan HM5 hematology analyzer (Abaxis). Analyte parameters for the two tests are shown in Table 1 and Table 2.

TABLE 1
Veterinary Hematology Parameters Measured on the Abaxis VetScan
HM5 Analyzer Results Table.

| Parameter | Abbreviation | Units |
|---|---|---|
| Sodium | NA+ | mmol/L |
| Potassium | K+ | mmol/L |
| Total Carbon Dioxide | tCO2 | mmol/L |
| Chloride | CL− | mmol/L |
| Glucose | GLU | mg/dL |
| Calcium | CA | mg/dL |
| Blood Urea Nitrogen | BUN | mg/dL |
| Creatinine | CRE | mg/dL |
| Alkaline Phosphatase | ALP | U/L |
| Alanine Aminotransferase | ALT | U/L |
| Aspartate Aminotransferase | AST | U/L |
| Total Bilirubin | TBIL | mg/dL |
| Albumin | ALB | g/dL |
| Total Protein | TP | g/dL |

TABLE 2
Blood Chemistry Parameters Measured on the Piccolo Xpress
Chemistry Analyzer.

| Parameter | Abbreviation | Units |
|---|---|---|
| Total White Blood Cell count | WBC | 10\hat{ }9 L |
| Lymphocyte count | LYM | 10\hat{ }9 L |
| Monocyte count | MON | 10\hat{ }9 L |
| Neutrophil count | NEU | 10\hat{ }9 L |
| Eosinophil count | EOS | 10\hat{ }9 L |
| Basophil count | BAS | 10\hat{ }9 L |
| Lymphocyte percentage | LYM % | % |
| Monocyte percentage | MON % | % |
| Neutrophil percentage | NEU % | % |
| Eosinophil percentage | EOS % | % |
| Basophil percentage | BAS % | % |
| Red Blood Cell count | RBC | 10\hat{ }12 L |
| Hemoglobin | HGB | g/dL |
| Hematocritpercentage | HCT | % |
| Mean Corpuscular Volume | MCV | fL |
| Mean Corpuscular Hemoglobin | MCH | pg |
| Mean Corpuscular Hemoglobin Concentration | MCHC | g/dl |
| Red Cell Distribution Width, coefficient of | RDWc | % |
| variation % | | |
| Red Cell Distribution Width | RDWs | fL |
| Platelet count | PLT | 10\hat{ }9 L |
| Mean Platelet Volume | MPV | fL |
| Platelet crit % | PCT | % |
| Platelet Distribution Width, coefficient of | PDWc | % |
| variation % | | |
| Platelet Distribution Width | PDWs | fL |

### Challenge with C. difficile Spores in Hamsters

Prior to challenge, hamsters were housed individually in ventilated cages. In the low dose challenge, hamsters were sensitized for infection using a clindamycin phosphate (Sigma-Aldrich) antibiotic solution (30 mg/kg of body weight) delivered orogastrically via a feeding needle. Five days later, the hamsters were challenged orogastrically with 200 spores from C. difficile strain UK1. Since low dose spore challenge did not induce symptoms in hamsters in our hands, a high dose challenge with modified clindamycin administration was used. In this challenge, hamsters were sensitized with clindamycin phosphate antibiotic solution (10 mg/kg of body weight) by the intraperitoneal route rather than the orogastric route. The hamsters were then challenged 24 hours later orogastrically with 10⁴ UK1 spores. In both the high and low dose challenge, the hamsters were monitored 4 times per day following infection by assessing them individually in a microbiological safety cabinet for several parameters, including presence and severity of wet tail, loose feces, diarrhea, weight loss, activity level, starey coat, sunken eyes, hunched posture and response to stimulus. A scoring system, based on severity of changes observed (ranging from 0-3 for each parameter), was used to quantify the condition of the animals. Animals were euthanized and considered to have succumbed to disease when they either reached a score≥15, were moribund, or suffered weight loss in excess of 20%.

### Statistical Analysis

Prism 8 Graphical software was used for all statistical analyses.

### Example 2: Single-Cycle Adenovirus Vectors Expressing a SARS-CoV-2 Polypeptide

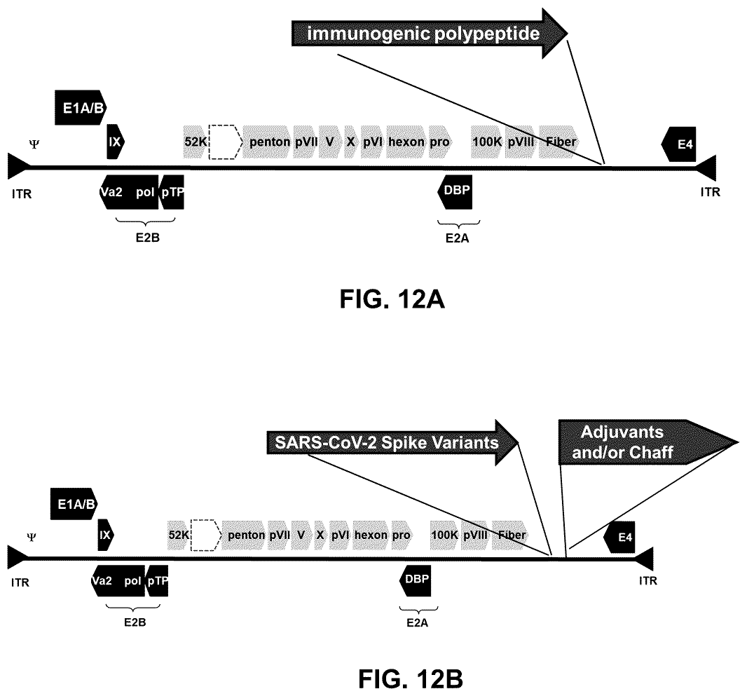

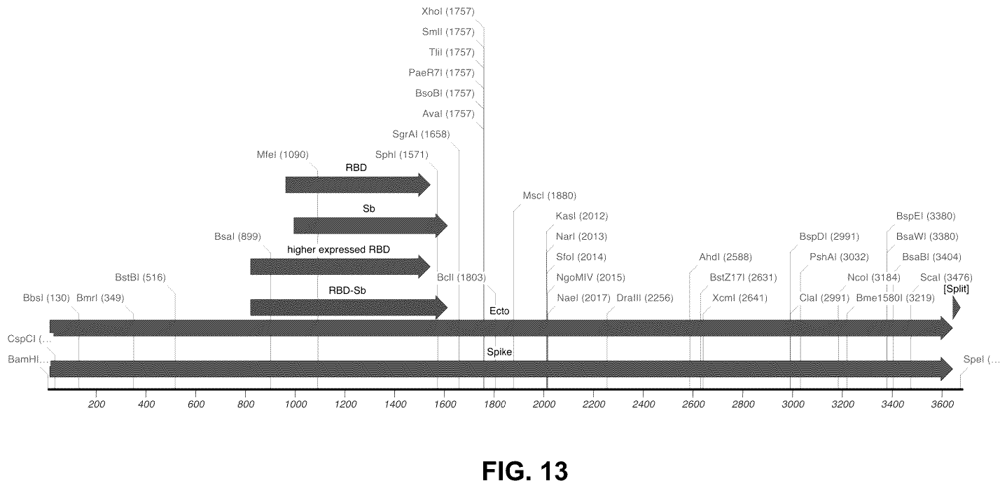

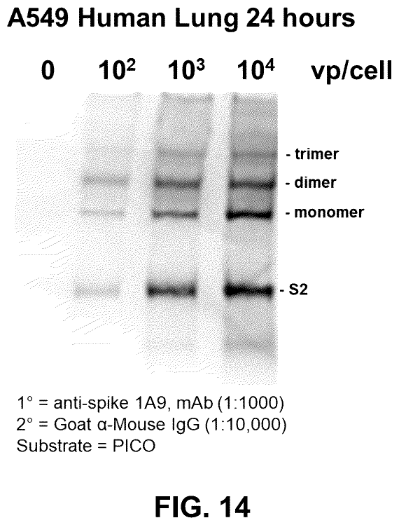

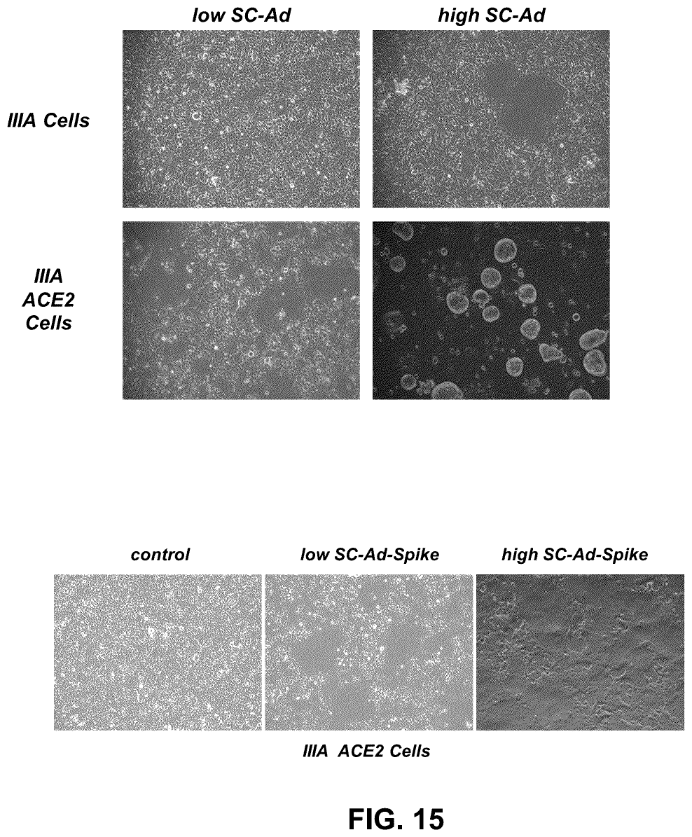

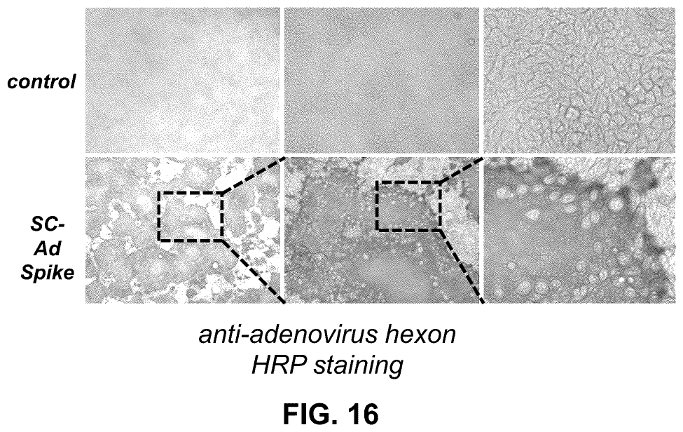

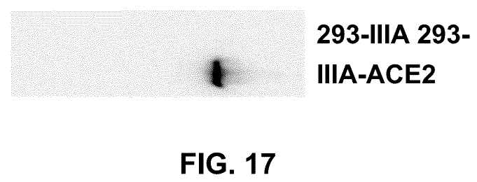

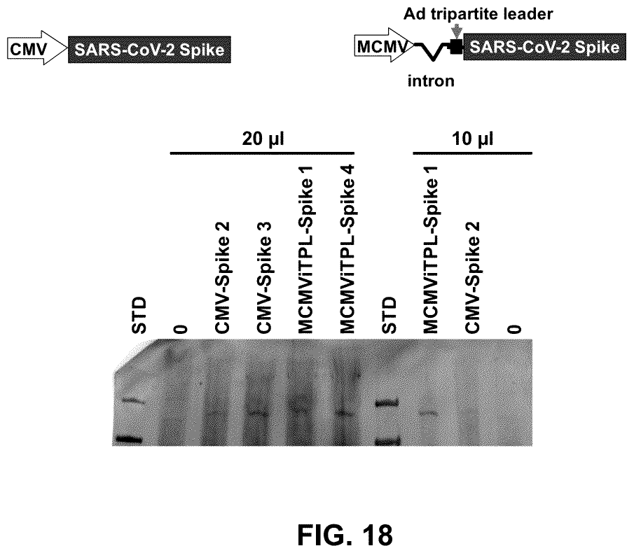

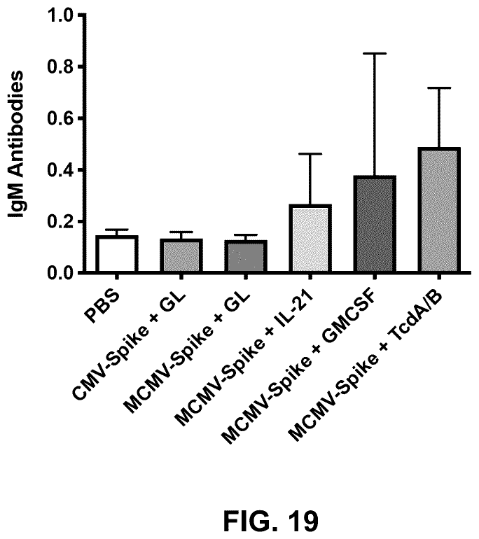

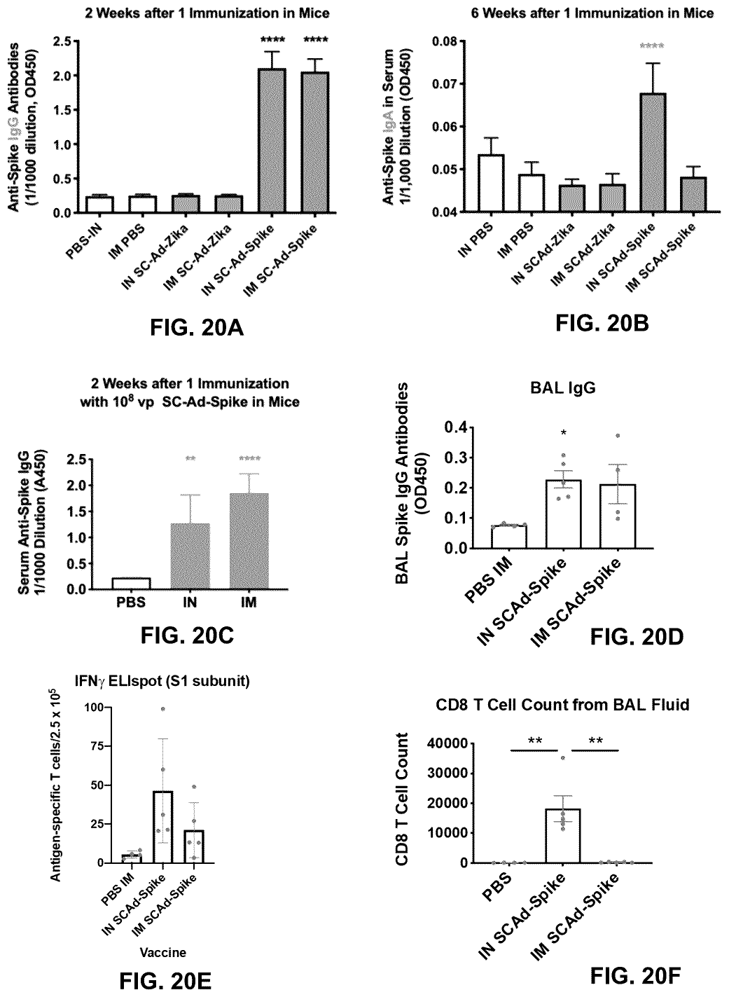

A full-length codon-optimized SARS-CoV-2 Spike cDNA (FIG. 13) or RBD-Sb subdomains were inserted into pAd6-□III-□E3 with and without genetic adjuvant or chaff genes (FIG. 12) and rescued in 293-IIIA cells. CsCl-purified SC-Ad6-Spike produced monomer, dimer, and trimers of spike as well as cleaves S2 domain after infection of A549 human lung cells as demonstrated by western blot with anti-spike antibody (FIG. 14). SC-Ad6-Spike induced cell-cell fusion and syncytia in cells engineered to express its receptor ACE2 (FIG. 15). When these syncytia were examined, they contained abundant amounts of adenovirus proteins as demonstrated by immunohistochemical staining for adenovirus hexon with AdenoX Rapid Titer reagent (FIG. 16). When SC-Ad6-Spike was used to immunize BALB/c mice by the intranasal (i.n.) or intramuscular (i.m.) route, the virus induced strong spike antibodies within 2 weeks as demonstrated by ELISA using 1/1000 dilutions of mouse sera (FIG. 20A). These were class-switched IgG antibodies and they were significantly higher than negative control mice immunized with PBS buffer or SC-Ad expressing Zika E (p<0.0001 by one-way ANOVA). At 6 weeks after immunization, IgG antibodies remained elevated in sera. Notably, intranasal immunization also generated IgA antibodies indicative of responses at mucosal barriers (FIG. 20B). Dose-finding studies in BALB/c mice demonstrated significant IgG antibodies responses were generated 2 weeks after a single i.n. or i.m. immunization with 10⁸ viral particles of SC-Ad-Spike (**p<0.01, ****p<0.0001 by ANOVA, FIG. 20C).

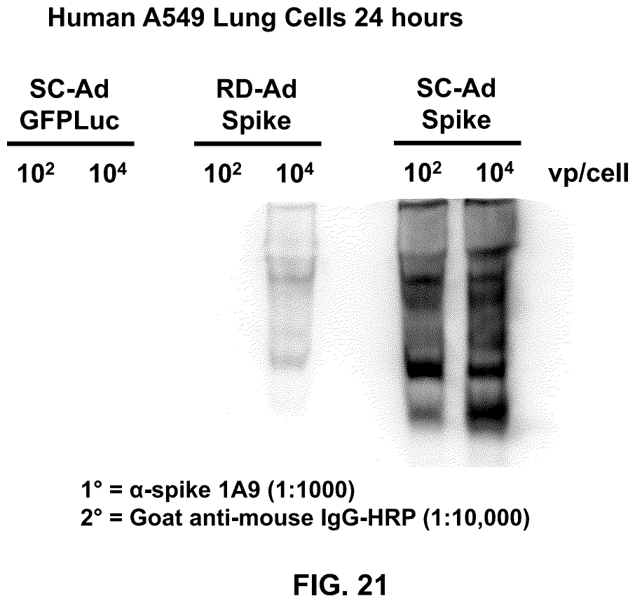

A replication-defective Ad6 (RD-Ad6) with an El deletion was constructed with the same Spike expression cassette. RD-Ad6-Spike and SC-Ad-Spike were used to infect A549 human lung cells with different numbers of virus particles (vp) per cell. When cell lystates were examined 24 hours later by western blot for the Spike protein, this revealed that SC-Ad expressed high levels of Spike protein when 10² or 10⁴ vp of the virus (FIG. 21). In contrast, RD-Ad-Spike produce no detectable Spike protein with 10² particles of virus and only low levels of Spike protein with 10⁴ vp of virus.

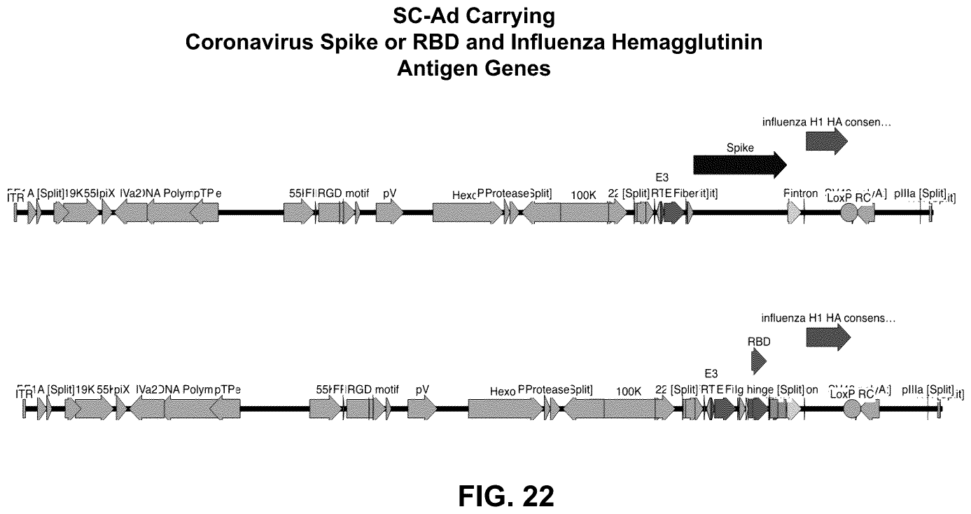

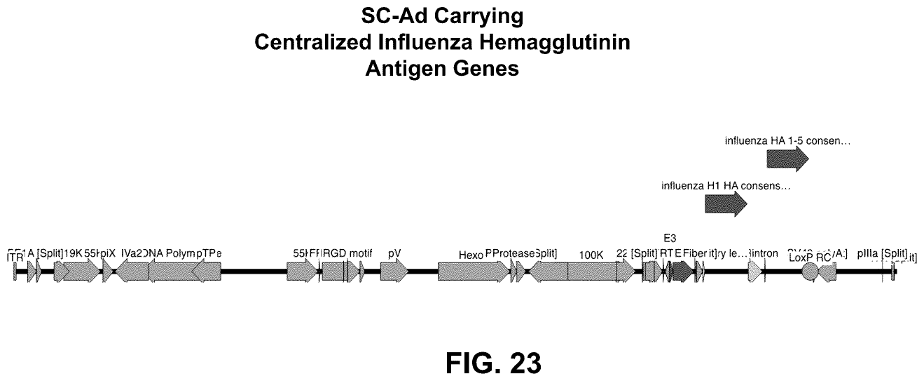

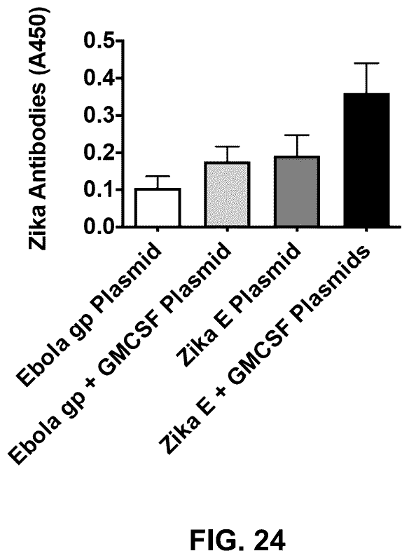

SC-Ad expressing coronavirus Spike or RBD proteins can be modified by the addition of influenza genes to generate a combined coronavirus and influenza virus vaccine. For example, SC-Ad6 containing Spike and a centralized H1 consensus influenza hemagglutinin (H1-CON) gene or SC-Ad6 containing the Spike RBD domain and a centralized H1 consensus influenza hemagglutinin (H1-CON) gene (FIG. 22). Alternatively, an SC-Ad expressing Spike or RBD could be co-immunized with an SC-Ad expressing two influenza consensus immunogens. For example, SC-Ad6 containing centralized H1 consensus influenza H1-CON and H1-5 centralized HA gene H1-5-CON (FIG. 23).

### Example 3: Single-Cycle Adenovirus Vectors Expressing Genetic Adjuvants

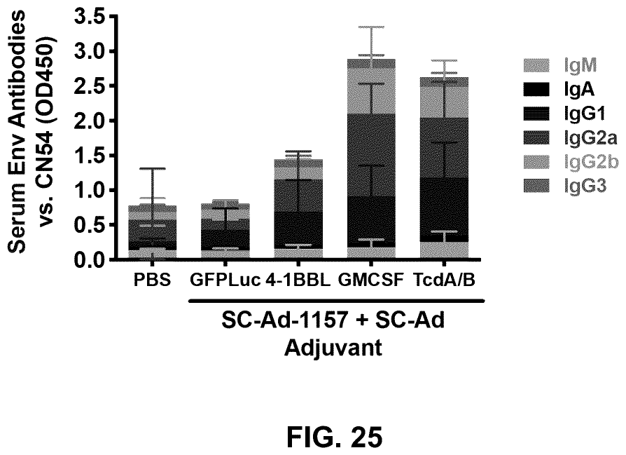

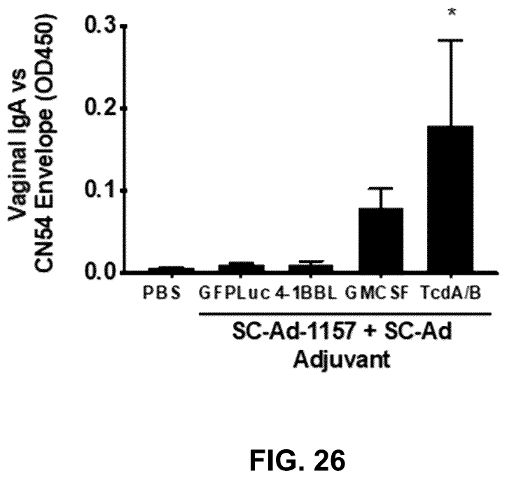

10⁹ viral particles (vp) of SC-Ad6 expressing clade C gp140 from SHIV-1157ipd3N4 was used to immunize BALB/c mice by the i.n. route in combination with 10⁹ SC-Ads expressing 4-1BBL, GMCSF, C. diff toxin fragment TcdA/B or a non-specific adenovirus control expressing GFP-Luciferase. ELISAs using serum collected 6 weeks after single immunization demonstrated significant increases in antibody isotypes by GMCSF and TcdA/B (p<0.05 or less for all IgGs) (FIG. 25). When vaginal washes were assayed for IgA at the same time point, this revealed similar trends with highest mucosal IgA mediated by i.n. co-delivery of TcdA/B adjuvant (FIG. 26).

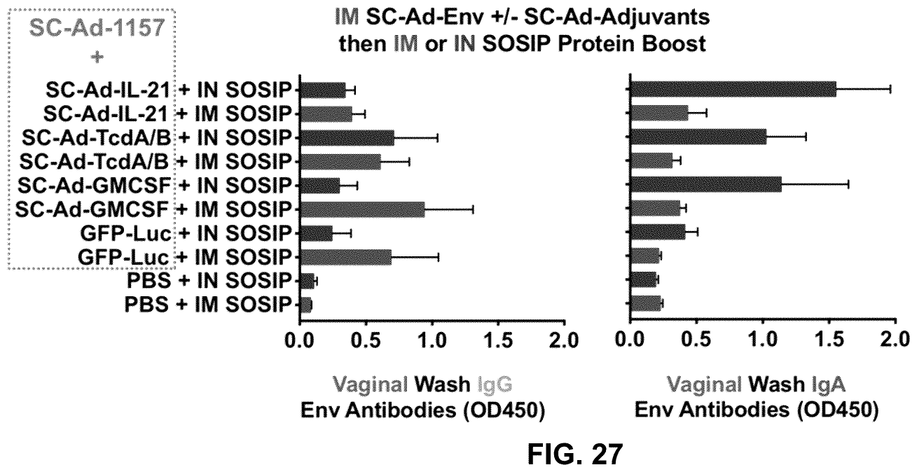

SC-Ad-GMCSF and TcdA/B were tested again by the i.m. route with 10-fold more SC-Ad. SC-Ad-IL-21 adjuvant was also added for its ability to stimulate Tfh and other T cells. In this case, i.m. injections were administered to the quadriceps muscles near to the vaginal sample site. 6 weeks after this single higher dose i.m. immunization, ELISA with 1/2000 dilutions of sera showed increased env IgG levels by SC-Ad-GMCSF, TcdA/B and IL-21 (p<0.05 vs PBS). SC-Ad-IL-21 provided even higher antibody levels than GMCSF or TcdA/B (p<0.0001 vs PBS). When vaginal wash samples were tested for IgG, all SC-Ad-1157 animals had increases, but only SC-Ad-IL-21 adjuvant reached significance (p<0.05 vs PBS). When vaginal washes were assayed for IgA at the same time point, this revealed similar trends with higher mucosal IgA in most animals in the GMCSF, TcdA/B, and IL-21 groups, only the IL-21 group reached p<0.05). Soluble HIV SOSIP envelope protein was used to boost the responses generated by SC-Ad. Each of the i.m. SC-Ad-1157+SC-Ad adjuvant immunized mice was boosted with 5 g of clade C CZA97 SOSIP.v4.2-M6.IT mixed with the NKT cell adjuvant alphaGalCer. One half of the mice were boosted by the i.m. route and one half were boosted by the i.n. route. 2 weeks later, vaginal washes were collected and assayed for IgA or IgG antibodies against clade C env (FIG. 27). These data showed a strong bias in antibody responses based on the route of delivery of the SOSIP protein boost. i.m. SOSIP increased vaginal IgG levels generated by i.m. SC-Ad-1157 and SC-Ad GFP-Luc or GMCSF better than i.n. protein. In contrast, i.n. SOSIP protein boost strongly amplified vaginal IgA levels in mice that were primed by the i.m. route with SC-Ad-1157 with the strongest SC-Ad adjuvants: GMCSF, TcdA/B, and IL-21. The SC-Ad-1157+SC-Ad-GFP-Luc group showed robust IgG responses when primed and boosted intramuscularly, but failed to generate a strong IgG response when the SOSIP was given i.n. Furthermore, either of these combinations failed to generate IgA responses. This would suggest that genetic adjuvants that are given in place of SC-Ad-GFP-Luc prime the animals to drive the IgA responses we observe when they are boosted i.n. also show that priming at mucosal surfaces. The protein administered to unprimed animals generated little IgG or IgA response in vaginal washes.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A SC-Ad, wherein said SC-Ad comprises a genome which lacks at least a portion of a nucleic acid sequence that encodes an adenovirus polypeptide, wherein said SC-Ad comprises said adenovirus polypeptide, and wherein said SC-Ad comprises (a) a nucleic acid sequence encoding an immunogen and (b) a nucleic acid sequence encoding a chaff polypeptide.

2. The SC-Ad of claim 1, wherein said adenovirus polypeptide is selected from the group consisting of a fiber polypeptide, a V polypeptide, a hexon polypeptide, a penton base polypeptide, and a pIIIa polypeptide.

3. The SC-Ad of claim 1, wherein said immunogen is an immunogenic polypeptide expressed by a pathogen.

4. The SC-Ad of claim 3, wherein said pathogen is a virus.

5. The SC-Ad of claim 4, wherein said virus is selected from the group consisting of SARS-CoV, HCoV NL63, HKU1, MERS-CoV, SARS-CoV-2, HIV-1, hepatitis B virus, hepatitis C virus, hepatitis D virus, hepatitis E virus, influenza, Ebola virus, Chiningunya virus, Zika virus, cytomegalovirus, and West Nile virus.

6. The SC-Ad of claim 3, wherein said pathogen is a bacterium.

7. The SC-Ad of claim 6, wherein said bacterium is selected from the group consisting of a Clostridium bacterium, a Staphylococcus aureus bacterium, a Campylobacter bacterium, a Mycobacteria bacterium, and a Borrelia bacterium.

8. The SC-Ad of claim 1, wherein said chaff polypeptide is a fragment of an ACE2 polypeptide.

9. The SC-Ad of claim 8, wherein said fragment of an ACE2 polypeptide comprises the extracellular region of an ACE2 polypeptide and lacks a transmembrane domain.

10. The SD-Ac of claim 9, wherein said chaff polypeptide consists essentially of or consists of an amino acid sequence set forth in SEQ ID NO:8 or SEQ ID NO:9.

11. The SC-Ad of claim 1, wherein said immunogen is fused to said chaff polypeptide.

12. A composition comprising the SC-Ad of claim 1.

13. A method for inducing an immune response in a mammal, wherein said method comprises administering to said mammal a SC-Ad, wherein said SC-Ad comprises a genome which lacks at least a portion of a nucleic acid sequence that encodes an adenovirus polypeptide, wherein said SC-Ad comprises said adenovirus polypeptide, and wherein said SC-Ad comprises (a) a nucleic acid sequence encoding an immunogen and (b) a nucleic acid sequence encoding a chaff polypeptide to said mammal under conditions wherein said SC-Ad infects a cell of said mammal, and wherein expression of said immunogen in said cell leads to induction of said immune response.

14. The method of claim 13, wherein said mammal is a human.

15. The method of claim 13, wherein said immunogen is an immunogenic polypeptide expressed by a pathogen.

16. The method of claim 15, wherein said pathogen is a virus.

17. The method of claim 16, wherein said virus is selected from the group consisting of SARS-CoV, HCoV NL63, HKU1, MERS-CoV, SARS-CoV-2, HIV-1, hepatitis B virus, hepatitis C virus, hepatitis D virus, hepatitis E virus, influenza, Ebola virus, Chiningunya virus, Zika virus, cytomegalovirus, and West Nile virus.

18. The method of claim 15, wherein said pathogen is a bacterium.

19. The method of claim 18, wherein said bacterium is selected from the group consisting of a Clostridium bacterium, a Staphylococcus aureus bacterium, a Campylobacter bacterium, a Mycobacteria bacterium, and a Borrelia bacterium.

20. The method of claim 13, wherein said chaff polypeptide is a fragment of an ACE2 polypeptide.

21. The method of claim 20, wherein said fragment of an ACE2 polypeptide comprises the extracellular region of an ACE2 polypeptide and lacks a transmembrane domain.

22. The method of claim 21, wherein said chaff polypeptide consists essentially of or consists of an amino acid sequence set forth in SEQ ID NO:8 or SEQ ID NO:9.

23. The method of claim 13, wherein said immunogen is fused to said chaff polypeptide.

24. The method of claim 13, wherein said administering comprising mucosal delivery of said SC-Ad.
